SEQUENCE LISTING (3 sequences)

SEQ ID NO: 1 - DNA, 20 bp, Artificial Sequence
  HBV primer 1
gtgtctgcggcgttttatca

SEQ ID NO: 2 - DNA, 21 bp, Artificial Sequence
  HBV primer 2
gacaaacgggcaacatacctt

SEQ ID NO: 3 - DNA, 28 bp, Artificial Sequence
  HBV probe 3
cctctkcatcctgctgctatgcctcatc

(19) United States
(12) Patent Application Publication
(10) Pub. No.: US 2021/0220356 A1
(43) Pub. Date: Jul. 22, 2021

(54) DIHYDROPYRIMIDINE DERIVATIVES AND USES THEREOF IN THE TREATMENT OF HBV INFECTION OR OF HBV-INDUCED DISEASES

(71) Applicant: Janssen Sciences Ireland Unlimited Company, Co Cork (IE)

(72) Inventors: Yimin JIANG, Londonderry, NH (US); Zhao-Kui WAN, Lexington, MA (US); Wing Shun CHEUNG, Hoboken, NJ (US); Qian LIU, Shanghai (CN); Gang DENG, Shanghai (CN); Chao LIANG, Shanghai (CN)

(21) Appl. No.: 17/053,737

(22) PCT Filed: May 7, 2019
(86) PCT No.: PCT/CN2019/085838

§ 371 Date: Nov. 6, 2020

(30) Foreign Application Priority Data
May 8, 2018 (EP) 18171262.1

(51) Int. Cl. A61K 31/506; A61K 38/21; A61K 39/395; A61K 45/06; A61K 31/7068; A61K 31/675; C07D 417/14; C07D 401/14; A61K 31/517

(52) U.S. Cl. CPC A61K 31/506; A61K 38/212; A61K 39/3955; A61K 45/06; A61K 31/517; A61K 31/675; C07D 417/14; C07D 401/14; A61K 31/7068

(57) ABSTRACT

Provided herein are dihydropyrimidine derivatives which are useful in the treatment of HBV infection or HBV-induced diseases, as well as pharmaceutical or medical applications thereof.

### BACKGROUND

Chronic hepatitis B virus (HBV) infection is a significant global health problem, affecting over 5% of the world population (over 350 million people worldwide and 1.25 million individuals in the U.S.).

Despite the availability of a prophylactic HBV vaccine, the burden of chronic HBV infection continues to be a significant unmet worldwide medical problem, due to suboptimal treatment options and sustained rates of new infections in most parts of the developing world.

Current treatments do not provide a cure and are limited to only two classes of agents (interferon alpha and nucleoside analogues/inhibitors of the viral polymerase); drug resistance, low efficacy, and tolerability issues limit their impact. The low cure rates of HBV are attributed at least in part to the fact that complete suppression of virus production is difficult to achieve with a single antiviral agent. However, persistent suppression of HBV DNA slows liver disease progression and helps to prevent hepatocellular carcinoma. Current therapy goals for HBV-infected patients are directed to reducing serum HBV DNA to low or undetectable levels, and to ultimately reducing or preventing the development of cirrhosis and hepatocellular carcinoma.

The HBV capsid protein plays essential functions during the viral life cycle. HBV capsid/core proteins form metastable viral particles or protein shells that protect the viral genome during intercellular passage, and also play a central role in viral replication processes, including genome encapsidation, genome replication, and virion morphogenesis and egress.

Capsid structures also respond to environmental cues to allow un-coating after viral entry.

Consistently, the appropriate timing of capsid assembly and dis-assembly, the appropriate capsid stability and the function of core protein have been found to be critical for viral infectivity.

There is a need in the art for therapeutic agents that can increase the suppression of virus production and that can treat, ameliorate, or prevent HBV infection. Administration of such therapeutic agents to an HBV infected patient, either as monotherapy or in combination with other HBV treatments or ancillary treatments, will lead to significantly reduced virus burden, improved prognosis, diminished progression of the disease and enhanced seroconversion rates.

### SUMMARY

Provided, in one aspect, is a compound of formula (I)

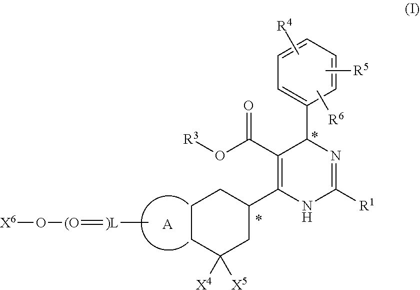

including the deuterated isomers, stereoisomers and the tautomeric forms thereof, wherein A is a 5 or 6-membered aromatic ring, which comprises heteroatoms independently chosen from among S, O and N, wherein the number of said heteroatoms independently chosen from among S, O and N is one or two, wherein said 5 or 6-membered aromatic ring is optionally substituted with one or more from C1-C4 alkyl and cyano,
wherein L is C1-C6 alkyl,
wherein X⁶ is H or C1-C6 alkyl,
wherein R⁴, R⁵ and R⁶ each independently are chosen from among halogen, H and C1-C3 alkyl,
wherein R³ is C1-C4 alkyl,
wherein R¹ is selected from thiazolyl and pyridyl, each optionally substituted with one or more halogen; and
wherein X⁴ and X⁵ each independently are chosen from among H and C1-C4 alkyl, or a pharmaceutically acceptable salt or a solvate thereof.

In another aspect, provided herein is a pharmaceutical composition comprising at least one compound of Formula I, or a pharmaceutically acceptable salt thereof, together with a pharmaceutically acceptable carrier.

In another aspect, provided herein is a pharmaceutical composition comprising at least one disclosed compound, together with a pharmaceutically acceptable carrier. In another aspect, provided herein is a method of treating an HBV infection in an individual in need thereof, comprising administering to the individual a therapeutically effective amount of a compound of Formula I or a pharmaceutically acceptable salt thereof.

In another aspect, provided herein is a method of inhibiting or reducing the formation or presence of HBV DNA-containing particles or HBV RNA-containing particles in an individual in need thereof, comprising administering to the individual a therapeutically effective amount of a compound of Formula I, or a pharmaceutically acceptable salt thereof.

In an embodiment, any of the methods provided herein can further comprise administering to the individual at least one additional therapeutic agent selected from the group consisting of an HBV polymerase inhibitor, immunomodulatory agents, interferon, viral entry inhibitor, viral maturation inhibitor, capsid assembly modulator, reverse transcriptase inhibitor, a cyclophilin/TNF inhibitor, a TLR-agonist, an HBV vaccine, and any combination thereof.

In a still further aspect, a process is provided for producing the compound of formula I, wherein said process comprises reacting the compound of formula III with the compound of formula IV and the compound of formula V to produce the compound of formula I:

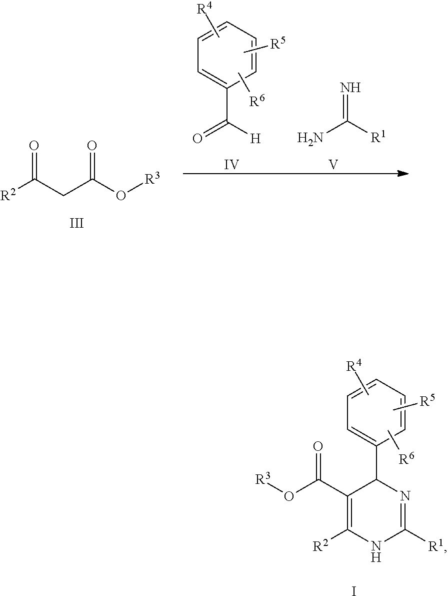

wherein R²— is the group:

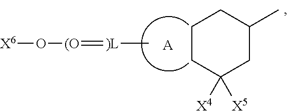

and wherein R¹, R³, R⁴, R⁵, R⁶, A, L, X⁴, X⁵, and X⁶ are as defined above.

### DESCRIPTION

The application provides a compound of formula (I)

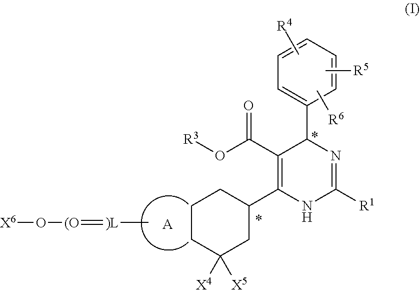

including the deuterated isomers, stereoisomers and the tautomeric forms thereof, wherein A is a 5 or 6-membered aromatic ring, which comprises heteroatoms independently chosen from among S, O and N, wherein the number of said heteroatoms independently chosen from among S, O and N is one or two, wherein said 5 or 6-membered aromatic ring is optionally substituted with one or more from C1-C4 alkyl and cyano,
wherein L is C1-C6 alkyl,
wherein X⁶ is H or C1-C6 alkyl,
wherein R⁴, R⁵ and R⁶ each independently are chosen from among halogen, H and C1-C3 alkyl,
wherein R³ is C1-C4 alkyl,
wherein R¹ is selected from thiazolyl and pyridyl, each optionally substituted with one or more halogen; and
wherein X⁴ and X⁵ each independently are chosen from among H and C1-C4 alkyl, or a pharmaceutically acceptable salt or a solvate thereof.

Provided herein are compounds, e.g., the compounds of formula (I), or pharmaceutically acceptable salts thereof, that are useful in the treatment and prevention of HBV infection in subject.

Without being bound to any particular mechanism of action, these compounds are believed to modulate or disrupt HBV assembly and other HBV core protein functions necessary for HBV replication or the generation of infectious particles and/or may disrupt HBV capsid assembly leading to empty capsids with greatly reduced infectivity or replication capacity. In other words, the compounds provided herein may act as capsid assembly modulators.

The compounds provided herein have potent antiviral activity, exhibit favorable metabolic properties, tissue distribution, safety and pharmaceutical profiles, and are suitable for use in humans. Disclosed compounds may modulate (e.g., accelerate, delay, inhibit, disrupt or reduce) normal viral capsid assembly or disassembly, bind capsid or alter metabolism of cellular polyproteins and precursors. The modulation may occur when the capsid protein is mature, or during viral infectivity. Disclosed compounds can be used in methods of modulating the activity or properties of HBV cccDNA, or the generation or release of HBV RNA particles from within an infected cell.

In one embodiment, the compounds described herein are suitable for monotherapy and are effective against natural or native HBV strains and against HBV strains resistant to currently known drugs. In another embodiment, the compounds described herein are suitable for use in combination therapy.

### Definitions

Listed below are definitions of various terms used to describe this invention. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e. to at least one) of the grammatical object of the article. By way of example, “an element” means one element or more than one element. Furthermore, use of the term “including” as well as other forms, such as “include”, “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, including ±5%, ±1%, and ±0.1% from the specified value, as such variations are appropriate to perform the disclosed methods.

As used herein, the term “capsid assembly modulator” refers to a compound that disrupts or accelerates or inhibits or hinders or delays or reduces or modifies normal capsid assembly (e.g., during maturation) or normal capsid disassembly (e.g., during infectivity) or perturbs capsid stability, thereby inducing aberrant capsid morphology and function. In one embodiment, a capsid assembly modulator accelerates capsid assembly or disassembly, thereby inducing aberrant capsid morphology. In another embodiment, a capsid assembly modulator interacts (e.g. binds at an active site, binds at an allosteric site, modifies or hinders folding and the like) with the major capsid assembly protein (CA), thereby disrupting capsid assembly or disassembly. In yet another embodiment, a capsid assembly modulator causes a perturbation in structure or function of CA (e.g., ability of CA to assemble, disassemble, bind to a substrate, fold into a suitable conformation, or the like), which attenuates viral infectivity or is lethal to the virus.

As used herein, the term “treatment” or “treating” is defined as the application or administration of a therapeutic agent, i.e., a disclosed compound (alone or in combination with another pharmaceutical agent), to a patient, or application or administration of a therapeutic agent to an isolated tissue or cell line from a patient (e.g., for diagnosis or ex vivo applications), who has an HBV infection, a symptom of HBV infection or the potential to develop an HBV infection, with the purpose to cure, heal, alleviate, relieve, alter, remedy, ameliorate, improve or affect the HBV infection, the symptoms of HBV infection, or the potential to develop an HBV infection. Such treatments may be specifically tailored or modified, based on knowledge obtained from the field of pharmacogenomics.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual” or “subject” refers to a human or a non-human mammal. Non-human mammals include, for example, livestock and pets, such as ovine, bovine, porcine, canine, feline and murine mammals. Preferably, the patient, subject, or individual is human.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary, and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, the term “alkyl,” by itself or as part of another substituent means, unless otherwise stated, a straight or branched chain hydrocarbon having the number of carbon atoms designated (i.e., C₁-C₃ alkyl means an alkyl having one to three carbon atoms, C₁-C₄ alkyl means an alkyl having one to four carbon) and includes straight and branched chains. Examples include methyl, ethyl, propyl, isopropyl, butyl, isobutyl, tert-butyl. Embodiments of alkyl include, but are not limited to, C₁-C₁₀ alkyl, such as C₁-C₆ alkyl, such as C₁-C₄ alkyl.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “3-7 membered saturated ring” refers to a mono cyclic non-aromatic saturated radical, wherein each of the atoms forming the ring (i.e., skeletal atoms) is a carbon atom, unless such ring contains one or more heteroatoms if so further defined. 3-7 Membered saturated rings include groups having 3 to 7 ring atoms. Monocyclic 3-7 membered saturated rings include, but are not limited to, cyclopropyl, cyclobutyl, cyclopentyl, cyclohexyl, cycloheptyl.

As used herein, 3-7 membered saturated ring optionally comprising one or more heteroatoms refers to a heteroalicyclic group containing one or more, more in particular, one, two or three, even more in particular, one or two, and most particular, one ring heteroatoms each selected from O, S, and N. In one embodiment, each heterocyclyl group has from 3 to 7 atoms in its ring system, with the proviso that the ring of said group does not contain two adjacent O or S atoms. The heterocyclic system may be attached to the remainder of the molecule, unless otherwise stated, at any heteroatom or carbon atom that affords a stable structure.

An example of a 3-membered heterocyclyl group includes, and is not limited to, aziridine. Examples of 4-membered heterocycloalkyl groups include, and are not limited to, azetidine and a beta lactam. Examples of 5-membered heterocyclyl groups include, and are not limited to, pyrrolidine, oxazolidine and thiazolidinedione. Examples of 6-membered heterocycloalkyl groups include, and are not limited to, piperidine, morpholine, and piperazine.

Other non-limiting examples of heterocyclyl groups include monocyclic groups such as aziridine, oxirane, thiirane, azetidine, oxetane, thietane, pyrrolidine, pyrroline, pyrazolidine, imidazoline, dioxolane, sulfolane, tetrahydrofuran, thiophane, piperidine, piperazine, morpholine, thiomorpholine.

As used herein, the term “aromatic” refers to a carbocycle or heterocycle with one or more polyunsaturated rings and having aromatic character, i.e., having (4n+2) delocalized π(pi) electrons, where n is an integer.

As used herein, the term “aryl,” employed alone or in combination with other terms, means, unless otherwise stated, a carbocyclic aromatic system containing one or more rings (typically one, two, or three rings), wherein such rings may be attached together in a pendent manner, such as a biphenyl, or may be fused, such as naphthalene. Examples of aryl groups include phenyl, anthracyl, and naphthyl. Preferred examples are phenyl (e.g., C₆-aryl) and biphenyl (e.g., C₁₂-aryl). In some embodiments, aryl groups have from six to sixteen carbon atoms. In some embodiments, aryl groups have from six to twelve carbon atoms (e.g., C₆-C₁₂-aryl). In some embodiments, aryl groups have six carbon atoms (e.g., C₆-aryl).

As used herein, the term “heteroaryl” or “heteroaromatic” refers to a heterocycle having aromatic character. Heteroaryl substituents may be defined by the number of carbon atoms, e.g., C₁-C₉-heteroaryl indicates the number of carbon atoms contained in the heteroaryl group without including the number of heteroatoms. For example, a C₁-C₉-heteroaryl will include an additional one to four heteroatoms. A polycyclic heteroaryl may include one or more rings that are partially saturated. Non-limiting examples of heteroaryls include pyridyl, pyrazinyl, pyrimidinyl (including, e.g., 2- and 4-pyrimidinyl), pyridazinyl, thienyl, furyl, pyrrolyl (including, e.g., 2-pyrrolyl), imidazolyl, thiazolyl, oxazolyl, pyrazolyl (including, e.g., 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Non-limiting examples of polycyclic heterocycles and heteroaryls include indolyl (including, e.g., 3-, 4-, 5-, 6- and 7-indolyl), indolinyl, quinolyl, tetrahydroquinolyl, isoquinolyl (including, e.g., 1- and 5-isoquinolyl), 1,2,3,4-tetrahydroisoquinolyl, cinnolinyl, quinoxalinyl (including, e.g., 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (including, e.g., 3-, 4-, 5-, 6- and 7-benzofuryl), 2,3-dihydrobenzofuryl, 1,2-benzisoxazolyl, benzothienyl (including, e.g., 3-, 4-, 5-, 6-, and 7-benzothienyl), benzoxazolyl, benzothiazolyl (including, e.g., 2-benzothiazolyl and 5-benzothiazolyl), purinyl, benzimidazolyl (including, e.g., 2-benzimidazolyl), benzotriazolyl, thioxanthinyl, carbazolyl, carbolinyl, acridinyl, pyrrolizidinyl, and quinolizidinyl.

As used herein, the term “substituted” means that an atom or group of atoms has replaced hydrogen as the substituent attached to another group.

As used herein, the terminology “selected from . . . ” (e.g., “R⁴ is selected from A, B and C”) is understood to be equivalent to the terminology “selected from the group consisting of . . . ” (e.g., “R⁴ is selected from the group consisting of A, B and C”).

In an embodiment of the compound of formula (I), L is a straight-chain hydrocarbon, or a branched-chain hydrocarbon, or a cyclic-chain hydrocarbon, or X⁶—O—(O═C)L′, wherein L′ is C1-C5 alkyl, including C3-C5 cycloalkyl.
In an embodiment of the compound of formula (I), L is a straight-chain hydrocarbon, or a branched-chain hydrocarbon, or a cyclic-chain hydrocarbon, or X⁶—O—(O═C)L′, wherein L′ is C1-C6 alkyl, including C3-C6 cycloalkyl.
In an embodiment of the compound of formula (I), ring A is pyrazolyl, pyrrolyl, pyrimidyl, oxazolyl or thiazolyl.
In an embodiment of the compound of formula (I), R¹ is thiazolyl, particularly in an embodiment wherein ring A is pyrazolyl, pyrrolyl, pyrimidyl, oxazolyl or thiazolyl.
In an embodiment, the compound of formula (I) is selected from the compounds satisfying the following formulae:

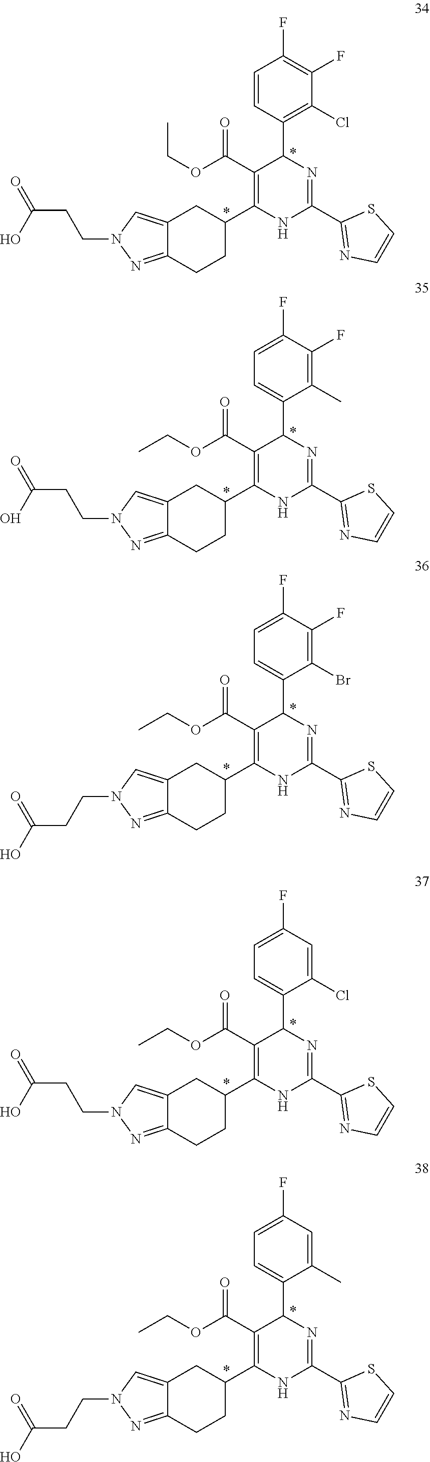

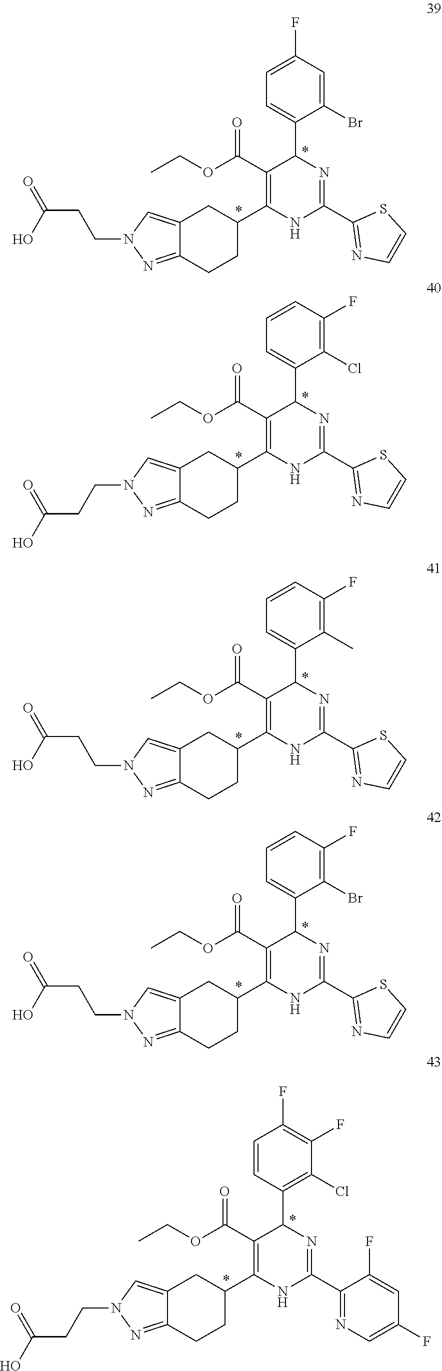

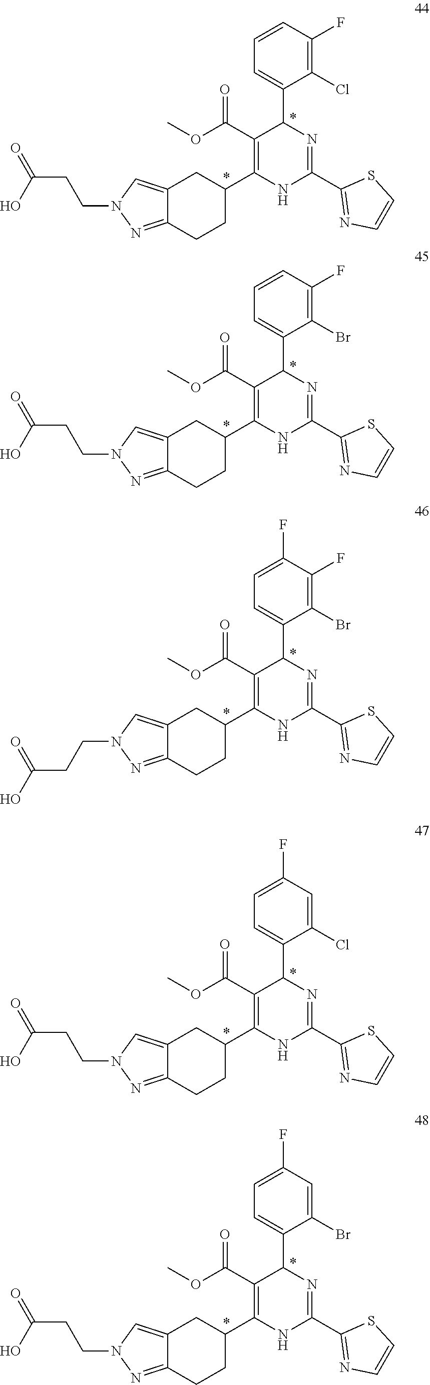

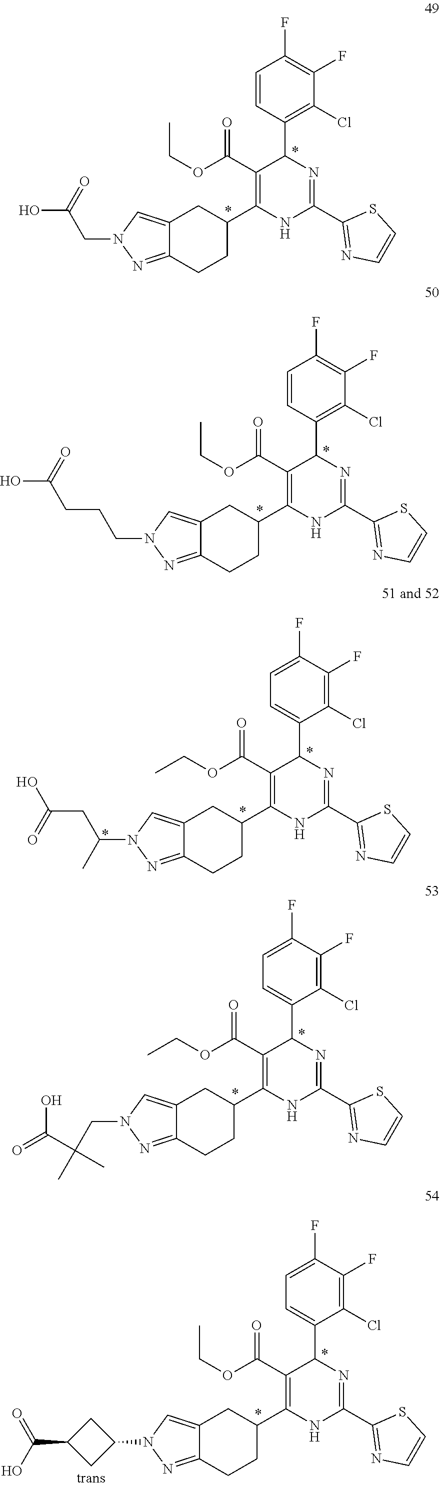

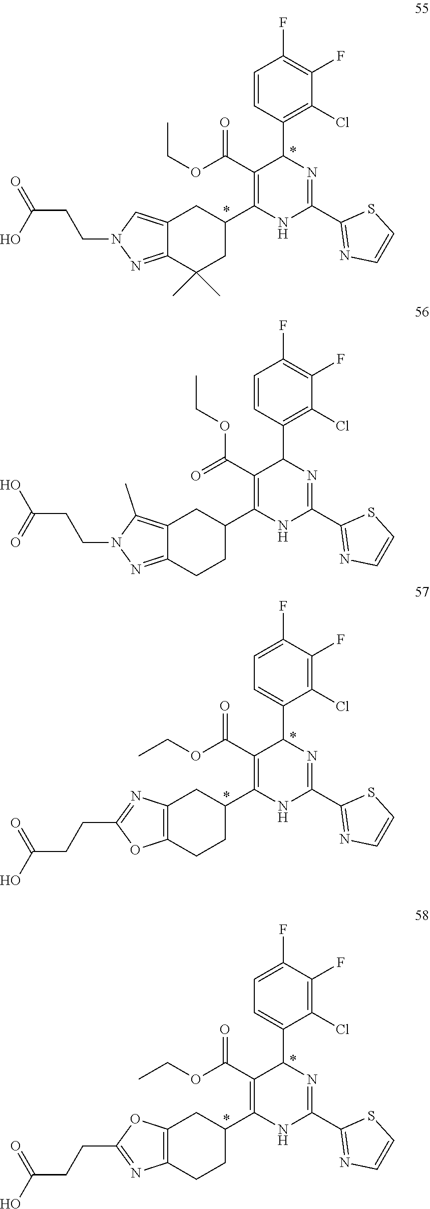

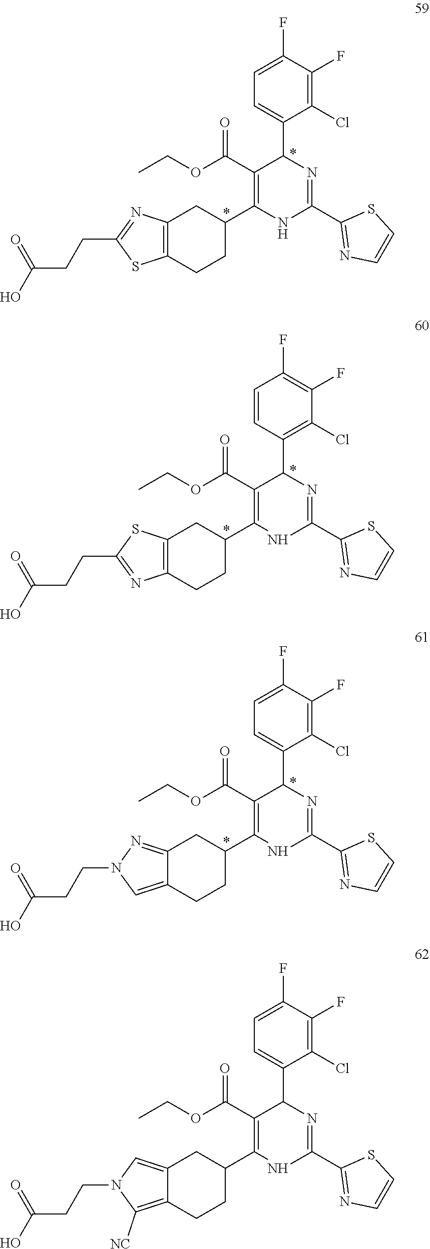 [SMILES: CCOC(=O)C1=C(C2CCc3sc(CCC(=O)O)nc3C2)NC(c2nccs2)=NC1c1ccc(F)c(F)c1Cl ; CCOC(=O)C1=C(C2CCc3nc(CCC(=O)O)sc3C2)NC(c2nccs2)=NC1c1ccc(F)c(F)c1Cl ; CCOC(=O)C1=C(C2CCc3cn(CCC(=O)O)nc3C2)NC(c2nccs2)=NC1c1ccc(F)c(F)c1Cl ; CCOC(=O)C1=C(C2CCc3c(cn(CCC(=O)O)c3C#N)C2)NC(c2nccs2)=NC1c1ccc(F)c(F)c1Cl]

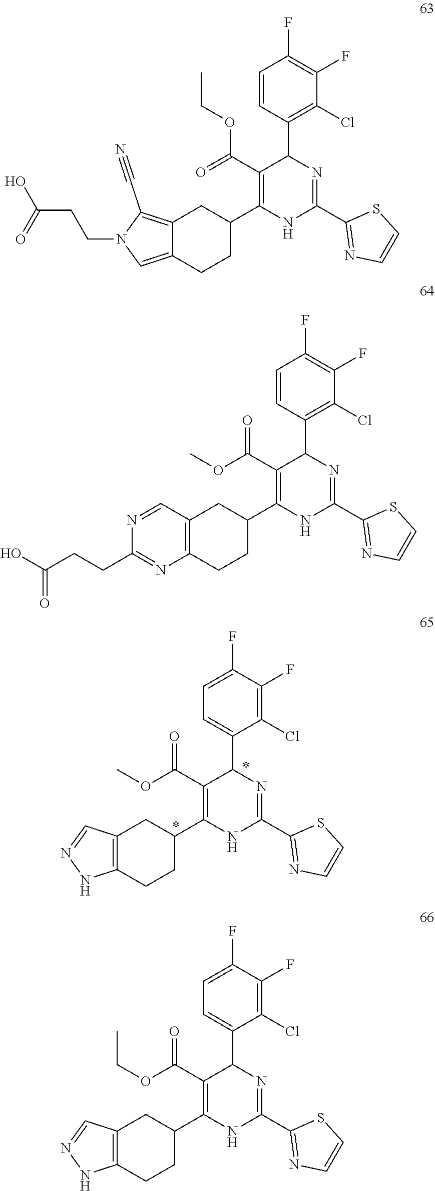 [SMILES: CCOC(=O)C1=C(C2CCc3cn(CCC(=O)O)c(C#N)c3C2)NC(c2nccs2)=NC1c1ccc(F)c(F)c1Cl ; COC(=O)C1=C(C2CCc3nc(CCC(=O)O)ncc3C2)NC(c2nccs2)=NC1c1ccc(F)c(F)c1Cl ; COC(=O)C1=C(C2CCc3[nH]ncc3C2)NC(c2nccs2)=NC1c1ccc(F)c(F)c1Cl ; CCOC(=O)C1=C(C2CCc3[nH]ncc3C2)NC(c2nccs2)=NC1c1ccc(F)c(F)c1Cl]

or a pharmaceutically acceptable salt or a solvate thereof.
In an embodiment, the compound of formula (I) is selected from compounds satisfying the following formulae:

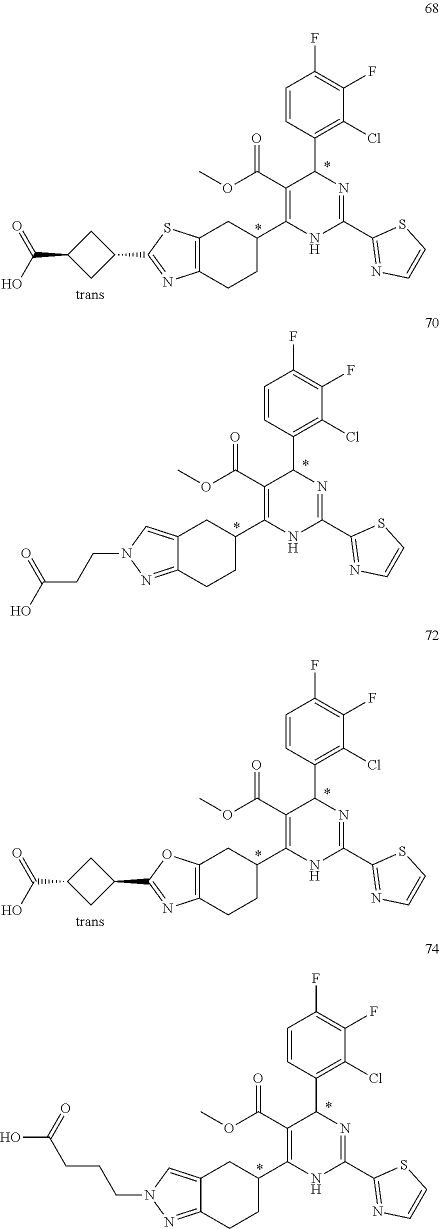 [SMILES: COC(=O)C1=C(C2CCc3nc([C@H]4C[C@H](C(=O)O)C4)sc3C2)NC(c2nccs2)=NC1c1ccc(F)c(F)c1Cl ; COC(=O)C1=C(C2CCc3nn(CCC(=O)O)cc3C2)NC(c2nccs2)=NC1c1ccc(F)c(F)c1Cl ; COC(=O)C1=C(C2CCc3nc([C@H]4C[C@H](C(=O)O)C4)oc3C2)NC(c2nccs2)=NC1c1ccc(F)c(F)c1Cl ; COC(=O)C1=C(C2CCc3nn(CCCC(=O)O)cc3C2)NC(c2nccs2)=NC1c1ccc(F)c(F)c1Cl]

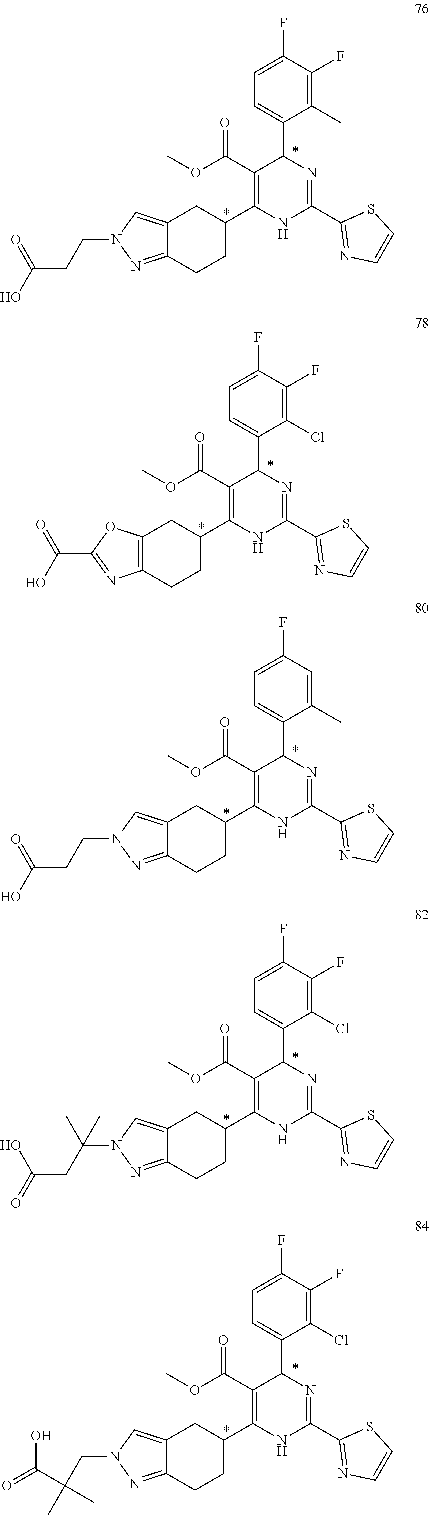 [SMILES: COC(=O)C1=C(C2CCc3nn(CCC(=O)O)cc3C2)NC(c2nccs2)=NC1c1ccc(F)c(F)c1C ; COC(=O)C1=C(C2CCc3nc(C(=O)O)oc3C2)NC(c2nccs2)=NC1c1ccc(F)c(F)c1Cl ; COC(=O)C1=C(C2CCc3nn(CCC(=O)O)cc3C2)NC(c2nccs2)=NC1c1ccc(F)cc1C ; COC(=O)C1=C(C2CCc3nn(C(C)(C)CC(=O)O)cc3C2)NC(c2nccs2)=NC1c1ccc(F)c(F)c1Cl ; COC(=O)C1=C(C2CCc3nn(CC(C)(C)C(=O)O)cc3C2)NC(c2nccs2)=NC1c1ccc(F)c(F)c1Cl]

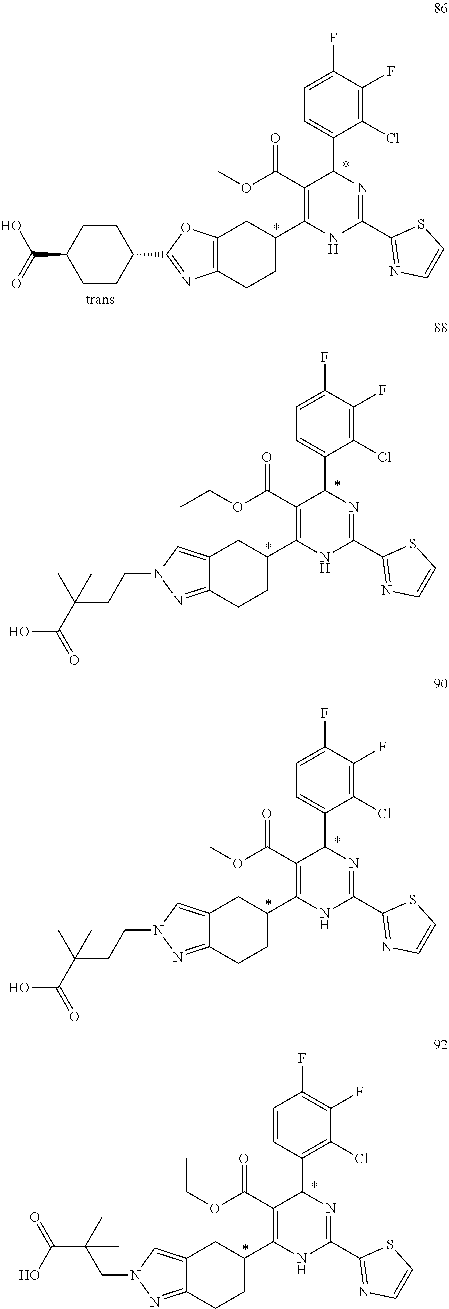 [SMILES: COC(=O)C1=C(C2CCc3nc([C@H]4CC[C@H](C(=O)O)CC4)oc3C2)NC(c2nccs2)=NC1c1ccc(F)c(F)c1Cl ; CCOC(=O)C1=C(C2CCc3nn(CCC(C)(C)C(=O)O)cc3C2)NC(c2nccs2)=NC1c1ccc(F)c(F)c1Cl ; COC(=O)C1=C(C2CCc3nn(CCC(C)(C)C(=O)O)cc3C2)NC(c2nccs2)=NC1c1ccc(F)c(F)c1Cl ; CCOC(=O)C1=C(C2CCc3nn(CC(C)(C)C(=O)O)cc3C2)NC(c2nccs2)=NC1c1ccc(F)c(F)c1Cl]

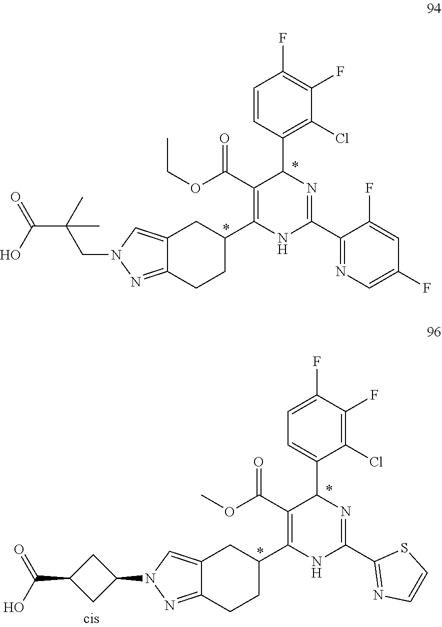 [SMILES: CCOC(=O)C1=C(C2CCc3nn(CC(C)(C)C(=O)O)cc3C2)NC(c2ncc(F)cc2F)=NC1c1ccc(F)c(F)c1Cl ; COC(=O)C1=C(C2CCc3nn([C@H]4C[C@@H](C(=O)O)C4)cc3C2)NC(c2nccs2)=NC1c1ccc(F)c(F)c1Cl]

or a pharmaceutically acceptable salt or a solvate thereof.
In an embodiment, the compound of formula (I) is selected from compounds satisfying the following formulae.

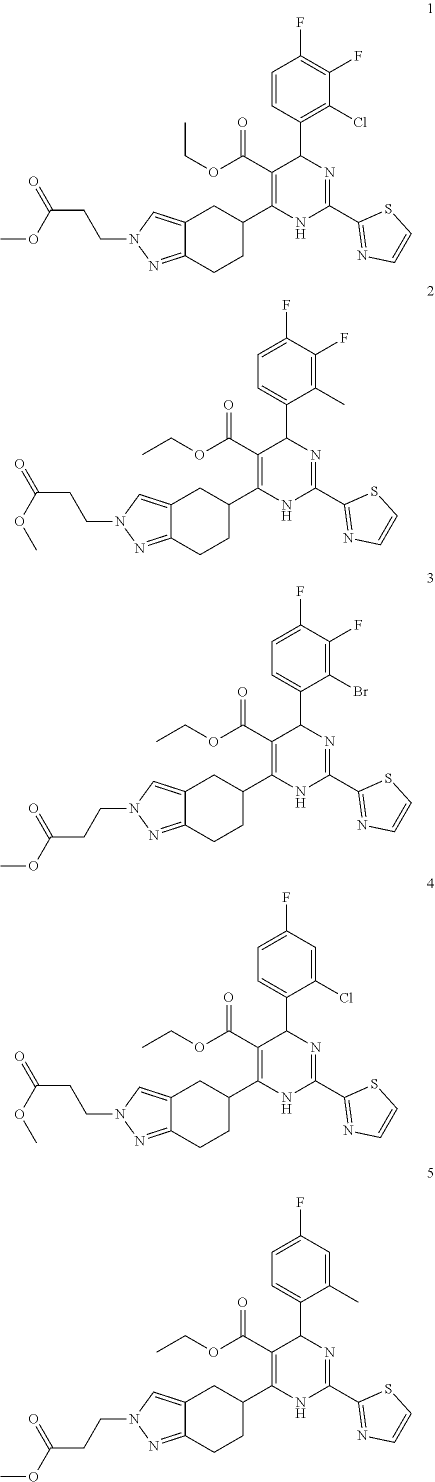 [SMILES: CCOC(=O)C1=C(C2CCc3nn(CCC(=O)OC)cc3C2)NC(c2nccs2)=NC1c1ccc(F)c(F)c1Cl ; CCOC(=O)C1=C(C2CCc3nn(CCC(=O)OC)cc3C2)NC(c2nccs2)=NC1c1ccc(F)c(F)c1C ; CCOC(=O)C1=C(C2CCc3nn(CCC(=O)OC)cc3C2)NC(c2nccs2)=NC1c1ccc(F)c(F)c1Br ; CCOC(=O)C1=C(C2CCc3nn(CCC(=O)OC)cc3C2)NC(c2nccs2)=NC1c1ccc(F)cc1Cl ; CCOC(=O)C1=C(C2CCc3nn(CCC(=O)OC)cc3C2)NC(c2nccs2)=NC1c1ccc(F)cc1C]

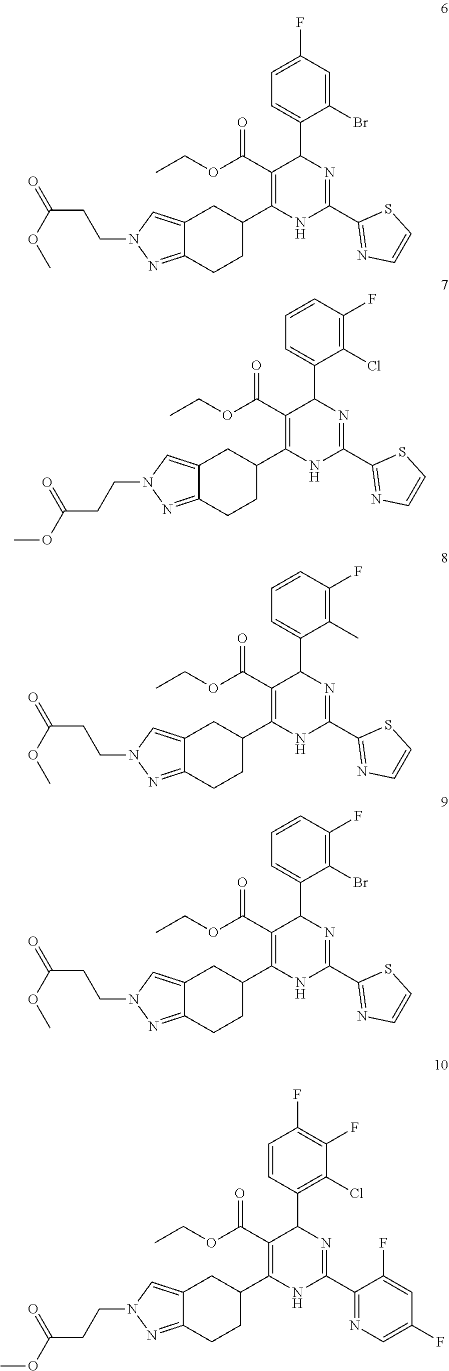

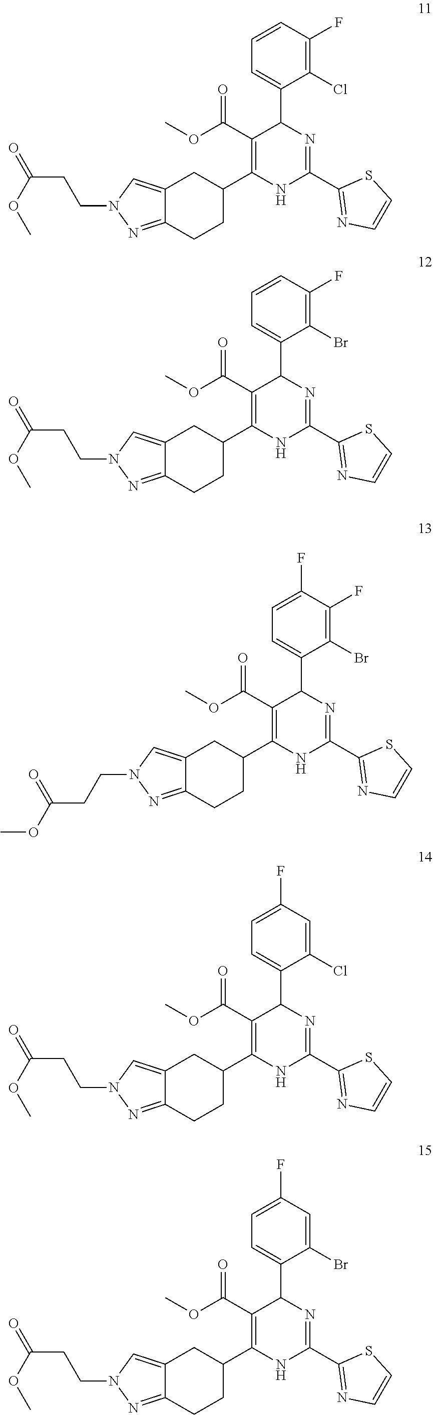 [SMILES: COC(=O)CCn1cc2c(n1)CCC(C1=C(C(=O)OC)C(c3cccc(F)c3Cl)N=C(c3nccs3)N1)C2 ; COC(=O)CCn1cc2c(n1)CCC(C1=C(C(=O)OC)C(c3cccc(F)c3Br)N=C(c3nccs3)N1)C2 ; COC(=O)CCn1cc2c(n1)CCC(C1=C(C(=O)OC)C(c3ccc(F)c(F)c3Br)N=C(c3nccs3)N1)C2 ; COC(=O)CCn1cc2c(n1)CCC(C1=C(C(=O)OC)C(c3ccc(F)cc3Cl)N=C(c3nccs3)N1)C2 ; COC(=O)CCn1cc2c(n1)CCC(C1=C(C(=O)OC)C(c3ccc(F)cc3Br)N=C(c3nccs3)N1)C2]

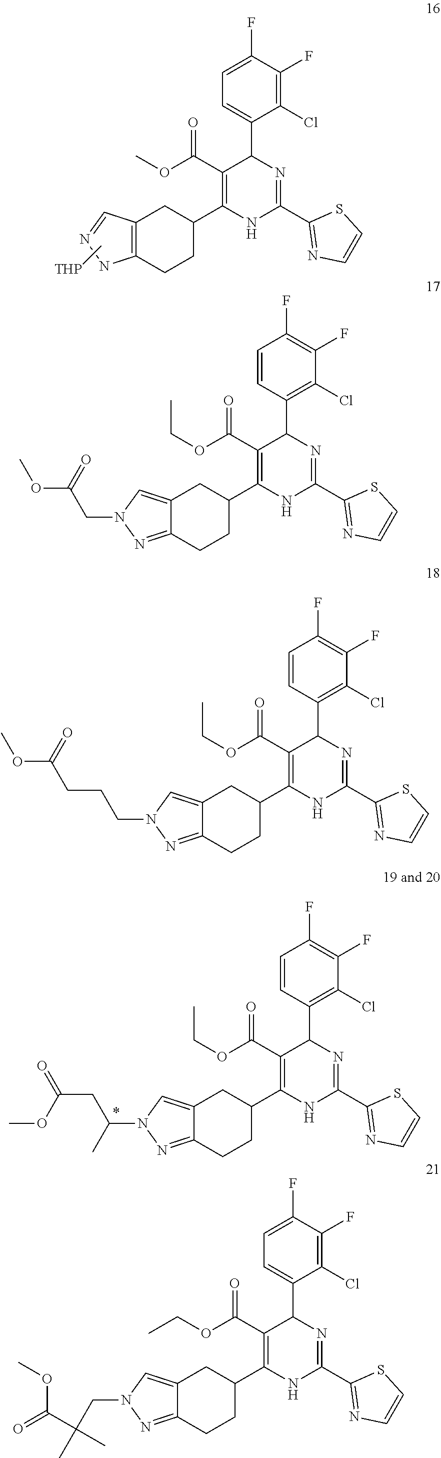

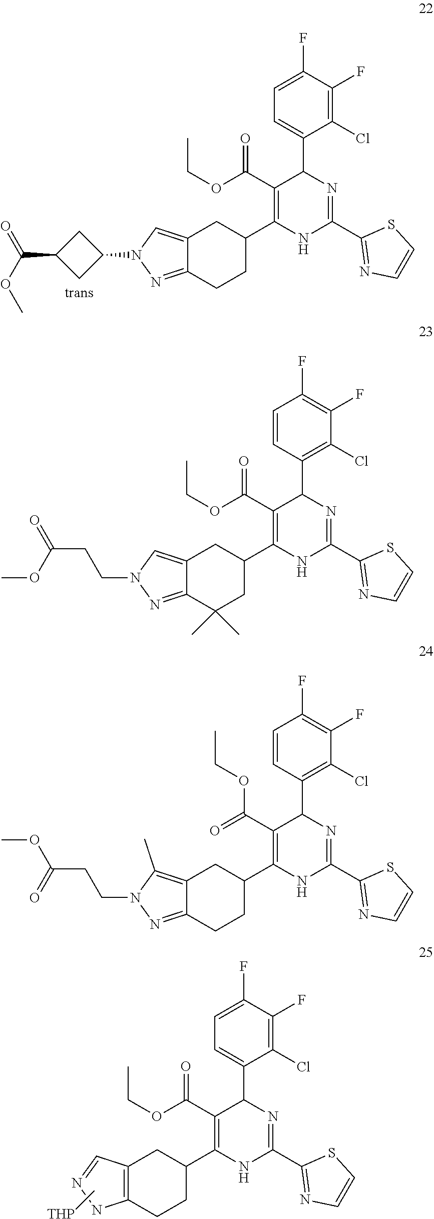

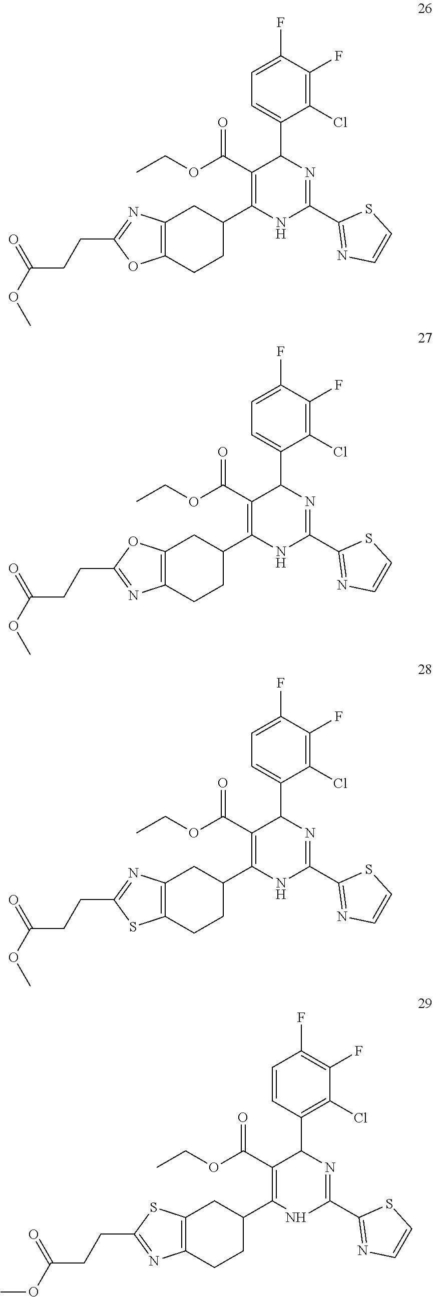 [SMILES: CCOC(=O)C1=C(C2CCc3oc(CCC(=O)OC)nc3C2)NC(c2nccs2)=NC1c1ccc(F)c(F)c1Cl ; CCOC(=O)C1=C(C2CCc3nc(CCC(=O)OC)oc3C2)NC(c2nccs2)=NC1c1ccc(F)c(F)c1Cl ; CCOC(=O)C1=C(C2CCc3sc(CCC(=O)OC)nc3C2)NC(c2nccs2)=NC1c1ccc(F)c(F)c1Cl ; CCOC(=O)C1=C(C2CCc3nc(CCC(=O)OC)sc3C2)NC(c2nccs2)=NC1c1ccc(F)c(F)c1Cl]

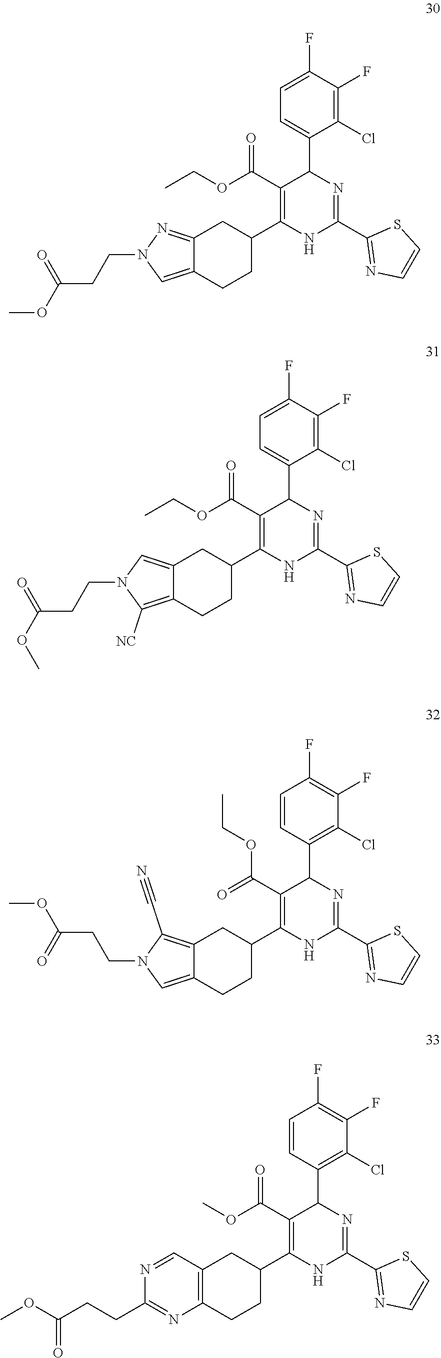 [SMILES: CCOC(=O)C1=C(C2CCc3cn(CCC(=O)OC)nc3C2)NC(c2nccs2)=NC1c1ccc(F)c(F)c1Cl ; CCOC(=O)C1=C(C2CCc3c(cn(CCC(=O)OC)c3C#N)C2)NC(c2nccs2)=NC1c1ccc(F)c(F)c1Cl ; CCOC(=O)C1=C(C2CCc3cn(CCC(=O)OC)c(C#N)c3C2)NC(c2nccs2)=NC1c1ccc(F)c(F)c1Cl ; COC(=O)CCc1ncc2c(n1)CCC(C1=C(C(=O)OC)C(c3ccc(F)c(F)c3Cl)N=C(c3nccs3)N1)C2]

or a pharmaceutically acceptable salt or a solvate thereof:
In an embodiment, the compound of formula (I) is selected from compounds satisfying the following formulae:

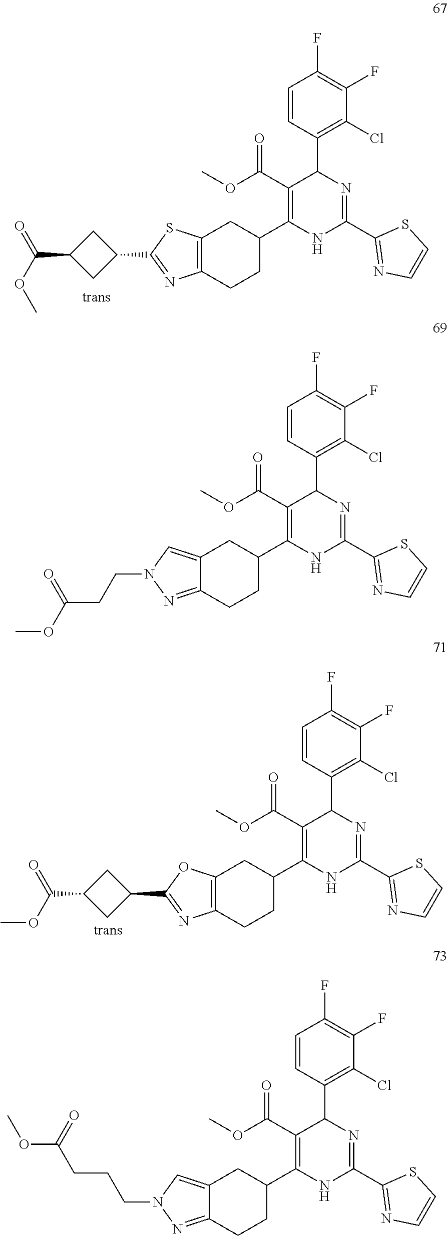 [SMILES: COC(=O)C1=C(C2CCc3nc([C@H]4C[C@H](C(=O)OC)C4)sc3C2)NC(c2nccs2)=NC1c1ccc(F)c(F)c1Cl ; COC(=O)CCn1cc2c(n1)CCC(C1=C(C(=O)OC)C(c3ccc(F)c(F)c3Cl)N=C(c3nccs3)N1)C2 ; COC(=O)C1=C(C2CCc3nc([C@H]4C[C@H](C(=O)OC)C4)oc3C2)NC(c2nccs2)=NC1c1ccc(F)c(F)c1Cl ; COC(=O)CCCn1cc2c(n1)CCC(C1=C(C(=O)OC)C(c3ccc(F)c(F)c3Cl)N=C(c3nccs3)N1)C2]

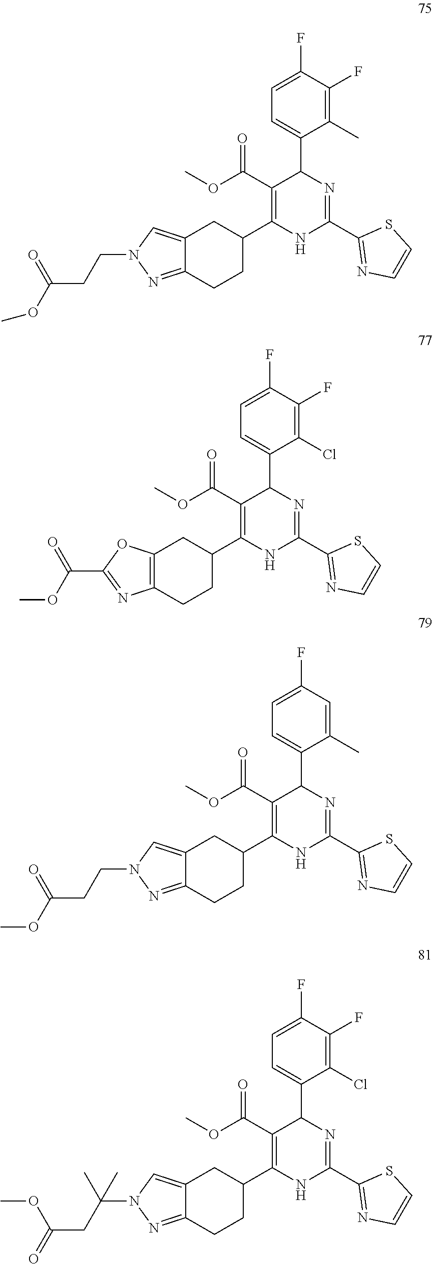 [SMILES: COC(=O)CCn1cc2c(n1)CCC(C1=C(C(=O)OC)C(c3ccc(F)c(F)c3C)N=C(c3nccs3)N1)C2 ; COC(=O)C1=C(C2CCc3nc(C(=O)OC)oc3C2)NC(c2nccs2)=NC1c1ccc(F)c(F)c1Cl ; COC(=O)CCn1cc2c(n1)CCC(C1=C(C(=O)OC)C(c3ccc(F)cc3C)N=C(c3nccs3)N1)C2 ; COC(=O)CC(C)(C)n1cc2c(n1)CCC(C1=C(C(=O)OC)C(c3ccc(F)c(F)c3Cl)N=C(c3nccs3)N1)C2]

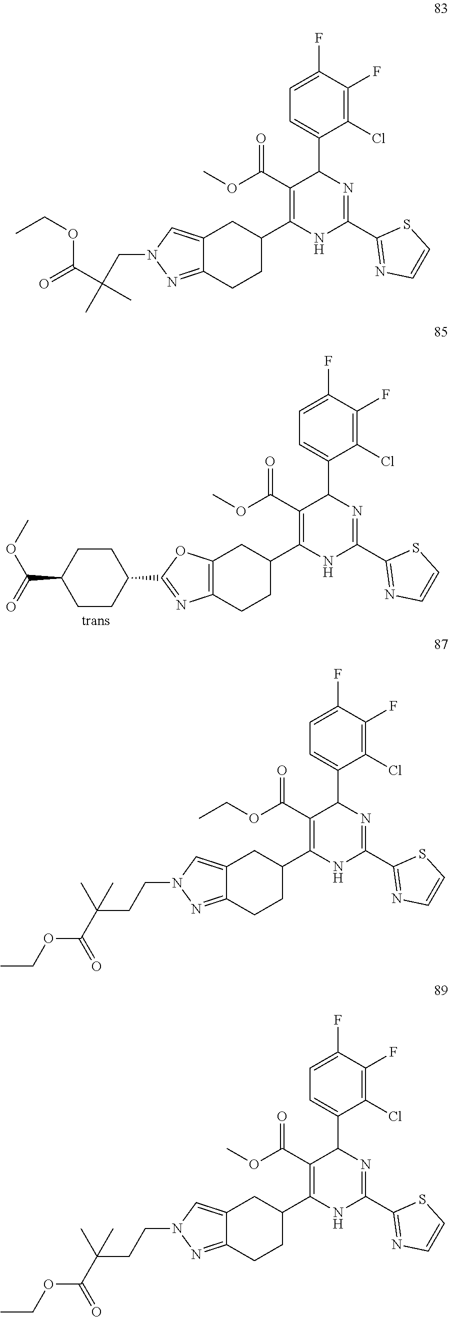

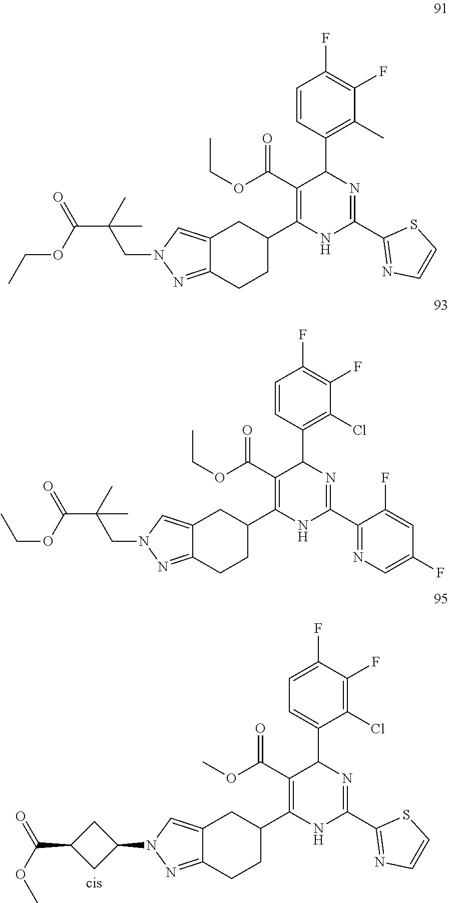

In an embodiment of the compound of formula (I), X⁴ and X⁵ are the same or different, and are H or methyl.
The compounds of formula I, as discussed hereinbefore and hereinafter, include a group indicated as X⁶—O—(O═)L. It will be understood that in this group, both the single bonded and the double bonded 0 are attached to a terminal carbon atom of L, thus defining a carboxyl group. Said group includes groups having the formula X⁶—O—(O═C)-L′, whereby L′ is C1-C5 alkyl, including C3-C5 cycloalkyl, such as cyclobutyl.
In an embodiment of the compound of formula (I), X⁶ is H or methyl.
In an embodiment, R³ is methyl or ethyl.
In an embodiment, at most one of R⁴, R⁵, and R⁶ is H and at least two of R⁴, R⁵, and R⁶ are halogen. In an embodiment thereof, at least one halogen is F and at least one halogen is F or Cl. In an embodiment, two of R⁴, R⁵, and R⁶ are F and one of R⁴, R⁵, and R⁶ is Cl or Br.
In a further embodiment, the compound is of formula (I-a), (I-b), (I-c), (I-d), (I-e), (I-f), (I-g) or (I-h):

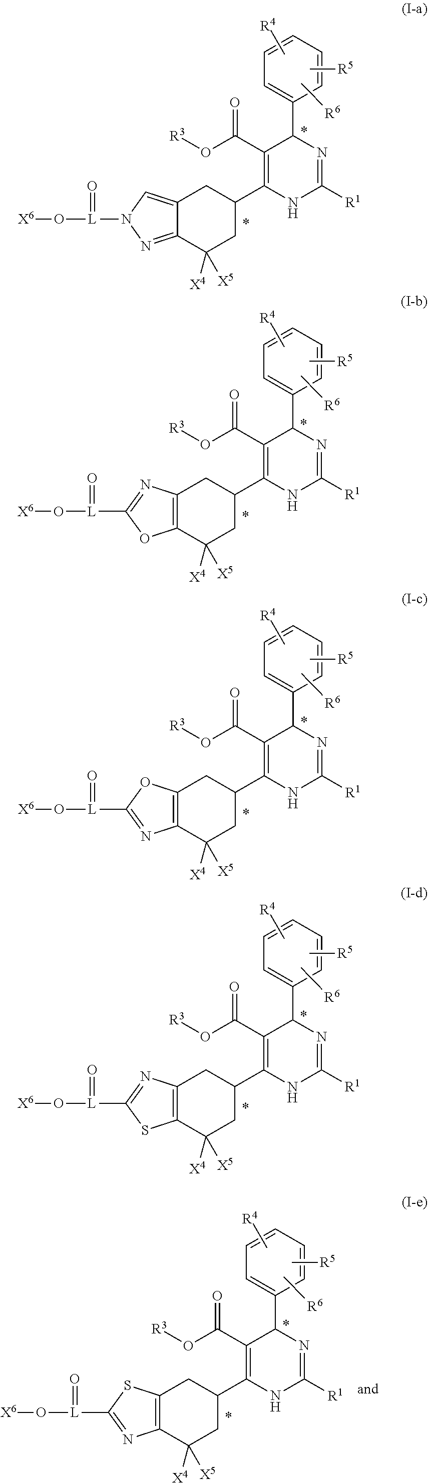

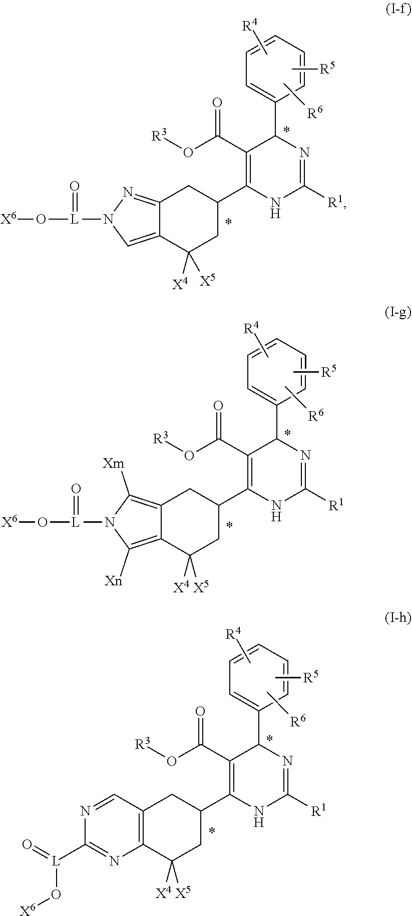

or a pharmaceutically acceptable salt or a solvate thereof. The indicated R, X, and L groups have the above-identified meaning.
In an embodiment of the compound of formula (I),
A is a 5-membered aromatic ring, which comprises N as heteroatoms, wherein the number of said N heteroatoms is two, wherein said 5-membered aromatic ring is optionally substituted with one or more substituents selected from C1-C4 alkyl and cyano,
L is C3 alkyl,

### X⁶ is H,

R⁴, R⁵ and R⁶ are each independently chosen from among CH₃, F, Cl and Br, more particularly from F and Cl,
R³ is C1-C3 alkyl, and
X⁴ and X⁵ are each independently chosen from among H and C1 alkyl.
In an embodiment, the compound of formula (I) is HBV inhibitor.
In an embodiment, the compound of formula (I) is HBV inhibitor with an EC50 equal or lower than 1 μM on Hep2.2.15 cell line.

The disclosed compounds may possess one or more stereocenters, and each stereocenter may exist independently in either the R or S configuration. In one embodiment, compounds described herein are present in optically active or racemic forms. It is to be understood that the compounds described herein encompass racemic, optically-active, regioisomeric and stereoisomeric forms, or combinations thereof that possess the therapeutically useful properties described herein.

Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In one embodiment, a mixture of one or more isomer is utilized as the disclosed compound described herein. In another embodiment, compounds described herein contain one or more chiral centers. These compounds are prepared by any means, including stereoselective synthesis, enantioselective synthesis or separation of a mixture of enantiomers or diastereomers. Resolution of compounds and isomers thereof is achieved by any means including, by way of non-limiting example, chemical processes, enzymatic processes, fractional crystallization, distillation, and chromatography.

When the absolute R or S stereochemistry of a compound cannot be determined, it can be identified by the retention time after chromatography under particular chromatographic conditions as determined by chromatography column, eluent etc.

In one embodiment, the disclosed compounds may exist as tautomers. All tautomers are included within the scope of the compounds presented herein.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In one embodiment, isotopically-labeled compounds are useful in drug or substrate tissue distribution studies. In another embodiment, substitution with heavier isotopes such as deuterium affords greater metabolic stability (for example, increased in vivo half-life or reduced dosage requirements).

In yet another embodiment, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In one embodiment, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and techniques known to a person skilled in the art. General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein. General synthesis schemes are given in the Examples below.

Accordingly, a process is provided for producing the compound of formula I, wherein said process comprises reacting the compound of formula III with the compound of formula IV and the compound of formula V to produce the compound of formula I:

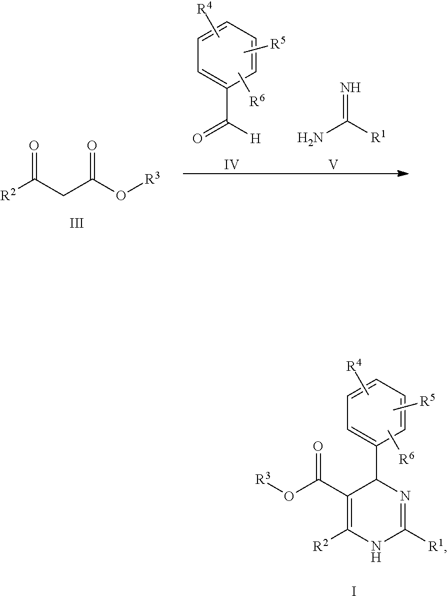

Such process can be carried out, e.g., under the influence of an acetate, such as sodium acetate, in a suitable solvent, such as ethanol, at elevated temperature, such as above the boiling point of ethanol, such as at 80° C. to 100° C.

The compounds of Formula (I) are active as inhibitors of the HBV replication cycle and can be used in the treatment and prophylaxis of HBV infection or of diseases or conditions, which are associated with or induced by HBV. Such diseases or conditions include progressive liver fibrosis, inflammation and necrosis leading to cirrhosis, end-stage liver disease, and hepatocellular carcinoma.

### Methods

Provided herein is a method of treating an HBV infection in an individual in need thereof, comprising administering to the individual a therapeutically effective amount of a disclosed compound.

Also provided herein is a method of eradicating an HBV infection in an individual in need thereof, comprising administering to the individual a therapeutically effective amount of a disclosed compound.

Provided herein is a method of reducing viral load associated with an HBV infection in an individual in need thereof, comprising administering to the individual a therapeutically effective amount of a disclosed compound.

Further, provided herein is a method of reducing reoccurrence of an HBV infection in an individual in need thereof, comprising administering to the individual a therapeutically effective amount of a disclosed compound.

Provided herein is a method of inhibiting or reducing the formation or presence of HBV DNA-containing particles or HBV RNA-containing particles in an individual in need thereof, comprising administering to the individual a therapeutically effective amount of a disclosed compound.

In certain aspects, the methods and/or compositions described herein are effective for inhibiting or reducing the formation or presence of HBV-associated particles in vitro or in vivo (e.g., in a cell, in a tissue, in an organ (e.g., in the liver), in an organism or the like). HBV-associated particles may contain HBV DNA (i.e., linear and/or covalently closed circular DNA (cccDNA)) and/or HBV RNA (i.e., pre-genomic RNA and/or sub-genomic RNA). Accordingly, HBV-associated particles include HBV DNA-containing particles or HBV RNA-containing particles.

As used herein, “HPV-associated particles” refer to both infectious HBV virions (i.e., Dane particles) and non-infectious HBV subviral particles (i.e., HBV filaments and/or HBV spheres). HBV virions comprise an outer envelope including surface proteins, a nucleocapsid comprising core proteins, at least one polymerase protein, and an HBV genome. HBV filaments and HBV spheres comprise HBV surface proteins, but lack core proteins, polymerase and an HBV genome. HBV filaments and HBV spheres are also known collectively as surface antigen (HBsAg) particles. HBV spheres comprise middle and small HBV surface proteins. HBV filaments also include middle, small and large HBV surface proteins.

HBV subviral particles can include the nonparticulate or secretory HBeAg, which serves as a marker for active replication of HBV.

Provided herein is a method of reducing an adverse physiological impact of an HBV infection in an individual in need thereof, comprising administering to the individual a therapeutically effective amount of a disclosed compound.

Also provided herein is a method of reducing, slowing, or inhibiting an HBV infection in an individual in need thereof, comprising administering to the individual a therapeutically effective amount of a disclosed compound.

Provided herein is a method of inducing reversal of hepatic injury from an HBV infection in an individual in need thereof, comprising administering to the individual a therapeutically effective amount of a disclosed compound.

Provided herein is a method of reducing the physiological impact of long-term antiviral therapy for HBV infection in an individual in need thereof, comprising administering to the individual a therapeutically effective amount of a disclosed compound.

Provided herein is a method of prophylactically treating an HBV infection in an individual in need thereof, wherein the individual is afflicted with a latent HBV infection, comprising administering to the individual a therapeutically effective amount of a disclosed compound.

In one embodiment, the individual is refractory to other therapeutic classes of HBV drugs (e.g, HBV polymerase inhibitors, interferons, viral entry inhibitors, viral maturation inhibitors, literature-described capsid assembly modulators, antiviral compounds of distinct or unknown mechanism, and the like, or combinations thereof). In another embodiment, the disclosed method reduces viral load in an individual suffering from an HBV infection to a greater extent or at a faster rate compared to the extent that other therapeutic classes of HBV drugs reduce viral load in the individual.

In one embodiment, the administering of a disclosed compound, or a pharmaceutically acceptable salt thereof, allows for administering of the at least one additional therapeutic agent at a lower dose or frequency as compared to the administering of the at least one additional therapeutic agent alone that is required to achieve similar results in prophylactically treating an HBV infection in an individual in need thereof.

In one embodiment, the administering of a disclosed compound, or a pharmaceutically acceptable salt thereof, reduces the viral load in the individual to a greater extent or at a faster rate compared to the administering of a compound selected from the group consisting of an HBV polymerase inhibitor, interferon, viral entry inhibitor, viral maturation inhibitor, distinct capsid assembly modulator, antiviral compounds of distinct or unknown mechanism, and any combination thereof.

In one embodiment, the disclosed method reduces viral load in an individual suffering from an HBV infection, thus allowing lower doses or varying regimens of combination therapies to be used.

In one embodiment, the disclosed method causes a lower incidence of viral mutation or viral resistance compared to other classes of HBV drugs, thereby allowing for long term therapy and minimizing the need for changes in treatment regimens.

In one embodiment, the administering of a compound the invention, or a pharmaceutically acceptable salt thereof, causes a lower incidence of viral mutation or viral resistance than the administering of a compound selected from the group consisting of an HBV polymerase inhibitor, interferon, viral entry inhibitor, viral maturation inhibitor, distinct capsid assembly modulator, antiviral compounds of distinct or unknown mechanism, and combination thereof.

In one embodiment, the disclosed method increases the seroconversion rate from HBV infected to non-HBV infected or from detectable HBV viral load to non-detectable HBV viral load beyond that of current treatment regimens. As used herein, “seroconversion” refers to the period of time during which HBV antibodies develop and become detectable.

In one embodiment, the disclosed method increases or normalizes or restores normal health, elicits full recovery of normal health, restores life expectancy, or resolves the viral infection in the individual in need thereof.

In one embodiment, the disclosed method eliminates or decreases the number of HBV RNA particles that are released from HBV infected cells thus enhancing, prolonging, or increasing the therapeutic benefit of the disclosed compounds.

In one embodiment, the disclosed method eradicates HBV from an individual infected with HBV, thereby obviating the need for long term or life-long treatment, or shortening the duration of treatment, or allowing for reduction in dosing of other antiviral agents.

In another embodiment, the disclosed method further comprises monitoring or detecting the HBV viral load of the subject, and wherein the method is carried out for a period of time including until such time that the HBV virus is undetectable.

Accordingly, in one embodiment, provided herein is a method of treating an HBV infection in an individual in need thereof, comprising administering to the individual a therapeutically effective amount of a compound of Formula I, or a pharmaceutically acceptable salt thereof.

Accordingly, in one embodiment, provided herein is a method of treating an HBV infection in an individual in need thereof, comprising administering to the individual a therapeutically effective amount of a compound of Formula I, or a pharmaceutically acceptable salt thereof.

In another embodiment, provided herein is a method of treating an HBV infection in an individual in need thereof, comprising administering to the individual a therapeutically effective amount of a compound of Table 1, or a pharmaceutically acceptable salt thereof.

In an embodiment of any of the methods provided herein, the method can further comprise monitoring the HBV viral load of the subject, wherein the method is carried out for a period of time such that the HBV virus is undetectable.

### Combination Therapies

The disclosed compounds may be useful in combination with one or more additional compounds useful for treating HBV infection. These additional compounds may comprise other disclosed compounds and/or compounds known to treat, prevent, or reduce the symptoms or effects of HBV infection. Such compounds include, but are not limited to, HBV polymerase inhibitors, interferons, viral entry inhibitors, viral maturation inhibitors, literature-described capsid assembly modulators, reverse transcriptase inhibitors, immunomodulatory agents, TLR-agonists, and other agents with distinct or unknown mechanisms that affect the HBV life cycle or affect the consequences of HBV infection.

In non-limiting examples, the disclosed compounds may be used in combination with one or more drugs (or a salt thereof) selected from the group comprising:

HBV reverse transcriptase inhibitors, and DNA and RNA polymerase inhibitors including, but not limited to, lamivudine (3TC, Zeffix, Heptovir, Epivir, and Epivir-HBV), entecavir (Baraclude, Entavir), adefovir dipivoxil (Hepsara, Preveon, bis-POM PMEA), tenofovir disoproxil fumarate (Viread, TDF or PMPA);

interferons including, but not limited to, interferon alpha (IFN-α), interferon beta (IFN-β), interferon lambda (IFN-λ), and interferon gamma (IFN-γ);

viral entry inhibitors;

viral maturation inhibitors;

literature-described capsid assembly modulators, such as, but not limited to, BAY 41-4109;

reverse transcriptase inhibitors;

immunomodulatory agents such as TLR-agonists; and

agents of distinct or unknown mechanisms, such as but not limited to AT-6 ((E)-N (1-chloro-3-oxo-1-phenyl-3-(piperidin-1-yl)prop-1-en-2-yl)benzamide), AT-130 ((E)-N-(1-bromo-1-(2-methoxyphenyl)-3-oxo-3-(piperidin-1-yl)prop-1-en-2-yl)-4-nitrobenzamide), and similar analogs.

In one embodiment, the additional therapeutic agent is an interferon. The term “interferon” or “IFN” refers to any member of the family of highly homologous species-specific proteins that inhibit viral replication and cellular proliferation and modulate immune response. Human interferons are grouped into three classes: Type I, which includes interferon-alpha (IFN-α), interferon-beta (IFN-β), and interferon-omega (IFN-ω), Type II, which includes interferon-gamma (IFN-γ), and Type III, which includes interferon-lambda (IFN-λ). Recombinant forms of interferons that have been developed and are commercially available are encompassed by the term “interferon” as used herein. Subtypes of interferons, such as chemically modified or mutated interferons, are also encompassed by the term “interferon” as used herein. Chemically modified interferons may include pegylated interferons and glycosylated interferons. Examples of interferons also include, but are not limited to, interferon-alpha-2a, interferon-alpha-2b, interferon-alpha-n1, interferon-beta-1a, interferon-beta-1b, interferon-lamda-1, interferon-lamda-2, and interferon-lamda-3. Examples of pegylated interferons include pegylated interferon-alpha-2a and pegylated interferon alpha-2b.

Accordingly, in one embodiment, the compounds of Formula I can be administered in combination with an interferon selected from the group consisting of interferon alpha (IFN-α), interferon beta (IFN-β), interferon lambda (IFN-λ), and interferon gamma (IFN-γ). In one specific embodiment, the interferon is interferon-alpha-2a, interferon-alpha-2b, or interferon-alpha-n1. In another specific embodiment, the interferon-alpha-2a or interferon-alpha-2b is pegylated. In a preferred embodiment, the interferon-alpha-2a is pegylated interferon-alpha-2a (PEGASYS).

In another embodiment, the additional therapeutic agent is selected from immune modulator or immune stimulator therapies, which includes biological agents belonging to the interferon class.

Further, the additional therapeutic agent may be an agent of distinct or unknown mechanism including agents that disrupt the function of other essential viral protein(s) or host proteins required for HBV replication or persistence.

In another embodiment, the additional therapeutic agent is an antiviral agent that blocks viral entry or maturation or targets the HBV polymerase such as nucleoside or nucleotide or non-nucleos(t)ide polymerase inhibitors. In a further embodiment of the combination therapy, the reverse transcriptase inhibitor or DNA or RNA polymerase inhibitor is Zidovudine, Didanosine, Zalcitabine, ddA, Stavudine, Lamivudine, Abacavir, Emtricitabine, Entecavir, Apricitabine, Atevirapine, ribavirin, acyclovir, famciclovir, valacyclovir, ganciclovir, valganciclovir, Tenofovir, Adefovir, PMPA, cidofovir, Efavirenz, Nevirapine, Delavirdine, or Etravirine.

In an embodiment, the additional therapeutic agent is an immunomodulatory agent that induces a natural, limited immune response leading to induction of immune responses against unrelated viruses. In other words, the immunomodulatory agent can effect maturation of antigen presenting cells, proliferation of T-cells and cytokine release (e.g., IL-12, IL-18, IFN-alpha, -beta, and -gamma and TNF-alpha among others),

In a further embodiment, the additional therapeutic agent is a TLR modulator or a TLR agonist, such as a TLR-7 agonist or TLR-9 agonist. In further embodiment of the combination therapy, the TLR-7 agonist is selected from the group consisting of SM360320 (9-benzyl-8-hydroxy-2-(2-methoxy-ethoxy)adenine) and AZD 8848 (methyl [3-({[3-(6-amino-2-butoxy-8-oxo-7,8-dihydro-9H-purin-9-yl)propyl][3-(4-morpholinyl)-propyl]amino}methyl)phenyl]acetate).

In any of the methods provided herein, the method may further comprise administering to the individual at least one HBV vaccine, a nucleoside HBV inhibitor, an interferon or any combination thereof. In an embodiment, the HBV vaccine is at least one of RECOMBIVAX HB, ENGERIX-B, ELOVAC B, GENEVAC-B, or SHANVAC B.

In one embodiment, the methods described herein further comprise administering at least one additional therapeutic agent selected from the group consisting of nucleotide/nucleoside analogs, entry inhibitors, fusion inhibitors, and any combination of these or other antiviral mechanisms.

In another aspect, provided herein is method of treating an HBV infection in an individual in need thereof, comprising reducing the HBV viral load by administering to the individual a therapeutically effective amount of a disclosed compound alone or in combination with a reverse transcriptase inhibitor; and further administering to the individual a therapeutically effective amount of HBV vaccine. The reverse transcriptase inhibitor may be at least one of Zidovudine, Didanosine, Zalcitabine, ddA, Stavudine, Lamivudine, Abacavir, Emtricitabine, Entecavir, Apricitabine, Atevirapine, ribavirin, acyclovir, famciclovir, valacyclovir, ganciclovir, valganciclovir, Tenofovir, Adefovir, PMPA, cidofovir, Efavirenz, Nevirapine, Delavirdine, or Etravirine.

In another aspect, provided herein is a method of treating an HBV infection in an individual in need thereof, comprising reducing the HBV viral load by administering to the individual a therapeutically effective amount of a disclosed compound alone or in combination with a antisense oligonucleotide or RNA interference agent that targets HBV nucleic acids; and further administering to the individual a therapeutically effective amount of HBV vaccine. The antisense oligonucleotide or RNA interference agent possesses sufficient complementarity to the target HBV nucleic acids to inhibit replication of the viral genome, transcription of viral RNAs, or translation of viral proteins.

In another embodiment, the disclosed compound and the at least one additional therapeutic agent are co-formulated. In yet another embodiment, the disclosed compound and the at least one additional therapeutic agent are co-administered.

For any combination therapy described herein, synergistic effect may be calculated, for example, using suitable methods such as the Sigmoid-Eₘₐₓ equation (Holford & Scheiner, 19981, Clin. Pharmacokinet. 6: 429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22: 27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

In an embodiment of any of the methods of administering combination therapies provided herein, the method can further comprise monitoring or detecting the HBV viral load of the subject, wherein the method is carried out for a period of time including until such time that the HBV virus is undetectable.

### Administration/Dosage/Formulations

In another aspect, provided herein is a pharmaceutical composition comprising at least one disclosed compound, or a pharmaceutically acceptable salt thereof, together with a pharmaceutically acceptable carrier.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In particular, the selected dosage level will depend upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could begin administration of the pharmaceutical composition to dose the disclosed compound at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of the disclosed compound calculated to produce the desired therapeutic effect in association with the required pharmaceutical vehicle. The dosage unit forms of the invention are dictated by and directly dependent on (a) the unique characteristics of the disclosed compound and the particular therapeutic effect to be achieved, and (b) the limitations inherent in the art of compounding/formulating such a disclosed compound for the treatment of HBV infection in a patient.

In one embodiment, the compositions of the invention are formulated using one or more pharmaceutically acceptable excipients or carriers. In one embodiment, the pharmaceutical compositions of the invention comprise a therapeutically effective amount of a disclosed compound and a pharmaceutically acceptable carrier.

In some embodiments, the dose of a disclosed compound is from about 1 mg to about 2,500 mg. In some embodiments, a dose of a disclosed compound used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., another drug for HBV treatment) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In one embodiment, the present invention is directed to a packaged pharmaceutical composition comprising a container holding a therapeutically effective amount of a disclosed compound, alone or in combination with a second pharmaceutical agent; and instructions for using the compound to treat, prevent, or reduce one or more symptoms of HBV infection in a patient.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For parenteral administration, the disclosed compounds may be formulated for injection or infusion, for example, intravenous, intramuscular or subcutaneous injection or infusion, or for administration in a bolus dose or continuous infusion. Suspensions, solutions or emulsions in an oily or aqueous vehicle, optionally containing other formulatory agents such as suspending, stabilizing or dispersing agents may be used.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

### Example 1

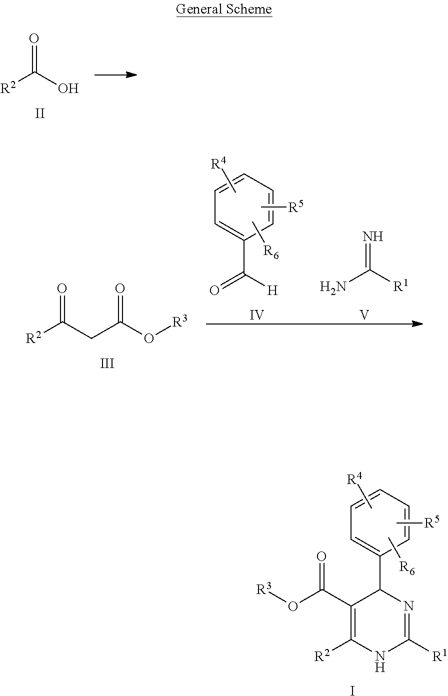

The general synthesis of compound of general formula I is described in scheme 1 and scheme 2. Compound of general formula III can be synthesized as described in Scheme 1 (Method A₁ or Method A₂), method selection is depending on the substituent R₃ on compound of general formula III. As described in Method A₁, an acid of general formula II is converted by reacting with N,N-carbonyldiimidazole CDI to an activated ester which then couples with ethyl potassium malonate under basic condition to generate an intermediate which in turn undergoes decarboxylation to yield the ketoester of general formula III.

The final product of general formula I can be synthesized as described in Scheme 2. The former is the chemical methodology of multiple component reaction with compounds of general formula III, IV and V in the presence of base (but not limited to sodium acetate NaOAc) in solvent of choice (but not limited to ethanol EtOH).

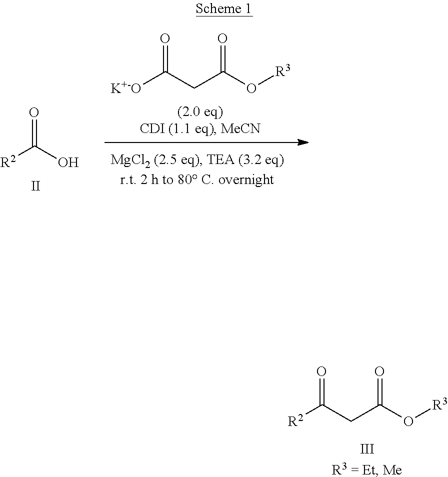

### Method A₁

To a solution of the acid of general formula II (1 equivalent) in acetonitrile was added N,N-carbonyldiimidazole (1.1-2 equivalents) at room temperature. The mixture was stirred at room temperature under nitrogen atmosphere for 2 hours (mixture A). To a suspension of ethyl potassium malonate (2-2.1 equivalents) in acetonitrile was added magnesium chloride (2.1-2.5 equivalents) and triethylamine (3-3.2 equivalents) at room temperature. After stirred under nitrogen atmosphere for 2 hours, the resulting mixture was added mixture A and stirring continued at 80-100° C.; in a range of three hours to overnight. It was then cooled down to room temperature and concentrated to give a residue, which was purified by silica gel column chromatography to afford the ketoester of general formula III.

### Method A2

By utilizing the analogous procedure of Method A₁, methyl potassium malonate was engaged to replace ethyl potassium malonate.

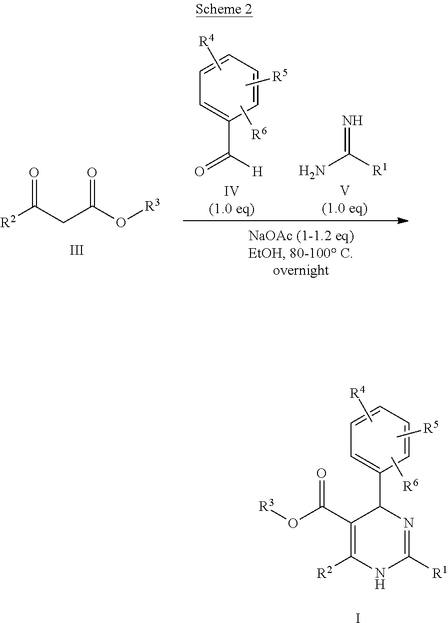

### Method B

To a solution of the ketoester of general formula III (1 equivalent) in ethanol was added the aldehyde of general formula IV (1 equivalent), the carboxamidine hydrochloride of general formula V (1 equivalent) and sodium acetate (1-1.2 equivalents). The mixture was brought up to 70-100° C.; and stirred under nitrogen atmosphere from sixteen hours to overnight. After cooled down to room temperature, it was concentrated to dryness. The residue was taken up in dichloromethane, washed with water, brine, dried over anhydrous Na₂SO₄ and filtered. The filtrate was concentrated under reduced pressure to give a residue, which was purified by silica gel column chromatography to afford the dihydropyrimidine product of general formula I. When applicable, the stereoisomers of the dihydropyrimidine product of general formula I were isolated and purified using chiral chromatography.

### Part I: Preparation of Acids of General Formula II

### Acid 1: 2-(3-Methoxy-3-oxopropyl)-4,5,6,7-tetrahydro-2H-indazole-5-carboxylic acid (A1)

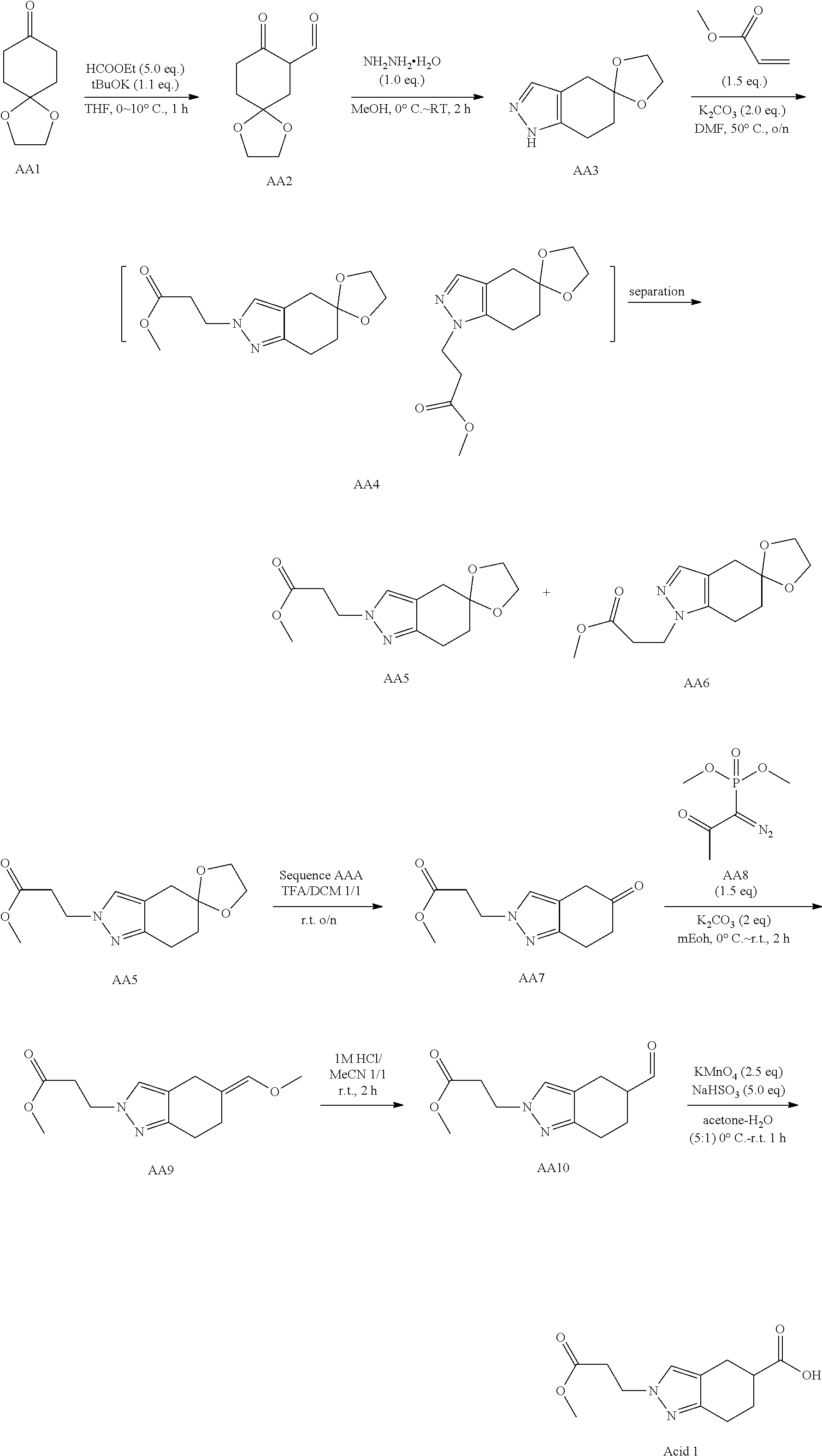

### Intermediate AA2

### 8-Oxo-1,4-dioxa-spiro[4.5]decane-7-carbaldehyde

To an ice-cold solution of potassium tert-butoxide (32.0 g, 282 mmol) in dry tetrahydrofuran (800 mL) was slowly added ethyl formate (60.0 mL, 742 mmol). The mixture was stirred at 0° C.; for 30 min before a solution of 1,4-dioxaspiro[4.5]decan-8-one AA1 (40.0 g, 256 mmol) and ethyl formate (44.0 mL, 544 mmol) in tetrahydrofuran (200 mL) was added over a period of 20 min under nitrogen atmosphere. After the addition, the resulting mixture was stirred at room temperature for 1 hour. It was then quenched with 10% wt. citric acid aqueous solution (160 mL). The mixture was concentrated under reduced pressure to remove volatiles and the residue was partitioned between ethyl acetate (400 mL) and water (100 mL). The aqueous layer was extracted with ethyl acetate (100 mL) twice. The combined organic layers were washed with brine (100 mL), dried over Na₂SO₄O) and filtered. The filtrate was concentrated under reduced pressure to give the title compound (45.0 g, 96% yield) as brown oil. ¹H NMR (300 MHz, CDCl₃) δ 14.37 (s, 0.5H), 8.51 (s, 0.5H), 4.04-3.99 (m, 4H), 2.59-2.46 (m, 4H), 1.99 (t, J=7.2 Hz, 2H), 1.84 (t, J=7.2 Hz, 1H).

### Intermediate AA3

### 1,4,6,7-tetrahydrospiro[indazole-5,2′-[1,3]dioxolane]

To a stirring solution of 8-oxo-1,4-dioxa-spiro[4.5]decane-7-carbaldehyde AA2 (45.0 g, 240 mmol) in methanol was added hydrazine hydrate (14.0 g, 240 mmol) at 0° C. After stirred at room temperature for 2 hours under nitrogen atmosphere, the mixture was concentrated under reduced pressure to leave a residue, which was partitioned between ethyl acetate (400 mL) and washed with water (100 mL). The aqueous layer was extracted with ethyl acetate (100 mL) for three times. The combined organic layers were washed with brine (100 mL), dried over Na₂SO₄₍ₛ₎ and filtered. The filtrate was concentrated under reduced pressure to give the title compound (40.0 g, 91% yield) as yellow oil. LC-MS (ESI): R_{T}=0.65 min, mass calcd. for C₉H₁₂N₂O₂ 180.1, m/z found 181.3 [M+H]⁺. ¹H NMR (300 MHz, CDCl₃) δ 7.29 (s, 1H), 4.03 (s, 4H), 2.87 (s, 2H), 2.79 (s, 2H), 1.99 (s, 2H).

### Intermediates AA4

### Mixture of methyl 3-(6′,7′-dihydrospiro[[1,3]dioxolane-2,5′-indazol]-2′(4′H)-yl)propanoate and methyl 3-(6′,7′-dihydrospiro[[1,3]dioxolane-2,5′-indazol]-1′(4′H)-yl)propanoate

To a solution of 1,4,6,7-tetrahydrospiro[indazole-5,2′-[1,3]dioxolane] AA3 (34.0 g, 189 mmol) in N,N-dimethylformamide (340 mL) was added acrylic acid methyl ester (24.0 g, 283 mmol) and potassium carbonate (52.0 g, 378 mmol) at room temperature. After stirred at 50° C.; under nitrogen atmosphere overnight, the mixture was cooled down to room temperature and concentrated under reduced pressure to remove volatiles. The residue was poured into water (100 mL) and extracted with ethyl acetate (100 mL) for three times. The combined organic layers were washed with water (100 mL), brine (100 mL), dried over Na₂SO₄₍ₛ₎ and filtered. The filtrate was concentrated under reduced pressure to give a crude product, which was purified by C18 column (acetonitrile:water=5% to 60%) to give the title compound (29.7 g, 59% yield) as light yellow oil. LC-MS (ESI): R_{T}=1.26 min, mass calcd. for C₁₃H₁₈N₂O₄ 266.1, m/z found 267.3 [M+H]⁺. ¹H NMR (300 MHz, DMSO-d₆) δ 7.34 (s, 0.6H), 7.13 (s, 0.4H), 4.23-4.12 (m, 2H), 3.90 (s, 4H), 3.64-3.55 (m, 3H), 2.80 (t, J=6.9 Hz, 2H), 2.74-2.59 (m, 4H), 1.88-1.81 (m, 2H).

A regio-isomeric mixture of AA4 (66.0 g, 248 mmol) was separated by SFC (separation condition: Column: Chiralpak ID 5 μm 20*250 mm; Mobile Phase: CO₂:IPA=70:30 at 50 g/min; Co-solvent: IPA; Col. Temp: 40° C.; Wavelength: 214 nm, Back pressure: 100 bar) to afford the title compounds AA5 (35.5 g, 54% yield) as light yellow oil and AA6 (21.0 g, 32% yield) as light yellow oil.

AA5: LC-MS (ESI): R_{T}=1.29 min, mass calcd. for C₁₃H₁₈N₂O₄ 266.1, m/z found 267.3 [M+H]⁺. SFC analytical condition: (Column: Chiralpak ID 5 μm 4.6*250 mm; Mobile Phase: CO₂:IPA=70:30 at 3 g/min; Col. Temp: 40° C.; Wavelength: 230 nm, Back pressure: 100 bar, R_{T}=3.38 min). ¹H NMR (300 MHz, DMSO-d₆) δ 7.37-7.30 (m, 1H), 4.24-4.18 (m, 2H), 3.94-3.86 (m, 4H), 3.63-3.55 (m, 3H), 2.84-2.78 (m, 2H), 2.65-2.60 (m, 4H), 1.86-1.80 (m, 2H).

### Sequence AAA (Utilized in Other Syntheses of Acids)

### Intermediate AA7

### Methyl 3-(5-oxo-4,5,6,7-tetrahydro-2H-indazol-2-yl)propanoate

To a solution of methyl 3-(6′,7′-dihydrospiro[[1,3]dioxolane-2,5′-indazol]-2′(4′H)-yl) propanoate AA5 (15.0 g, 56.3 mmol) in dichloromethane (150 mL) was added trifluoroacetic acid (150 mL) at 0° C. After stirred at room temperature overnight, the mixture was concentrated under reduced pressure to give a brown residue, which was purified by C18 column (acetonitrile:water=5% to 60%) to give the title compound (10.0 g, 80% yield) as white solids. LC-MS (ESI): R_{T}=1.15 min, mass calcd. for C₁₁H₁₄N₂O₃ 222.1, m/z found 223.1 [M+H]⁺. ¹H NMR (300 MHz, DMSO-d₆) δ 7.47 (s, 1H), 4.27 (t, J=6.6 Hz, 2H), 3.60 (s, 3H), 3.34 (s, 2H), 2.91-2.83 (m, 4H), 2.56 (t, J=6.6 Hz, 2H).

### Intermediate AA9

### Methyl 3-(5-(methoxymethylene)-4,5,6,7-tetrahydro-2H-indazol-2-yl)propanoate

To the solution of methyl 3-(5-oxo-4,5,6,7-tetrahydro-2H-indazol-2-yl)propanoate AA7 (5.70 g, 25.1 mmol) and dimethyl (1-diazo-2-oxopropyl)phosphonate AA8 (7.39 g, 37.7 mmol) in dry methanol (80 mL) was added potassium carbonate (7.09 g, 50.3 mmol) at 0° C. After stirred at 0° C.; for 30 minutes and then at room temperature for 2 hours under nitrogen atmosphere, the reaction was quenched with saturated ammonium chloride aqueous solution (250 mL) at 0° C.; and extracted with ethyl acetate (200 mL) twice. The combined organic layers were dried over Na₂SO₄(s) and filtered. The filtrate was concentrated and purified by silica gel column chromatography (petroleum ether:ethyl acetate=5:1 to 3:1) to give a residue, which was purified by C18 column (acetonitrile:water=30% to 60%) to give the title compound (4.80 g, 72% yield) as white solids. LC-MS (ESI): R_{T}=1.47 min, mass calcd. for C₁₃H₁₈N₂O₃ 250.1, m/z found 251.2 [M+H]⁺. ¹H NMR (300 MHz, DMSO-d₆) δ 7.36 (s, 1H), 6.08 (s, 0.4H), 6.04 (s, 0.6H), 4.21 (t, J=6.6 Hz, 2H), 3.59 (s, 3H), 3.51 (s, 2.1H), 3.50 (s, 0.9H), 3.15 (s, 1.2H), 3.01 (s, 0.8H), 2.81 (t, J=6.6 Hz, 2H), 2.54-2.50 (m, 2H), 2.37 (t, J=6.3 Hz, 0.8H), 2.21 (t, J=8.4 Hz, 1.2H).

### Intermediate AA10

### Methyl 3-(5-formyl-4,5,6,7-tetrahydro-2H-indazol-2-yl)propanoate

To the solution of methyl 3-(5-(methoxymethylene)-4,5,6,7-tetrahydro-2H-indazol-2-yl)propanoate AA9 (8.30 g, 31.5 mmol) in acetonitrile (83 mL) was added 1 M hydrochloride aqueous solution (83 mL) at 0° C. After stirred at room temperature for 3 hours, the mixture was diluted with brine (160 mL) and then pH was adjusted to ˜8 with saturated sodium bicarbonate aqueous solution. The resulting mixture was extracted with ethyl acetate (200 mL) twice. The combined organic layers were dried over Na₂SO₄(s) and concentrated to give the title compound (7.20 g, 95% yield) as light yellow oil. LC-MS (ESI): R_{T}=1.33 min, mass calcd. for C₁₂H₁₆N₂O₃ 236.1, m/z found 237.3 [M+H]⁺. ¹H NMR (400 MHz, DMSO-d₆) δ 9.69 (s, 1H), 7.41 (s, 1H), 4.22 (t, J=6.8 Hz, 2H), 3.59 (s, 3H), 2.82 (t, J=6.8 Hz, 2H), 2.72-2.54 (m, 5H), 2.15-2.09 (m, 1H), 1.75-1.66 (m, 1H).

### Acid 1:

### 2-(3-Methoxy-3-oxopropyl)-4,5,6,7-tetrahydro-2H-indazole-5-carboxylic acid

To a solution of methyl 3-(5-formyl-4,5,6,7-tetrahydro-2H-indazol-2-yl)propanoate AA10 (5.45 g, 21.9 mmol) in acetone (165 mL) and water (33 mL) was added potassium permanganate (8.70 g, 54.8 mmol) at 0° C. After stirred in a range from 0° C.; to room temperature for 1 hour, the reaction mixture was quenched with an addition of sodium bisulfite (11.5 g, 110 mmol) followed by dilution with acetone (220 mL) and water (220 mL). The resulting suspension was stirred at room temperature for 15 minutes and filtered through a pad of celite. The filtrate was concentrated under reduced pressure at room temperature to remove acetone. The residue was then acidified with citric acid(s) to pH 3 and extracted with ethyl acetate (250 mL) twice. The combined organic layers were dried over Na₂SO₄(s) and concentrated to give the title compound (4.50 g, 77% yield) as white solids. LC-MS (ESI): R_{T}=0.29 min, mass calcd. for C₁₂H₁₆N₂O₄ 252.1, m/z found 253.2 [M+H]⁺. ¹H NMR (400 MHz, DMSO-d₆) δ 12.21 (s, 1H), 7.37 (s, 1H), 4.21 (t, J=6.8 Hz, 2H), 3.60 (s, 3H), 2.81 (t, J=6.8 Hz, 2H), 2.76-2.68 (m, 1H), 2.65-2.52 (m, 4H), 2.11-2.05 (m, 1H), 1.75-1.66 (m, 1H).

### Acid 2:

### 2-(tetrahydro-2H-pyran-2-yl)-4,5,6,7-tetrahydro-2H-indazole-5-carboxylic acid and 1-(tetrahydro-2H-pyran-2-yl)-4,5,6,7-tetrahydro-2H-indazole-5-carboxylic acid

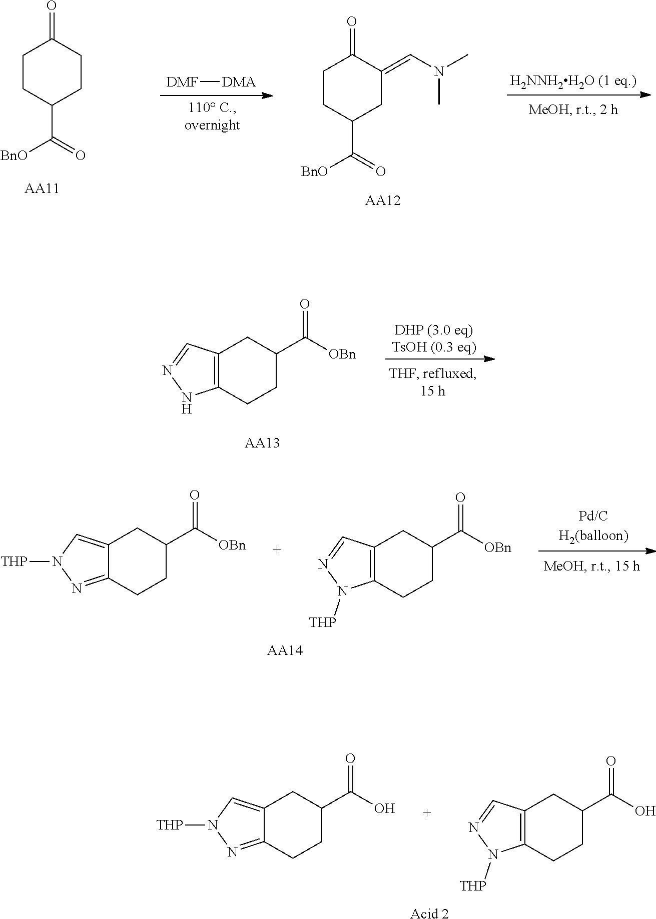

### Intermediate AA12

### Benzyl 3-((dimethylamino)methylene)-4-oxocyclohexanecarboxylate

A solution of benzyl 4-oxocyclohexanecarboxylate AA11 (30.0 g, 129 mmol) in 1,1-dimethoxy-N,N-dimethylmethanamine (100 mL) was stirred at 110° C.; overnight. After cooling down to room temperature, the mixture was concentrated under reduced pressure to give a residue, which was purified by silica gel column chromatography (petroleum ether:ethyl acetate=5:1 to 3:1 and then dichloromethane:methanol=100:1) to give the title compound (14.0 g, 38% yield) as yellow oil. LC-MS (ESI): R_{T}=1.207 min, mass calcd. for C₁₇H₂₁NO₃ 287.2, m/z found 288.0 [M+H]⁺.

### Intermediate AA13

### Benzyl 4,5,6,7-tetrahydro-1H-indazole-5-carboxylate

To a solution of benzyl 3-((dimethylamino)methylene)-4-oxocyclohexanecarboxylate AA12 (13.5 g, 74.0 mmol) in methanol (100 mL) was added hydrazine hydrate (2.40 g, 74.0 mmol). After stirred at room temperature for 2 hours under nitrogen atmosphere, the mixture was poured into water (200 mL) and extracted with ethyl acetate (100 mL) for three times. The combined organic layers were dried over Na₂SO₄₍ₛ₎ and filtered. The filtrate was concentrated under reduced pressure to give a residue, which was purified by C18 column (acetonitrile:water=30% to 80%) to give the title compound (5.60 g, 47% yield) as yellow oil. LC-MS (ESI): R_{T}=1.271 min, mass calcd. for C₁₅H₁₆N₂O₂ 256.3, m/z found 257.0 [M+H]⁺. ¹H NMR (400 MHz, CDCl₃) δ 7.37-7.34 (m, 6H), 5.18 (s, 2H), 2.95-2.65 (m, 5H), 2.32-2.26 (m, 1H), 2.03-1.90 (m, 1H).

### Intermediate AA14

### Mixture of benzyl 2-(tetrahydro-2H-pyran-2-yl)-4,5,6,7-tetrahydro-2H-indazole-5-carboxylate and benzyl 1-(tetrahydro-2H-pyran-2-yl)-4,5,6,7-tetrahydro-2H-indazole-5-carboxylate

To a solution of benzyl 4,5,6,7-tetrahydro-1H-indazole-5-carboxylate AA13 (2.85 g, 11.1 mmol) and p-toluenesulfonic acid (0.570 g, 3.34 mmol) in dry tetrahydrofuran (50 mL) was added 3,4-dihydro-2H-pyran (2.81 g, 33.4 mmol). After refluxed for 15 hours, the mixture was cooled down to room temperature and concentrated under reduced pressure to remove volatiles. The residue was dissolved in ethyl acetate (50 mL), washed with saturated sodium bicarbonate aqueous solution (20 mL) and brine (20 mL), dried over Na₂SO₄₍ₛ₎ and filtered. The filtrate was concentrated under reduced pressure to give a residue, which was purified by silica gel column chromatography (petroleum ether:ethyl acetate=3:1) to give the title compound (2.51 g, 66% yield) as yellow oil. LC-MS (ESI): R_{T}=1.69 and 1.71 min, mass calcd. for C₂₀H₂₄N₂O₃ 340.2, m/z found 341.2[M+H]⁺. ¹H NMR (300 MHz, CDCl₃) δ 7.35-7.27 (m, 6H), 5.27-5.21 (m, 1H), 5.15 (s, 2H), 4.13-4.00 (m, 2H), 3.70-3.62 (m, 2H), 2.91-2.73 (m, 5H), 2.29-2.24 (m, 1H), 1.67-1.54 (m, 5H).

### Acid 2:

### Mixture of 2-(tetrahydro-2H-pyran-2-yl)-4,5,6,7-tetrahydro-2H-indazole-5-carboxylic acid and 1-(tetrahydro-2H-pyran-2-yl)-4,5,6,7-tetrahydro-2H-indazole-5-carboxylic acid

To a mixture of AA14 (2.51 g, 7.37 mmol) in methanol (20 mL) was added 10% wt palladium on charcoal (200 mg) at room temperature under nitrogen atmosphere. After stirred at room temperature for 15 hours under balloon pressure of hydrogen gas, the mixture was filtered. The filtrate was concentrated under reduced pressure to give the title compound (1.82 g, 99% yield) as colorless oil. LC-MS (ESI): R_{T}=0.52 min, mass calcd. for C₁₃H₁₈N₂O₃ 250.1, m/z found 249.1 [M−H]⁺. ¹H NMR (300 MHz, CDCl₃) δ 7.35 (s, 1H), 5.30-5.29 (m, 1H), 4.09-3.89 (m, 2H), 3.71-3.49 (m, 2H), 2.91-2.72 (m, 5H), 2.30-2.26 (m, 1H), 1.68-1.56 (m, 5H).

### Acid 3:

### 2-(2-Methoxy-2-oxoethyl)-4,5,6,7-tetrahydro-2H-indazole-5-carboxylic acid

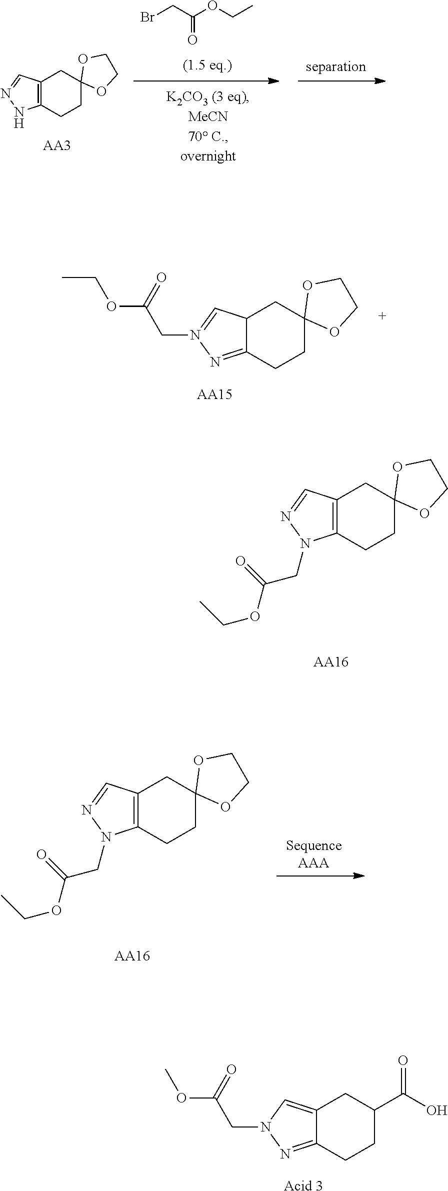

### Intermediate AA15

### Ethyl 2-(6′,7′-dihydrospiro[[1,3]dioxolane-2,5′-indazol]-2′ (4′H)-yl)acetate

To a solution of 1′,4′,6′,7′-tetrahydrospiro[[1,3]dioxolane-2,5′-indazole] AA3 (1.60 g, 8.89 mmol) in acetonitrile (40 mL) was added ethyl 2-bromoacetate (2.21 g, 13.3 mmol), potassium carbonate (3.67 g, 26.6 mmol). After stirred at 70° C.; overnight, the reaction mixture was filtered and the filtrate was concentrated under reduced pressure to give a residue, which was purified by C18 column (acetonitrile:water=5% to 100%) followed by chiral prep. HPLC (separation condition: column: Chiralpak IC 5 μm 20*250 mm, Mobile Phase: Hex:EtOH=70:30 at 12 mL/min, Temp: 30° C., Wavelength: 230 nm) to afford the title compound AA15 (520 mg, 43% yield) as brown oil and AA16 (460 mg, 38% yield) as brown oil.

AA15: LC-MS (ESI): R_{T}=2.924 min, mass calcd. for C₁₃H₁₈N₂O₄ 266.1, m/z found 267.1 [M+H]⁺. Chiral HPLC (Column: Chiralpak IC 5 μm 4.6*250 mm; Mobile Phase: Hex:EtOH=70:30 at 1.0 mL/min; Temp: 30° C.; Wavelength: 230 nm, R_{T}=9.189 min). ¹H NMR (400 MHz, DMSO-d₆) δ 7.37 (s, 1H), 4.90 (s, 2H), 4.13 (q, J=7.2 Hz, 2H), 3.91 (s, 4H), 2.66-2.63 (m, 4H), 1.85 (t, J=6.4 Hz, 2H), 1.20 (t, J=7.2 Hz, 3H).

### Acid 3:

### 2-(2-Methoxy-2-oxoethyl)-4,5,6,7-tetrahydro-2H-indazole-5-carboxylic acid

By ultilizing the analogous procedure of Sequence AAA, AA16 was converted to the title compound.

LC-MS (ESI): R_{T}=0.352 min, mass calcd. for C₁₁H₁₄N₂O₄ 238.1, m/z found 239.1 [M+H]⁺. ¹H NMR (400 MHz, DMSO-d₆) δ 12.22 (s, 1H), 7.41 (s, 1H), 4.93 (s, 2H), 3.66 (s, 3H), 2.80-2.72 (m, 1H), 2.67-2.54 (m, 4H), 2.16-2.07 (m, 1H), 1.78-1.68 (m, 1H).

### Acid 4:

### 2-(4-Methoxy-4-oxobutyl)-4,5,6,7-tetrahydro-2H-indazole-5-carboxylic acid

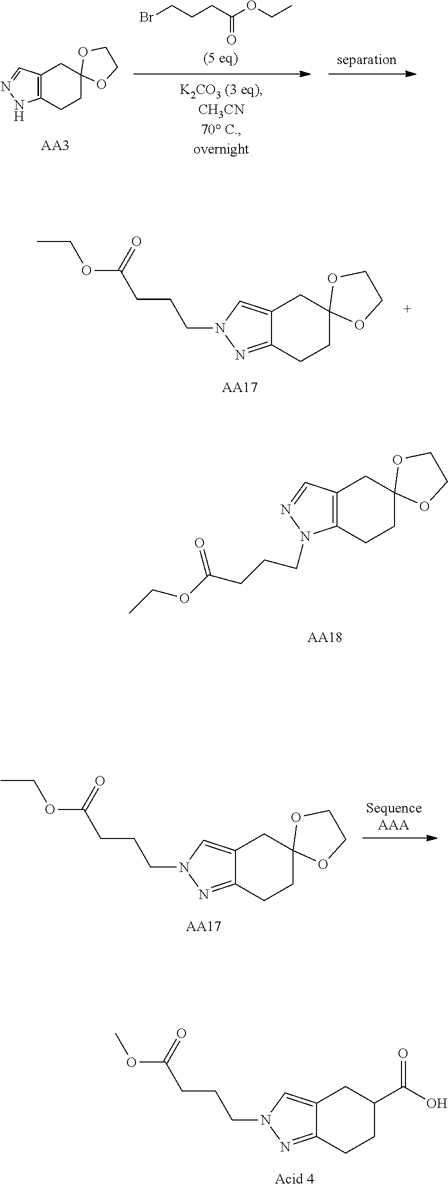

### Intermediate AA17

### Ethyl 4-(6′,7′-dihydrospiro[[1,3]dioxolane-2,5′-indazol]-2′ (4′H)-yl)butanoate

To a solution of 1′,4′,6′,7′-tetrahydrospiro[[1,3]dioxolane-2,5′-indazole] AA3 (15.0 g, 83.3 mmol) in acetonitrile (250 mL) was added ethyl 4-bromobutanoate (81.2 g, 416.5 mmol), potassium carbonate (34.5 g, 249.9 mmol) at room temperature. After stirred at 70° C. overnight, the reaction mixture was filtered and concentrated under reduced pressure to give a residue, which was purified by C18 column (acetonitrile:water=5% to 80%) to give a crude mixture of regio-isomers, which was separated by SFC (separation condition: Column: Chiralpak IG 5 μm 20*250 mm; Mobile Phase: CO₂:MeOH=75:25 at 50 g/min; Co-solvent: MeOH; Col. Temp 41.1° C.; Wavelength: 214 nm; Back pressure: 100 bar) to afford the title compounds AA17 (2.70 g, 45% yield) as colorless oil and AA18 (1.50 g, 25% yield) as colorless oil.

AA17: LC-MS (ESI): R_{T}=1.37 min, mass calcd. for C₁₅H₂₂N₂O₄ 294.2, m/z found 295.4 [M+H]⁺. ¹H NMR (300 MHz, DMSO-d₆) δ 7.33 (s, 1H), 4.08-3.88 (m, 8H), 2.67-2.63 (m, 4H), 2.28-2.23 (m, 2H), 2.01-1.91 (m, 2H), 1.87-1.82 (m, 2H), 1.17 (t, J=7.2 Hz, 3H).

### Acid 4:

### 2-(4-Methoxy-4-oxobutyl)-4,5,6,7-tetrahydro-2H-indazole-5-carboxylic acid

By ultilizing the analogous procedure of Sequence AAA, AA17 was converted to the title compound.

LC-MS (ESI): R_{T}=0.25 min, mass calcd. for C₁₃H₁₈N₂O₄ 266.1, m/z found 267.3 [M+H]⁺.

### Acid 5 and Acid 6 (Pair of Enantiomers):

### 2-(4-Methoxy-4-oxobutan-2-yl)-4,5,6,7-tetrahydro-2H-indazole-5-carboxylic acid

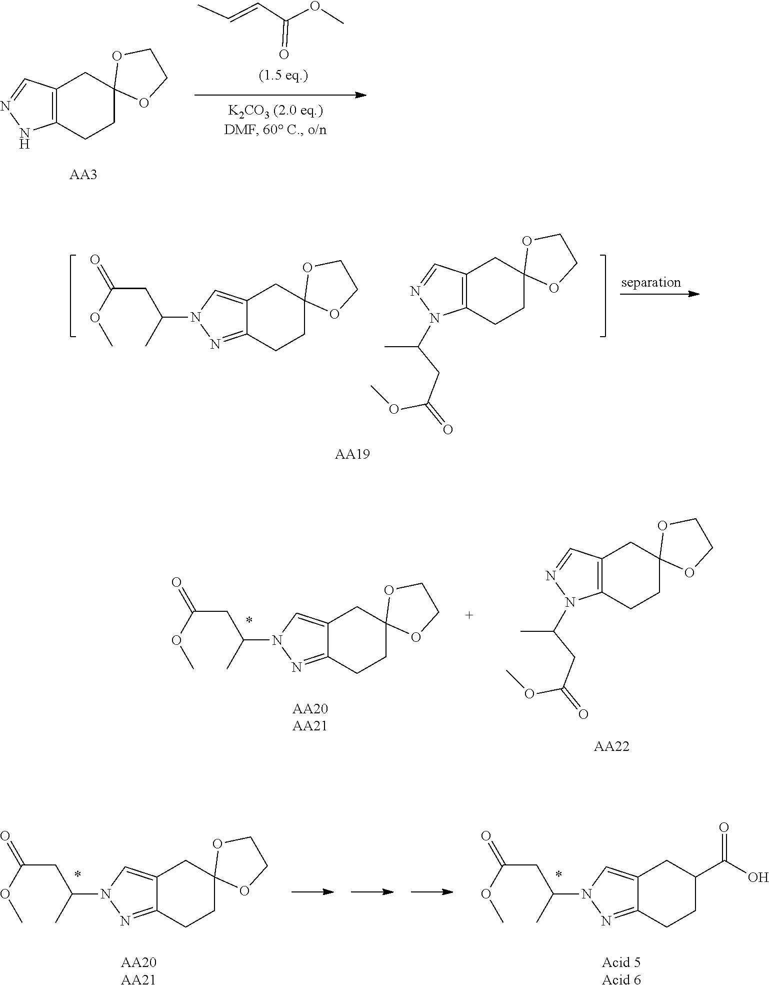

### Intermediate AA19: (Mixture of two regioisomers, each contains a pair of enantiomers)

### Mixture of methyl 3-(6′,7′-dihydrospiro[[1,3]dioxolane-2,5′-indazol]-2′ (4′H)-yl)butanoate and methyl 3-(6′,7′-dihydrospiro[[1,3]dioxolane-2,5′-indazol]-1′(4′H)-yl)butanoate

To a solution of 1′,4′,6′,7′-tetrahydrospiro[[1,3]dioxolane-2,5′-indazole] AA3 (12.7 g, 70.5 mmol) in N,N-dimethylformide (120 mL) was added (E)-methyl but-2-enoate (10.6 g, 105.8 mmol) and potassium carbonate (19.5 g, 141 mmol) at room temperature. After stirred at 60° C.; under nitrogen atmosphere overnight, the reaction mixture was cooled down to room temperature and concentrated under reduced pressure to remove the volatiles. The residue was poured into water (60 mL) and extracted with ethyl acetate (60 mL) for three times. The combined organic layers were washed with water (100 mL) twice, brine (100 mL) twice, dried over Na₂SO₄₍ₛ₎) and filtered. The filtrate was concentrated under reduced pressure to give a crude product, which was purified by C18 column (acetonitrile:water=5% to 60%) to give the title mixture (7.90 g, 40% yield) as light yellow oil. LC-MS (ESI): R_{T}=3.049 min, mass calcd. for C₁₄H₂₀N₂O₄ 280.1, m/z found 281.1 [M+H]⁺. ¹H NMR (300 MHz, CDCl₃) δ 7.27-7.25 (m, 0.3H), 7.13 (s, 0.7H), 4.71-4.60 (m, 1H), 4.00 (s, 4H), 3.64-3.61 (m, 3H), 3.10-3.01 (m, 1H), 2.93-2.64 (m, 5H), 2.03-1.93 (m, 2H), 1.52-1.44 (m, 3H).

A mixture of methyl 3-(6′,7′-dihydrospiro[[1,3]dioxolane-2,5′-indazol]-2′(4′H)-yl) butanoate and methyl 3-(6′,7′-dihydrospiro[[1,3]dioxolane-2,5′-indazol]-1′(4′H)-yl)butanoate AA19 (7.90 g, 28.2 mmol) was separated by chiral prep. HPLC (separation condition: Column: Chiralpak IF 5 μm 20*250 mm; Mobile Phase: Hex:EtOH=60:40 at 14 mL/min; Col. Temp: 39.8° C.; Wavelength: 230 nm, Back pressure: 100 bar) to afford AA20 (2.40 g, 30% yield, 99.5% stereopure), AA21 (2.40 g, 30% yield, 98.0% stereopure) and AA22 (1.40 g, 18% yield, 100% stereopure).

AA20: Chiral HPLC (Column: Chiralpak IF 5 μm 4.6*250 mm; Mobile Phase: Hex:EtOH=60:40 at 1.0 mL/min; Temp: 30° C.; Wavelength: 230 nm, R_{T}=7.845 min). ¹H NMR (300 MHz, CDCl₃) δ 7.15 (s, 1H), 4.73-4.66 (m, 1H), 4.02 (s, 4H), 3.66 (s, 3H), 3.03-2.95 (m, 1H), 2.86 (t, J=6.6 Hz, 2H), 2.77-2.67 (m, 3H), 1.98 (t, J=6.6 Hz, 2H), 1.52 (d, J=6.6 Hz, 3H).

AA21: Chiral HPLC (Column: Chiralpak IF 5 μm 4.6*250 mm; Mobile Phase: Hex:EtOH=60:40 at 1.0 mL/min; Temp: 30° C.; Wavelength: 230 nm, R_{T}=9.898 min). ¹H NMR (300 MHz, CDCl₃) δ 7.14 (s, 1H), 4.72-4.65 (m, 1H), 4.02 (s, 4H), 3.65 (s, 3H), 3.02-2.94 (m, 1H), 2.86 (t, J=6.6 Hz, 2H), 2.76-2.66 (m, 3H), 1.97 (t, J=6.6 Hz, 2H), 1.52 (d, J=6.9 Hz, 3H).

### Acid 5 and Acid 6:

### 2-(4-Methoxy-4-oxobutan-2-yl)-4,5,6,7-tetrahydro-2H-indazole-5-carboxylic acid

By ultilizing the analogous procedure of Sequence AAA, AA20 and AA21 were converted to the title compounds Acid 5 and Acid 6 respectively.

Acid 5: LC-MS (ESI): R_{T}=0.642 min, mass calcd. for C₁₃H₁₈N₂O₄ 266.1, m/z found 267.1 [M+H]⁺. ¹H NMR (300 MHz, CDCl₃) δ 8.08 (br s, 1H), 7.18 (d, J=6.3 Hz, 1H), 4.76-4.65 (m, 1H), 3.65 (s, 3H), 3.04-2.95 (m, 1H), 2.87-2.66 (m, 6H), 2.28-2.25 (m, 1H), 1.96-1.89 (m, 1H), 1.56-1.52 (m, 3H).

Acid 6: LC-MS (ESI): R_{T}=0.673 min, mass calcd. for C₁₃H₁₈N₂O₄ 266.1, m/z found 267.1 [M+H]⁺. ¹H NMR (300 MHz, CDCl₃) δ 7.80 (br s, 1H), 7.17 (s, 1H), 4.77-4.65 (m, 1H), 3.64 (s, 3H), 3.18-2.66 (m, 7H), 2.31-2.24 (m, 1H), 1.97-1.83 (m, 1H), 1.53 (d, J=6.9 Hz, 3H).

### Acid 7:

### 2-(3-Methoxy-2,2-dimethyl-3-oxopropyl)-4,5,6,7-tetrahydro-2H-indazole-5-carboxylic acid

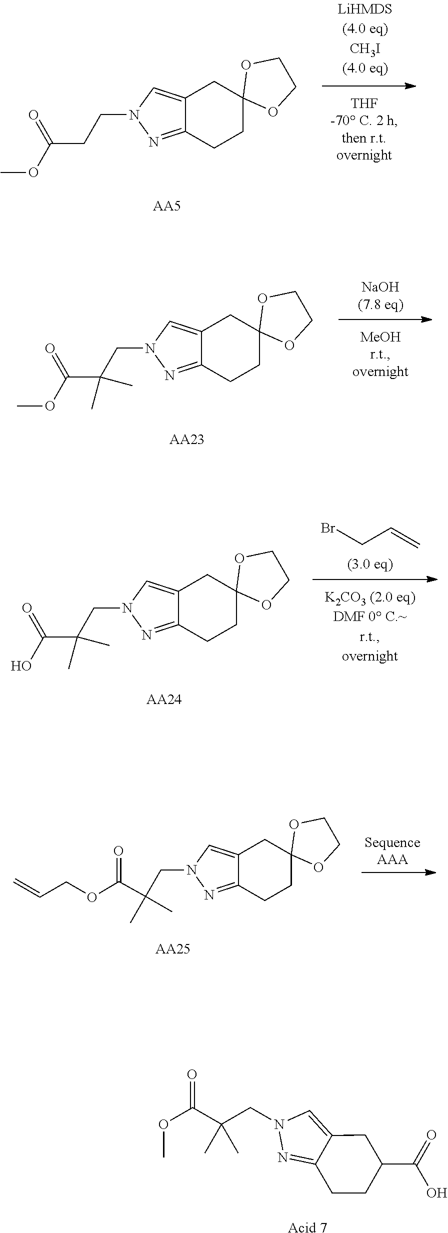

### Intermediate AA23:

### Methyl 3-(6′,7′-dihydrospiro[[1,3]dioxolane-2,5′-indazol]-2′ (4′H)-yl)-2,2-dimethylpropanoate

To a solution of methyl 3-(6′,7′-dihydrospiro[[1,3]dioxolane-2,5′-indazol]-2′(4′H)-yl)propanoate AA5 (2.0 g, 7.12 mmol) in dry tetrahydrofuran (50 mL) was added dropwise 1.0 M lithium hexamethyldisilazide in tetrahydrofuran (28.4 mL, 28.4 mmol) at −70° C. After stirred at −70° C.; for 2 hours, iodomethane (4.0 g, 28.4 mmol) was added dropwise at −70° C. The obtained reaction mixture was allowed to warm to room temperature and stirring continued overnight. Then it was basified with saturated ammonium chloride aqueous solution to pH 7-8 and extracted with ethyl acetate (50 mL) twice. The combined organic layers were washed with water (50 mL) twice, brine (30 mL) twice, dried over Na₂SO₄₍ₛ₎, filtered and concentrated under reduced pressure to give the title compound (1.4 g, 67% yield) as a yellow oil. LC-MS (ESI): R_{T}=2.461 min, mass calcd. for C₁₅H₂₂N₂O₄ 294.2, m/z found 295.1 [M+H]⁺. ¹H NMR (400 MHz, CDCl₃) δ 7.06 (s, 1H), 4.18 (s, 2H), 4.01 (s, 4H), 3.70 (s, 3H), 2.84 (t, J=6.8 Hz, 2H), 2.75 (s, 2H), 1.96 (t, J=6.4 Hz, 2H), 1.21 (s, 6H).

### Intermediate AA24

### 3-(6′,7′-Dihydrospiro[[1,3]dioxolane-2,5′-indazol]-2′ (4′H)-yl)-2,2-dimethylpropanoic acid

To a solution of methyl 3-(6′,7′-dihydrospiro[[1,3]dioxolane-2,5′-indazol]-2′(4′H)-yl)-2,2-dimethylpropanoate AA23 (1.4 g, 4.76 mmol) in methanol (15 mL) was added a solution of sodium hydroxide in water (5 mL, 30% w/w, 37.5 mmol) at room temperature. After stirred at room temperature overnight, the reaction mixture was acidified to pH=5-6 with 1 M hydrochloride aqueous solution and extracted with ethyl acetate (30 mL) twice. The combined organic layers were washed with water (20 mL) and brine (20 mL) twice, dried over Na₂SO₄₍ₛ₎ and filtered. The filtrate was concentrated under reduced pressure to give the title compound (1.0 g, 77% yield) as yellow solids. ¹H NMR (400 MHz, CDCl₃) δ 7.13 (s, 1H), 4.22 (s, 2H), 4.01 (s, 4H), 2.86 (t, J=6.4 Hz, 2H), 2.76 (s, 2H), 1.96 (t, J=6.8 Hz, 2H), 1.21 (s, 6H).

### Intermediate AA25:

### Allyl 3-(6′,7′-dihydrospiro[[1,3]dioxolane-2,5′-indazol]-2′ (4′H)-yl)-2,2-dimethylpropanoate

To a solution of 3-(6′,7′-dihydrospiro[[1,3]dioxolane-2,5′-indazol]2′(4′H)-yl)-2,2-dimethylpropanoic acid AA24 (3.9 g, 13.9 mmol) and potassium carbonate (3.8 g, 27.8 mmol) in dry N,N-dimethylformamide (50 mL) was added dropwise allyl bromide (5.0 g, 41.6 mmol) at 0° C.; under nitrogen atmosphere. After stirred at 0° C.; for 30 minutes and then at room temperature overnight, the mixture was quenched with brine (10 mL) and extracted with ethyl acetate (10 mL) twice. The combined organic layers were washed with water (10 mL), brine (10 mL) twice, dried over Na₂SO₄₍ₛ₎, filtered and concentrated under reduced pressure to give a residue, which was purified by silica gel column chromatography (petroleum ether:ethyl acetate=4:1 to 2:1) to give the title compound (3.2 g, 73% yield) as colorless oil. ¹H NMR (400 MHz, CDCl₃) δ 7.07 (s, 1H), 5.96-5.86 (m, 1H), 5.32-5.22 (m, 2H), 4.60 (d, J=5.6 Hz, 2H), 4.19 (s, 2H), 4.01 (s, 4H), 2.84 (t, J=6.4 Hz, 2H), 2.75 (s, 2H), 1.96 (t, J=6.8 Hz, 2H), 1.23 (s, 6H).

### Acid 7:

### 2-(3-Methoxy-2,2-dimethyl-3-oxopropyl)-4,5,6,7-tetrahydro-2H-indazole-5-carboxylic acid

By ultilizing the analogous procedure of Sequence AAA, AA25 was converted to the title compound.

LC-MS (ESI): R_{T}=1.489 min, mass calcd. for C₁₄H₂₀N₂O₄ 280.1, m/z found 281.0 [M+H]⁺.

### Acid 8:

### 2-((trans)-3-(Methoxycarbonyl)cyclobutyl)-4,5,6,7-tetrahydro-2H-indazole-5-carboxylic acid

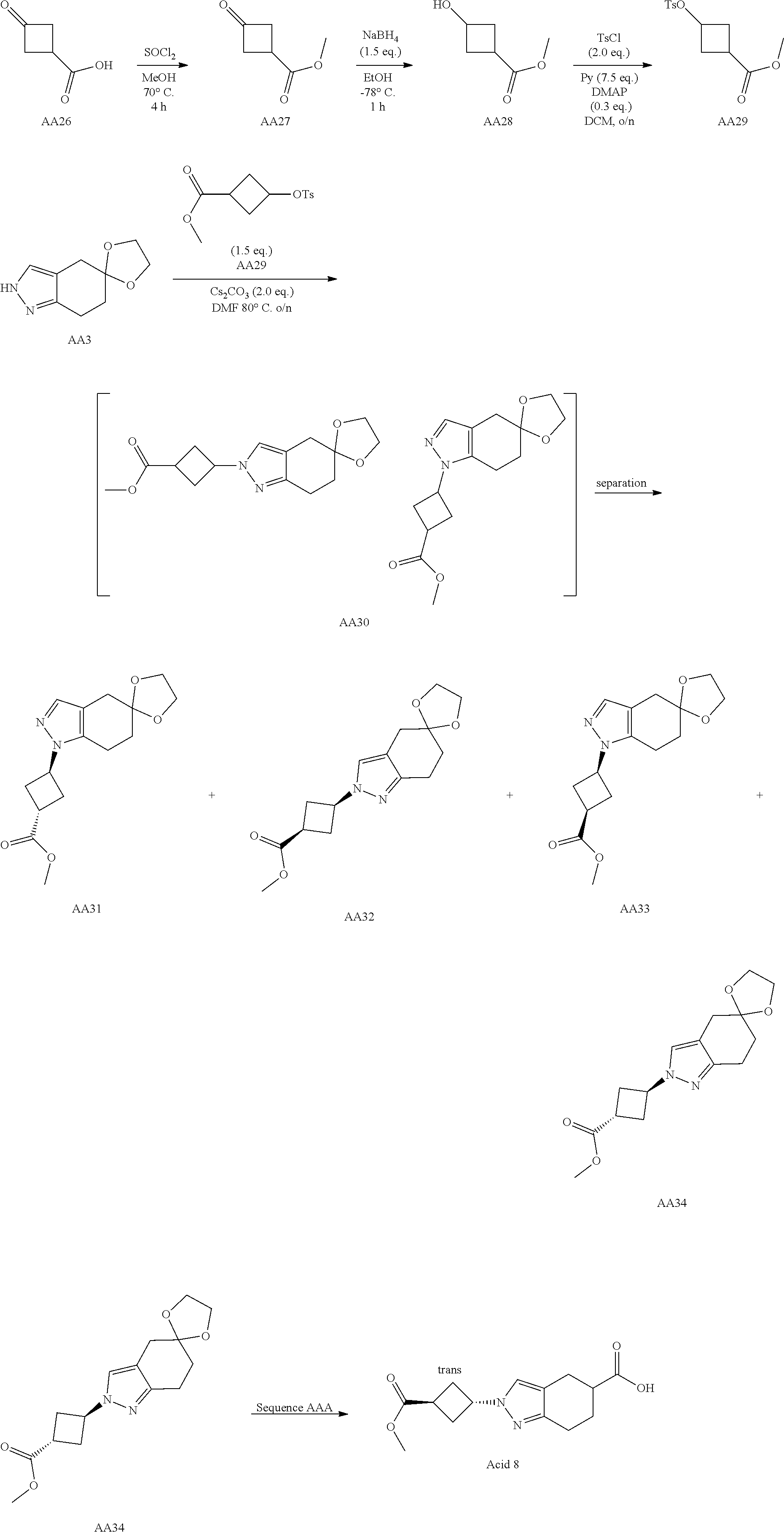

### Intermediate AA27

### Methyl 3-oxocyclobutanecarboxylate

To a solution of 3-oxocyclobutanecarboxylic acid AA26 (20.0 g, 175 mmol), in methanol (200 mL) was added thionyl chloride (25.0 g, 210 mmol) at 0° C. After stirred at 70° C.; for 4 hours, the mixture was allowed to cool down to room temperature and concentrated under reduced pressure to give a residue, which was purified by silica gel column chromatography (petroleum ether:ethyl acetate=10:1) to afford the title compound (19.5 g, 87% yield) as colorless oil. ¹H NMR (300 MHz, CDCl₃) δ 3.82-3.60 (m, 3H), 3.48-3.09 (m, 5H).

### Intermediate AA28

### Methyl 3-hydroxycyclobutanecarboxylate

To a solution of methyl 3-oxocyclobutanecarboxylate AA27 (21.6 g, 169 mmol) in ethanol (220 mL) was sodium borohydride (9.6 g, 253 mmol) at −78° C.; under nitrogen atmosphere. After stirred at −78° C.; for 1 hour, the mixture was quenched with saturated ammonium chloride aqueous solution (200 mL) at −78° C. The resulting mixture was then allowed to warm to room temperature, concentrated under reduced pressure to remove the volatiles and extracted with ethyl acetate (500 mL) for three times. The combined organic layers were washed with brine (300 mL), dried over Na₂SO₄₍ₛ₎, filtered and the filtrate was concentrated at room temperature under reduced pressure to afford the title compound (18.3 g, 85% yield) as yellow oil. ¹H NMR (300 MHz, CDCl₃) δ 4.21-4.08 (m, 1H), 3.69-3.63 (m, 3H), 2.74-2.49 (m, 3.7H), 2.25-2.04 (m, 2.3H).

### Intermediate AA29

### Methyl 3-(tosyloxy)cyclobutanecarboxylate

To a solution of methyl 3-hydroxycyclobutanecarboxylate AA28 (19.7 g, 152 mmol) in dichloromethane (200 mL) was added pyridine (90.0 g, 1.14 mol), 4-dimethylaminopyridine (5.60 g, 45.6 mmol) and tosyl chloride (58.0 g, 303 mmol) at room temperature. After stirred at room temperature overnight, the mixture was poured water (200 mL) and extracted with ethyl ester (200 mL) twice. The combined organic layers were washed with 0.5 M hydrochloride aqueous solution (200 mL), saturated sodium bicarbonate aqueous solution (200 mL), water (200 mL) and brine (200 mL), dried over Na₂SO₄₍ₛ₎, filtered and the filtrate was concentrated under reduced pressure at room temperature to afford the title compound (42.7 g, 99% yield) as yellow oil. ¹H NMR (300 MHz, CDCl₃) δ 7.77-7.73 (m, 2H), 7.34-7.26 (m, 2H), 4.77-4.64 (m, 1H), 3.66-3.59 (m, 3H), 2.67-2.54 (m, 1H), 2.50-2.37 (m, 7H).

### Intermediate AA30: (Mixture of two regioisomers, each contains trans/cis) Mixture of methyl 3-(6′,7′-dihydrospiro[[1,3]dioxolane-2,5′-indazol]-2′(4′H)-yl) cyclobutanecarboxylate and methyl 3-(6′,7′-dihydrospiro[[1,3]dioxolane-2,5′-indazol]-1′ (4′H)-yl)cyclobutanecarboxylate

To a solution of 2′,4′,6′,7′-tetrahydrospiro[[1,3]dioxolane-2,5′-indazole] AA3 (10.0 g, 55.6 mmol) in N,N-dimethylformamide (100 mL) was added cesium carbonate (35.9 g, 110 mmol) and methyl 3-(tosyloxy)cyclobutanecarboxylate AA29 (23.7 g, 83.4 mmol) at room temperature. After stirred at 80° C.; overnight, the mixture was cooled down to room temperature and filtered. The filtrate was concentrated under reduced pressure to give a residue, which was purified by C 18 (acetonitrile:water=30% to 50%) to afford the title compound (8.0 g, 50% yield) as yellow oil. ¹H NMR (300 MHz, CDCl₃) δ 7.35-7.29 (m, 0.5H), 7.23 (m, 0.3H), 7.11 (m, 0.2H), 4.95-4.83 (m, 0.3H), 4.64-4.44 (m, 0.7H), 4.01-3.98 (m, 4H), 3.74-3.69 (m, 3H), 3.19-3.10 (m, 0.5H), 3.01-2.84 (m, 3H), 2.76-2.63 (m, 4.5H), 2.01-1.91 (m, 3H).

A mixture of AA30 (10.0 g, 3.42 mmol) was separated by SFC (the first separation condition: Column: Chiralpak IE 5 μm 20*250; Mobile Phase: CO₂:MeOH=70:30 at 50 g/min; Co-solvent: MeOH; Wavelength: 214 nm; back pressure: 100 bar; the second separation condition: Column: Chiralpak IF 5 μm 20*250; Mobile Phase: CO₂: MeOH=75:25 at 50 g/min; Co-solvent: MeOH; Wavelength: 214 nm; back pressure: 100 bar) to afford the title compounds AA31 (1.10 g, 11% yield), AA32 (1.10 g, 11% yield), AA33 (1.10 g, 11% yield) and AA34 (1.40 g, 14% yield) as yellow oil.

AA34: Chiral HPLC (Column: Chiralpak IE 5 μm 4.6*250 mm; Mobile Phase: CO2:MeOH=70:30 at 3.0 mL/min; Col. Temp: 40° C.; Wavelength: 230 nm, Back pressure: 100 bar, R_{T}=5.12 min). ¹H NMR (300 MHz, CDCl₃) δ 7.11-7.09 (m, 1H), 4.96-4.85 (m, 1H), 4.02-3.97 (m, 4H), 3.73-3.66 (m, 3H), 3.18-3.05 (m, 1H), 2.91-2.84 (m, 3H), 2.78-2.63 (m, 4H), 2.00-1.90 (m, 3H).

### Acid 8: 2-((trans)-3-(Methoxycarbonyl)cyclobutyl)-4,5,6,7-tetrahydro-2H-indazole-5-carboxylic acid

By ultilizing the analogous procedure of Sequence AAA, AA34 was converted to the title compound.

LC-MS (ESI): R_{T}=0.25 min, mass calcd. for C₁₄H₁₈N₂O₄ 278.1, m/z found 279.2 [M+H]⁺. ¹H NMR (400 MHz, DMSO-d₆) δ 12.08 (br s, 1H), 7.49 (s, 1H), 4.91-4.83 (m, 1H), 3.66 (s, 3H), 3.17-3.10 (m, 1H), 2.77-2.64 (m, 4H), 2.69-2.62 (m, 1H), 2.59-2.52 (m, 4H), 2.11-2.07 (m, 1H), 1.78-1.67 (m, 1H).

### Acid 9: 2-(3-Methoxy-3-oxopropyl)-7,7-dimethyl-4,5,6,7-tetrahydro-2H-indazole-5-carboxylic acid

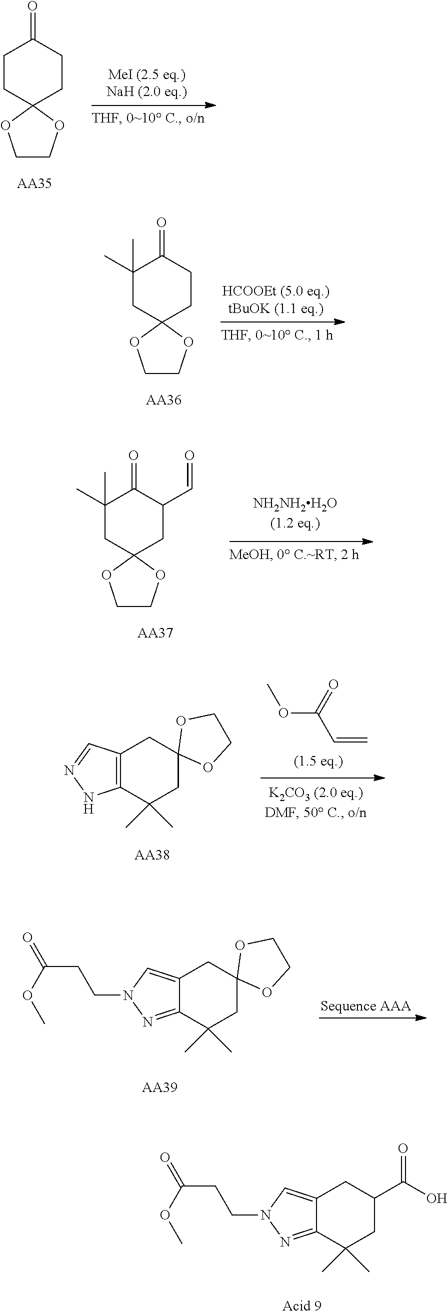

### Intermediate AA36

### 7,7-dimethyl-1,4-dioxaspiro[4.5]decan-8-one

To an ice-cold solution of 1,4-dioxaspiro[4.5]decan-8-one AA35 (10.0 g, 64 mmol) in dry tetrahydrofuran (100 mL) was added slowly 60% wt sodium hydride in mineral oil (5.10 g, 128 mmol). After stirring at room temperature for 1 hour, iodomethane (22.8 g, 160 mmol) was added over 10 minutes under nitrogen atmosphere and stirring continued at room temperature overnight. The mixture was quenched with saturated ammonium chloride aqueous solution (100 mL), concentrated under reduced pressure to remove volatiles and then taken up into ethyl acetate (400 mL). The separated aqueous layer was extracted with ethyl acetate (100 mL) twice. The combined organic layers were washed with brine (100 mL), dried over Na₂SO₄₍ₛ₎ and filtered. The filtrate was concentrated and purified by silica gel column chromatography (petroleum ether:ethyl acetate=20:1 to 10:1) to give the title compound (6.60 g, 56% yield) as colorless oil. ¹H NMR (300 MHz, CDCl₃) δ 3.99-3.96 (m, 4H), 2.60-2.52 (m, 2H), 1.98-1.85 (m, 4H), 1.20-1.13 (m, 6H).

### Intermediate AA37

### 9,9-dimethyl-8-oxo-1,4-dioxaspiro[4.5]decane-7-carbaldehyde

To an ice-cold solution of potassium tert-butylate (2.70 g, 23.9 mmol) in dry tetrahydrofuran (60 mL) was added slowly ethyl formate (5.0 mL, 65.0 mmol). After stirring at 0° C.; for 30 min, a solution of 7,7-dimethyl-1,4-dioxaspiro[4.5]decan-8-one AA36 (4.00 g, 21.7 mmol) and ethyl formate (3.7 mL, 44.0 mmol) in tetrahydrofuran (40 mL) was added over 20 minutes under nitrogen atmosphere. The resulting mixture was stirred at room temperature for 1 hour. It was then quenched with 10% wt citric acid aqueous solution (20 mL), concentrated under reduced pressure to remove volatiles, and partitioned between ethyl acetate (60 mL) and water (20 mL). The separated aqueous layer was extracted with ethyl acetate (30 mL) twice. The combined organic layers were washed with brine (20 mL), dried over Na₂SO₄₍ₛ₎ and filtered. The filtrate was concentrated under reduced pressure to give the title compound (4.10 g, 89% yield) as yellow oil. ¹H NMR (300 MHz, CDCl₃) δ 14.77 (s, 1H), 8.55 (s, 1H), 3.99 (s, 4H), 2.57 (s, 2H), 1.78 (s, 2H), 1.28 (s, 6H).

### Intermediate AA38

### 7′,7′-dimethyl-1′,4′,6′,7′-tetrahydrospiro[[1,3]dioxolane-2,5′-indazole]

To a stirring solution of 9,9-dimethyl-8-oxo-1,4-dioxaspiro[4.5]decane-7-carbaldehyde AA37 (4.10 g, 19.0 mmol) in methanol (90 mL) was added hydrazine hydrate (1.40 g, 23.0 mmol) at 0° C. After stirred at room temperature for 2 hours under nitrogen atmosphere, the mixture was concentrated under reduced pressure to leave a residue. It was then partitioned between ethyl acetate (50 mL) and water (20 mL). The aqueous layer was extracted with ethyl acetate (50 mL) for three times. The combined organic layers were washed with brine (20 mL), dried over Na₂SO₄₍ₛ₎ and filtered. The filtrate was concentrated under reduced pressure to give the title compound (3.50 g, 88% yield) as yellow oil. LC-MS (ESI): R_{T}=1.34 min, mass calcd. for C₁₁H₁₆N₂O₂ 208.1, m/z found 209.3 [M+H]⁺. ¹H NMR (300 MHz, CDCl₃) δ 7.27 (s, 1H), 3.98 (s, 4H), 2.77 (s, 2H), 1.87 (s, 2H), 1.38 (s, 6H).

### Intermediates AA39

### Methyl 3-(7′,7′-dimethyl-6′,7′-dihydrospiro[[1,3]dioxolane-2,5′-indazol]-2′ (4′H)-yl)propanoate

To a solution of 7′,7′-dimethyl-1′,4′,6′,7′-tetrahydrospiro[[1,3]dioxolane-2,5′-indazole] AA38 (3.30 g, 15.8 mmol) in N,N-dimethylformide (33 mL) was added acrylic acid methyl ester (2.10 g, 23.8 mmol) and potassium carbonate (4.40 g, 31.6 mmol) at room temperature. After stirred at 50° C.; under nitrogen atmosphere overnight, the reaction mixture was cooled down to room temperature. It was concentrated under reduced pressure to remove volatiles, poured into water (30 mL) and extracted with ethyl acetate (100 mL) for three times. The combined organic layers were washed with water (40 mL) followed by brine (40 mL), dried over Na₂SO₄₍ₛ₎ and filtered. The filtrate was concentrated under reduced pressure to give a crude product, which was purified by C18 column (acetonitrile:water=5% to 60%) to give the title compound (2.90 g, 62% yield) as light yellow oil. LC-MS (ESI): R_{T}=1.45 min, mass calcd. for C₁₅H₂₂N₂O₄ 294.2, m/z found 295.4 [M+H]⁺. ¹H NMR (400 MHz, DMSO-d₆) δ 7.29 (s, 1H), 4.23 (t, J=6.8 Hz, 2H), 3.89 (s, 4H), 3.60 (s, 3H), 2.79 (t, J=6.8 Hz, 2H), 2.63 (s, 2H), 1.75 (s, 2H), 1.24 (s, 6H).

### Acid 9: 2-(3-Methoxy-3-oxopropyl)-7,7-dimethyl-4,5,6,7-tetrahydro-2H-indazole-5-carboxylic acid

By ultilizing the analogous procedure of Sequence AAA, AA39 was converted to the title compound.

LC-MS (ESI): R_{T}=0.28 min, mass calcd. for C₁₄H₂₀N₂O₄ 280.1, m/z found 281.3 [M+H]⁺. ¹H NMR (400 MHz, DMSO-d₆) δ 7.12 (s, 1H), 4.37 (t, J=6.6 Hz, 2H), 3.69 (s, 3H), 2.98-2.78 (m, 4H), 2.67-2.58 (m, 1H), 1.82-1.73 (m, 1H), 1.63-1.61 (m, 0.4H), 1.47-1.43 (m, 0.6H), 1.37 (s, 3H), 1.27 (s, 3H).

### Acid 10: 2-(3-Methoxy-3-oxopropyl)-3-methyl-4,5,6,7-tetrahydro-2H-indazole-5-carboxylicacid

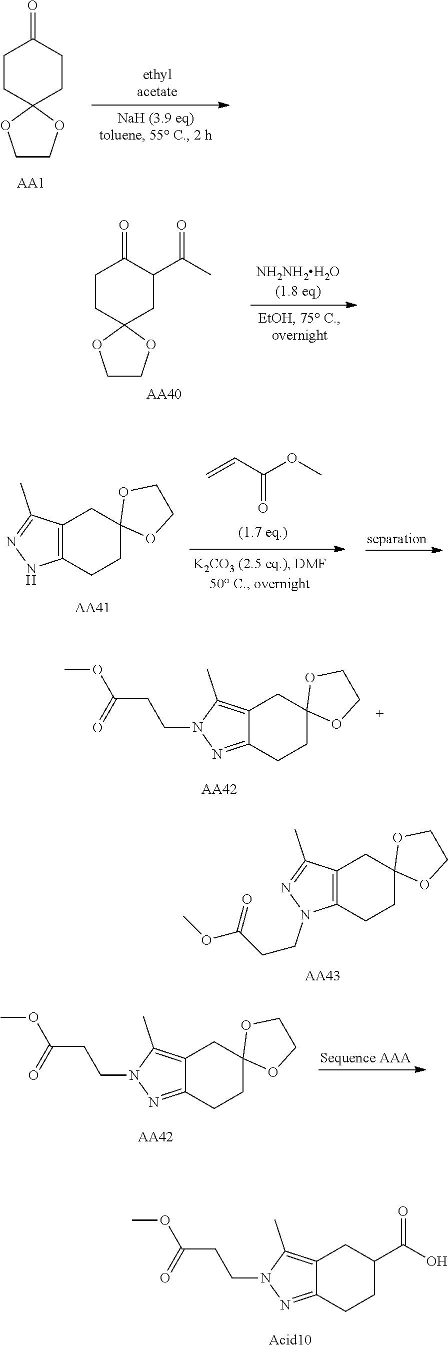

### Intermediate AA40

### 7-Acetyl-1,4-dioxaspiro[4.5]decan-8-one

To a solution of ethyl acetate (30 ml) in toluene (225 mL) was slowly added sodium hydride (15.0 g, 60% wt. in mineral oil, 375 mmol) at room temperature. After stirring at room temperature for 10 minutes, a solution of 1,4-dioxaspiro[4.5]decan-8-one AA1 (15.0 g, 96.2 mmol) in ethyl acetate (30 mL) was added slowly. The resulting mixture was stirred at 55° C.; under nitrogen atmosphere for 2 hours. Then it was poured into ice water (400 mL), acidified with saturated citric acid aqueous solution to pH 3 and then extracted with ethyl acetate (300 mL) for three times. The combined organic layers were washed with brine (100 mL) twice, dried over Na₂SO₄₍ₛ₎, filtered and concentrated under reduced pressure to give a residue. The same procedure cycle was repeated 4 times to extract the organic materials from the citric acid treated aqueous layer. The combined residues were purified by silica gel column chromatography (petroleum ether:ethyl acetate=8:1) to give the title compound (32.0 g, 42% yield) as yellow oil. ¹H NMR (400 MHz, CDCl₃) δ 4.04-4.00 (m, 4H), 3.99-3.97 (m, 1H), 2.58-2.54 (m, 4H), 2.11 (s, 3H), 1.86 (t, J=7.2 Hz, 2H).

### Intermediate AA41

### 3′-Methyl-1′,4′,6′,7′-tetrahydrospiro[[1,3]dioxolane-2,5′-indazole]

To a stirring solution of 7-acetyl-1,4-dioxaspiro[4.5]decan-8-one AA40 (32.0 g, 162 mmol) in ethanol (300 mL) was added hydrazine hydrate (16.9 g, 288 mmol) at room temperature. After stirred at 75° C.; under nitrogen atmosphere overnight, the mixture was concentrated under reduced pressure to leave a residue, which was partitioned between ethyl acetate (200 mL) and water (50 mL). The aqueous layer was separated and extracted with ethyl acetate (50 mL) for three times. The combined organic layers were washed with brine (50 mL), dried over Na₂SO₄₍ₛ₎ and filtered. The filtrate was concentrated under reduced pressure to give the title compound (25.9 g, 82% yield) as yellow oil. LC-MS (ESI): R_{T}=0.408 min, mass calcd. for C₁₀H₁₄N₂O₂ 194.1, m/z found 195.1 [M+H]⁺. ¹H NMR (400 MHz, CDCl₃) δ 4.09-4.01 (m, 4H), 2.83 (t, J=6.4 Hz, 2H), 2.66 (s, 2H), 2.16 (s, 3H), 1.96 (t, J=6.4 Hz, 2H).

### Intermediate AA42

### Methyl 3-(3′-methyl-6′,7′-dihydrospiro[[1,3]dioxolane-2,5′-indazol]-2′ (4′H) yl)propanoate

To a solution of 3′-methyl-1′,4′,6′,7′-tetrahydrospiro[[1,3]dioxolane-2,5′-indazole] AA41 (25.9 g, 134 mmol) in N,N-dimethylformamide (400 mL) was added acrylic acid methyl ester (20.0 g, 232 mmol) and potassium carbonate (45.6 g, 330 mmol) at room temperature. After stirred at 50° C.; under nitrogen atmosphere overnight, the mixture was cooled down to room temperature and concentrated under reduced pressure to remove volatiles. The residue was poured into water (100 mL) and extracted with ethyl acetate (100 mL) for three times. The combined organic layers were washed with water (100 mL), brine (100 mL), dried over Na₂SO₄₍ₛ₎ and filtered. The filtrate was concentrated under reduced pressure to give a crude product, which was purified by C18 column (acetonitrile:water=% to 80%) followed by chiral prep. HPLC (Column: Chiralpak IE 5 μm 20*250 mm; Mobile Phase: IPA:CO₂=70:30 at 50 g/min; Co-solvent: IPA; Col. Temp: 40° C.; Wavelength: 230 nm, Back pressure: 100 bar) to afford the title compounds AA42 (8.73 g, 46% yield) and AA43 (8.81 g, 46% yield) as colorless oil.

AA42: Chiral HPLC (Column: Chiralpak IE 5 μm 4.6*250 mm; Mobile Phase: IPA:CO₂=70:30 at 3 g/min; Col. Temp: 40° C.; Wavelength: 230 nm, Back pressure: 100 bar; R_{T}=3.68 min). ¹H NMR (400 MHz, CDCl₃) δ 4.23 (t, J=7.2 Hz, 2H), 4.03 (s, 4H), 3.68 (s, 3H), 2.87 (t, J=7.2 Hz, 2H), 2.81 (t, J=6.8 Hz, 2H), 2.65 (s, 2H), 2.16 (s, 3H), 1.95 (t, J=6.4 Hz, 2H).

### Acid 10: 2-(3-Methoxy-3-oxopropyl)-3-methyl-4,5,6,7-tetrahydro-2H-indazole-5-carboxylicacid

By ultilizing the analogous procedure of Sequence AAA, AA42 was converted to the title compound.

LC-MS (ESI): R_{T}=0.299 min, mass calcd. for C₁₃H₁₈N₂O₄ 266.1, m/z found 267.1 [M+H]⁺. ¹H NMR (400 MHz, DMSO-d₆) δ 12.23 (s, 1H), 4.12 (t, J=6.4 Hz, 2H), 3.59 (s, 3H), 2.80 (t, J=6.8 Hz, 2H), 2.64-2.52 (m, 3H), 2.48-2.37 (m, 2H), 2.13 (s, 3H), 2.10-2.05 (m, 1H), 1.71-1.61 (m, 1H).

### Acid 11: 2-(3-Methoxy-3-oxopropyl)-4,5,6,7-tetrahydrobenzo[d]oxazole-5-carboxylic acid

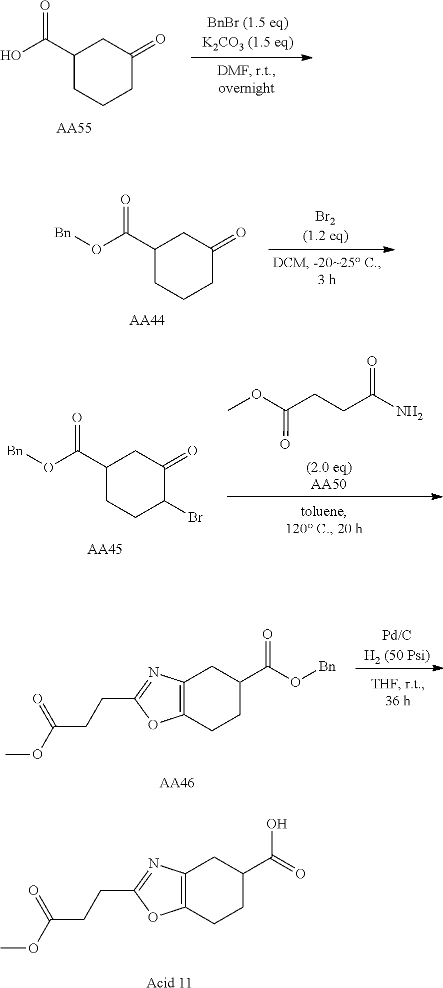

### Intermediate AA44

### Benzyl 3-oxocyclohexane-1-carboxylate

To a solution of 3-oxocyclohexanecarboxylic acid AA55 (13.2 g, 93.0 mmol) in N, N-dimethylformamide (100 mL) was added potassium carbonate (19.2 g, 139 mmol) and benzyl bromide (23.8 g, 139 mmol) at room temperature. After stirred at room temperature overnight, the mixture was poured into water (200 mL) and extracted with ethyl acetate (100 mL) for three times. The combined organic phases were washed with water (100 mL) twice followed by brine (100 mL), dried over Na₂SO₄₍ₛ₎, filtered and concentrated to give a residue, which was purified by silica gel column chromatography (petroleum ether:ethyl acetate=15:1 to 8:1) to give the title compound (14.3 g, 46% yield) as colorless oil. ¹H NMR (300 MHz, CDCl₃) δ 7.42-7.29 (m, 5H), 5.15 (s, 2H), 2.91-2.81 (m, 1H), 2.57 (d, J=8.1 Hz, 2H), 2.43-2.26 (m, 2H), 2.19-2.00 (m, 2H), 1.92-1.80 (m, 1H), 1.75-1.69 (m, 1H).

### Intermediate AA45

### Benzyl 4-bromo-3-oxocyclohexane-1-carboxylate

To a solution of tert-butyl 3-oxocyclohexanecarboxylate AA44 (5.0 g, 21.5 mmol) in dichloromethane (40 mL) was added bromine (4.1 g, 25.3 mmol) in dichloromethane (20 mL) dropwise at −20° C. The mixture was stirred in a range of temperature from −20° C.; to 20° C.; for 3 hours. After the reaction completed, the mixture was concentrated and the obtained residue was added water (50 mL). It was extracted with ethyl acetate (50 mL) for three times. The combined organic phases were washed with water (50 mL) twice followed by brine (50 mL), dried over Na₂SO₄₍ₛ₎, filtered and concentrated to give the crude title compound (5.7 g, 86% yield) as yellow oil, which was used for the next step without further purification. ¹H NMR (300 MHz, CDCl₃) δ 7.41-7.33 (m, 5H), 5.25-5.12 (m, 2.2H), 4.94-4.87 (m, 0.2H), 4.79-4.64 (m, 0.6H), 3.36-3.23 (m, 0.5H), 3.21-3.09 (m, 0.5H), 3.06-2.88 (m, 1H), 2.83-2.54 (m, 1H), 2.49-1.93 (m, 4H).

### Intermediate AA46

### Benzyl 2-(3-methoxy-3-oxopropyl)-4,5,6,7-tetrahydrobenzo[d]oxazole-5-carboxylate

To a solution of benzyl 4-bromo-3-oxocyclohexane-1-carboxylate AA46 (5.7 g, 18.3 mmol) in toluene (50 mL) was added methyl 4-amino-4-oxobutanoate AA50 (4.85 g, 36.6 mmol). After stirred at 120° C.; for 20 hours, the mixture was concentrated to give a residue, which was purified by silica gel column chromatography (petroleum ether:ethyl acetate=15:1 to 5:1) followed by C18 column (acetonitrile:water=5% to 75%) to give the title compound (3.0 g, 29% yield) as colorless oil. LC-MS (ESI): R_{T}=1.60 min, mass calcd. for C₁₉H₂₁NO₅ 343.1, m/z found 344.3 [M+H]⁺. ¹H NMR (300 MHz, CDCl₃) δ 7.42-7.32 (m, 5H), 5.15 (s, 2H), 3.70 (s, 3H), 3.04 (t, J=7.2 Hz, 2H), 2.85-2.47 (m, 7H), 2.27-2.17 (m, 1H), 2.06-1.94 (m, 1H).

### Acid 11: 2-(3-Methoxy-3-oxopropyl)-4,5,6,7-tetrahydrobenzo[d]oxazole-5-carboxylic acid

To the solution of benzyl 2-(3-methoxy-3-oxopropyl)-4,5,6,7-tetrahydrobenzo [d]oxazole-5-carboxylate AA46 (3.0 g, 8.75 mmol) in tetrahydrofuran (100 mL) was added 5% wt. palladium on activated carbon (837 mg) under nitrogen atmosphere. After stirred at room temperature under hydrogen atmosphere (50 psi) for 36 hours, the mixture was filtered through a pad of celite. The filtrate was concentrated under reduced pressure to give the title compound (2.0 g, 90% yield) as yellow oil. LC-MS (ESI): R_{T}=0.29 min, mass calcd. for C₁₂H₁₅NO₅ 253.1, m/z found 254.2 [M+H]⁺. ¹H NMR (400 MHz, DMSO-d₆) δ 12.46 (s, 1H), 3.60 (s, 3H), 2.92 (t, J=7.2 Hz, 2H), 2.76-2.64 (m, 2.5H), 2.64-2.56 (m, 3.5H), 2.44-2.38 (m, 1H), 2.14-2.08 (m, 1H), 1.87-1.81 (m, 1H).

### Acid 12: 2-(3-Methoxy-3-oxopropyl)-4,5,6,7-tetrahydrobenzo[d]oxazole-6-carboxylic acid

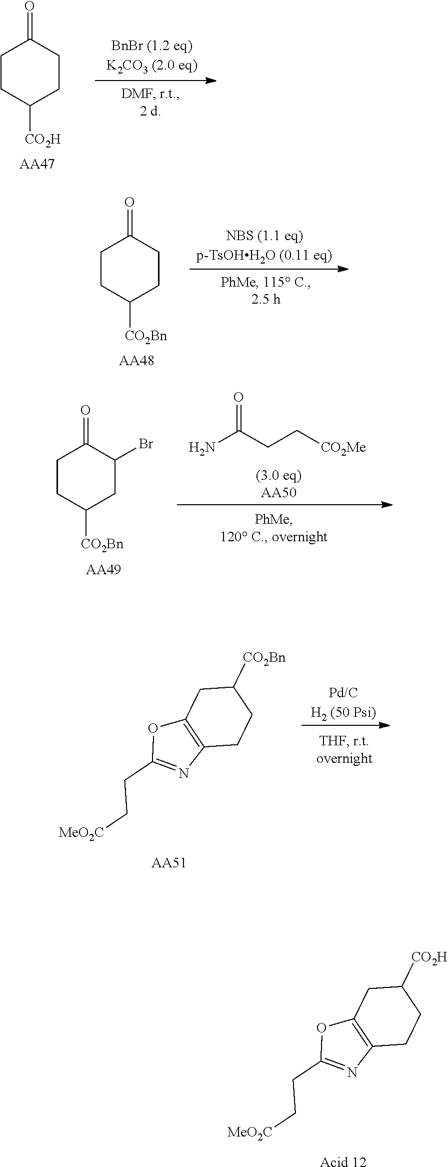

### Intermediate AA48

### Benzyl 4-oxocyclohexanecarboxylate

To a solution of 4-oxocyclohexanecarboxylic acid AA47 (20.0 g, 0.141 mol), potassium carbonate (38.9 g, 0.282 mol) in N,N-dimethylformamide (100 mL) was added (bromomethyl)benzene (28.8 g, 0.169 mol). The mixture was stirred at room temperature for 2 days. The reaction mixture was poured into water (450 mL) and extracted with ethyl acetate (200 mL) for three times. The combined organic layers were washed with water (100 mL), brine (100 mL), dried over Na₂SO₄₍ₛ₎, filtered and concentrated to give the crude product, which was purified by silica gel column chromatography (petroleum ether:ethyl acetate=10:1 to 5:1) to give the title compound (30.0 g, 92% yield) as yellow oil. ¹H NMR (300 MHz, DMSO-d₆) δ 7.37-7.26 (m, 5H), 5.12-5.05 (m, 2H), 2.91-2.79 (m, 1H), 2.41-2.31 (m, 2H), 2.23-2.08 (m, 4H), 1.87-1.72 (m, 2H).

### Intermediate AA49

### Benzyl 3-bromo-4-oxocyclohexanecarboxylate

To a solution of benzyl 4-oxocyclohexanecarboxylate AA48 (10.0 g, 43.1 mmol) in toluene (200 mL) were added 1-bromopyrrolidine-2,5-dione (8.4 g, 47.4 mmol) and 4-methylbenzenesulfonic acid monohydrate (920 mg, 4.74 mmol). The reaction mixture was stirred at 115° C.; for 2.5 hours. The solvent was removed to give a residue, which was purified by silica gel column chromatography (petroleum ether:ethyl acetate=10:1 to 9:1) to give the title compound (8.5 g, 63% yield) as yellow oil. ¹H NMR (400 MHz, DMSO-d₆) δ 7.40-7.35 (m, 5H), 5.16-5.12 (m, 2H), 3.14-3.07 (m, 1H), 2.93-2.85 (m, 0.5H), 2.79-2.73 (m, 0.5H), 2.69-2.52 (m, 2H), 2.41-2.31 (m, 1H), 2.29-2.14 (m, 2H), 2.02-1.96 (m, 0.5H), 1.88-1.78 (m, 0.5H).

### Intermediate AA51

### Benzyl 2-(3-methoxy-3-oxopropyl)-4,5,6,7-tetrahydrobenzo[d]oxazole-6-carboxylate

To a solution of benzyl 3-bromo-4-oxocyclohexanecarboxylate AA49 (8.5 g, 27.3 mmol) in toluene (100 mL) was added methyl 4-amino-4-oxobutanoate AA50 (10.7 g, 81.9 mmol). After stirred at 120° C.; overnight, the reaction mixture was cooled down to room temperature and concentrated. The obtained residue was diluted with ethyl acetate (100 mL), adjusted with saturated sodium bicarbonate aqueous solution to pH=7-8, washed with water (100 mL), brine (50 mL), dried over Na₂SO₄₍ₛ₎, filtered and concentrated to give a residue, which was purified by silica gel column chromatography (petroleum ether:ethyl acetate=10:1 to 4:1) to give the title compound (3.7 g, 39% yield) as yellow oil. ¹H NMR (300 MHz, DMSO-d₆) δ 7.37-7.31 (m, 5H), 5.12-5.11 (m, 2H), 3.58 (s, 3H), 2.93-2.88 (m, 2H), 2.81-2.78 (m, 2H), 2.74-2.69 (m, 2H), 2.64-2.55 (m, 1H), 2.44-2.37 (m, 2H), 2.09-2.01 (m, 1H), 1.88-1.77 (m, 1H).

### Acid 12: 2-(3-Methoxy-3-oxopropyl)-4,5,6,7-tetrahydrobenzo[d]oxazole-6-carboxylic acid

To a solution of benzyl 2-(3-methoxy-3-oxopropyl)-4,5,6,7-tetrahydrobenzo[d]oxazole-6-carboxylate AA51 (3.7 g, 10.8 mmol) in tetrahydrofuran (150 mL) was added 10% palladium on charcoal (wetted with ca. 55% water). The reaction mixture was stirred at room temperature under hydrogen atmosphere (50 psi) overnight. The reaction mixture was filtered through the celite and concentrated to give a residue, which was purified by silica gel column chromatography (100% petroleum, then 100% dichloromethane, then dichloromethane:methanol=20:1) to give the title compound (3.4 g, 90% yield) as yellow solids. ¹H NMR (400 MHz, DMSO-d₆) δ 12.45 (s, 1H), 3.60 (s, 3H), 2.93 (t, J=7.2 Hz, 2H), 2.80-2.72 (m, 4H), 2.67-2.63 (m, 0.5H), 2.59-2.55 (m, 0.5H), 2.42-2.38 (m, 2H), 2.08-2.02 (m, 1H), 1.83-1.74 (m, 1H).

### Acid 13: 2-(3-Methoxy-3-oxopropyl)-4,5,6,7-tetrahydrobenzo[d]thiazole-5-carboxylic acid

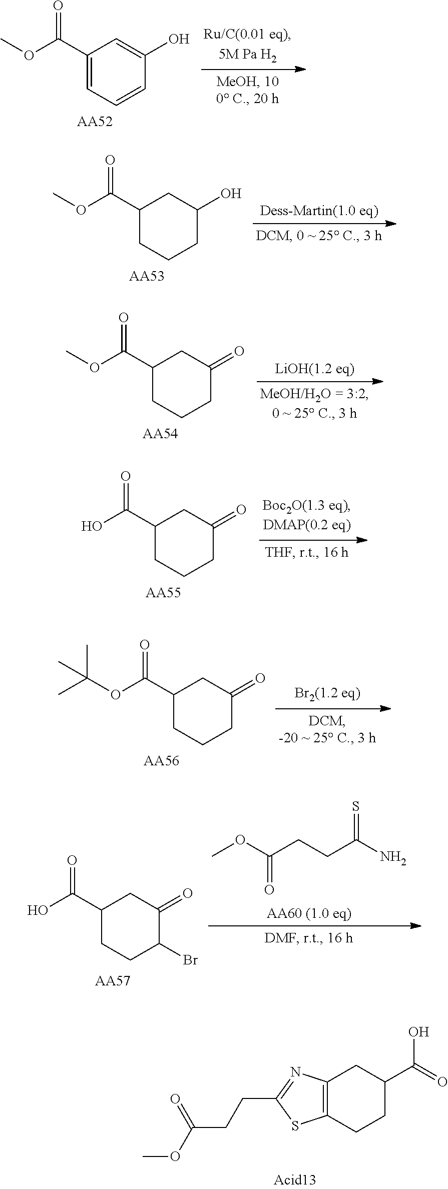

### Intermediate AA53

### Methyl 3-hydroxycyclohexanecarboxylate

To the solution of methyl 3-hydroxybenzoate AA52 (25.0 g, 164 mmol) in dried methanol (800 mL) was added 5% wt. rhodium on carbon (3.38 g, 1.64 mmol) under nitrogen atmosphere at room temperature. After stirred at 100° C.; for 20 hours under hydrogen atmosphere (5 MPa), the mixture was cooled to room temperature and filtered through a pad of celite. The filtrate was concentrated under reduced pressure to give the title compound (25.7 g, 99% yield) as colourless oil. ¹H NMR (400 MHz, DMSO-d₆) δ 4.63 (d, J=4.4 Hz, 0.7H), 4.48 (d, J=3.2 Hz, 0.3H), 3.83-3.78 (m, 0.3H), 3.58 (s, 3H), 3.42-3.34 (m, 0.7H), 2.72-2.65 (m, 0.3H), 2.36-2.83 (tt, J=12.0, 3.6 Hz, 0.7H), 2.03-1.97 (m, 0.7H), 1.80-1.57 (m, 3.3H), 1.46-1.36 (m, 1H), 1.29-0.98 (m, 3H).

### Intermediate AA54

### Methyl 3-oxocyclohexanecarboxylate

To the solution of methyl 3-hydroxycyclohexanecarboxylate AA53 (25.7 g, 0.163 mmol) in dichloromethane (250 mL) was added Dess-Martin periodinane (69.0 g, 0.163 mmol) over 20 minutes at 0° C. After stirred at room temperature for 3 hours, the mixture was filtered. The filtrate was washed with saturated sodium bicarbonate aqueous solution (200 mL) for three times, brine (200 mL), dried over Na₂SO₄₍ₛ₎, filtered and concentrated to give a residue, which was purified by silica gel column chromatography (petroleum ether:ethyl acetate=15:1 to 6:1) to give the title compound (20.5 g, 82% yield) as colorless oil. ¹H NMR (400 MHz, CDCl₃) δ 3.70 (s, 3H), 2.84-2.76 (m, 1H), 2.54 (d, J=10.4 Hz, 2H), 2.37-2.27 (m, 2H), 2.13-2.02 (m, 2H), 1.90-1.70 (m, 2H).

### Intermediate AA55

### 3-Oxocyclohexanecarboxylic acid

To the solution of methyl 3-oxocyclohexanecarboxylate AA54 (10.0 g, 64.1 mmol) in methanol (60 mL) and water (40 mL) was added lithium hydroxide monohydrate (3.23 g, 76.9 mmol) over 20 minutes at 0° C. After stirred at room temperature for 3 hours, the mixture was acidified with 1 M hydrochloride aqueous solution to pH ˜3 and extracted with ethyl acetate (80 mL) for five times. The combined organic phases were washed with brine (100 mL), dried over Na₂SO₄₍ₛ₎, filtered and concentrated to give the title compound (8.5 g, 93% yield) as colorless oil. ¹H NMR (300 MHz, DMSO-d₆) δ 12.39 (s, 1H), 2.81-2.72 (m, 1H), 2.45-2.16 (m, 4H), 1.99-1.63 (m, 4H).

### Intermediate AA56

### tert-Butyl 3-oxocyclohexanecarboxylate

To the solution of 3-oxocyclohexanecarboxylic acid AA55 (8.5 g, 59.8 mmol) and 4-dimethylaminopyridine (1.5 g, 11.9 mmol) in tetrahydrofuran (80 mL) was added di-tert-butyl dicarbonate (16.9 g, 77.8 mmol) dropwise at 0° C. After stirred at room temperature for 16 hours, the mixture was concentrated and the obtained residue was dissolved in ethyl acetate (100 mL). It was washed with water (50 mL) twice followed by brine (50 mL), dried over Na₂SO₄₍ₛ₎, filtered and concentrated to give a residue, which was purified by silica gel column chromatography (petroleum ether:ethyl acetate=20:1 to 10:1) to give the title compound (9.0 g, 77% yield) as colorless oil. ¹H NMR (300 MHz, CDCl₃) δ 2.73-2.66 (m, 1H), 2.49 (d, J=7.5 Hz, 2H), 2.35-2.30 (m, 2H), 2.08-2.01 (m, 2H), 1.87-1.70 (m, 2H), 1.44 (s, 9H).

### Intermediate AA57

### 4-Bromo-3-oxocyclohexanecarboxylic acid

To the solution of tert-butyl 3-oxocyclohexanecarboxylate AA56 (9.0 g, 45.5 mmol) in dichloromethane (60 mL) was added bromine (8.7 g, 54.5 mmol) in dichloromethane (40 mL) dropwise at −20° C. The mixture was stirred from −20° C.; to 20° C.; for 3 hours. After the reaction completed, the mixture was concentrated and the obtained residue was dissolved in water (50 mL). It was extracted with ethyl acetate (50 mL) for four times. The combined organic phases were washed with brine (50 mL), dried over Na₂SO₄₍ₛ₎, filtered and concentrated to give the crude title compound (8.6 g, 86% yield), which was used for the next step without further purification. ¹H NMR (300 MHz, DMSO-d₆) δ 5.31-5.25 (m, 0.2H), 5.03-4.96 (m, 0.4H), 4.83-4.74 (m, 0.4H), 3.10-2.81 (m, 2H), 2.74-2.61 (m, 1H), 2.46-2.35 (m, 1H), 2.15-2.03 (m, 2H), 1.93-1.89 (m, 1H).

### Acid 13: 2-(3-Methoxy-3-oxopropyl)-4,5,6,7-tetrahydrobenzo[d]thiazole-5-carboxylic acid

To the solution of 4-bromo-3-oxocyclohexanecarboxylic acid AA57 (8.6 g, 38.9 mmol) in N,N-dimethylformamide (80 mL) was added methyl 4-amino-4-thioxobutanoate AA60 (5.7 g, 38.9 mmol). The mixture was stirred at room temperature for 20 hours. It was poured into water (200 mL) and then extracted with ethyl acetate (150 mL) for four times. The combined organic phases were washed with brine (150 mL), dried over Na₂SO₄₀), filtered and concentrated to give a residue, which was purified by C18 column (acetonitrile:water=5% to 50%) to give the title compound (3.0 g, 29% yield) as white solids. LC-MS (ESI): R_{T}=1.51 min, mass calcd. for C₁₂H₁₅NO₄S 269.1, m/z found 268.0 [M−H]⁻. ¹H NMR (300 MHz, CDCl₃) δ 3.70 (s, 3H), 3.27 (t, J=7.5 Hz, 2H), 3.15-2.91 (m, 2H), 2.84-2.67 (m, 5H), 2.33-2.21 (m, 1H), 2.06-1.91 (m, 1H).

### Acid 14: 2-(3-Methoxy-3-oxopropyl)-4,5,6,7-tetrahydrobenzo[d]thiazole-6-carboxylic acid

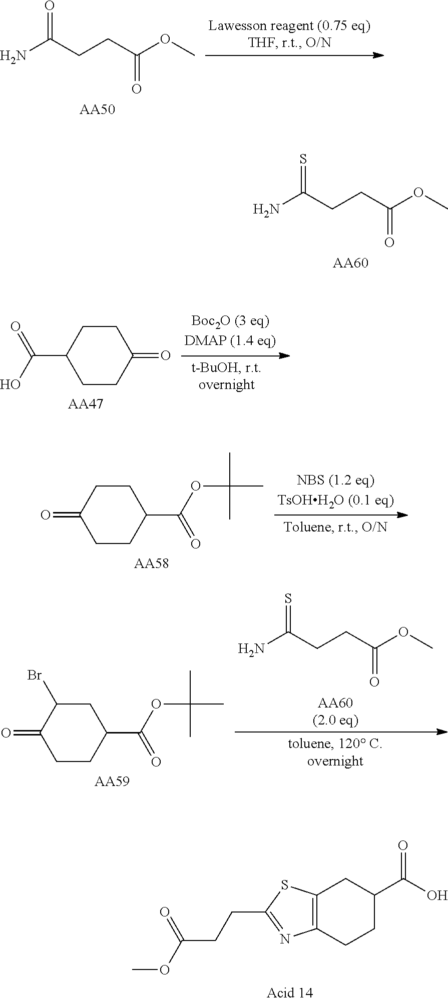

### Intermediate AA58

### tert-Butyl 4-oxocyclohexanecarboxylate

To a solution of 4-oxocyclohexanecarboxylic acid AA47 (10.2 g, 98% purity, 70.3 mmol) in tert-butanol (100 mL) was added N,N-dimethylpyridin-4-amine (12.7 g, 95% purity, 98.5 mmol) and di-tert-butyldicarbonate (46.5 g, 99% purity, 211 mmol). The reaction mixture was stirred at room temperature under nitrogen atmosphere overnight. It was then concentrated in vacuo and the obtained residue was purified by silica gel column chromatography (petroleum:ethyl acetate=10:1) to give the title compound (13.3 g, 99% purity, 94% yield) as colorless oil. ¹H NMR (300 MHz, DMSO-d₆) δ 2.74-2.64 (m, 1H), 2.44-2.33 (m, 2H), 2.26-2.18 (m, 2H), 2.11-2.03 (m, 2H), 1.83-1.70 (m, 2H), 1.41 (s, 9H).

### Intermediate AA59

### tert-Butyl 3-bromo-4-oxocyclohexanecarboxylate

A mixture of tert-butyl 4-oxocyclohexanecarboxylate AA58 (2.00 g, 99% purity, 10.0 mmol), N-bromosuccinimide (2.16 g, 99% purity, 12.0 mmol) and toluene-4-sulfonic monohydrate (0.19 g, 99% purity, 1.00 mmol) in toluene (20 mL) was stirred at room temperature under nitrogen atmosphere overnight. The reaction mixture was concentrated in vacuo and the obtained residue was purified by silica gel column chromatography (petroleum:ethyl acetate=10:1) to give the title compound (1.77 g, 80% purity, 51% yield) as yellow oil. ¹H NMR (300 MHz, DMSO-d₆) δ 4.67 (t, J=4.5 Hz, 1H), 2.92-2.85 (m, 2H), 2.45-2.42 (m, 1H), 2.31-2.24 (m, 2H), 2.08-2.02 (m, 1H), 1.93-1.86 (m, 1H), 1.40 (s, 9H)

### Intermediate AA60

### Methyl 4-amino-4-thioxobutanoate

To a solution of methyl 4-amino-4-oxobutanoate AA50 (5.00 g, 98% purity, 37.4 mmol) in tetrahydrofuran (50 mL) was added Lawesson reagent (11.7 g, 97% purity, 28.0 mmol) at 0° C. The reaction mixture was stirred at room temperature under nitrogen atmosphere overnight. It was then quenched with saturated aqueous sodium bicarbonate solution (200 mL) slowly and extracted with ethyl acetate (150 mL) for three times. The combined organic layers were washed with brine (200 mL), dried over NaSO₄₍ₛ₎, filtered and concentrated under reduced pressure to give a residue, which was purified by silica gel column chromatography (petroleum:ethyl acetate=5:1 to 2:1) to give the title compound (5.10 g, 99% purity, 92% yield) as colorless solids. LC-MS (ESI): R_{T}=0.49 min, mass calcd. for C₅H₉NO₂S 147.0, m/z found 148.0 [M+H]⁺. ¹H NMR (300 MHz, DMSO-d₆) δ 9.45 (br s, 1H), 9.28 (br s, 1H), 3.59 (s, 3H), 2.76-2.67 (m, 4H).

### Acid 14: 2-(2-Methoxycarbonyl-ethyl)-4,5,6,7-tetrahydro-2H-indazole-6-carboxylic acid

To a solution of tert-butyl 3-bromo4-oxocyclohexanecarboxylate AA59 (1.25 g, 80% purity, 3.62 mmol) in toluene (15 mL) was added methyl 4-amino-4-thioxobutanoate AA60 (1.08 g, 99% purity, 7.23 mmol) at room temperature. After stirred at 120° C.; under nitrogen atmosphere overnight, the mixture was cooled down to room temperature and concentrated under reduced pressure to give a residue, which was purified by silica gel column chromatography (dichloromethane:methanol=20:1 to 10:1) to give the title compound (660 mg, 84% purity, 57% yield) as red solids. LC-MS (ESI): R_{T}=0.97 min, mass calcd. for C₁₂H₁₅NO₄S 269.1, m/z found 268.1 [M−H]⁻. ¹H NMR (300 MHz, DMSO-d₆) δ 3.55 (s, 3H), 3.12-3.06 (m, 2H), 2.93-2.86 (m, 1H), 2.79-2.63 (m, 4H), 2.46-2.45 (m, 2H), 2.09-2.04 (m, 1H), 1.80-1.73 (m, 1H).

### Acid 15: 2-(3-Methoxy-3-oxopropyl)-4,5,6,7-tetrahydrobenzo[d]thiazole-6-carboxylic acid

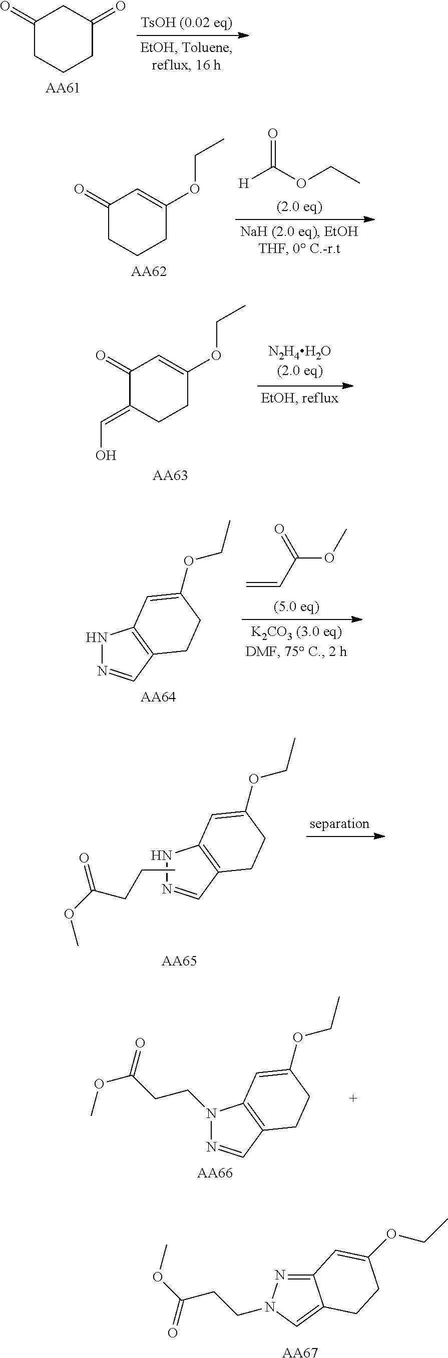

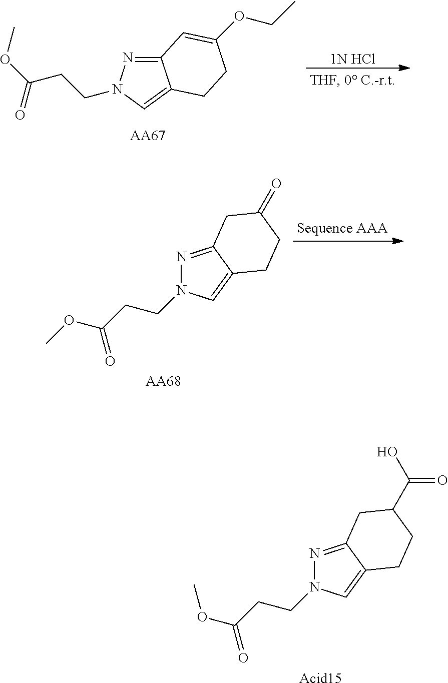

### Intermediate AA62

### 3-Ethoxy-cyclohex-2-enone

To a solution of cyclohexane-1,3-dione AA61 (40.0 g, 350 mmol) in ethanol (160 mL) and toluene (500 mL) was added toluene-4-sulfonic acid (1.35 g, 7.0 mmol) at room temperature. After refluxed with azeotropic removal of water for 16 hours, the reaction mixture was cooled down, quenched by saturated sodium bicarbonate aqueous solution (20 mL) and concentrated under reduced pressure to give a residue. It was diluted with water (100 mL) and extracted with ethyl acetate (100 mL) twice. The combined organic layers were washed with water (100 mL) twice followed by brine (100 mL), dried over Na₂SO₄₍ₛ₎ and filtered. The filtrate was concentrated under reduced pressure to give the title compound (3.50 g, 93% yield, 90% purity) as brown oil. ¹H NMR (300 MHz, CDCl₃) δ 5.34 (s, 1H), 3.92-3.87 (m, 2H), 2.41-2.35 (m, 4H), 2.03-1.94 (m, 2H), 1.37-1.35 (m, 3H).

### Intermediate AA63

### 3-Ethoxy-6-hydroxymethylene-cyclohex-2-enone

To a solution of 60% wt sodium hydride in mineral oil (5.40 g, 136 mmol) in ethanol (18 mL) and tetrahydrofuran (150 mL) was added 3-ethoxycyclohex-2-enone AA62 (10.0 g, 67.8 mmol, 95% purity) at 0° C.; under nitrogen atmosphere. After stirring for 1 hour at 0° C., ethyl formate (10.2 g, 136 mmol) was added. Having stirred at room temperature for 18 hours, the mixture was partitioned between water (500 mL) and ethyl acetate (500 mL). The separated aqueous layer was acidified to pH 7 with 2 M hydrochloride aqueous solution and then extracted with dichloromethane (100 mL) twice. The combined dichloromethane layers were washed with water (100 mL) twice followed by brine (100 mL), dried over Na₂SO₄₍ₛ₎ and filtered. The filtrate was concentrated under reduced pressure to leave a residue, which was purified by silica gel column chromatography (petroleum ether:ethyl acetate=4:1 to 2:1) to give the title compound (7.0 g, 58%, 95% purity) as yellow oil. LC-MS (ESI): R_{T}=2.933 min, mass calcd. for C₉H₁₂O₃ 168.1, m/z found 167.1 [M−H]⁻. ¹H NMR (300 MHz, CDCl₃) δ 13.89 (s, 1H), 7.17 (s, 1H), 5.31 (s, 1H), 3.97-3.87 (m, 2H), 2.47-2.36 (m, 4H), 1.39-1.32 (m, 3H).

### Intermediate AA64

### 6-Ethoxy-4,5-dihydro-1H-indazole

To a solution of 3-ethoxy-6-(hydroxymethylene)cyclohex-2-enone AA63 (17.0 g, 96.0 mmol, 95% purity) in ethanol (500 mL) was added hydrazine hydrate (11.3 g, 192 mmol) at room temperature. After refluxed for 2 hours, the mixture was concentrated under reduced pressure to give the title compound (15.5 g, 89% yield, 90% purity) as colorless solids. LC-MS (ESI): R_{T}=3.027 min, mass calcd. for C₉H₁₂N₂O 164.1, m/z found 165.1 [M+H]⁺. ¹H NMR (300 MHz, CDCl₃) δ 7.19 (s, 1H), 5.62 (s, 1H), 3.93-3.85 (m, 2H), 2.75 (t, J=8.1 Hz, 2H), 2.47 (t, J=8.1 Hz, 2H), 1.39-1.33 (m, 3H).

### Intermediate AA65

### Mixture of methyl 3-(6-ethoxy-4,5-dihydro-1H-indazol-1-yl)propanoate and methyl 3-(6-ethoxy-4,5-dihydro-2H-indazol-2-yl)propanoate

To a solution of 6-ethoxy-4,5-dihydro-1H-indazole AA64 (10.0 g, 57.9 mmol, 90% purity) in N,N-dimethylformamide (150 mL) was added methyl acrylate (26.6 g, 305 mmol) and potassium carbonate (25.8 g, 183 mmol) at room temperature. After stirred at 75° C.; for 2 hours, the mixture was cooled down, quenched with saturated potassium hydrogensulphate aqueous solution (150 mL) and extracted with ethyl acetate (200 mL) twice. The combined organic layers were washed with water (100 mL) twice followed by brine (100 mL), dried over Na₂SO₄₍ₛ₎ and filtered. The filtrate was concentrated under reduced pressure to leave a residue, which was purified with silica gel column chromatography (petroleum ether:ethyl acetate=2:1) to give the title compound (12.0 g, 85% yield, mixture of region-isomers) as yellow oil. LC-MS (ESI): R_{T}=1.597 min, mass calcd. for C₁₃H₁₈N₂O₃ 250.1, m/z found 251.1 [M+H]⁺. ¹H NMR (300 MHz, DMSO-d₆) δ 7.23 (s, 0.6H), 7.05 (s, 0.4H), 5.69 (s, 0.4H), 5.49 (s, 0.6H), 4.20-4.13 (m, 2H), 3.88-3.79 (m, 2H), 3.57-3.54 (m, 3H), 2.79-2.72 (m, 2H), 2.60-2.53 (m, 2H), 2.36-2.26 (m, 2H), 1.29-1.21 (m, 3H).

A regioisomeric mixture of AA65 (17.0 g, 68 mmol, 90% purity) was separated by chiral prep. HPLC (Column: Chiralpak IC 5 μm 20*250 mm; Mobile Phase: Hex:EtOH=80:20 at 15 mL/min; Temp: 30° C.; Wavelength: 230 nm) to afford AA66 (5.40 g, 35% yield, 95% purity) as brown oil and AA66 (3.70 g, 21% yield, 95% purity) as white solids. Intermediate AA66: LC-MS (ESI): R_{T}=1.605 min, mass calcd. for C₁₃H₁₈N₂O₃ 250.1, m/z found 251.1 [M+H]⁺. ¹H NMR (300 MHz, DMSO-d₆) δ 7.21 (s, 1H), 5.48 (s, 1H), 4.14 (t, J=6.6 Hz, 2H), 3.79 (q, J=6.9 Hz, 2H), 3.56 (s, 3H), 2.76 (t, J=6.6 Hz, 2H), 2.57 (t, J=8.1 Hz, 2H), 1.23 (t, J=6.9 Hz, 3H).

### Intermediate AA68

### Methyl 3-(6-oxo-4,5,6,7-tetrahydro-2H-indazol-2-yl)propanoate

To a solution of methyl 3-(6-ethoxy-4,5-dihydro-2H-indazol-2-yl)propanoate AA67 (3.70 g, 14.0 mmol, 95% purity) in tetrahydrofuran (7.4 mL) was added 1 M hydrochloride aqueous solution (36 mL, 36.0 mmol) at 0° C. After stirred at room temperature for 2 hours, the mixture was poured into ice water (100 mL) and extracted with ethyl acetate (150 mL) twice. The combined organic layers were washed with saturated sodium bicarbonate aqueous solution (100 mL) twice followed by water (50 mL) twice and brine (50 mL), dried over Na₂SO₄₍ₛ₎ and filtered. The filtrate was concentrated under reduced pressure to leave a residue, which was purified by silica gel column chromatography (petroleum ether:ethyl acetate=4:1 to 2:1) to give the title compound (2.70 g, 74% yield, 85% purity) as yellow solids. LC-MS (ESI): R_{T}=0.503 min, mass calcd. for C₁₁H₁₄N₂O₃ 222.1, m/z found 223.1 [M+H]⁺. ¹H NMR (300 MHz, CDCl₃) δ 7.26 (s, 1H), 4.36 (t, J=6.6 Hz, 2H), 3.70 (s, 3H), 3.56 (s, 2H), 2.91-2.83 (m, 4H), 2.61 (t, J=6.9 Hz, 2H).

### Acid 15: 2-(2-Methoxycarbonyl-ethyl)-4,5,6,7-tetrahydro-2H-indazole-6-carboxylic acid

By ultilizing the analogous procedure of Sequence AAA (the last three steps), AA68 was converted to the title compound.

LC-MS (ESI): R_{T}=0.738 min, mass calcd. for C₁₂H₁₆N₂O₄ 252.1, m/z found 253.1 [M+H]⁺. ¹H NMR (300 MHz, DMSO-d₆) δ 12.23 (br s, 1H), 7.32 (s, 1H), 4.21-4.16 (m, 2H), 3.56 (s, 3H), 2.81-2.53 (m, 6H), 2.44-2.40 (m, 1H), 2.02-1.97 (m, 1H), 1.66-1.56 (m, 1H).

### Acid 16: 1-cyano-2-(3-methoxy-3-oxopropyl)-4,5,6,7-tetrahydro-2H-isoindole-5-carboxylic acid

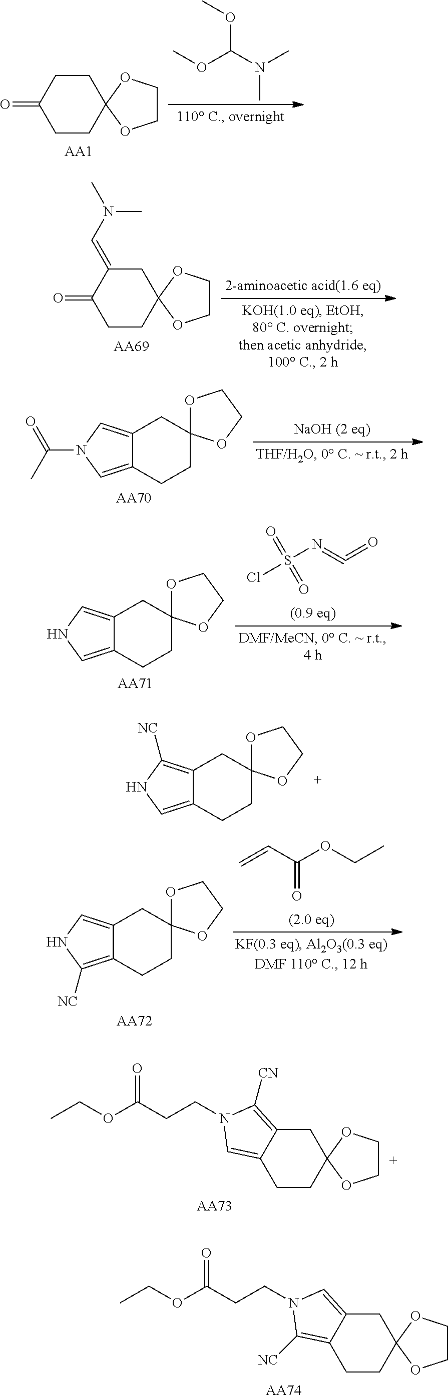

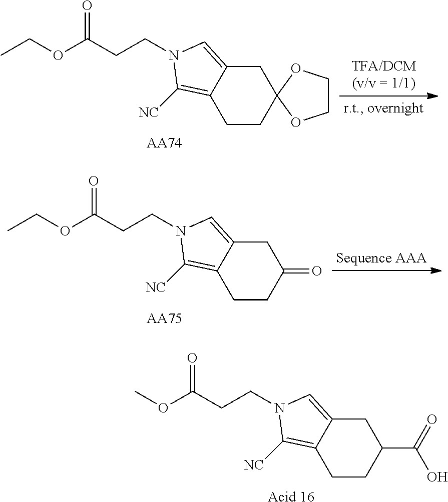

### Intermediate AA69

### 7-((dimethylamino)methylene)-1,4-dioxaspiro[4.5]decan-8-one

A solution of 1,4-dioxaspiro[4.5]decan-8-one AA1 (30.0 g, 188 mmol) in 1,1-dimethoxy-N,N-dimethylmethanamine (67.5 g, 538 mmol) was stirred at 110° C. overnight under nitrogen atmosphere. Then the mixture was cooled down to room temperature and concentrated. The obtained residue was purified by silica gel column chromatography (dichloromethane:methanol=200:1 to 50:1) to give the title compound (15.5 g, 35% yield) as yellow oil. ¹H NMR (300 MHz, DMSO-d₆) δ 7.24 (s, 1H), 3.88 (s, 4H), 2.99 (s, 6H), 2.79 (s, 2H), 2.24-2.19 (m, 2H), 1.82-1.77 (m, 2H).

### Intermediate AA70

### 1-(6′,7′-Dihydrospiro[[1,3]dioxolane-2,5′-isoindol]-2′(4′H)-yl)ethanone

To a solution of 7-((dimethylamino)methylene)-1,4-dioxaspiro[4.5]decan-8-one AA69 (15.5 g, 66.0 mmol) in ethanol (155 mL) was added 2-aminoacetic acid (7.97 g, 106 mmol) and potassium hydroxide (4.36 g, 66.0 mmol) at room temperature. After stirred at 80° C. overnight under nitrogen atmosphere, the mixture was cooled down to room temperature and concentrated. The residue was added acetic anhydride (100 mL) at room temperature. After stirred at 100° C.; for 2 hours, the mixture was cooled down to room temperature, basified with saturated sodium bicarbonate aqueous solution to pH 8-9 and extracted with ethyl acetate (200 mL) for three times. The combined organic layers were washed with brine (100 mL), dried over Na₂SO₄(s) and filtered. The filtrate was concentrated to give a residue, which was purified by silica gel column chromatography (petroleum ether:ethyl acetate=10:1 to 5:1) to give the title compound (2.10 g, 14% yield) as yellow oil. LC-MS (ESI): R_{T}=1.482 min, mass calcd. for C₁₂H₁₅NO₃ 221.1, m/z found 222.1 [M+H]⁺. ¹H NMR (400 MHz, CDCl₃) δ 7.00 (br s, 2H), 4.03-3.99 (m, 4H), 2.80-2.72 (m, 4H), 2.45 (s, 3H), 1.90 (t, J=7.2 Hz, 2H).

### Intermediate AA71

### 2′,4′,6′,7′-Tetrahydrospiro[[1,3]dioxolane-2,5′-isoindole]

A solution of 1-(6′,7′-dihydrospiro[[1,3]dioxolane-2,5′-isoindol]-2′(4′H)-yl)ethanone AA70 (2.10 g, 9.11 mmol) in tetrahydrofuran (21 mL) was added water (21 mL) and sodium hydroxide (746 mg, 17.9 mmol) at 0° C. After stirred at room temperature for 2 hours under nitrogen atmosphere, the mixture was quenched with water (30 mL) slowly and extracted with ethyl acetate (30 mL) for three times. The combined organic layers were washed with brine (20 mL), dried over Na₂SO₄₍ₛ₎, filtered and concentrated under reduced pressure to give a residue, which was purified by silica gel column chromatography (petroleum ether:ethyl acetate=10:1 to 5:1) to give the title compound (1.60 g, 97% yield) as brown oil. LC-MS (ESI): R_{T}=1.304 min, mass calcd. for C₁₀H₁₃NO₂ 179.1, m/z found 180.1 [M+H]⁺. ¹H NMR (300 MHz, CDCl₃) δ 8.02 (br s, 1H), 6.54-6.46 (m, 2H), 4.06-3.99 (m, 4H), 2.84-2.75 (m, 4H), 1.94 (t, J=6.6 Hz, 2H).

### Intermediate AA72

### Mixture of 2′,4′,6′,7′-tetrahydrospiro[[1,3]dioxolane-2,5′-isoindole]-3′-carbonitrile and 2′,4′,6′,7′-tetrahydrospiro[[1,3]dioxolane-2,5′-isoindole]-1′-carbonitrile

To a suspension of 2′,4′,6′,7′-tetrahydrospiro[[1,3]dioxolane-2,5′-isoindole] AA71 (2.00 g, 10.6 mmol) in N,N-dimethylformamide (8 mL) and acetonitrile (40 mL) was added a solution of sulfurisocyanatidic chloride (1.38 g, 9.54 mmol) in acetonitrile (2 mL) dropwise at 0° C. Then the reaction mixture was allowed to warm up to room temperature and stirred at this temperature for 4 hours. The mixture was quenched by pouring into 10% sodium carbonate aqueous solution (100 mL) and extracted with ethyl acetate (100 mL) for three times. The combined organic layers were washed with water (100 mL) and brine (100 mL), dried over Na₂SO₄₍ₛ₎ and filtered. The filtrate was concentrated under reduced pressure to give a residue, which was purified by C18 column (acetonitrile:water=75% to 85%) to give the title compound (900 mg, 42% yield) as brown oil. ¹H NMR (400 MHz, CDCl₃) δ 8.70 (br s, 1H), 6.66-6.63 (m, 1H), 4.05-4.03 (m, 4H), 2.90-2.87 (m, 2H), 2.77-2.74 (m, 2H), 1.95-1.90 (m, 2H).

### Intermediate AA73 and AA74

### Ethyl 3-(3′-cyano-6′,7′-dihydrospiro[[1,3]dioxolane-2,5′-isoindol]-2′ (4′H)-yl)propanoate and ethyl 3-(1′-cyano-6′,7′-dihydrospiro[[1,3]dioxolane-2,5′-isoindol]-2′(4′H)-yl)propanoate

A solution of 2′,4′,6′,7′-tetrahydrospiro[[1,3]dioxolane-2,5′-isoindole]-3′-carbonitrile and 2′,4′,6′,7′-tetrahydrospiro[[1,3]dioxolane-2,5′-isoindole]-1′-carbonitrile AA72 (3.50 g, 95% purity, 16.3 mmol), ethyl acrylate (3.26 g, 32.6 mmol), potassium fluoride (0.284 g, 4.88 mmol) and aluminium oxide (0.498 g, 4.88 mmol) in N,N-dimethylformamide (30 mL) was heated at 110° C.; for 12 hours. Then it was cooled down to room temperature and diluted with water (120 mL) and extracted with ethyl acetate (100 mL) twice. The combined organic layers were washed with brine (50 mL), dried over Na₂SO₄₍ₛ₎ and filtered. The filtrate was concentrated under reduced pressure to give a residue, which was purified by C18 column (acetonitrile:water=55% to 65%) followed by chiral SFC (Column: Chiralpak IE 5 μm 20*250 mm; Mobile Phase: CO₂:EtOH=70:30 at 50 g/min; Temp 30° C.; Wavelength: 254 nm; Back pressure: 100 bar) to afford the title compounds AA73 (2.5 g, 49% yield) and AA74 (1.5 g, 31% yield) as brown oil.

Intermediate AA73: LC-MS (ESI): R_{T}=1.57 min, mass calcd. for C₁₆H₂₀N₂O₄ 304.1, m/z found 305.1 [M+H]⁺. ¹H NMR (400 MHz, CDCl₃) δ 6.63 (s, 1H), 4.25 (t, J=6.8 Hz, 2H), 4.14 (q, J=6.8 Hz, 2H), 4.04-4.00 (m, 4H), 2.85 (s, 2H), 2.77 (t, J=6.4 Hz, 2H), 2.70 (t, J=6.8 Hz, 2H), 1.88 (t, J=6.8 Hz, 2H), 1.27-1.23 (m, 3H).

Intermediate AA74: LC-MS (ESI): R_{T}=1.56 min, mass calcd. for C₁₆H₂₀N₂O₄ 304.1, m/z found 305.1 [M+H]⁺. ¹H NMR (400 MHz, CDCl₃) δ 6.67 (s, 1H), 4.28 (t, J=6.4 Hz, 2H), 4.19-4.18 (m, 2H), 4.05 (s, 4H), 2.88 (t, J=6.8 Hz, 2H), 2.81 (t, J=6.8 Hz, 2H), 2.74 (s, 2H), 1.94 (t, J=6.8 Hz, 2H), 1.31-1.27 (m, 3H).

### Intermediate AA75

### Ethyl 3-(1-cyano-5-oxo-4,5,6,7-tetrahydro-2H-isoindol-2-yl)propanoate

To a solution of ethyl 3-(1′-cyano-6′,7′-dihydrospiro[[1,3]dioxolane-2,5′-isoindol]-2′(4′H)-yl)propanoate AA74 (1.50 g, 4.68 mmol) in dichloromethane (10 mL) was added trifluoroacetic acid (10 mL) at 0° C. After stirred at room temperature overnight, the reaction mixture was concentrated under reduced pressure, basified with saturated sodium carbonate aqueous solution to pH 7-8 and extracted with ethyl acetate (20 mL) twice. The combined organic layers were washed with brine (20 mL) twice, dried over Na₂SO₄₍ₛ₎ and filtered. The filtrate was concentrated under reduced pressure to give the crude product, which was purified by C18 column (acetonitrile:water=45% to 60%) to give the title compound (950 mg, 63% yield) as yellow oil. LC-MS (ESI): R_{T}=1.540 min, mass calcd. for C₁₄H₁₆N₂O₃ 260.1, m/z found 261.1 [M+H]⁺. ¹H NMR (400 MHz, CDCl₃) δ 6.71 (s, 1H), 4.31 (t, J=6.8 Hz, 2H), 4.16 (q, J=7.2 Hz, 2H), 3.38 (s, 2H), 3.01 (t, J=6.4 Hz, 2H), 2.82 (t, J=6.4 Hz, 2H), 2.61 (t, J=7.2 Hz, 2H), 1.26 (t, J=5.6 Hz, 3H).

### Acid 16: 1-cyano-2-(3-methoxy-3-oxopropyl)-4,5,6,7-tetrahydro-2H-isoindole-5-carboxylic acid

By ultilizing the analogous procedure of Sequence AAA (the last three steps), AA75 was converted to the title compound.

LC-MS (ESI): R_{T}=0.51 min, mass calcd. for C₁₄H₁₆N₂O₄ 276.1, m/z found 277.1 [M+H]⁺. ¹H NMR (400 MHz, CDCl₃) δ 6.64 (s, 1H), 4.27 (t, J=6.4 Hz, 2H), 3.71 (s, 3H), 2.87-2.79 (m, 4H), 2.71-2.61 (m, 3H), 2.25-2.19 (m, 1H), 1.89-1.82 (m, 1H).

### Acid 17: 3-cyano-2-(3-methoxy-3-oxopropyl)-4,5,6,7-tetrahydro-2H-isoindole-5-carboxylic acid

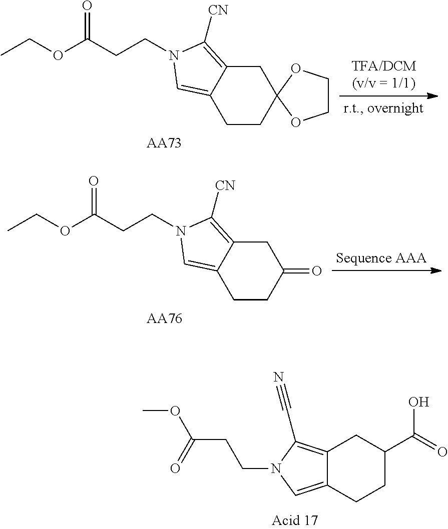

### Intermediate AA76

### Ethyl 3-(1-cyano-6-oxo-4,5,6,7-tetrahydro-2H-isoindol-2-yl)propanoate

To a solution of ethyl 3-(3′-cyano-6′,7′-dihydrospiro[[1,3]dioxolane-2,5′-isoindol]-2′(4′H)-yl)propanoate AA73 (2.50 g, 84.0% purity, 6.90 mmol) in dichloromethane (10 mL) was added trifluoroacetic acid (10 mL) at 0° C. After stirred at room temperature overnight, the reaction mixture was concentrated under reduced pressure, basified with saturated sodium carbonate aqueous solution to pH 7-8, and extracted with ethyl acetate (20 mL) twice. The combined organic layers were washed with brine (20 mL) twice, dried over Na₂SO₄₍ₛ₎ and filtered. The filtrate was concentrated under reduced pressure to give the crude product, which was purified by C18 column (acetonitrile:water=45% to 60%) to give the title compound (950 mg, 90.7% purity, 48% yield) as yellow oil. LC-MS (ESI): R_{T}=1.506 min, mass calcd. for C₁₄H₁₆N₂O₃ 260.1, m/z found 261.1 [M+H]⁺. ¹H NMR (400 MHz, CDCl₃) δ 6.74 (s, 1H), 4.31 (t, J=6.4 Hz, 2H), 4.17 (q, J=7.2 Hz, 2H), 3.51 (s, 2H), 2.87 (t, J=6.4 Hz, 2H), 2.82 (t, J=6.8 Hz, 2H), 2.58 (t, J=7.2 Hz, 2H), 1.26 (t, J=7.2 Hz, 3H).

### Acid 17: 3-Cyano-2-(3-methoxy-3-oxopropyl)-4,5,6,7-tetrahydro-2H-isoindole-5-carboxylic acid

By ultilizing the analogous procedure of Sequence AAA (the last three steps), AA76 was converted to the title compound.

LC-MS (ESI): R_{T}=1.569 min, mass calcd. for C₁₄H₁₆N₂O₄ 276.1, m/z found 277.1 [M+H]⁺. ¹H NMR (400 MHz, DMSO-d₆) δ 12.30 (s, 1H), 6.93 (s, 1H), 4.18 (t, J=7.2 Hz, 2H), 3.61 (s, 3H), 2.84 (t, J=6.8 Hz, 2H), 2.76-2.56 (m, 3H), 2.50-2.42 (m, 2H), 2.06-2.01 (m, 1H), 1.69-1.60 (m, 1H).

### Acid 18: 2-(3-methoxy-3-oxopropyl)-5,6,7,8-tetrahydroquinazoline-6-carboxylic acid

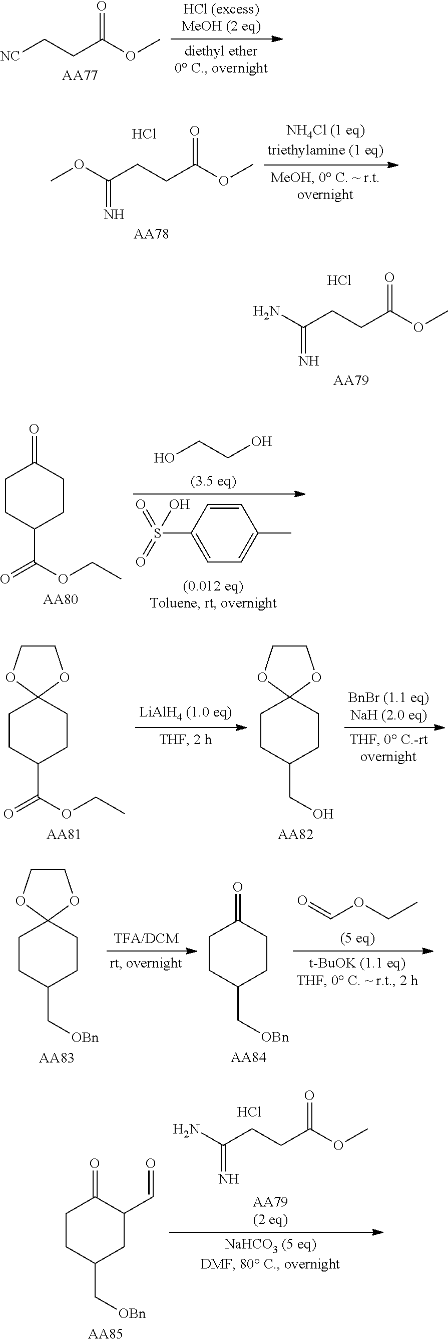

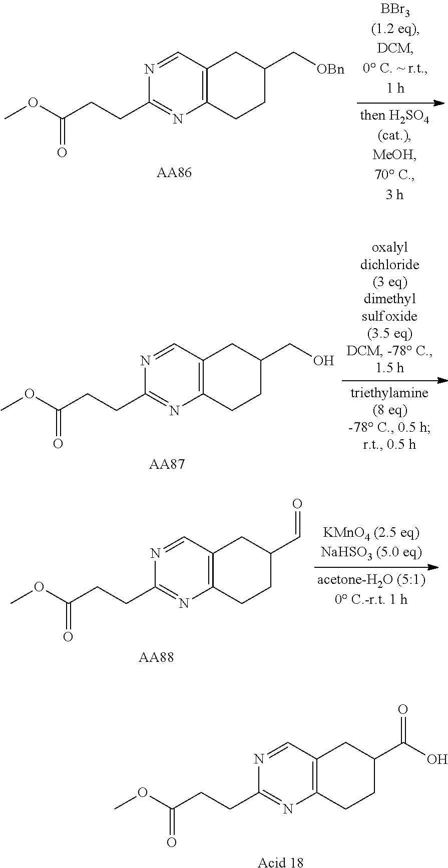

Intermediate AA78:

### Methyl 4-imino-4-methoxybutanoate hydrochloride

To a solution of methyl 3-cyanopropanoate (10.0 g, 88.5 mmol) AA77 and methanol (5.50 g, 172 mmol) in diethyl ether (20 mL) was bubbled hydrogen chloride gas at 0° C.; over 30 minutes. After stirred at 0° C.; overnight, the mixture was diluted with dry diethyl ether (100 mL). The white solids were collected by filtration to give the title compound (15.5 g, 92% yield). ¹H NMR (400 MHz, DMSO-d₆) δ 12.02-11.34 (m, 2H), 4.07 (s, 3H), 3.63 (s, 3H), 2.91 (t, J=7.2 Hz, 2H), 2.77 (t, J=7.2 Hz, 2H).

### Intermediate AA79

### Methyl 4-amino-4-iminobutanoate hydrochloride

To a solution of methyl 4-imino-4-methoxybutanoate hydrochloride AA78 (11.4 g, 95% purity, 59.6 mmol) and ammonium chloride (3.19 g, 59.6 mmol) in dry methanol (15 mL) was added triethylamine (6.03 g, 59.6 mmol) dropwise at 0° C. After addition, the mixture was stirred at room temperature overnight. Then it was concentrated at room temperature and added ethyl acetate (100 mL). The white solids were collected by filtration to give the title compound (18.2 g, 92% yield). ¹H NMR (400 MHz, DMSO-d₆) δ 9.08 (s, 2H), 8.81 (s, 2H), 3.63 (s, 3H), 2.81 (t, J=7.2 Hz, 2H), 2.65 (t, J=7.2 Hz, 2H).

### Intermediate AA81

### Ethyl 1,4-dioxaspiro[4.5]decane-8-carboxylate

To a solution of ethyl 4-oxocyclohexanecarboxylate AA80 (30.0 g, 176 mmol) and ethane-1,2-diol (38.0 g, 618 mmol) in toluene (90 mL) was added 4 methylbenzenesulfonic acid (400 mg, 2.1 mmol). After stirred at room temperature overnight, the mixture was concentrated and redissolved in ethyl acetate (100 mL), and washed with water (80 mL). The separated organic layer was concentrated under reduced pressure to give the title compound (37.0 g, 98% yield) as colorless oil. ¹H NMR (300 MHz, CDCl₃) δ 4.11 (q, J=6.9 Hz, 2H), 3.93 (s, 4H), 2.37-2.27 (m, 1H), 1.95-1.75 (m, 6H), 1.59-1.49 (m, 2H), 1.23 (q, J=6.9 Hz, 3H).

### Intermediate AA82

### 1,4-Dioxaspiro[4.5]decan-8-ylmethanol

To a solution of ethyl 1,4-dioxaspiro[4.5]decane-8-carboxylate AA81 (5.00 g, 23.4 mmol) in tetrahydrofuran (30 mL) was added lithium aluminium hydride (890 mg, 23.4 mmol) at 0° C. After stirred at room temperature for 2 hours, the reaction mixture was quenched with water (0.9 mL) and 15% wt. sodium hydroxide aqueous solution (0.9 mL). The mixture was filtered and the filtrate was concentrated under reduced pressure to give the title compound (3.90 g, 98% yield) as colorless oil. ¹H NMR (400 MHz, CDCl₃) δ 3.95 (s, 4H), 3.49 (s, 2H), 1.80-1.77 (m, 4H), 1.59-1.52 (m, 4H), 1.31-1.23 (m, 2H).

### Intermediate AA83

### 8-Benzyloxymethyl-1,4-dioxa-spiro[4.5]decane

To a solution of 1,4-dioxaspiro[4.5]decan-8-ylmethanol AA82 (3.90 g, 22.7 mmol) in tetrahydrofuran (60 mL) was added 60% wt. sodium hydride in mineral oil (1.80 g, 45.4 mmol) at 0° C. After stirred at this temperature for 20 minutes, benzylbromide (4.27 g, 25.0 mmol) was added dropwise. After stirred at room temperature overnight, the reaction mixture was quenched with water (20 mL) and extracted with ethyl acetate (50 mL) twice. The combined organic layers were concentrated under reduced pressure to give a crude product, which was purified by silica gel column chromatography (petroleum ether:ethyl acetate=30:1 to 5:1) to give the title compound (3.00 g, 51% yield) as colorless oil. ¹H NMR (300 MHz, CDCl₃) δ 7.38-7.25 (m, 5H), 4.54-4.49 (m, 2H), 3.98-3.91 (m, 4H), 3.36-3.29 (m, 2H), 1.84-1.50 (m, 7H), 1.34-1.22 (m, 2H).

### Intermediate AA84

### 4-((Benzyloxy)methyl)cyclohexanone

To a solution of 8-benzyloxymethyl-1,4-dioxa-spiro[4.5]decane AA83 (1.00 g, 3.82 mmol) in dichloromethane (10 mL) was added trifluoroacetic acid (10 mL) at room temperature. After stirred at room temperature overnight, the reaction mixture was concentrated and redissolved in ethyl acetate (20 mL). The solvent was washed with saturated sodium bicarbonate aqueous solution (30 mL), brine (15 mL), dried over Na₂SO₄₍ₛ₎ and filtered. The filterate was concentrated under reduced pressure to give the title compound (560 mg, 67% yield) as colorless oil. ¹H NMR (300 MHz, CDCl₃) δ 7.39-7.24 (m, 5H), 4.53 (s, 2H), 3.39 (d, J=6.0 Hz, 2H), 2.45-2.28 (m, 4H), 2.17-2.04 (m, 3H), 1.54-1.40 (m, 2H).

### Intermediate AA85

### 5-((Benzyloxy)methyl)-2-oxocyclohexanecarbaldehyde

To a suspension of potassium tert-butoxide (4.90 g, 42.8 mmol, 98% purity) in dry tetrahydrofuran (130 mL) was added ethyl formate (9.6 mL, 116 mmol) dropwise at 0° C. After addition, the mixture was stirred at 0° C.; for 20 minutes. A solution of ethyl formate (6.4 mL, 77.6 mmol) and 4-((benzyloxy)methyl)cyclohexanone AA84 (8.90 g, 38.7 mmol, 95% purity) in dry tetrahydrofuran (50 mL) was added into the reaction mixture dropwise at 0° C. After stirred at room temperature for 2 hours, the reaction mixture was quenched with 10% wt. citric acid aqueous solution (45 mL) and diluted with ethyl acetate (300 mL) and water (200 mL). The organic layer was separated and the aqueous layer was extracted with ethyl acetate (150 mL) twice. The combined organic layers were washed with brine (300 mL), dried over Na₂SO₄₍ₛ₎ and filtered. The filtrate was concentrated to give the title compound (9.50 g, 90% purity, 90% yield) as yellow oil. ¹H NMR (400 MHz, CDCl₃) δ 14.39 (s, 1H), 8.64 (s, 1H), 7.38-7.27 (m, 5H), 4.53 (s, 2H), 3.42-3.38 (m, 2H), 2.54-2.30 (m, 3H), 2.17-1.89 (m, 3H), 1.48-1.34 (m, 1H).

### Intermediate AA86

### Methyl 3-(6-((benzyloxy)methyl)-5,6,7,8-tetrahydroquinazolin-2-yl)propanoate

To a solution of 5-((benzyloxy)methyl)-2-oxocyclohexanecarbaldehyde AA85 (2.00 g, 7.71 mmol, 95% purity) and methyl 4-amino-4-iminobutanoate hydrochloride (5.14 g, 15.4 mmol, 50% purity) in N,N-dimethylformamide (20 mL) was added sodium bicarbonate (3.24 g, 38.6 mmol). The mixture was stirred at 80° C.; under nitrogen atmosphere overnight. After cooling down to room temperature, the mixture was poured into water (200 mL) and extracted with ethyl acetate (150 mL) twice. The combined organic layers were washed with water (150 mL) and brine (150 mL), dried over Na₂SO₄₍ₛ₎ and filtered. The filtrate was concentrated and purified by silica gel column chromatography (petroleum ether:ethyl acetate=2:1 to 3:2) to give the desired compound (500 mg, 90% purity, 17% yield) as yellow oil. LC-MS (ESI): R_{T}=1.45 min, mass calcd. for C₂₀H₂₄N₂O₃ 340.2, m/z found 341.0 [M+H]⁺. ¹H NMR (400 MHz, CDCl₃) δ 8.32 (s, 1H), 7.39-7.29 (m, 5H), 4.55 (s, 2H), 3.68 (s, 3H), 3.50-3.43 (m, 2H), 3.22 (t, J=7.2 Hz, 2H), 2.95-2.78 (m, 5H), 2.48-2.41 (m, 1H), 2.12-2.07 (m, 2H), 1.61-1.50 (m, 1H).

### Intermediate AA87

### Methyl 3-(6-(hydroxymethyl)-5,6,7,8-tetrahydroquinazolin-2-yl)propanoate

To a solution of methyl 3-(6-((benzyloxy)methyl)-5,6,7,8-tetrahydroquinazolin-2-yl)propanoate AA86 (1.00 g, 2.64 mmol, 90% purity) in dichloromethane (25 mL) was added 1 M boron tribromide in dichloromethane (3.2 mL, 3.2 mmol) at 0° C. After the addition, the mixture was stirred at room temperature for 1 hour. LCMS detected de-tBu byproduct mainly. It was quenched with dry methanol (50 mL) and concentrated to give a brown residue, which was dissolved in dry methanol (50 mL). To this solution was added three drops of concentrated sulfuric acid. The mixture was stirred at 70° C.; under nitrogen atmosphere for 3 hours. After cooling down to room temperature, triethylamine (2 mL) was added into the mixture. The resulting solution was concentrated and purified by C18 column (acetonitrile:water=25% to 35%) to give the title compound (290 mg, 42% yield, 95% purity from ¹H NMR) as yellow solids. LC-MS (ESI): R_{T}=1.11 min, mass calcd. for C₁₃H₁₈N₂O₃ 250.1, m/z found 251.2 [M+H]⁺. ¹H NMR (400 MHz, CDCl₃) δ 8.33 (s, 1H), 3.68 (s, 3H), 3.67 (d, J=6.8 Hz, 2H), 3.23 (t, J=7.2 Hz, 2H), 2.98-2.79 (m, 5H), 2.49-2.42 (m, 1H), 2.13-2.06 (m, 1H), 2.02-1.94 (m, 1H), 1.60-1.49 (m, 1H).

### Intermediate AA88

### Methyl 3-(6-formyl-5,6,7,8-tetrahydroquinazolin-2-yl)propanoate

To a solution of oxalyl dichloride (0.2 mL, 2.48 mmol) in dry dichloromethane (3 mL) was added dimethyl sulfoxide (0.2 mL, 2.96 mmol) at −78° C.; dropwise. The mixture was stirred at −78° C.; for 15 minutes and then a solution of methyl 3-(6-(hydroxymethyl)-5,6,7,8-tetrahydroquinazolin-2-yl)propanoate AA87 (220 mg, 0.835 mmol, 95% purity) in dry dichloromethane (1 mL) was added dropwise. The mixture was stirred at −78° C.; for 1.5 hours before a solution of triethylamine (0.93 mL, 6.69 mmol) in dry dichloromethane (1 mL) was added. The mixture was stirred at −78° C.; for 30 minutes and warmed up to room temperature for another 30 minutes. The mixture was quenched with saturated sodium bicarbonate aqueous solution (15 mL) and extracted with dichloromethane (20 mL) for three times. The combined organic layers were dried over Na₂SO₄₍ₛ₎ and filtered. The filtrate was concentrated to give the crude title compound (240 mg, 85% purity, 98% yield) as yellow oil, which was directly used in the next step without further purification. LC-MS (ESI): R_{T}=1.21 min, mass calcd. for C₁₃H₁₆N₂O₃ 248.1, m/z found 249.0 [M+H]⁺.

### Acid 18: 2-(3-Methoxy-3-oxopropyl)-5,6,7,8-tetrahydroquinazoline-6-carboxylic acid

To a solution of methyl 3-(6-formyl-5,6,7,8-tetrahydroquinazolin-2-yl)propanoate AA88 (240 mg, 0.822 mmol, 85% purity) in acetone (6.5 mL) and water (1.3 mL) was added potassium permanganate (325 mg, 2.06 mmol) at 0° C. The mixture was stirred at 0° C.; to room temperature for 1 hour. Sodium bisulfite (428 mg, 4.11 mmol) was added, then the mixture was diluted with acetone (8 mL) and water (8 mL). The resulting suspension was stirred at room temperature for 15 minutes and filtered through celite. The filtrate was concentrated under reduced pressure at room temperature to remove acetone. The resulting aqueous solution was acidified with citric acid₍ₛ₎ to pH ˜3 and extracted with ethyl acetate (20 mL) for three times. The combined organic layers were dried over Na₂SO₄₍ₛ₎, filtered and concentrated to give the title compound (180 mg, 90% purity, 75% yield) as white solids. LC-MS (ESI): R_{T}=0.28 min, mass calcd. for C₁₃H₁₆N₂O₄ 264.1, m/z found 265.0 [M+H]⁺.

### Acid 19: 2-((trans)-3-(methoxycarbonyl)cyclobutyl)-4,5,6,7-tetrahydrobenzo[d]thiazole-6-carboxylic acid

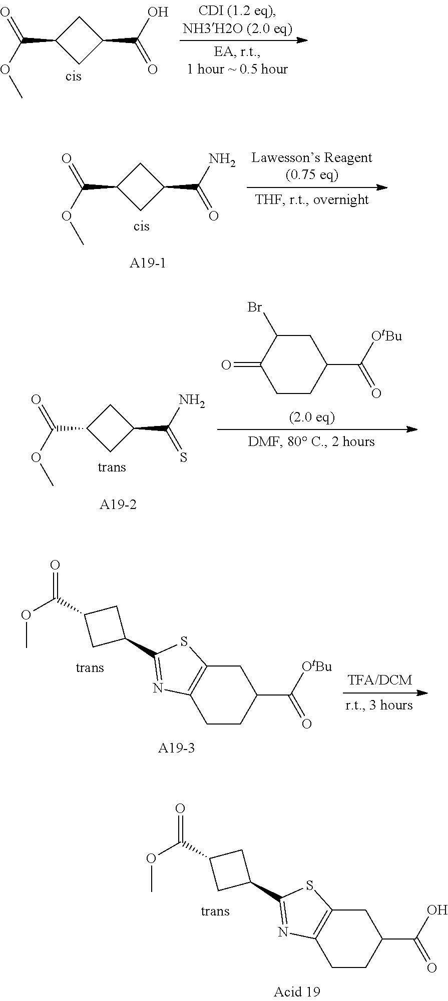 [SMILES: COC(=O)[C@H]1C[C@@H](C(=O)O)C1 ; COC(=O)[C@H]1C[C@@H](C(N)=O)C1 ; CC(C)(C)OC(=O)C1CCC(=O)C(Br)C1 ; COC(=O)[C@H]1C[C@H](C(N)=S)C1 ; COC(=O)[C@H]1C[C@H](c2nc3c(s2)CC(C(=O)OC(C)(C)C)CC3)C1 ; COC(=O)[C@H]1C[C@H](c2nc3c(s2)CC(C(=O)O)CC3)C1]

### Intermediate A19-1: (cis)-methyl 3-carbamoylcyclobutanecarboxylate

To a solution of (cis)-3-(methoxycarbonyl)cyclobutanecarboxylic acid E08495_12.6a (3.76 g, 98% purity, 23.3 mmol) in ethyl acetate (30 mL) was added N,N′-carbonyldiimidazole (4.53 g, 27.9 mmol) at room temperature. After stirred at room temperature for 1 hour, ammonium hydroxide (5.82 g, 28% purity, 46.5 mmol) was added dropwise. The mixture was stirred at room temperature for 30 minutes. The reaction mixture was acidified to pH=4-5 with 1 M hydrochloride aqueous solution, extracted with ethyl acetate (50 mL) for five times. The combined organic layers were concentrated to give the title compound (3.1 g, 98% purity, 83% yield) as white solids. ¹H NMR (300 MHz, CDCl₃) δ 6.30 (br s, 1H), 5.68 (br s, 1H), 3.68 (s, 3H), 3.21-3.13 (m, 2H), 2.58-2.39 (m, 4H).

### Intermediate A19-2: (trans)-methyl 3-carbamothioylcyclobutanecarboxylate

To a solution of (cis)-methyl 3-carbamoylcyclobutanecarboxylate A19-1 (2.6 g, 98% purity, 16.2 mmol) in tetrahydrofuran (50 mL) was added Lawesson's reagent (4.9 g, 12.1 mmol) at 0° C.; over 10 minutes. After stirred at room temperature for 16 hours, the mixture was concentrated to give a residue, which was purified by silica gel column chromatography (petroleum ether:ethyl acetate=3:1) to give the title compound A19-2 (2.45 g, 97% purity, 85% yield) and the cis isomer (280 mg, 90% purity, 9% yield) as white solids.

Intermediate A19-2: LC-MS (ESI): R_{T}=1.00 min, mass calcd. For C₇H₁₁NO₂S 173.1, m/z found 174.0 [M+H]⁺. ¹H NMR (300 MHz, CDCl₃) δ 7.67 (br s, 1H), 6.93 (br s, 1H), 3.70 (s, 3H), 3.59-3.48 (m, 1H), 3.20-3.10 (m, 1H), 2.74-2.64 (m, 2H), 2.5-2.49 (m, 2H).

### Intermediate A19-3: tert-butyl 2-((trans)-3-(methoxycarbonyl)cyclobutyl)-4,5,6,7-tetrahydrobenzo[d]thiazole-6-carboxylate

To a solution of (trans)-methyl 3-carbamothioylcyclobutanecarboxylate A19-2 (2.45 g, 97% purity, 13.7 mmol) in N,N-dimethylformamide (30 mL) was added tert-butyl 3-bromo-4-oxocyclohexanecarboxylate (8.4 g, 90% purity, 27.3 mmol). After stirred at 80° C.; for 2 hours, the reaction mixture was cooled down to room temperature and concentrated under reduced pressure to give a residue, which was purified by silica gel column chromatography (petroleum ether:ethyl acetate=10:1) to give the title compound A19-3 (2.7 g, 67% purity, 38% yield) and cis isomer (1 g, 95% purity, 20% yield) as yellow oil.

Intermediate A19-3: LC-MS (ESI): R_{T}=1.69 min, mass calcd. For C₁₈H₂₅NO₄S 351.2, m/z found 352.1 [M+H]⁺. ¹H NMR (400 MHz, CDCl₃) δ 3.96-3.87 (m, 1H), 3.72 (s, 3H), 3.28-3.21 (m, 1H), 2.96-2.85 (m, 3H), 2.78-2.59 (m, 6H), 2.24-2.20 (m, 1H), 1.94-1.88 (m, 1H), 1.45 (s, 9H).

### Acid 19: 2-((trans)-3-(methoxycarbonyl)cyclobutyl)-4,5,6,7-tetrahydrobenzo[d]thiazole-6-carboxylic acid

To a solution of tert-butyl 2-((trans)-3-(methoxycarbonyl)cyclobutyl)-4,5,6,7-tetrahydrobenzo[d]thiazole-6-carboxylate A19-3 (2.7 g, 67% purity, 5.15 mmol) in dichloromethane (20 mL) was added trifluoroacetic acid (10 mL). The reaction mixture was stirred at room temperature for 3 hours and concentrated under reduced pressure to give a residue which was purified by silica gel column chromatography (dichloromethane:methanol=20:1) to give the title compound (1.56 g, 94% yield) as yellow oil. Mass calcd. for C₁₄H₁₇NO₄S 295.1, m/z found 296.1 [M+H]⁺. ¹H NMR (400 MHz, CDCl₃) δ 3.98-3.90 (m, 1H), 3.72 (s, 3H), 3.27-3.20 (m, 1H), 3.03-3.01 (m, 2H), 2.94-2.92 (m, 1H), 2.87-2.81 (m, 2H), 2.77-2.70 (m, 2H), 2.63-2.55 (m, 2H), 2.31-2.28 (m, 1H), 2.05-1.99 (m, 1H).

### Acid 20: 2-((trans)-3-(methoxycarbonyl)cyclobutyl)-4,5,6,7-tetrahydrobenzo[d]-oxazole-6-carboxylic acid

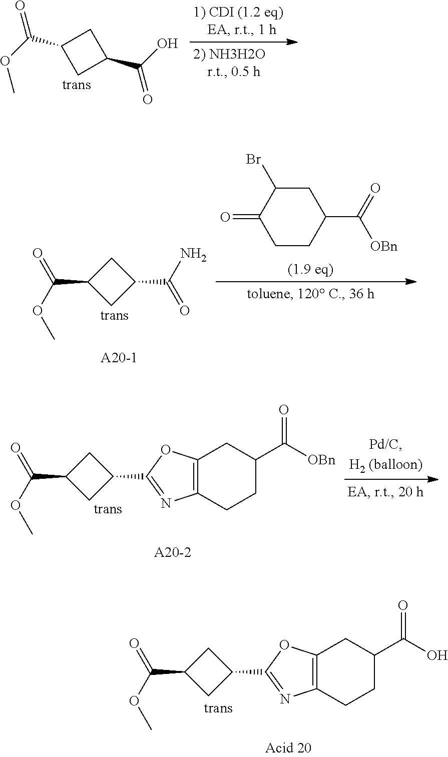 [SMILES: COC(=O)[C@H]1C[C@H](C(=O)O)C1 ; O=C1CCC(C(=O)OCc2ccccc2)CC1Br ; COC(=O)[C@H]1C[C@H](C(N)=O)C1 ; COC(=O)[C@H]1C[C@H](c2nc3c(o2)CC(C(=O)OCc2ccccc2)CC3)C1 ; COC(=O)[C@H]1C[C@H](c2nc3c(o2)CC(C(=O)O)CC3)C1]

### Intermediate A20-1: (trans)-methyl 3-carbamoylcyclobutanecarboxylate

To a solution of (trans)-3-(methoxycarbonyl)cyclobutanecarboxylic acid (3 g, 96% purity, 18.2 mmol) in ethyl acetate (20 mL) was added 1,1′-carbonyldiimidazole (3.54 g, 21.8 mmol) at room temperature. After stirred at room temperature for 1 hour, ammonium hydroxide solution (3.42 g, 28% purity, 27.3 mmol) was added dropwise. The mixture was stirred at room temperature for 30 minutes. The reaction was quenched with concentrated hydrochloric acid at 0° C. The aqueous phase was extracted with ethyl acetate (30 mL) for five times. The combined organic phases were concentrated to give a residue, which was purified by silica gel column chromatography (petroleum ether:ethyl acetate=1:1 to 1:5) to give the title compound (2.2 g, 95% purity, 73% yield) as white solids. ¹H NMR (400 MHz, DMSO-d₆) δ 7.20 (s, 1H), 6.77 (s, 1H), 3.58 (s, 3H), 3.11-2.99 (m, 1H), 2.94-2.82 (m, 1H), 2.32-2.15 (m, 4H).

### Intermediate A20-2: Benzyl 2-((trans)-3-(methoxycarbonyl)cyclobutyl)-4,5,6,7-tetrahydrobenzo[d]oxazole-6-carboxylate

To a solution of benzyl 3-bromo-4-oxocyclohexanecarboxylate (2.8 g, 90% purity, 8.10 mmol) in toluene (20 mL) was added (trans)-methyl 3-carbamoylcyclobutanecarboxylate A20-1 (700 mg, 95% purity, 4.23 mmol) at room temperature. After stirred at 120° C.; for 36 hours, the mixture was concentrated to give a residue, which was purified by silica gel column chromatography (petroleum ether:ethyl acetate=10:1 to 3:1) to give the title compound (930 mg, 54% purity, 32% yield) as yellow oil. LC-MS (ESI): R_{T}=1.59 min, mass calcd. for C₂₁H₂₃NO₅ 369.2, m/z found 370.1 [M+H]⁺. ¹H NMR (400 MHz, CDCl₃) δ 7.42-7.29 (m, 5H), 5.18-5.10 (m, 2H), 3.73-3.64 (m, 3H), 3.54-3.43 (m, 1H), 3.17-3.06 (m, 1H), 2.93-2.71 (m, 3H), 2.69-2.37 (m, 6H), 2.27-2.15 (m, 1H), 2.01-1.86 (m, 1H).

### Acid 20: 2-((trans)-3-(methoxycarbonyl)cyclobutyl)-4,5,6,7-tetrahydrobenzo[d]oxazole-6-carboxylic acid

To a solution of benzyl 2-((trans)-3-(methoxycarbonyl)cyclobutyl)-4,5,6,7-tetrahydrobenzo[d]oxazole-6-carboxylate A20-2 (930 mg, 1.36 mmol) in ethyl acetate (30 mL) was added palladium on charcoal (10% w.t., 144 mg, 0.135 mmol) at room temperature. After stirred at room temperature under hydrogen atmosphere (balloon) for 20 hours, the mixture was filtered through a pad of celite. The filtrate was concentrated under reduced pressure to give the crude title compound (730 mg, 50% purity (estimated value), 96% yield) as yellow oil which was directly used in next step without further purification. LC-MS (ESI): R_{T}=0.27 min, mass calcd. for C₁₄H₁₇NO₅ 279.1, m/z found 279.9 [M+H]⁺.

### Acid 21: 2-(methoxycarbonyl)-4,5,6,7-tetrahydrobenzo[d]oxazole-6-carboxylic acid

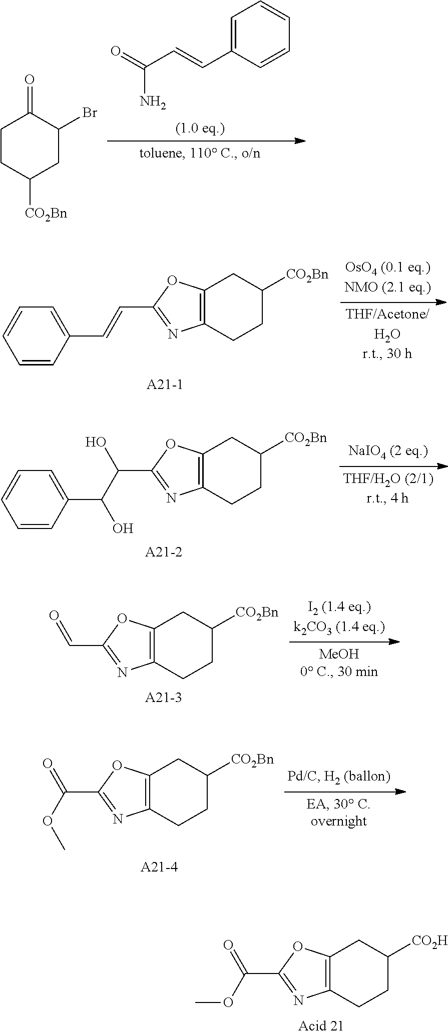

### Intermediate A21-1: (E)-benzyl 2-styryl-4,5,6,7-tetrahydrobenzo[d]oxazole-6-carboxylate

To the solution of benzyl 3-bromo-4-oxocyclohexanecarboxylate (5.0 g, 90% purity, 14.5 mmol) in toluene (80 mL) was added cinnamamide (2.16 g, 14.7 mmol) under nitrogen atmosphere at room temperature. After stirred at 110° C.; overnight and cooled down to room temperature, the reaction mixture was poured into water (50 mL) and extracted with ethyl acetate (80 mL) for three times. The combined organic layers were washed with brine (30 mL) and dried over Na₂SO₄₍ₛ₎ and filtered. The filtrate was concentrated under reduced pressure to give a crude product, which was purified by C18 column (acetonitrile:water=40% to 90%) to give the title compound (2.1 g, 95% purity, 38% yield) as light yellow solids. LC-MS (ESI): R_{T}=1.867 min, mass calcd. for C₂₃H₂₁NO₃ 359.2, m/z found 360.1 [M+H]⁺. ¹H NMR (300 MHz, CDCl₃) δ 7.54-7.48 (m, 2H), 7.43-7.33 (m, 9H), 6.89 (d, J=16.5 Hz, 1H), 5.19 (s, 2H), 3.03-2.90 (m, 3H), 2.67-2.60 (m, 2H), 2.31-2.23 (m, 1H), 2.05-1.94 (m, 1H).

### Intermediate A21-2: Benzyl 2-(1,2-dihydroxy-2-phenylethyl)-4,5,6,7-tetrahydrobenzo[d]oxazole-6-carboxylate

To the solution of (E)-benzyl 2-styryl-4,5,6,7-tetrahydrobenzo[d]oxazole-6-carboxylate A20-1 (4.0 g, 95% purity, 10.6 mmol) in tetrahydrofuran (80 mL), acetone (40 mL) and water (20 mL) was added 4-methylmorpholine 4-oxide (3.0 g, 85% purity, 21.8 mmol) and osmium(VIII) oxide (300 mg, 1.18 mmol) under nitrogen atmosphere at room temperature. After stirred at room temperature 30 hours, the reaction mixture was poured into water (100 mL) and extracted with ethyl acetate (200 mL) for three times. The combined organic layers were washed with water (200 mL) and brine (200 mL), dried over Na₂SO₄(s) and filtered. The filtrate was concentrated under reduced pressure to give a crude product (4.8 g, 85% purity, 98% yield) as dark solids, which was directly used in next step without further purification. LC-MS (ESI): R_{T}=1.38 min, mass calcd. for C₂₃H₂₃NO₅ 393.2, m/z found 394.0 [M+H]⁺.

### Intermediate A21-3: Benzyl 2-formyl-4,5,6,7-tetrahydrobenzo[d]oxazole-6-carboxylate

To the solution of benzyl 2-(1,2-dihydroxy-2-phenylethyl)-4,5,6,7-tetrahydrobenzo[d]oxazole-6-carboxylate A21-2 (3.3 g, 85% purity, 7.13 mmol) in tetrahydrofuran (90 mL) and water (45 mL) was added sodium periodate (3.05 g, 14.3 mmol) under nitrogen atmosphere at room temperature. After stirred at room temperature for 4 hours, the reaction mixture was poured into water (80 mL) and extracted with ethyl acetate (150 mL) for three times. The combined organic layers were washed with brine (200 mL), dried over Na₂SO₄₍ₛ₎ and filtered. The filtrate was concentrated under reduced pressure to give a crude product, which was purified by C18 column (acetonitrile:water=30% to 70%, 300 nm) to give the title compound (1.9 g, 90% purity from NMR, 84% yield) as dark oil. LC-MS (ESI): R_{T}=1.44 min, mass calcd. for C₁₆H₁₅NO₄ 285.1, m/z found 286.0 [M+H]⁺. ¹H NMR (300 MHz, CDCl₃) δ 9.67 (s, 1H), 7.43-7.33 (m, 5H), 5.19 (s, 2H), 3.16-2.95 (m, 3H), 2.74-2.68 (m, 2H), 2.34-2.25 (m, 1H), 2.12-1.99 (m, 1H).

### A21-4: 6-benzyl 2-methyl 4,5,6,7-tetrahydrobenzo[d]oxazole-2,6-dicarboxylate

To the solution of benzyl 2-formyl-4,5,6,7-tetrahydrobenzo[d]oxazole-6-carboxylate A21-3 (1.5 g, 90% purity, 4.73 mmol) in methanol (90 mL) was added potassium carbonate (940 mg, 6.80 mmol) and iodine (1.72 g, 6.78 mmol) under nitrogen atmosphere at 0° C. After stirred at 0° C.; for 30 minutes, the mixture was quenched with 10% sodium thiosulfate aqueous (100 mL) at 0° C. Then the mixture was extracted with ethyl acetate (150 mL) for three times. The combined organic layers were washed with water (200 mL) and brine (200 mL), dried over Na₂SO₄₍ₛ₎ and filtered. The filtrate was concentrated under reduced pressure to give a crude product, which was purified by C18 column (acetonitrile:water=30% to 70%) to give the title compound (1.17 g, 95% purity, 74% yield) as light yellow oil. LC-MS (ESI): R_{T}=1.44 min, mass calcd. for C₁₇H₁₇NO₅ 315.1, m/z found 316.0 [M+H]⁺. ¹H NMR (300 MHz, CDCl₃) δ 7.43-7.34 (m, 5H), 5.18 (s, 2H), 4.00 (s, 3H), 3.12-2.93 (m, 3H), 2.74-2.64 (m, 2H), 2.33-2.23 (m, 1H), 2.08-1.95 (m, 1H).

### Acid 21: 2-(methoxycarbonyl)-4,5,6,7-tetrahydrobenzo[d]oxazole-6-carboxylic acid

To the solution of 6-benzyl 2-methyl 4,5,6,7-tetrahydrobenzo[d]oxazole-2,6-dicarboxylate A21-4 (1.8 g, 95% purity, 5.42 mmol) in ethyl acetate (60 mL) was added 10% palladium on activated carbon (600 mg). The mixture was stirred at 30° C.; under hydrogen atmosphere overnight. 10% palladium on activated carbon was filtered off and the filtrate was concentrated to give the title compound (1.1 g, 95% purity, 85% yield) as white solids. LC-MS (ESI): R_{T}=0.28 min, mass calcd. for C₁₀H₁₁NO₅ 225.1, m/z found 226.0 [M+H]⁺.

### Acid 22: 2-(4-methoxy-2-methyl-4-oxobutan-2-yl)-4,5,6,7-tetrahydro-2H-indazole-5-carboxylic acid

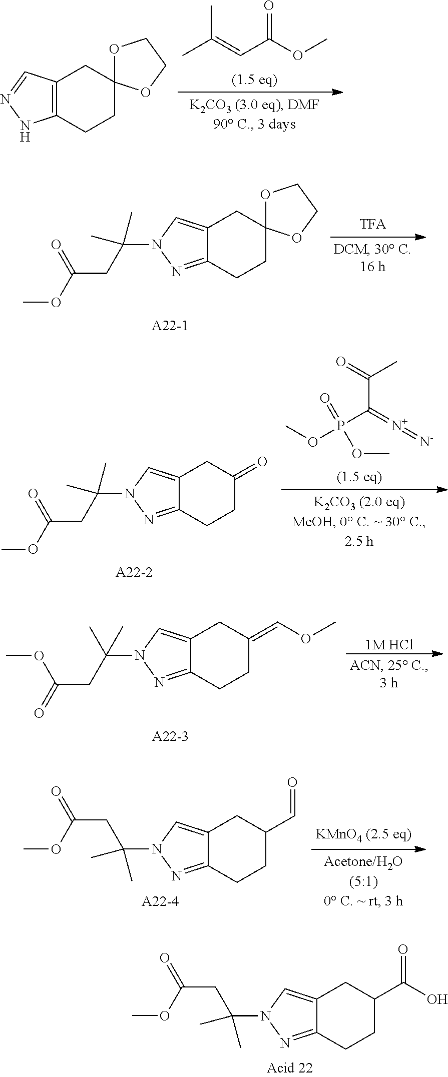

### Intermediate A22-1: methyl 3-(6′,7′-dihydrospiro[[1,3]dioxolane-2,5′-indazol]-2′(4′H)-yl)-3-methylbutanoate

To a solution of 1′,4′,6′,7′-tetrahydrospiro[[1,3]dioxolane-2,5′-indazole] (10 g, 90% purity, 49.9 mmol) and methyl 3-methyl-2-butenoate (8.7 g, 98% purity, 74.7 mmol) in N,N-dimethylformamide (100 mL) was added potassium carbonate (20.9 g, 99% purity, 149 mmol) at room temperature. The mixture was stirred at 90° C.; for 3 days. The reaction mixture was diluted with water (500 mL) and then extracted with ethyl acetate (100 mL) for three times. The combined organic layers were washed with brine (100 mL), dried over Na₂SO₄₍ₛ₎ and filtered. The filtrate was concentrated to give a residue, which was purified by C-18 column (acetonitrile:water=30% to 55%) to give the title compound (4.1 g, 90% purity from HNMR, 25% yield) as yellow oil. LC-MS (ESI): R_{T}=1.498 min, mass calcd. for C₁₅H₂₂N₂O₄ 294.2, m/z found 295.1 [M+H]⁺. ¹H NMR (400 MHz, DMSO-d₆) δ 7.45 (s, 1H), 3.91 (s, 4H), 3.51 (s, 3H), 2.83 (s, 2H), 2.65-2.62 (m, 4H), 1.84 (t, J=6.8 Hz, 2H), 1.55 (s, 6H).

### Intermediate A22-2: Methyl 3-methyl-3-(5-oxo-4,5,6,7-tetrahydro-2H-indazol-2-yl)butanoate

To a solution of methyl 3-(6′,7′-dihydrospiro[[1,3]dioxolane-2,5′-indazol]-2′(4′H)-yl)-3-methylbutanoate A22-1 (3.6 g, 90% purity, 11.0 mmol) in dichloromethane (35 mL) was added trifluoroacetic acid (35 mL) at 0° C. After stirred at 30° C.; for 16 hours, the reaction mixture was concentrated to give a residue, which was purified by C-18 column (acetonitrile:water=20% to 40%) to give the title compound (2 g, 95% purity from NMR, 70% yield) as yellow oil. ¹H NMR (400 MHz, CDCl₃) δ 7.32 (s, 1H), 3.60 (s, 3H), 3.43 (s, 2H), 3.06 (t, J=7.2 Hz, 2H), 2.91 (s, 2H), 2.69 (t, J=7.2 Hz, 2H), 1.70 (s, 6H).

### Intermediate A22-3: Methyl 3-(5-(methoxymethylene)-4,5,6,7-tetrahydro-2H-indazol-2-yl)-3-methylbutanoate

To a solution of methyl 3-methyl-3-(5-oxo-4,5,6,7-tetrahydro-2H-indazol-2-yl)butanoate A22-22 (2 g, 95% purity, 7.59 mmol) and dimethyl(1-diazo-2-oxopropyl)phosphanate (2.2 g, 11.5 mmol) in methanol (20 mL) was added potassium carbonate (2.1 g, 15.2 mmol) at 0° C. After stirred at 0° C.; for 30 minutes and then at 30° C.; for 2 hours under nitrogen atmosphere, the reaction was quenched with saturated ammonium chloride aqueous solution (100 mL) at 0° C.; and extracted with ethyl acetate (30 mL) for three times. The combined organic layers were washed with brine (30 mL), dried over Na₂SO₄₍ₛ₎ and filtered. The filtrate was concentrated to give a residue, which was purified by C18 column (acetonitrile:water=30% to 60%) to give the title compound (1.7 g, 98.0% purity, 79% yield) as colorless oil. LC-MS (ESI): R_{T}=1.740 min, mass calcd. for C₁₅H₂₂N₂O₃ 278.2, m/z found 279.1 [M+H]⁺. ¹H NMR (400 MHz, CDCl₃) δ 7.24 (s, 0.6H), 7.23 (s, 0.4H), 6.00 (s, 0.4H), 5.94 (s, 0.6H), 3.59-3.57 (m, 6H), 3.32 (s, 1.3H), 3.10 (s, 0.7H), 2.88 (s, 2H), 2.74-2.69 (m, 2H), 2.53 (t, J=6.4 Hz, 0.7H), 2.31 (t, J=6.4 Hz, 1.3H), 1.67 (s, 6H).

### Intermediate A22-4: Methyl 3-(5-formyl-4,5,6,7-tetrahydro-2H-indazol-2-yl)-3-methylbutanoate

To a solution of methyl 3-(5-(methoxymethylene)-4,5,6,7-tetrahydro-2H-indazol-2-yl)-3-methylbutanoate A22-3 (1.7 g, 98.0% purity, 5.99 mmol) in acetonitrile (17 mL) was added 1 M hydrochloride aqueous solution (17 mL, 17 mmol, 1 M) at 0° C. After stirred at 25° C.; for 3 hours, the mixture was diluted with brine (30 mL) and then pH was basified to 8-9 with saturated bicarbonate aqueous solution. The resulting mixture was extracted with ethyl acetate (20 mL) for three times. The combined organic layers were dried over Na₂SO₄₍ₛ₎ and filtered. The filtrate was concentrated to give the title compound (1.38 g, 90% purity from NMR, 79% yield) as yellow oil. ¹H NMR (400 MHz, CDCl₃) δ 9.79 (s, 1H), 7.29 (s, 1H), 3.59 (s, 3H), 2.88-2.80 (m, 4H), 2.75-2.59 (m, 3H), 2.29-2.22 (m, 1H), 1.88-1.78 (m, 1H), 1.68-1.67 (m, 6H).

### Acid 22: 2-(4-methoxy-2-methyl-4-oxobutan-2-yl)-4,5,6,7-tetrahydro-2H-indazole-5-carboxylic acid

To a solution of methyl 3-(5-formyl-4,5,6,7-tetrahydro-2H-indazol-2-yl)-3-methylbutanoate A22-4 (1.38 g, 90% purity, 4.70 mmol) in acetone (45 mL) and water (9 mL) was added potassium permangnate (1.86 g, 11.8 mmol) at 0° C. After stirred in a range from 0° C.; to room temperature for 1 hour, the reaction mixture was quenched with an addition of sodium bisulfite (2.44 g, 23.5 mmol) followed by dilution with acetone (40 mL) and water (40 mL). The resulting suspension was stirred at room temperature for 15 minutes and filtered through a pad of celite. The filtrate was concentrated under reduced pressure at room temperature to remove acetone. The residue was then acidified with citric acid(s) to pH ˜3 and extracted with ethyl acetate (50 mL) for three times. The combined organic layers were dried over Na₂SO₄₍ₛ₎ and filtered. The filtrate was concentrated to give the title compound (1.4 g, 86% purity, 91% yield) as yellow oil. LC-MS (ESI): R_{T}=1.336 min, mass calcd. for C₁₄H₂₀N₂O₄ 280.1, m/z found 281.1 [M+H]⁺. ¹H NMR (400 MHz, CDCl₃) δ 7.27 (s, 1H), 3.59 (s, 3H), 2.94-2.84 (m, 4H), 2.78-2.64 (m, 3H), 2.31-2.24 (m, 1H), 1.97-1.84 (m, 1H), 1.68 (s, 6H).

### Acid 23: 2-(3-ethoxy-2,2-dimethyl-3-oxopropyl)-4,5,6,7-tetrahydro-2H-indazole-5-carboxylic acid

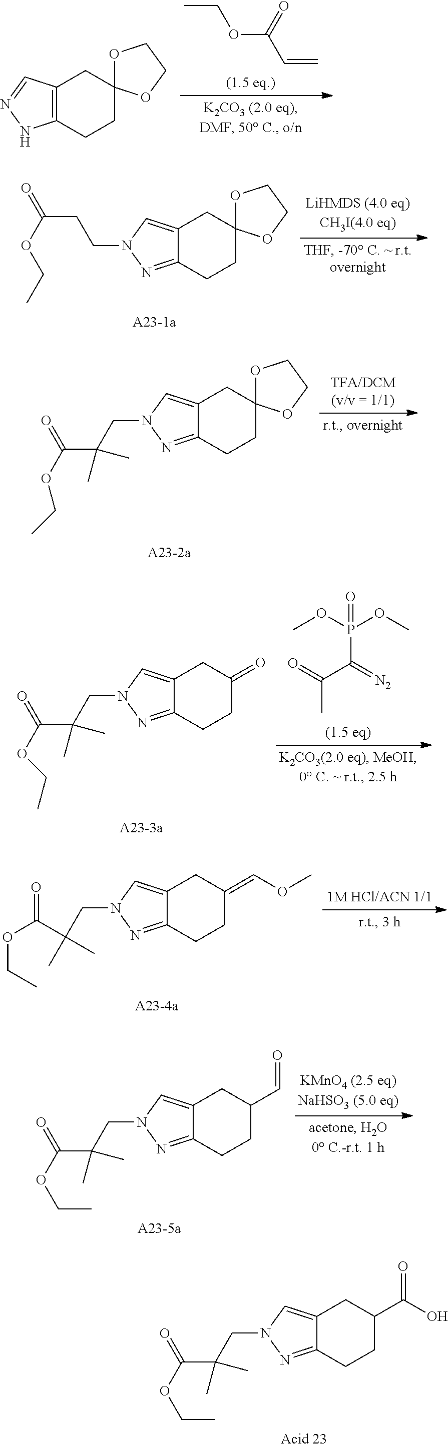

### Intermediate A23-1a: ethyl 3-(6,7-dihydrospiro[indazole-5,2′-[1,3]dioxolan]-2(4H)-yl)propanoate

To a solution of 1′,4′,6′,7′-tetrahydrospiro[[1,3]dioxolane-2,5′-indazole] (23.0 g, 128 mmol) in N,N-dimethylformide (230 mL) was added ethyl acrylate (19.2 g, 192 mmol) and potassium carbonate (35.3 g, 256 mmol) at room temperature. After stirred at 50° C.; under nitrogen atmosphere overnight and cooled down to room temperature, the mixture was concentrated under reduced pressure to remove the volatile, then poured into water (100 mL) and extracted with ethyl acetate (100 mL) for three times. The combined organic layers were washed with water (100 mL), brine (100 mL), dried over Na₂SO₄₍ₛ₎ and filtered. The filtrate was concentrated under reduced pressure to give a crude product, which was purified by C18 column (acetonitrile:water=5% to 60%) to give a mixture of compound A23-1a and its regioisomer (32 g, 89% yield) as light yellow oil.

The mixture (33 g, 95% purity, 112 mmol) was further separated by SFC (separation condition: Column: Chiralpak IG 5 μm 20*250 mm; Mobile Phase: CO₂:IPA=75:25 at 50 g/min; Temp: 40° C.; Wavelength: 230 nm, Back pressure: 100 bar) to afford the title compound A23-1a (15.8 g, 90% purity from NMR, 45% yield) as light yellow oil and A23-1b (13.8 g, 90% purity from NMR, 39% yield) as light yellow oil.

Intermediate A23-1a: LC-MS (ESI): R_{T}=1.37 min, mass calcd. for C₁₄H₂₀N₂O₄ 280.1, m/z found 281.0 [M+H]⁺. Chiral analysis: (Column: Chiralpak IG 5 μm 4.6*250 mm; Mobile Phase: CO₂:IPA=75:25 at 3 g/min; Col. Temp: 40° C.; Wavelength: 214 nm, Back pressure: 100 bar, R_{T}=2.89 min). ¹H NMR (400 MHz, DMSO-d₆) δ 7.34 (s, 1H), 4.21 (t, J=6.8 Hz, 2H), 4.05 (q, J=7.2 Hz, 2H), 3.90 (s, 4H), 2.79 (t, J=6.4 Hz, 2H), 2.65-2.62 (m, 4H), 1.83 (t, J=6.4 Hz, 2H), 1.16 (t, J=6.8 Hz, 3H).

### Intermediate A23-2a: Ethyl 3-(6′,7′-dihydrospiro[[1,3]dioxolane-2,5′-indazol]-2′(4′H)-yl)-2,2-dimethylpropanoate

To a solution of ethyl 3-(6′,7′-dihydrospiro[[1,3]dioxolane-2,5′-indazol]-2′(4′H)-yl)propanoate A23-1a (5.90 g, 95% purity, 20.0 mmol) in dry tetrahydrofuran (100 mL) was added 1.0 M lithium hexamethyldisilazide in tetrahydrofuran (80 mL, 80 mmol) dropwise at −70° C. The resulting mixture was stirred at −70° C.; for 2 hours, and then iodomethane (5 mL, 80.3 mmol) was added at the same temperature. After stirred at room temperature overnight, the mixture was quenched with saturated ammonium chloride aqueous solution (100 mL) and extracted with ethyl acetate (150 mL) twice. The combined organic layers were washed with brine (100 mL), dried over Na₂SO₄₍ₛ₎, filtered and concentrated under reduced pressure to give the title compound (5.9 g, 95% purity from NMR, 91% yield) as a yellow oil. LC-MS (ESI): R_{T}=1.50 min, mass calcd. for C₁₆H₂₄N₂O₄ 308.2, m/z found 309.0 [M+H]⁺. ¹H NMR (400 MHz, CDCl₃) δ 7.08 (s, 1H), 4.19-4.13 (m, 4H), 4.02 (s, 4H), 2.84 (t, J=6.8 Hz, 2H), 2.75 (s, 2H), 1.96 (t, J=6.8 Hz, 2H), 1.26 (t, J=7.2 Hz, 3H), 1.20 (s, 6H).

### Intermediate A23-3a: Ethyl 2,2-dimethyl-3-(5-oxo-4,5,6,7-tetrahydro-2H-indazol-2-yl)propanoate

To a solution of ethyl 3-(6′,7′-dihydrospiro[[1,3]dioxolane-2,5′-indazol]-2′(4′H)-yl)-2,2-dimethylpropanoate A23-2a (5.90 g, 95% purity, 18.2 mmol) in dichloromethane (50 mL) was added trifluoroacetic acid (50 mL) at 0° C. After stirred at room temperature overnight, the reaction mixture was concentrated under reduced pressure, the residue was basified with saturated sodium carbonate aqueous solution to pH ˜8 and then extracted with ethyl acetate (150 mL) twice. The combined organic layers were washed with saturated sodium carbonate aqueous solution (100 mL) twice and brine (100 mL), dried over Na₂SO₄₍ₛ₎ and filtered. The filtrate was concentrated under reduced pressure to give the title compound (5.05 g, 90% purity from NMR, 95% yield) as yellow oil. LC-MS (ESI): R_{T}=1.47 min, mass calcd. for C₁₄H₂₀N₂O₃ 264.1, m/z found 265.0 [M+H]⁺. ¹H NMR (400 MHz, CDCl₃) δ 7.16 (s, 1H), 4.23 (s, 2H), 4.17 (q, J=7.2 Hz, 2H), 3.41 (s, 2H), 3.04 (t, J=7.2 Hz, 2H), 2.68 (t, J=7.2 Hz, 2H), 1.27 (t, J=7.2 Hz, 3H), 1.21 (s, 6H).

### Intermediate A23-4a: Ethyl 3-(5-(methoxymethylene)-4,5,6,7-tetrahydro-2H-indazol-2-yl)-2,2-dimethylpropanoate

To a solution of ethyl 2,2-dimethyl-3-(5-oxo-4,5,6,7-tetrahydro-2H-indazol-2-yl)propanoate A23-3a (5.05 g, 90% purity, 17.2 mmol) and dimethyl (1-diazo-2-oxopropyl)phosphonate (4.95 g, 25.8 mmol) in dry methanol (50 mL) was added potassium carbonate (4.76 g, 34.4 mmol) at 0° C.; under nitrogen atmosphere. After stirred at 0° C.; for 30 minutes and then at room temperature for 2 hours, the reaction mixture was diluted with water (100 mL) and extracted with ethyl acetate (100 mL) twice. The combined organic layers were washed with brine (50 mL), dried over Na₂SO₄₍ₛ₎ and filtered. The filtrate was concentrated under reduced pressure to give a residue, which was purified by silica gel column chromatography (petroleum ether:ethyl acetate=5:1 to 3:1) to give the title compound (3.3 g, 80% purity from NMR, 53% yield) as yellow oil. LC-MS (ESI): R_{T}=1.67 min, mass calcd. for C₁₆H₂₄N₂O₃ 292.2, m/z found 293.1 [M+H]⁺. ¹H NMR (400 MHz, CDCl₃) δ 7.06 (s, 0.7H), 7.04 (s, 0.3H), 6.00 (s, 0.3H), 5.94 (s, 0.7H), 4.18-4.13 (m, 4H), 3.58 (s, 2.1H), 3.57 (s, 0.9H), 3.30 (s, 1.4H), 3.08 (s, 0.6H), 2.71-2.64 (m, 2H), 2.53-2.50 (m, 0.6H), 2.31-2.22 (m, 1.4H), 1.26 (t, J=7.2 Hz, 3H), 1.19 (s, 6H).

### Intermediate A23-5a: Ethyl 3-(5-formyl-4,5,6,7-tetrahydro-2H-indazol-2-yl)-2,2-dimethylpropanoate

To a solution of ethyl 3-(5-(methoxymethylene)-4,5,6,7-tetrahydro-2H-indazol-2-yl)-2,2-dimethylpropanoate A23-4a (3.29 g, 80% purity, 9.00 mmol) in acetonitrile (50 mL) was added 2 M hydrochloride aqueous solution (50 mL) at 0° C. After stirred at room temperature for 3 hours, the reaction mixture was basified with saturated sodium bicarbonate aqueous solution to pH ˜8. The resulting solution was extracted with ethyl acetate (50 mL) twice. The combined organic layers were washed with brine (40 mL), dried over Na₂SO₄₍ₛ₎ and filtered. The filtrate was concentrated under reduced pressure to give the title compound (3.00 g, 80% purity from NMR, 96% yield) as green oil. LC-MS (ESI): R_{T}=1.53 min, mass calcd. for C₁₅H₂₂N₂O₃ 278.2, m/z found 279.0 [M+H]⁺. ¹H NMR (400 MHz, CDCl₃) δ 9.78 (s, 1H), 7.12 (s, 1H), 4.20-4.10 (m, 4H), 2.85-2.78 (m, 2H), 2.73-2.58 (m, 3H), 2.28-2.22 (m, 1H), 1.86-1.79 (m, 1H), 1.26 (t, J=7.2 Hz, 3H), 1.20 (s, 6H).

### Acid 23: 2-(3-ethoxy-2,2-dimethyl-3-oxopropyl)-4,5,6,7-tetrahydro-2H-indazole-5-carboxylic acid

To a solution of ethyl 3-(5-formyl-4,5,6,7-tetrahydro-2H-indazol-2-yl)-2,2-dimethylpropanoate A23-5a (3.00 g, 80% purity, 8.62 mmol) in acetone (80 mL) and water (16 mL) was added potassium permanganate (3.41 g, 21.6 mmol) at 0° C. After stirred at 0° C.; for 1 hour, sodium bisulfite (4.49 g, 43.1 mmol) was added. Then the mixture was diluted with acetone (80 mL) and water (80 mL). The resulting suspension was stirred at room temperature for 15 minutes and filtered. The filtrate was concentrated under reduced pressure at room temperature to remove acetone. The resulting aqueous solution was acidified with citric acid(s) to pH ˜3 and extracted with ethyl acetate (80 mL) twice. The combined organic layers were washed with brine (50 mL), dried over Na₂SO₄₍ₛ₎ and filtered. The filtrate was concentrated under reduced pressure to give the title compound (3.00 g, 80% purity from NMR, 95% yield) as white solids. ¹H NMR (400 MHz, CDCl₃) δ 7.11 (s, 1H), 4.23 (s, 2H), 4.19-4.10 (m, 2H), 2.90-2.82 (m, 2H), 2.77-2.63 (m, 3H), 2.30-2.23 (m, 1H), 1.96-1.86 (m, 1H), 1.26 (t, J=7.2 Hz, 3H), 1.20 (s, 6H).

### Acid 24: 2-((trans)-4-(methoxycarbonyl)cyclohexyl)-4,5,6,7-tetrahydrobenzo[d]oxazole-6-carboxylic acid

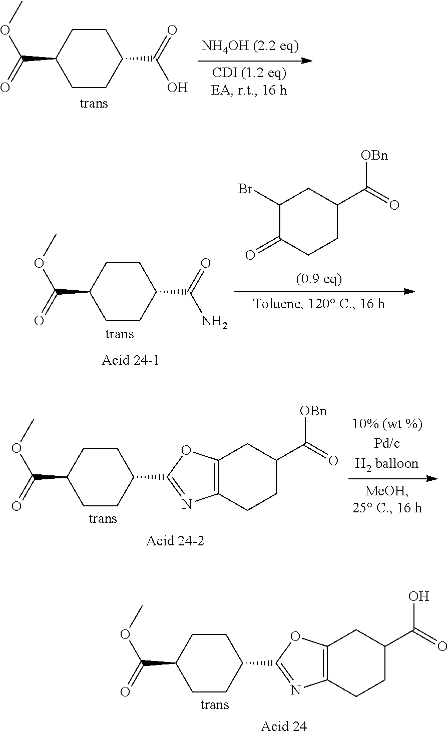

### Intermediate A24-1: (trans)-methyl 4-carbamoylcyclohexanecarboxylate

To a solution of (trans)-4-(methoxycarbonyl)cyclohexanecarboxylic acid (5 g, 26.9 mmol) in ethyl acetate (100 mL) was added 1,1′-carbonyldiimidazole (5.35 g, 33.0 mmol) at room temperature. The reaction mixture was stirred at room temperature for 1 hour, and then 28% ammonia aqueous solution (7.5 g, 60.0 mmol) was added at 0° C. After stirred at room temperature for 16 hours, the reaction mixture was quenched with concentrated hydrochloride aqueous solution (20 mL) and extracted with ethyl acetate (100 mL) twice. The combined organic layers were dried over Na₂SO₄₍ₛ₎ and filtered. The filtrate was concentrated under reduced pressure to give the title compound (2.3 g, 46% yield) as white solids. ¹H NMR (400 MHz, DMSO-d₆) δ 7.20 (s, 1H), 6.69 (s, 1H), 3.57 (s, 3H), 2.30-2.20 (m, 1H), 2.09-1.72 (m, 5H), 1.40-1.13 (m, 4H).

### Intermediate A24-2: benzyl 2-((trans)-4-(methoxycarbonyl)cyclohexyl)-4,5,6,7-tetrahydrobenzo[d]oxazole-6-carboxylate

To a solution of benzyl 3-bromo-4-oxocyclohexanecarboxylate (2 g, 6.43 mmol) in toluene (50 mL) was added (trans)-methyl 4-carbamoylcyclohexanecarboxylate A24-1 (1.3 g, 7.02 mmol) at room temperature under nitrogen atmosphere. After stirred at 120° C. for 16 hours, the reaction mixture was poured into water (100 mL) and then extracted with ethyl acetate (100 mL) twice. The combined organic layers were dried over Na₂SO₄₍ₛ₎ and filtered. The filtrate was concentrated and purified by silica gel column chromatography (petroleum ether:ethyl acetate=10:1 to 3:1) to give the title compound (1.4 g, 55% yield) as brown solids. ¹H NMR (300 MHz, CDCl₃) δ 7.38-7.35 (m, 5H), 5.17 (s, 2H), 3.69 (s, 3H), 2.95-2.85 (m, 3H), 2.76-2.67 (m, 1H), 2.59-2.51 (m, 2H), 2.35-2.06 (m, 7H), 1.97-1.91 (m, 1H), 1.64-1.60 (m, 1H), 1.55-1.43 (m, 2H).

### Acid 24: 2-((trans)-4-(methoxycarbonyl)cyclohexyl)-4,5,6,7-tetrahydrobenzo[d]oxazole-6-carboxylic acid

To a solution of benzyl 2-((trans)-4-(methoxycarbonyl)cyclohexyl)-4,5,6,7-tetrahydrobenzo[d]oxazole-6-carboxylate A24-2 (1.4 g, 3.52 mmol)) in methanol (50 mL) was added 10% palladium on charcoal (300 mg) under nitrogen atmosphere at room temperature. After replacing the inert nitrogen atmosphere with hydrogen gas, the mixture was stirred at 25° C.; under hydrogen atmosphere for 16 hours. The catalyst was filtered off and the filtrate was concentrated under reduced pressure to give the title product (1.1 g, 100% yield) as white solids. LC-MS (ESI): R_{T}=1.17 min, mass calcd. for C₁₆H₂₁NO₅ 307.1, m/z found 306.0 [M−H]⁻.

### Acid 25: 2-(4-Ethoxy-3,3-dimethyl-4-oxobutyl)-4,5,6,7-tetrahydro-2H-indazole-5-carboxylic acid

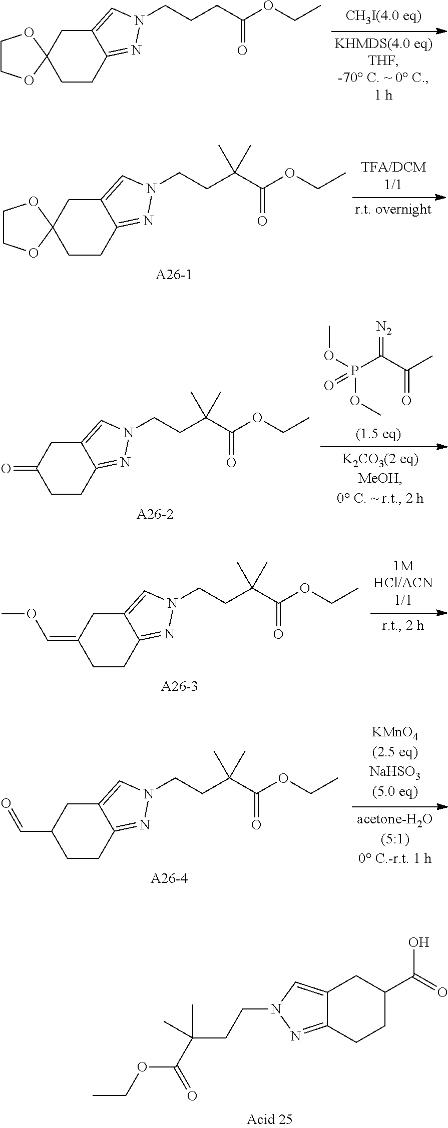

### Intermediate A25-1: Ethyl 4-(6′,7′-dihydrospiro[[1,3]dioxolane-2,5′-indazol]-2′ (4′H)-yl)-2,2-dimethylbutanoate

To a solution of ethyl 4-(6′,7′-dihydrospiro[[1,3]dioxolane-2,5′-indazol]-2′(4′H)-yl)butanoate (7.30 g, 90% purity, 22.3 mmol) in tetrahydrofuran (73 mL) was added dropwise 1 M potassium bis(trimethylsilyl)amide in tetrahydrofuran (88 mL) at −70° C. After stirred at −70° C.; for 1 hour under nitrogen atmosphere, iodomethane (12.7 g, 89.4 mmol) was added dropwise at −70° C., and then the reaction mixture was slowly warmed to 0° C. The reaction mixture was quenched with ammonium chloride aqueous solution (100 mL) and extracted with ethyl acetate (100 mL) for three times. The separated organic layer was washed with brine (100 mL), dried over Na₂SO₄₍ₛ₎, filtered and concentrated under reduced pressure to give a residue, which was purified by C18 column (acetonitrile:water=20% to 60%) to afford a crude product which was further purified by Prep. HPLC (Column: Xtimate C18 (10 μm 50*250 mm), Mobile phase A: water (0.1% ammonium bicarbonate), Mobile phase B: acetonitrile, UV: 214 nm, Flow rate: 80 mL/min, Gradient: 15-55% (% B)) to give the title compound (6.90 g, 95% purity from ¹H NMR, 67% yield) as yellow oil. LC-MS (ESI): R_{T}=1.649 min, mass calcd. for C₁₇H₂₆N₂O₄ 322.2, m/z found 323.1 [M+H]⁺. ¹H NMR (400 MHz, CDCl₃) δ 7.08 (s, 1H), 4.11 (d, J=7.2 Hz, 2H), 4.04-4.00 (m, 6H), 2.86 (t, J=6.8 Hz, 2H), 2.76 (s, 2H), 2.11-2.06 (m, 2H), 1.97 (t, J=6.8 Hz, 2H), 1.27-1.23 (m, 9H).

### Intermediate A25-2: Ethyl 2,2-dimethyl-4-(5-oxo-4,5,6,7-tetrahydro-2H-indazol-2-yl)butanoate

To a solution of ethyl 4-(6′,7′-dihydrospiro[[1,3]dioxolane-2,5′-indazol]-2′(4′H)-yl)-2,2-dimethylbutanoate A25-1 (6.90 g, 95% purity, 20.3 mmol) in dichloromethane (69 mL) was added 2,2,2-trifluoroacetic acid (69 mL) at 0° C. After stirred at room temperature overnight under nitrogen atmosphere, the reaction mixture was concentrated, then added saturated sodium carbonate aqueous solution (100 mL) and extracted with ethyl acetate (100 mL) twice. The combined organic layers were washed with brine (50 mL), dried over Na₂SO₄₍ₛ₎ and filtered. The filtrate was concentrated to give title compound (6.50 g, 86% purity, 99% yield) as yellow oil. LC-MS (ESI): R_{T}=1.479 min, mass calcd. for C₁₅H₂₂N₂O₃ 278.2, m/z found 279.1 [M+H]⁺. ¹H NMR (400 MHz, CDCl₃) δ 7.15 (s, 1H), 4.13-4.05 (m, 4H), 3.41 (s, 2H), 3.04 (t, J=7.2 Hz, 2H), 2.68 (t, J=7.2 Hz, 2H), 2.12-2.08 (m, 2H), 1.26-1.22 (m, 9H).

### Intermediate A25-3: Ethyl 4-(5-(methoxymethylene)-4,5,6,7-tetrahydro-2H-indazol-2-yl)-2,2-dimethylbutanoate

To a solution of ethyl 2,2-dimethyl-4-(5-oxo-4,5,6,7-tetrahydro-2H-indazol-2-yl)butanoate A25-2 (6.50 g, 86% purity, 20.1 mmol) and dimethyl (1-diazo-2-oxopropyl)phosphonate (5.79 g, 30.1 mmol) in dry methanol (65 mL) was added potassium carbonate (5.55 g, 40.2 mmol) at 0° C.; under nitrogen atmosphere. After stirred at 0° C.; for 30 minutes and then at room temperature for 2 hours, the mixture was poured into water (200 mL) and extracted with ethyl acetate (100 mL) twice. The combined organic layers were washed with water (50 mL), brine (50 mL) twice, dried over Na₂SO₄₍ₛ₎, filtered and concentrated to give a crude product which was purified by C18 column (acetonitrile:water=20% to 95%) to give the title compound (4.60 g, 95% purity from ¹H NMR, 71% yield) as brown oil. ¹H NMR (400 MHz, CDCl₃) δ 7.08 (s, 0.6H), 7.05 (s, 0.4H), 6.00 (s, 0.4H), 5.94 (s, 0.6H), 4.14-4.08 (m, 2H), 4.04-4.00 (m, 2H), 3.58-3.56 (m, 3H), 3.31 (s, 1.3H), 3.09 (s, 0.7H), 2.73-2.68 (m, 2H), 2.52 (t, J=6.4 Hz, 0.7H), 2.30 (t, J=6.4 Hz, 1.3H), 2.10-2.06 (m, 2H), 1.27-1.23 (m, 9H).

### Intermediate A25-4: Ethyl 4-(5-formyl-4,5,6,7-tetrahydro-2H-indazol-2-yl)-2,2-dimethylbutanoate

To a solution of ethyl 4-(5-(methoxymethylene)-4,5,6,7-tetrahydro-2H-indazol-2-yl)-2,2-dimethylbutanoate A25-3 (4.60 g, 95% purity, 14.262 mmol) in acetonitrile (46 mL) was added 1 M hydrochloric acid aqueous solution (46 mL) at 0° C. After stirred at room temperature for 2 hours, to the reaction mixture was added saturated sodium bicarbonate aqueous solution (100 mL), and the resulting solution was extracted with ethyl acetate (100 mL) twice. The combined organic layers were washed with water (50 mL) twice, brine (50 mL) twice, dried over Na₂SO₄₍ₛ₎ and concentrated to give the crude title compound (4.50 g, 77% purity, 83% yield) as red oil. LC-MS (ESI): R_{T}=1.568 min, mass calcd. for C₁₆H₂₄N₂O₃ 292.2, m/z found 293.1 [M+H]⁺. ¹H NMR (400 MHz, CDCl₃) δ 9.78 (s, 1H), 7.13 (s, 1H), 4.12 (q, J=7.2 Hz, 2H), 4.05-4.01 (m, 2H), 2.86-2.78 (m, 2H), 2.74-2.61 (m, 3H), 2.27-2.23 (m, 1H), 2.11-2.07 (m, 2H), 1.88-1.78 (m, 1H), 1.27-1.23 (m, 9H).

### Acid 25: 2-(4-Ethoxy-3,3-dimethyl-4-oxobutyl)-4,5,6,7-tetrahydro-2H-indazole-5-carboxylic acid

To a solution of ethyl 4-(5-formyl-4,5,6,7-tetrahydro-2H-indazol-2-yl)-2,2-dimethylbutanoate A25-4 (4.50 g, 77% purity, 11.9 mmol) in acetone (100 mL) and water (20 mL) was added potassium permanganate (4.68 g, 29.6 mmol) at 0° C. After stirred at 0° C.; for 1 hour, sodium bisulfite (6.17 g, 59.3 mmol) was added, and then the mixture was diluted with acetone (100 mL) and water (100 mL). The resulting suspension was stirred at room temperature for 15 minutes and filtered. The filtrate was concentrated under reduced pressure at room temperature to remove acetone. The resulting aqueous solution was acidified with citric acid (50 mL) to pH 3-4 and extracted with ethyl acetate (100 mL) twice. The combined organic layers were washed with brine (100 mL), dried over Na₂SO₄₍ₛ₎ and concentrated to give the compound (7.80 g crude) as red oil. 5.8 g crude compound was purified by C18 column (acetonitrile: 0.1% hydrogen chloride in water=% to 60%) to give the title compound (2.60 g, 92% purity from HNMR, 65% yield) as yellow solids. LC-MS (ESI): R_{T}=0.967 min, mass calcd. For C₁₆H₂₄N₂O₄ 308.2, m/z found 309.1 [M+H]⁺. ¹H NMR (400 MHz, CDCl₃) δ 7.48 (s, 1H), 4.58-4.44 (m, 2H), 4.13 (q, J=7.2 Hz, 2H), 3.09-3.01 (m, 1H), 2.91-2.81 (m, 4H), 2.24-2.20 (m, 3H), 2.10-2.05 (m, 1H), 1.28-1.26 (m, 9H).

### Acid 26: (cis)-2-(3-(Methoxycarbonyl)cyclobutyl)-4,5,6,7-tetrahydro-2H-indazole-5-carboxylic acid

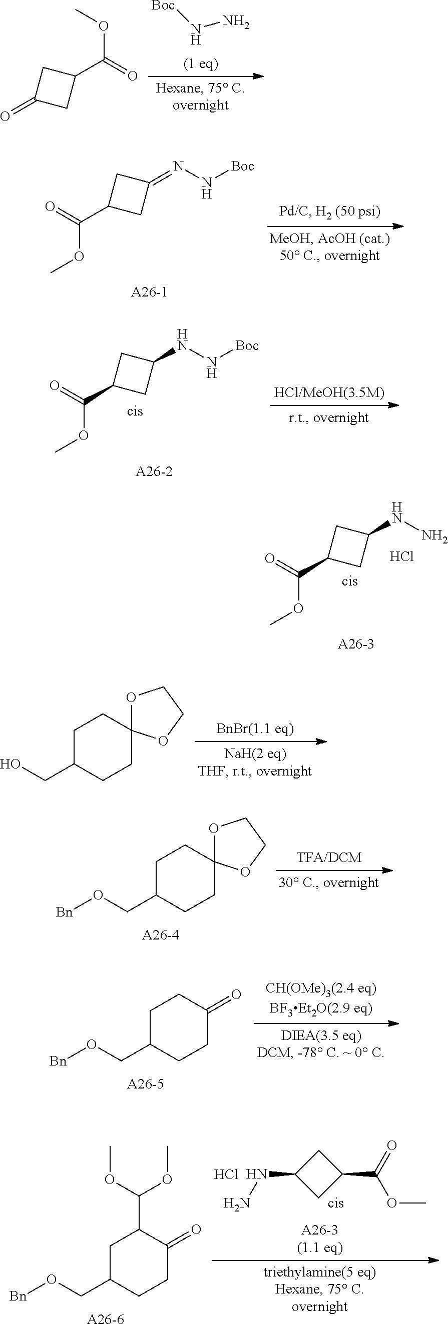

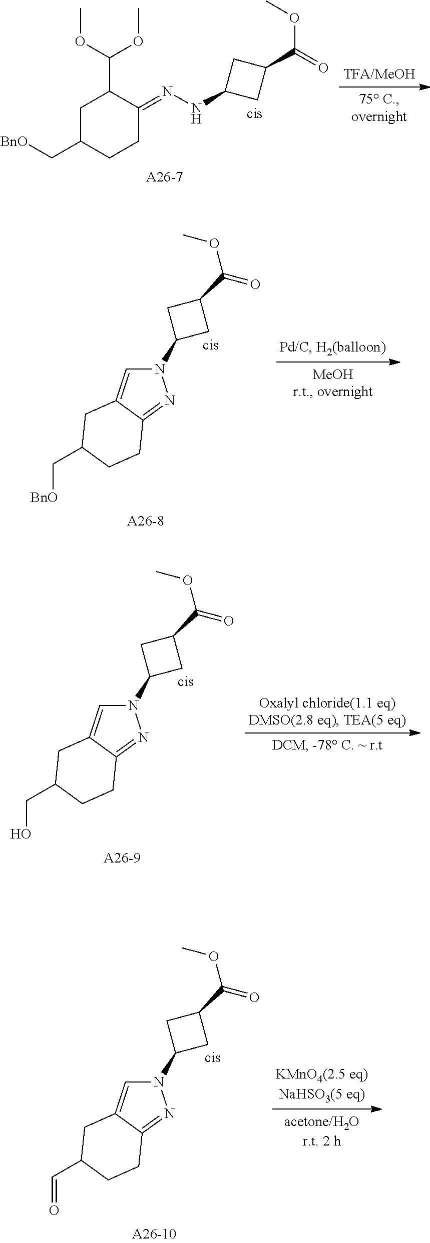 [SMILES: COC(=O)[C@H]1C[C@@H](N/N=C2\CCC(COCc3ccccc3)CC2C(OC)OC)C1 ; COC(=O)[C@H]1C[C@@H](n2cc3c(n2)CCC(COCc2ccccc2)C3)C1 ; COC(=O)[C@H]1C[C@@H](n2cc3c(n2)CCC(CO)C3)C1 ; COC(=O)[C@H]1C[C@@H](n2cc3c(n2)CCC(C=O)C3)C1]

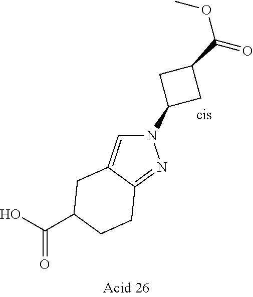 [SMILES: COC(=O)[C@H]1C[C@@H](n2cc3c(n2)CCC(C(=O)O)C3)C1]

### Intermediate A26-1: tert-Butyl 2-(3-(methoxycarbonyl)cyclobutylidene)hydrazinecarboxylate

To the solution of methyl 3-oxocyclobutanecarboxylate (8.4 g, 95% purity, 62.3 mmol) in hexane (150 mL) was added tert-butyl hydrazinecarboxylate (8.3 g, 62.8 mmol) at room temperature. After stirred at 75° C.; under nitrogen atmosphere overnight, the mixture was filtered hot. The filtration was collected to give the title compound (8 g, 95% purity from NMR, 50% yield) as white solids. ¹H NMR (300 MHz, CDCl₃) δ 7.45 (br s, 1H), 3.73 (s, 3H), 3.27-3.00 (m, 5H), 1.5 (s, 9H).

### Intermediate A26-2: (cis)-tert-Butyl 2-(3-(methoxycarbonyl)cyclobutyl)hydrazinecarboxylate

To the solution of tert-butyl 2-(3-(methoxycarbonyl)cyclobutylidene) hydrazinecarboxylate A26-1 (23.0 g, 95% purity, 90.2 mmol) in methanol (300 mL) was added 10% palladium on charcoal (5 g). The mixture was stirred at room temperature under hydrogen atmosphere overnight. 10% palladium on charcoal was filtered off and the filtrate was concentrated to give the title compound (23.0 g, 95% purity from NMR, 99% yield) as light yellow solids. ¹H NMR (300 MHz, CDCl₃) δ 3.71-3.69 (m, 3H), 3.47-3.37 (m, 1H), 2.80-2.68 (m, 1H), 2.82-2.23 (m, 4H), 1.49 (s, 9H).

### Intermediate A26-3: (cis)-Methyl 3-hydrazinylcyclobutanecarboxylate hydrochloride

To a solution of (cis)-tert-butyl 2-(3-(methoxycarbonyl)cyclobutyl) hydrazinecarboxylate A26-2 (18 g, 95% purity, 70 mmol) in methanol (50 mL) was added 3.5 M hydrochloride in methanol (150 mL) dropwise. After addition, the mixture was stirred at room temperature overnight. The white solid was collected by filtration to give the title compound (11 g, 90% purity from NMR, 78% yield). ¹H NMR (300 MHz, DMSO-d₆) δ 3.74-3.53 (m, 4H), 3.22-3.14 (m, 0.5H), 2.95-2.85 (m, 0.5H), 2.39-2.31 (m, 2.7H), 2.20-2.12 (m, 1.3H).

### Intermediate A26-4: 8-((Benzyloxy)methyl)-1,4-dioxaspiro[4.5]decane

To a solution of 1,4-dioxaspiro[4.5]decan-8-ylmethanol (65 g, 95% purity, 359 mmol) in dry tetrahydrofuran (700 mL) was added sodium hydride (60% in mineral oil, 28.7 g, 718 mmol) slowly at 0° C. After addition, the suspension was stirred at 0° C.; for 20 minutes. After benzyl bromide (68.8 g, 98% purity, 394 mmol) was added dropwise, the mixture was stirred at room temperature overnight. The reaction mixture was quenched with water (200 mL) and extracted with ethyl acetate (200 mL) twice. The combined organic layers were washed with brine (500 mL), dried over Na₂SO₄₍ₛ₎ and filtered. The filtrate was concentrated and purified by silica gel column chromatography (petroleum ether:ethyl acetate=15:1) to give the title compound (82 g, 95% purity from NMR, 83% yield) as yellow oil. ¹H NMR (300 MHz, DMSO-d₆) δ 7.35-7.22 (m, 5H), 4.42-4.40 (m, 2H), 3.81-3.79 (m, 4H), 3.25-3.20 (m, 2H), 1.68-1.60 (m, 5H), 1.51-1.36 (m, 2H), 1.23-1.10 (m, 2H).

### Intermediate A26-5: 4-((Benzyloxy)methyl)cyclohexanone

The solution of 8-((benzyloxy)methyl)-8-methyl-1,4-dioxaspiro[4.5]decane A26-4 (82 g, 95% purity, 297 mmol) in dichloromethane (400 mL) and trifluoroacetic acid (400 mL) was stirred at 30° C.; overnight. The mixture was concentrated under reduced pressure to remove dichloromethane and trifluoroacetic acid. The residue was dissolved in dichloromethane (300 mL) and washed with saturated sodium bicarbonate aqueous solution (300 mL) twice and brine (300 mL), dried over Na₂SO₄₍ₛ₎ and filtered. The filtrate was concentrated and purified by silica gel column chromatography (petroleum ether:ethyl acetate=20:1) to give the title compound (64 g, 94% purity from NMR, 95% yield) as colorless oil. ¹H NMR (300 MHz, CDCl₃) δ 7.40-7.27 (m, 5H), 4.54 (s, 2H), 3.40 (d, J=6.3 Hz, 2H), 3.46-2.29 (m, 4H), 2.19-2.03 (m, 3H), 1.55-1.40 (m, 2H).

### Intermediate A26-6: 4-((Benzyloxy)methyl)-2-(dimethoxymethyl)cyclohexanone

To A solution of diethylether-bortrifluorid (36.0 g, 254 mmol) in dichloromethane (200 ml) was added trimethoxymethane (22.0 g, 207 mmol) dropwise with stirring at −30° C.; under nitrogen atmosphere over a period of 10 minutes. The reaction mixture was warmed to 0° C.; and stirred at 0° C.; for 15 minutes. After the mixture was cooled back to −78° C., a solution of 4-((benzyloxy)methyl)cyclohexanone A26-5 (20 g, 95% purity, 87 mmol) in dichloromethane (50 ml) was added, followed by N-ethyl-N-isopropylpropan-2-amine (40.0 g, 309 mmol) over 30 minutes. The resulting mixture was stirred at −78° C.; for 1 hour and then poured into cold saturated sodium bicarbonate aqueous solution (450 ml) and dichloromethane (200 ml) with vigorous stirring. The organic phase was separated, washed with water (350 ml), dried over Na₂SO₄₍ₛ₎ and filtered. The filtrate was concentrated and purified by silica gel column chromatography (petroleum ether:ethyl acetate=10:1) to give the title compound (12 g, 90% purity from NMR, 42% yield) as yellow oil. ¹H NMR (400 MHz, DMSO-d₆) δ 7.35-7.22 (m, 5H), 4.9 (d, J=10.4 Hz, 0.6H), 4.52 (d, J=17.5 Hz, 0.4H), 4.44-4.40 (m, 2H), 3.30-3.19 (m, 8H), 2.76-2.69 (m, 0.2H), 2.59-2.54 (m, 0.5H), 2.42-2.34 (m, 0.8H), 2.23-1.88 (m, 3.5H), 1.52-1.13 (m, 3H).

### Intermediate A26-7: (cis)-Methyl 3-(2-(4-((benzyloxy)methyl)-2-(dimethoxymethyl)cyclohexylidene)hydrazinyl)cyclobutanecarboxylate

To the solution of 4-((benzyloxy)methyl)-2-(dimethoxymethyl)cyclohexanone A26-6 (2.00 g, 90% purity, 6.16 mmol) in hexane (100 mL) was added (cis)-methyl 3-hydrazinylcyclobutanecarboxylate A26-3 (1.4 g, 90% purity, 6.98 mmol) and triethylamine (3.1 g, 30.6 mmol) at room temperature. After stirred at 75° C.; overnight, the mixture was cooled down and filtered. The filtrate was concentrated under reduced pressure to give the crude title compound (3 g, 80% purity, 84% yield) as white oil which was directly used without further purification. LC-MS (ESI): R_{T}=1.60 min, mass calcd. for C₂₃H₃₄N₂O₅ 418.2, m/z found 419.3 [M+H]⁺.

### Intermediate A26-8: (cis)-Methyl 3-(5-((benzyloxy)methyl)-4,5,6,7-tetrahydro-2H-indazol-2-yl)cyclobutanecarboxylate

To a solution of (cis)-methyl 3-(2-(4-((benzyloxy)methyl)-2-(dimethoxymethyl) cyclohexylidene)hydrazinyl)cyclobutanecarboxylate A26-7 (4.00 g, 80% purity, 7.65 mmol) in methanol (40 ml) was added trifluoroacetic acid (4 mL) at room temperature. After stirred at 75° C.; overnight, the mixture was cooled down, poured into cold saturated sodium bicarbonate aqueous solution (80 ml) and extracted with ethyl acetate (70 ml). The organic layer was concentrated and purified by silica gel column chromatography (petroleum ether:ethyl acetate=8:1 to 6:1) to give the title compound (900 mg, 90% purity from NMR, 30% yield) as white oil. ¹H NMR (400 MHz, CDCl₃) δ 7.42-7.30 (m, 6H), 4.71-4.63 (m, 1H), 4.58 (s, 2H), 3.75 (s, 3H), 3.49 (d, J=6.8 Hz, 2H), 3.00-2.91 (m, 1H), 2.85-2.63 (m, 7H), 2.29-2.23 (m, 1H), 2.14-2.05 (m, 2H), 1.59-1.48 (m, 1H).

### Intermediate A26-9: (cis)-Methyl 3-(5-(hydroxymethyl)-4,5,6,7-tetrahydro-2H-indazol-2-yl)cyclobutanecarboxylate

To the solution of (cis)-methyl 3-(5-((benzyloxy)methyl)-4,5,6,7-tetrahydro-2H-indazol-2-yl)cyclobutanecarboxylate A26-8 (3.1 g, 90% purity, 7.87 mmol) in methanol (50 mL) was added 10% palladium on charcoal (1.3 g). The mixture was stirred at room temperature under hydrogen atmosphere overnight. 10% palladium on charcoal was filtered off and the filtrate was concentrated to give the title compound (2.3 g, 90% purity from NMR, 99% yield) as light yellow solids. LC-MS (ESI): R_{T}=1.31 min, mass calcd. for C₁₄H₂₀N₂O₃ 264.1, m/z found 265 [M+H]⁺. ¹H NMR (400 MHz, DMSO-d₆) δ 7.26 (s, 1H), 4.72-4.64 (m, 1H), 3.71 (s, 3H), 3.64-3.63 (d, J=6.4 Hz, 2H), 2.97-2.89 (m, 1H), 2.86-2.60 (m, 7H), 2.26-2.20 (m, 1H), 2.06-2.02 (m, 1H), 1.97-1.87 (m, 1H), 1.54-1.43 (m, 1H).

### Intermediate A26-10: (cis)-Methyl 3-(5-formyl-4,5,6,7-tetrahydro-2H-indazol-2-yl)cyclobutanecarboxylate

To the solution of oxalyl dichloride (1.12 g, 8.82 mmol) in dry dichloromethane (20 mL) was added dimethyl sulfoxide (1.7 g, 21.8 mmol) at −78° C.; dropwise. The mixture was stirred at −78° C.; for 15 minutes and then a solution of (cis)-methyl 3-(5-(hydroxymethyl)-4,5,6,7-tetrahydro-2H-indazol-2-yl)cyclobutanecarboxylate A26-9 (2.3 g, 90% purity, 7.83 mmol) in dry dichloromethane (5 mL) was added dropwise. The mixture was stirred at −78° C.; for 1.5 hours before a solution of triethylamine (4.00 g, 39.5 mmol) in dry dichloromethane (5 mL) was added. The mixture was stirred at −78° C.; for 30 minutes and warmed to room temperature for another 30 minutes. The mixture was quenched with saturated sodium bicarbonate (25 mL) and extracted with dichloromethane (20 mL) for three times. The combined organic layers were dried over Na₂SO₄₍ₛ₎ and filtered. The filtrate was concentrated to give the title compound (2.25 g, 90% purity from NMR, 99% yield) as yellow oil. LC-MS (ESI): R_{T}=1.39 min, mass calcd. for C₁₄H₁₈N₂O₃ 262.1, m/z found 263 [M+H]⁺. ¹H NMR (400 MHz, CDCl₃) δ 9.79 (s, 1H), 7.30 (s, 1H), 4.68-4.60 (m, 1H), 3.71 (s, 3H), 3.14-3.10 (m, 0.5H), 2.95-2.80 (m, 3H), 2.76-2.67 (m, 5H), 2.66-2.60 (m, 1.5H), 2.30-2.23 (m, 1H), 1.89-1.71 (m, 1H).

### Acid 26: (cis)-2-(3-(Methoxycarbonyl)cyclobutyl)-4,5,6,7-tetrahydro-2H-indazole-5-carboxylic acid

To the solution of (cis)-methyl 3-(5-formyl-4,5,6,7-tetrahydro-2H-indazol-2-yl)cyclobutanecarboxylate A26-10 (2.25 g, 90% purity, 7.72 mmol) in acetone (50 mL) and water (15 mL) was added potassium permanganate (3 g, 19.0 mmol) at 0° C. The mixture was stirred at 0° C.; to room temperature for 1 hour. Sodium bisulfite (7.00 g, 58% purity, 39.0 mmol) was added, then the mixture was diluted with acetone (50 mL) and water (50 mL). The resulting suspension was stirred at room temperature for 15 minutes and filtered through celite. The filtrate was concentrated under reduced pressure at room temperature to remove acetone. The resulting aqueous solution was acidified with citric acid(s) to pH ˜3 and extracted with ethyl acetate (20 mL) for three times. The combined organic layers were dired over Na₂SO₄₍ₛ₎ and filtered. The filtrate was concentrated to give the title compound (2.00 g, 90% purity from NMR, 84% yield) as white solids. LC-MS (ESI): R_{T}=0.25 min, mass calcd. For C₁₄H₁₈N₂O₄ 278.1, m/z found 277 [M−H]⁻. ¹H NMR (400 MHz, DMSO-d₆) δ 12.4 (br s, 1H), 7.45 (s, 1H), 4.69-4.61 (m, 1H), 3.63 (s, 3H), 3.00-2.90 (m, 1H), 2.76-2.51 (m, 9H), 2.12-1.99 (m, 1H), 1.77-1.67 (m, 1H).

### Part II: Preparation of Aryl Aldehydes of General Formula IV (P1)

Aldehyde 1, AL1: 2-Chloro-4-fluoro-benzaldehyde
Aldehyde 2, AL2: 2-Chloro-3-fluoro-benzaldehyde
Aldehyde 3, AL3: 4-Fluoro-2-methylbenzaldehyde
Aldehyde 4, AL4: 2-Bromo-4-fluorobenzaldehyde
Aldehyde 5, AL5: 3-Fluoro-2-methyl-benzaldehyde
Aldehyde 8, AL8: 2-Bromo-3,4-difluorobenzaldehyde
Aldehyde 9, AL9: 3-Fluoro-2-methyl-benzaldehyde
Aldehyde 10, AL10: 2-Bromo-3-fluoro-benzaldehyde
Aldehyde 6, AL6: 2-Chloro-3,4-difluorobenzaldehyde

### Intermediate BB1: 2-Chloro-3,4-difluorobenzoic acid

A solution of N¹,N¹,N²,N²-tetramethylethane-1,2-diamine (3.7 g, 69.6 mmol) in tetrahydrofuran (45 mL) was cooled to −70° C.; under nitrogen atmosphere before dropwise addition of 1.3 M sec-butyllithium in hexane (60 mL, 75.9 mmol) followed by a solution of 3,4-difluorobenzoic acid (5.0 g, 31.6 mmol) in tetrahydrofuran (20 mL) over 10 minutes. The resulting mixture was stirred at −70° C.; for 1 hour and then a solution of 1,1,1,2,2,2-hexachloroethane (26 g, 110.8 mmol) in THE (45 mL) was added dropwise. Stirring continued at −70° C.; for 2 hours. The mixture was warmed to −10° C., quenched with water (125 mL), added diethyl ether (60 mL) and then separated two phases. The aqueous layer was acidified to pH 1 by using concentrated hydrochloride aqueous solution and extracted with diethyl ether (125 mL) twice. The combined organic extracts were concentrated in vacuo to give yellow solids, which was recrystallized with ethyl acetate (30 mL) to afford the title compound (2.7 g, 45% yield) as yellow solids. ¹H NMR (400 MHz, DMSO-d₆) δ 13.69 (br s, 1H), 7.75-7.71 (m, 1H), 7.55-7.48 (m, 1H).

### Intermediate BB2: 2-Chloro-3,4-difluoro-N-methoxy-N-methyl-benzamide

To a solution of 2-chloro-3,4-difluorobenzoic acid Intermediate BB1 (1.0 g, 5.2 mmol) in N, N-dimethylformamide (10 mL) were added 1-hydroxybenzotriazole (1.1 g, 7.8 mmol), N,N-diisopropylethylamine (4.6 mL, 26 mmol) and N-(3-Dimethylaminopropyl)-N′-ethylcarbodiimide hydrochloride (1.5 g, 7.8 mmol) under nitrogen atmosphere at room temperature. The resulting mixture was stirred at room temperature for 10 minutes. 0, N-dimethyl-hydroxylamine hydrochloride (0.5 g, 5.2 mmol) was added and stirring continued at room temperature overnight. After quenched with water (20 mL), the mixture was extracted with ethyl acetate (20 mL) for three times. The combined organic layers were washed with water (20 mL), brine (20 mL), dried over Na₂SO₄₍ₛ₎, filtered and concentrated to leave a residue, which was purified by silica gel column chromatography (petroleum ether:ethyl acetate=4:1 to 2:1) to give the title compound (1.06 g, 87% yield) as yellow solids. ¹H NMR (400 MHz, DMSO-d₆) δ 7.60-7.53 (m, 1H), 7.42-7.38 (m, 1H), 3.80-3.45 (m, 3H), 3.39-3.06 (m, 3H).

### Aldehyde 6: 2-Chloro-3,4-difluorobenzaldehyde

To a solution of 2-chloro-3,4-difluoro-N-methoxy-N-methyl-benzamide Intermediate BB2 (500 mg, 2.13 mmol) in tetrahydrofuran (8 mL) was added 1 M diisobutylaluminium hydride in toluene (2.8 mL, 2.8 mmol) dropwise at −78° C.; under nitrogen atmosphere. After the addition, the mixture was stirred at −78° C.; for 1 hour. It was then quenched with water (15 mL) and extracted with ethyl acetate (25 mL) for three times. The combined organic layers were washed with 1 M hydrochloric acid aqueous solution (10 mL), dried over Na₂SO₄₍ₛ₎, filtered and evaporated under reduced pressure to leave a yellow residue, which was purified by silica gel column chromatography (petroleum ether:ethyl acetate=20:1) to give the title compound (200 mg, 53% yield) as yellow solids. ¹H NMR (400 MHz, DMSO-d₆) δ 10.23 (s, 1H), 7.80-7.76 (m, 1H), 7.69-7.62 (m, 1H).

### Aldehyde 7, AL7: 3,4-Difluoro-2-methylbenzaldehyde

### Intermediate BB3: 3,4-Difluoro-N-methoxy-N,2-dimethylbenzamide

To a solution of 3,4-difluoro-2-methylbenzoic acid (3.0 g, 17.4 mmol) in N, N-dimethylformamide (30 mL) were added 1-hydroxybenzotriazole (3.5 g, 26.2 mmol), N,N-diisopropylethylamine (15.4 mL, 87.0 mmol) and N¹-((ethylimino)methylene)-N³, N³-dimethylpropane-1,3-diamine hydrochloride (5.0 g, 26.2 mmol) under nitrogen atmosphere at room temperature. Having stirred at room temperature for 10 minutes, the resulting mixture was added N,O-dimethylhydroxylamine hydrochloride (1.7 g, 17.4 mmol) and stirring continued at room temperature overnight. After quenched with water (50 mL), the mixture was extracted with ethyl acetate (50 mL) for three times. The combined organic layers were washed with water (50 mL), brine (50 mL), dried over Na₂SO₄₍ₛ₎, and concentrated to leave a residue, which was purified by silica gel column chromatography (petroleum ether:ethyl acetate=8:1 to 5:1) to give the title compound (3.1 g, 84% yield) as yellow oil. ¹H NMR (300 MHz, CDCl₃) δ 7.07-6.96 (m, 2H), 3.47 (s, 3H), 3.30 (s, 3H), 2.26 (s, 3H).

### Aldehyde 7: 3,4-Difluoro-2-methylbenzaldehyde

To a solution of 3,4-difluoro-N-methoxy-N,2-dimethylbenzamide Intermediate BB3 (3.1 g, 14.4 mmol) in tetrahydrofuran (40 mL) was added 1.5 M diisobutylaluminium hydride in toluene (12.5 mL, 18.7 mmol) dropwise at −78° C.; under nitrogen atmosphere. After the addition, the mixture was stirred at −78° C.; for 1.5 hour. It was then quenched with water (15 mL) and extracted with ethyl acetate (50 mL) for three times. The combined organic layers were washed with water (50 mL) for three times, brine (50 mL), dried over Na₂SO₄₍ₛ₎ and evaporated under reduced pressure to leave a yellow residue, which was purified by silica gel column chromatography (petroleum ether:ethyl acetate=20:1 to 10:1) to give the title compound (1.87 g, 85% yield) as colorless oil. ¹H NMR (400 MHz, CDCl₃) δ 10.15 (s, 1H), 7.61-7.57 (m, 1H), 7.18-7.12 (m, 1H), 2.61 (s, 3H).

### Part III: Preparation of Carboxamidines of General Formula V (P2)

### Carboxamidine 1, Ca1: Thiazole-2-carboxamidine hydrochloride

### Carboxamidine 2, Ca2: 3,5-Difluoropicolinimidamide hydrochloride

To a stirred suspension of ammonium chloride (1.89 g, 35.7 mmol) in toluene (100 mL) was added 2M trimethylaluminum in toluene (21 mL, 42.8 mmol) dropwise at 0° C.; under nitrogen atmosphere. The resulting mixture was then brought up to room temperature and stirring continued for 30 minutes. A solution of 3,5-difluoropicolinonitrile (5.00 g, 35.7 mmol) in toluene (50 mL) was added and the reaction mixture was subsequently stirred at 80° C.; overnight. After cooled down to room temperature, the mixture was poured into slurry of silica gel in dichloromethane (50 mL). After stirring for 10 minutes, the slurry was filtered and washed with methanol. The filtrate was concentrated in vacuum to give the title compound (1.90 g, 34% yield) as white solids. LC-MS (ESI): R_{T}=0.357 min, mass calcd. for C₆H₆ClF₂N₃ 193.0, m/z found 157.9 [M+H-HCl]⁺. ¹H NMR (400 MHz, DMSO-d₆) δ 9.77 (br s, 2H), 9.60 (br s, 2H), 8.79 (d, J=1.6 Hz, 1H), 8.41-8.35 (m, 1H).

### Part IV: Preparation of Ketoesters of General Formula III

### Intermediate KT1: (exemplified with Method A₁) 3-(2-(3-Methoxy-3-oxopropyl)-4,5,6,7-tetrahydro-2H-indazol-5-yl)-3-oxopropanoate

To the solution of 2-(3-methoxy-3-oxopropyl)-4,5,6,7-tetrahydro-2H-indazole-5-carboxylic acid Acid 1 (4.50 g, 16.9 mmol) in acetonitrile (36 mL) was added 1,1′-carbonyldiimidazole (3.33 g, 20.3 mmol) at room temperature. The solution was stirred at room temperature under nitrogen atmosphere for 1 hour (mixture A). To the suspension of ethyl potassium malonate KT2 (6.12 g, 35.6 mmol) and magnesium chloride (4.07 g, 42.4 mmol) in acetonitrile (72 mL) was added triethylamine (5.54 g, 54.2 mmol). After stirred at room temperature under nitrogen atmosphere for 1 hour, the suspension was added mixture A and stirring continued at 80° C.; under nitrogen atmosphere overnight. Then it was cooled down and concentrated under reduced pressure to give a residue, which was taken up into water (150 mL) and ethyl acetate (150 mL). The mixture was acidified with potassium bisulfate₍ₛ₎ to pH 8 and then the organic phase was separated. The aqueous layer was extracted with ethyl acetate (150 mL) twice. The combined organic layers were washed with brine (150 mL), dried over Na₂SO₄(s) and filtered. The filtrate was concentrated under reduced pressure to give the crude title compound (6.00 g, 99% yield, including 1,1′-carbonyldiimidazole by NMR) as brown oil which was directly used without further purification. LC-MS (ESI): R_{T}=1.488 min, mass calcd. for C₁₆H₂₂N₂O₅ 322.2, m/z found 323.2 [M+H]⁺. ¹H NMR (400 MHz, DMSO-d₆) δ 7.39 (s, 1H), 4.22 (t, J=6.8 Hz, 2H), 4.09 (q, J=7.2 Hz, 2H), 3.78-3.69 (m, 2H), 3.59 (s, 3H), 2.83-2.77 (m, 3H), 2.74-2.69 (m, 1H), 2.66-2.59 (m, 1H), 2.56-2.51 (m, 1H), 2.49-2.45 (m, 1H), 2.13-2.10 (m, 1H), 1.64-1.54 (m, 1H), 1.21-1.17 (m, 3H).

### Intermediate KT3: (exemplified with Method A₂)

### Methyl 3-(2-(3-methoxy-3-oxopropyl)-4,5,6,7-tetrahydro-2H-indazol-5-yl)-3-oxopropanoate

By ultilizing the analogous procedure of Method A₁ with an employment of methyl potassium malonate to substitute ethyl potassium malonate KT4, the title compound was synthesized. LC-MS (ESI): R_{T}=1.36 min, mass calcd. for C₁₅H₂₀N₂O₅ 308.1, m/z found 309.1 [M+H]⁺. ¹H NMR (300 MHz, DMSO-d₆) δ 7.01 (m, 1H), 4.24-4.18 (m, 2H), 3.77 (t, J=4.8 Hz, 2H), 3.63-3.58 (m, 6H), 2.84-2.72 (s, 3H), 2.69-2.54 (m, 3H), 2.47-2.41 (m, 1H), 2.13-2.08 (m, 1H), 1.66-1.51 (m, 1H).

### Part V: Assembly of Dihydropyrimidines of General Formula I

### Compound 1: (exemplified with Method B)

### 4-(2-Chloro-3,4-difluoro-phenyl)-6-[2-(2-methoxycarbonyl-ethyl)-4,5,6,7-tetrahydro-2H-indazol-5-yl]-2-thiazol-2-yl-1,4-dihydro-pyrimidine-5-carboxylic acid ethyl ester

To a solution of ethyl 3-(2-(3-methoxy-3-oxopropyl)-4,5,6,7-tetrahydro-2H-indazol-5-yl)-3-oxopropanoate KT1 (1.00 g, 2.79 mmol), 2-chloro-3,4-difluoro-benzaldehyde AL6 (519 mg, 2.79 mmol) and 2-thiazolecarboxamidine hydrochloride Ca1 (466 mg, 2.79 mmol) in ethanol (56 mL) was added sodium acetate (233 mg, 2.79 mmol) at room temperature. After stirred at 85° C.; under nitrogen atmosphere overnight, the mixture was concentrated under reduced pressure to leave a residue, which was purified by silica gel column chromatography (petroleum ether:ethyl acetate=5:1 to 1:1) followed by C18 column (acetonitrile:water=50% to 80%) to give the title compound (480 mg, 28% yield) as yellow solids. LC-MS (ESI): R_{T}=1.53 min, mass calcd. for C₂₇H₂₆ClF₂N₅O₄S 589.1, m/z found 590.3 [M+H]⁺. ¹H NMR (400 MHz, DMSO-d₆) δ 9.60 (d, J=3.6 Hz, 0.35 H), 9.57 (d, J=3.6 Hz, 0.35 H), 9.05 (s, 0.15H), 9.00 (s, 0.15H), 8.01-7.92 (m, 2H), 7.52-7.37 (m, 2H), 7.29-7.21 (m, 1H), 6.07 (s, 0.15H), 6.06 (s, 0.15H), 5.97-5.95 (m, 0.7H), 4.28-4.22 (m, 2H), 4.11 (br s, 0.3H), 3.99-3.87 (m, 2.7H), 3.61 (s, 3H), 2.93-2.65 (m, 5H), 2.60-2.53 (m, 1H), 2.14-1.92 (m, 1.7H), 1.83-1.79 (m, 0.3H), 1.06-0.98 (m, 3H).

A stereoisomeric mixture of Compound 1 (1.34 g, 2.16 mmol) was separated by chiral prep. HPLC (first separation condition: Column: Chiralpak IE 5 μm 20*250 mm; Mobile Phase: Hex:EtOH=70:30 at 15 mL/min; Temp: 30° C.; Wavelength: 214 nm; second separation condition: Column: Chiralpak IG 5 μm 20*250 mm; Mobile Phase: Hex:EtOH:DEA=60:40:0.3 at 15 mL/min; Temp: 30° C.; Wavelength: 214 nm; third separation condition: Column: Chiralpak IG 5 μm 20*250 mm; Mobile Phase: Hex:EtOH:DEA=80:20:0.3 at 15 mL/min; Temp: 30° C.; Wavelength: 214 nm) to give Compound 1a (170 mg, 13% yield, 100% stereopure), Compound 1b (280 mg, 21% yield, 99.9% stereopure), Compound 1c (310 mg, 23% yield, 100% stereopure) and Compound 1d (200 mg, 15% yield, 97% purity, 97.2% stereopure) as yellow solids.

Compound 1a: LC-MS (ESI): R_{T}=1.24 min, mass calcd. for C₂₇H₂₆ClF₂N₅O₄S 589.1, m/z found 590.0 [M+H]⁺. Chiral HPLC (Column: Chiralpak IG 5 μm 4.6*250 mm; Mobile Phase: Hex:EtOH:DEA=80:20:0.2 at 1.0 mL/min; Temp: 30° C.; Wavelength: 230 nm, R_{T}=13.044 min). ¹H NMR (400 MHz, DMSO-d₆) δ 9.60 (d, J=3.6 Hz, 0.7H), 9.07 (s, 0.3H), 8.01-7.98 (m, 1.3H), 7.97-7.93 (m, 0.7H), 7.52-7.46 (m, 1H), 7.45 (s, 0.3H), 7.40 (s, 0.7H), 7.26-7.21 (m, 1H), 6.07 (s, 0.3H), 5.96 (d, J=3.6 Hz, 0.7H), 4.28-4.23 (m, 2H), 4.18-4.11 (m, 0.3H), 3.98-3.87 (m, 2.7H), 3.61 (s, 3H), 2.98-2.56 (m, 6H), 2.17-2.08 (m, 0.3H), 2.03-1.92 (m, 1H), 1.83-1.78 (m, 0.7H), 1.04 (t, J=7.2 Hz, 2.1H), 1.00 (t, J=7.2 Hz, 0.9H).

Compound 1c: LC-MS (ESI): R_{T}=1.53 min, mass calcd. for C₂₇H₂₆ClF₂N₅O₄S 589.1, m/z found 590.5 [M+H]⁺. Chiral HPLC (Column: Chiralpak IE 5 μm 4.6*250 mm; Mobile Phase: Hex:EtOH=70:30 at 1.0 mL/min; Temp: 30° C.; Wavelength: 230 nm, R_{T}=14.684 min). ¹H NMR (400 MHz, DMSO-d₆) δ 9.60 (d, J=3.2 Hz, 0.7H), 9.00 (s, 0.3H), 8.01-7.92 (m, 2H), 7.52-7.45 (m, 1H), 7.44 (s, 0.3H), 7.37 (s, 0.7H), 7.29-7.24 (m, 1H), 6.06 (s, 0.3H), 5.95 (d, J=3.2 Hz, 0.7H), 4.28-4.22 (m, 2H), 4.19-4.12 (m, 0.3H), 3.99-3.88 (m, 2.7H), 3.61 (s, 3H), 2.84 (t, J=6.8 Hz, 2H), 2.78-2.53 (m, 4H), 2.28-1.97 (m, 2H), 1.06-0.99 (m, 3H).

Compound 1d: LC-MS (ESI): R_{T}=1.16 min, mass calcd. for C₂₇H₂₆ClF₂N₅O₄S 589.1, m/z found 589.9 [M+H]⁺. Chiral HPLC (Column: Chiralpak IG 5 μm 4.6*250 mm; Mobile Phase: Hex:EtOH:DEA=80:20:0.2 at 1.0 mL/min; Temp: 30° C.; Wavelength: 254 nm, R_{T}=15.077 min). ¹H NMR (400 MHz, DMSO-d₆) δ 9.61 (d, J=3.2 Hz, 0.7H), 9.08 (s, 0.3H), 8.01-7.98 (m, 1.3H), 7.97-7.93 (m, 0.7H), 7.52-7.46 (m, 1H), 7.45 (s, 0.3H), 7.40 (s, 0.7H), 7.26-7.21 (m, 1H), 6.07 (s, 0.3H), 5.96 (d, J=3.2 Hz, 0.7H), 4.28-4.23 (m, 2H), 4.18-4.11 (m, 0.3H), 3.98-3.87 (m, 2.7H), 3.61 (s, 3H), 2.98-2.54 (m, 6H), 2.18-2.07 (m, 0.3H), 2.03-1.91 (m, 1H), 1.83-1.77 (m, 0.7H), 1.04 (t, J=7.2 Hz, 2.1H), 1.00 (t, J=7.2 Hz, 0.9H).

Assembles of dihydropyrimidines of general formula I incorporated with acids of general formula II, aryl aldehydes (P1) and carboxamidines (P2) via sequential two reaction steps selected either one Method from Scheme 1 and Scheme 2 individually are shown below in Table 1:

| acid of general formula | | | | | dihydropyrimidine of general |
|---|---|---|---|---|---|
| II | Method | ketoester of general formula III | P1 | P2 | formula I |
| 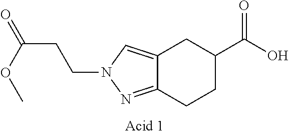 | A₁ | 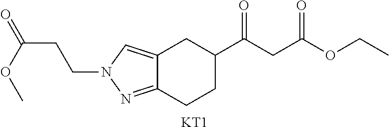 | AL6 | Ca1 | 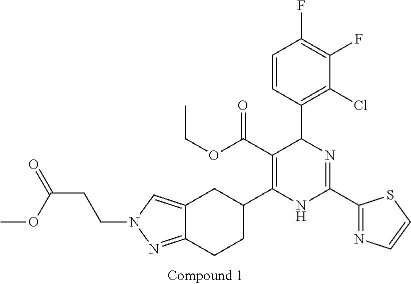 [SMILES: CCOC(=O)C1=C(C2CCc3nn(CCC(=O)OC)cc3C2)NC(c2nccs2)=NC1c1ccc(F)c(F)c1Cl] |
| | | 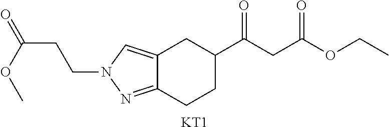 | AL7 | Ca1 | 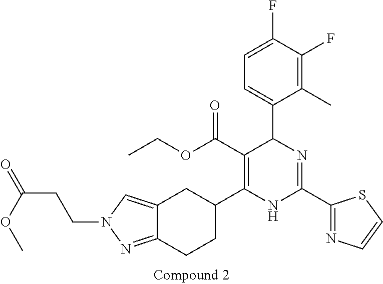 [SMILES: CCOC(=O)C1=C(C2CCc3nn(CCC(=O)OC)cc3C2)NC(c2nccs2)=NC1c1ccc(F)c(F)c1C] |
| | | 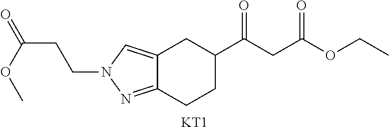 | AL8 | Ca1 | 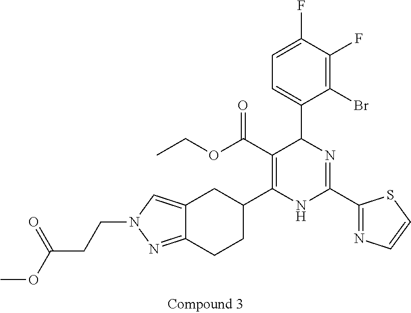 [SMILES: CCOC(=O)C1=C(C2CCc3nn(CCC(=O)OC)cc3C2)NC(c2nccs2)=NC1c1ccc(F)c(F)c1Br] |
| | | 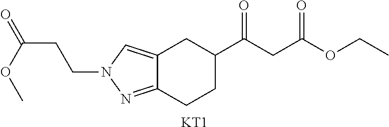 | AL1 | Ca1 | 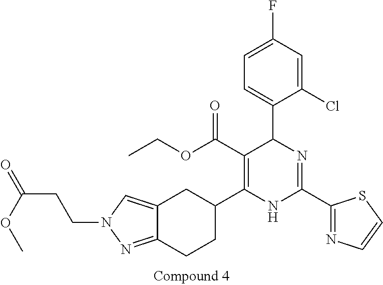 [SMILES: CCOC(=O)C1=C(C2CCc3nn(CCC(=O)OC)cc3C2)NC(c2nccs2)=NC1c1ccc(F)cc1Cl] |
| | | 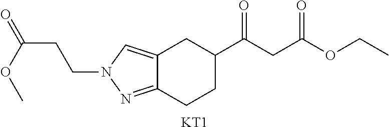 | AL3 | Ca1 | 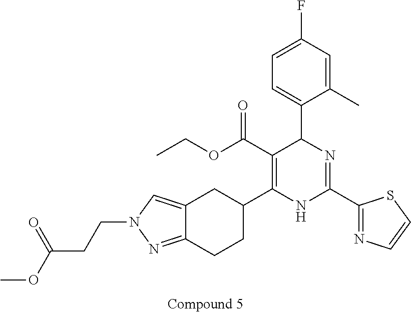 [SMILES: CCOC(=O)C1=C(C2CCc3nn(CCC(=O)OC)cc3C2)NC(c2nccs2)=NC1c1ccc(F)cc1C] |
| | | 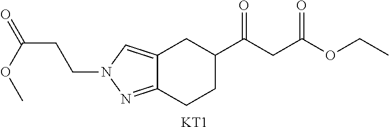 | AL4 | Ca1 | 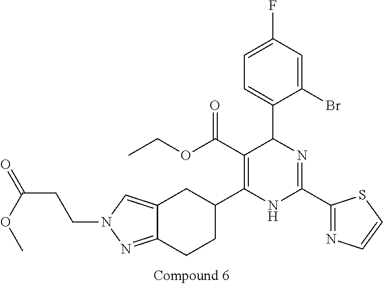 [SMILES: CCOC(=O)C1=C(C2CCc3nn(CCC(=O)OC)cc3C2)NC(c2nccs2)=NC1c1ccc(F)cc1Br] |
| | | 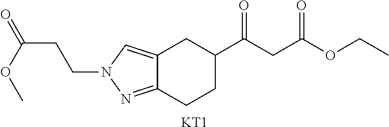 | AL2 | Ca1 | 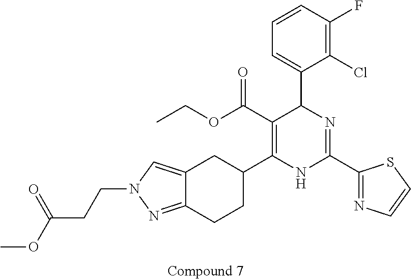 [SMILES: CCOC(=O)C1=C(C2CCc3nn(CCC(=O)OC)cc3C2)NC(c2nccs2)=NC1c1cccc(F)c1Cl] |
| | | 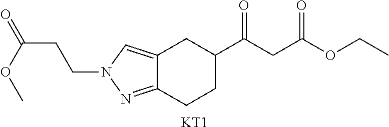 | AL9 | Ca1 | 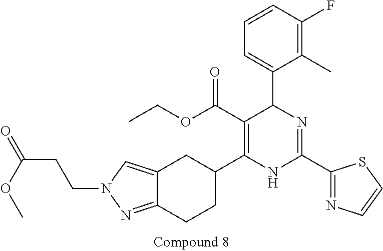 [SMILES: CCOC(=O)C1=C(C2CCc3nn(CCC(=O)OC)cc3C2)NC(c2nccs2)=NC1c1cccc(F)c1C] |
| | | 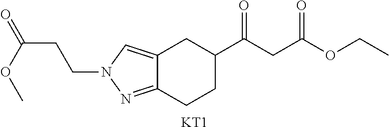 | AL10 | Ca1 | 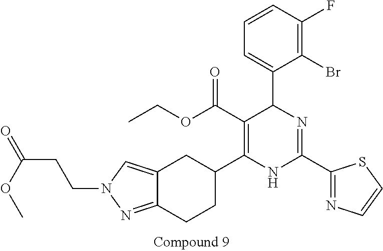 [SMILES: CCOC(=O)C1=C(C2CCc3nn(CCC(=O)OC)cc3C2)NC(c2nccs2)=NC1c1cccc(F)c1Br] |
| | | 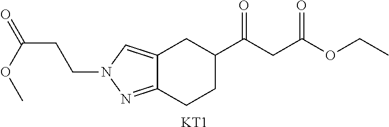 | AL6 | Ca2 | 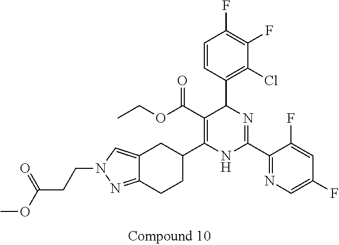 [SMILES: CCOC(=O)C1=C(C2CCc3nn(CCC(=O)OC)cc3C2)NC(c2ncc(F)cc2F)=NC1c1ccc(F)c(F)c1Cl] |
| 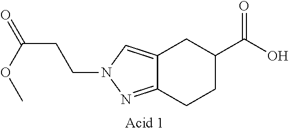 | A₂ | 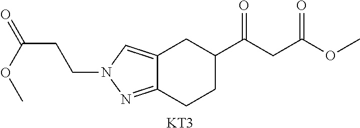 | AL2 | Ca1 | 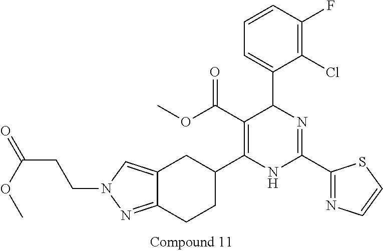 [SMILES: COC(=O)CCn1cc2c(n1)CCC(C1=C(C(=O)OC)C(c3cccc(F)c3Cl)N=C(c3nccs3)N1)C2] |
| | | 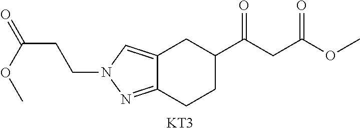 | AL10 | Ca1 | 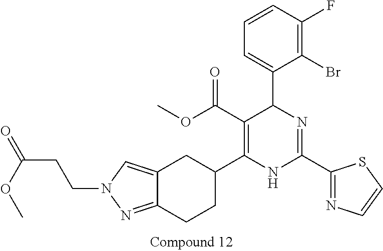 [SMILES: COC(=O)CCn1cc2c(n1)CCC(C1=C(C(=O)OC)C(c3cccc(F)c3Br)N=C(c3nccs3)N1)C2] |
| | | 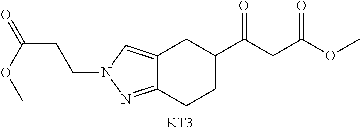 | AL8 | Ca1 | 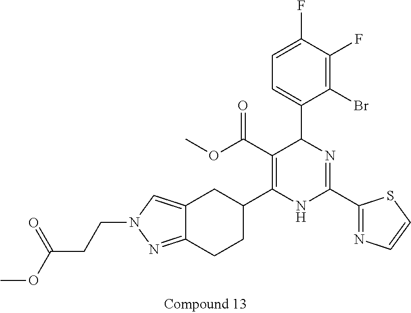 [SMILES: COC(=O)CCn1cc2c(n1)CCC(C1=C(C(=O)OC)C(c3ccc(F)c(F)c3Br)N=C(c3nccs3)N1)C2] |
| | | 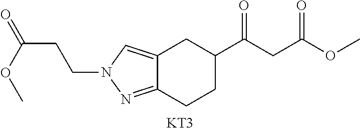 | AL1 | Ca1 | 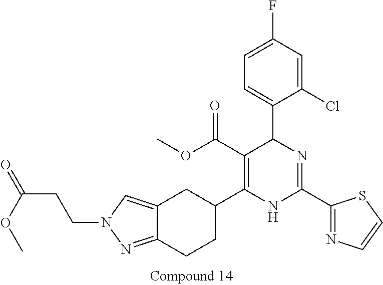 [SMILES: COC(=O)CCn1cc2c(n1)CCC(C1=C(C(=O)OC)C(c3ccc(F)cc3Cl)N=C(c3nccs3)N1)C2] |
| | | 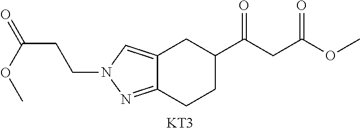 | AL4 | Ca1 | 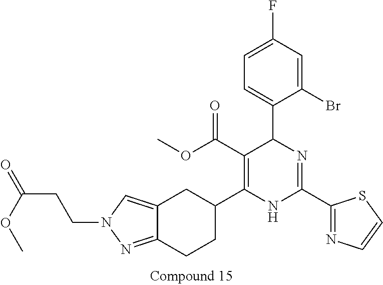 [SMILES: COC(=O)CCn1cc2c(n1)CCC(C1=C(C(=O)OC)C(c3ccc(F)cc3Br)N=C(c3nccs3)N1)C2] |
| 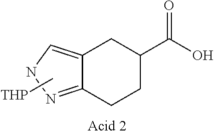 | A₂ | 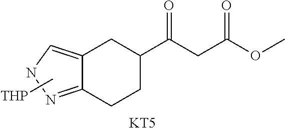 | AL6 | Ca1 | 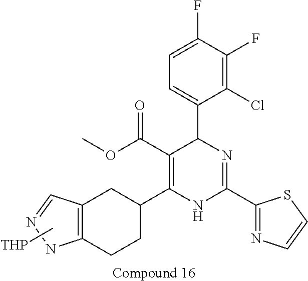 |
| 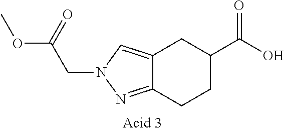 | A₁ | 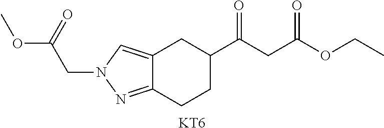 | AL6 | Ca1 | 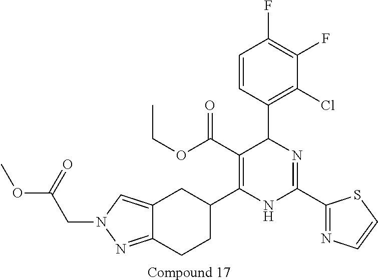 |
| 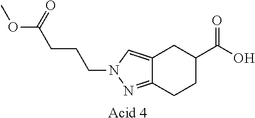 | A₁ | 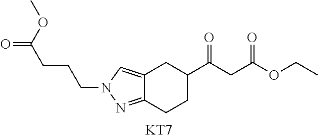 | AL6 | Ca1 | 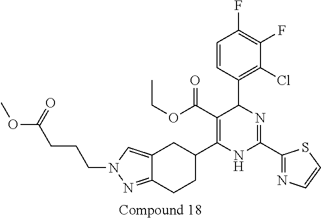 |
| 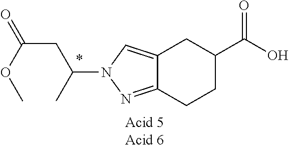 | A₁ | 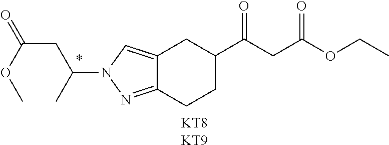 | AL6 | Ca1 | 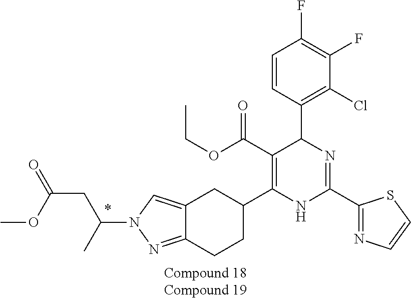 |
| 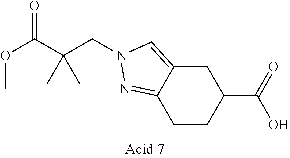 | A₁ | 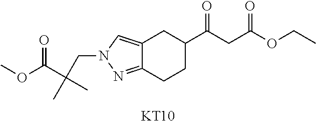 | AL6 | Ca1 | 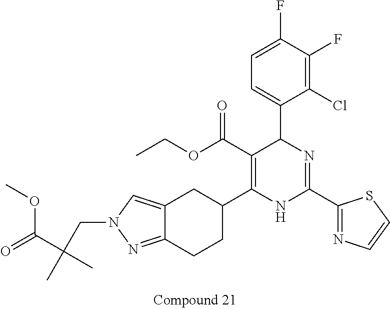 [SMILES: CCOC(=O)C1=C(C2CCc3nn(CC(C)(C)C(=O)OC)cc3C2)NC(c2nccs2)=NC1c1ccc(F)c(F)c1Cl] |
| 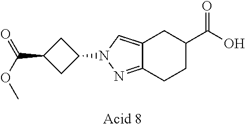 | A₁ | 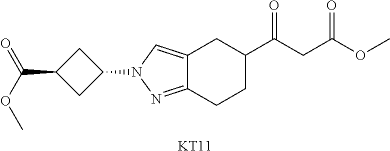 | AL6 | Ca1 | 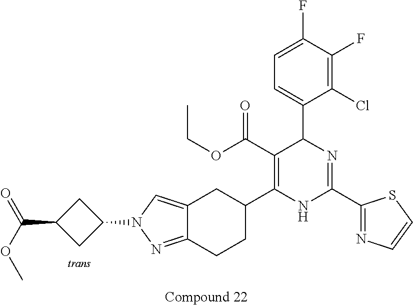 [SMILES: CCOC(=O)C1=C(C2CCc3nn([C@H]4C[C@H](C(=O)OC)C4)cc3C2)NC(c2nccs2)=NC1c1ccc(F)c(F)c1Cl ; "Compound 22"] |
| 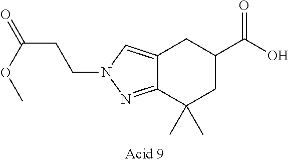 | A₁ | 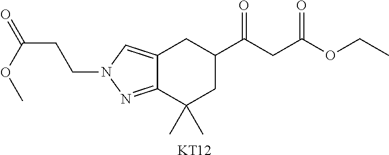 | AL6 | Ca1 | 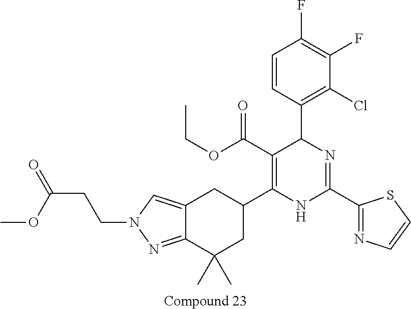 |
| 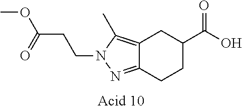 | A₁ | 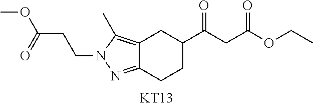 | AL6 | Ca1 | 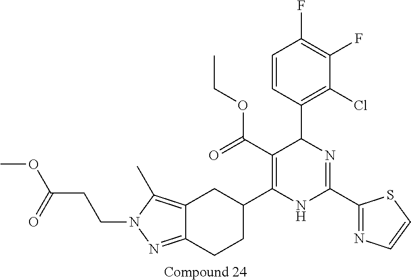 |
| 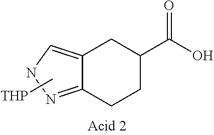 | A₁ | 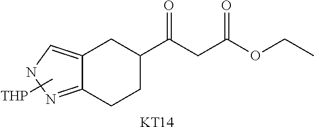 | AL6 | Ca1 | 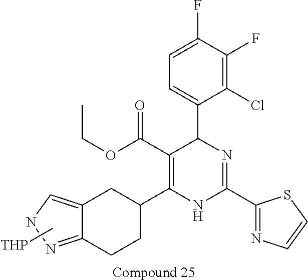 |
| 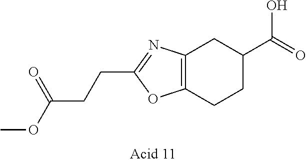 | A₁ | 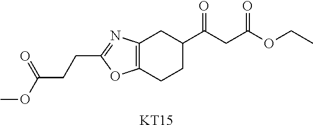 | AL6 | Ca1 | 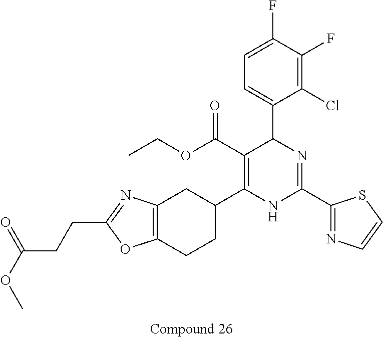 [SMILES: CCOC(=O)C1=C(C2CCc3oc(CCC(=O)OC)nc3C2)NC(c2nccs2)=NC1c1ccc(F)c(F)c1Cl] |
| 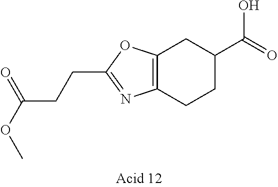 | A₁ | 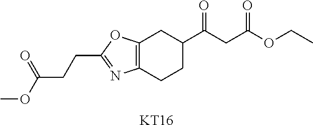 | AL6 | Ca1 | 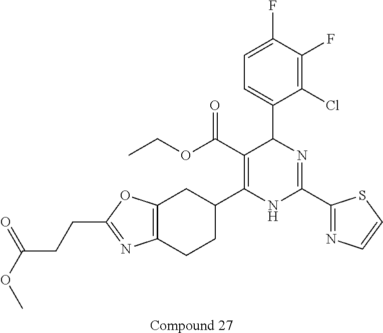 [SMILES: CCOC(=O)C1=C(C2CCc3nc(CCC(=O)OC)oc3C2)NC(c2nccs2)=NC1c1ccc(F)c(F)c1Cl ; "Compound 27"] |
| 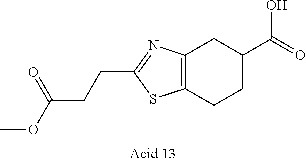 | A₁ | 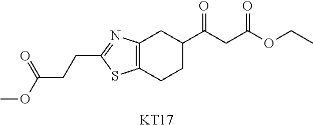 | AL6 | Ca1 | 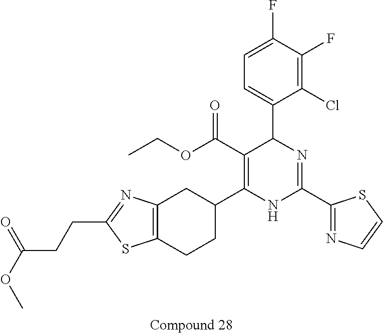 [SMILES: CCOC(=O)C1=C(C2CCc3sc(CCC(=O)OC)nc3C2)NC(c2nccs2)=NC1c1ccc(F)c(F)c1Cl ; "Compound 28"] |
| 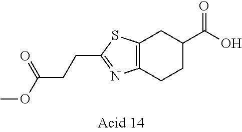 | A₁ | 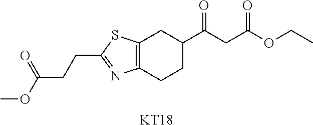 | AL6 | Ca1 | 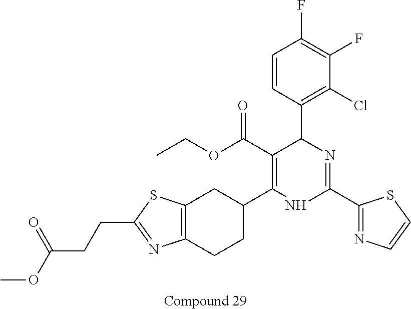 [SMILES: CCOC(=O)C1=C(C2CCc3nc(CCC(=O)OC)sc3C2)NC(c2nccs2)=NC1c1ccc(F)c(F)c1Cl ; "Compound 29"] |
| 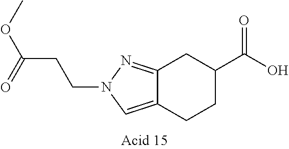 | A₁ | 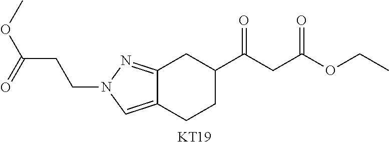 | AL6 | Ca1 | 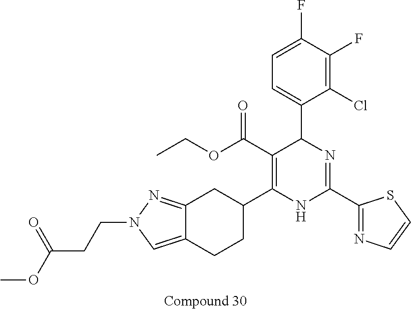 [SMILES: CCOC(=O)C1=C(C2CCc3cn(CCC(=O)OC)nc3C2)NC(c2nccs2)=NC1c1ccc(F)c(F)c1Cl ; "Compound 30"] |
| 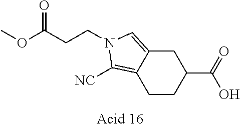 | A1 | 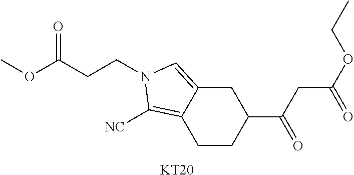 | AL6 | Ca1 | 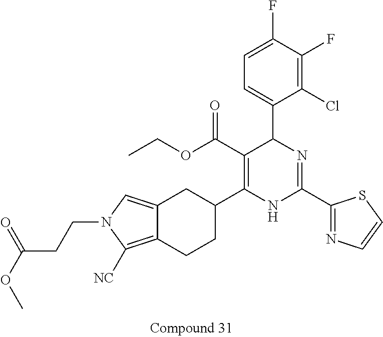 [SMILES: CCOC(=O)C1=C(C2CCc3c(cn(CCC(=O)OC)c3C#N)C2)NC(c2nccs2)=NC1c1ccc(F)c(F)c1Cl] |
| 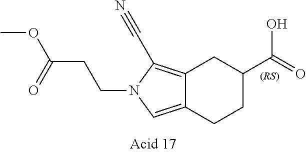 | A1 | 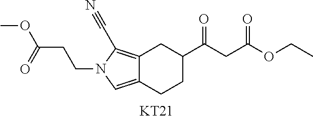 | AL6 | Ca1 | 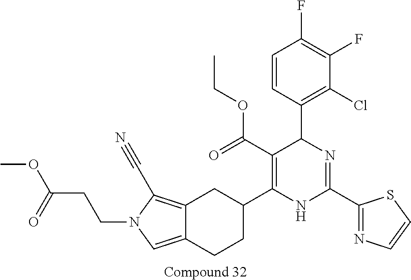 |
| 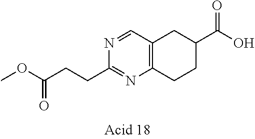 | A2 | 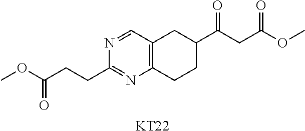 | AL6 | Ca1 | 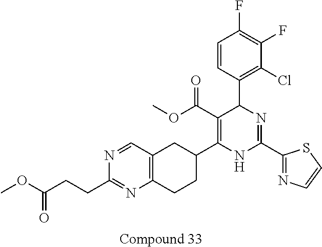 [SMILES: COC(=O)CCc1ncc2c(n1)CCC(C1=C(C(=O)OC)C(c3ccc(F)c(F)c3Cl)N=C(c3nccs3)N1)C2 ; "Compound 33"] |
| 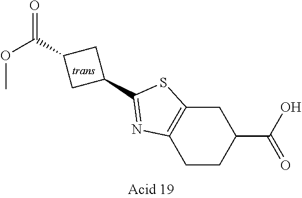 | A2 | 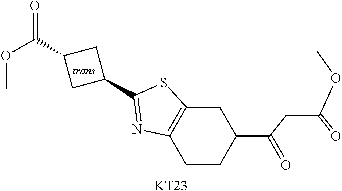 | AL6 | Ca1 | 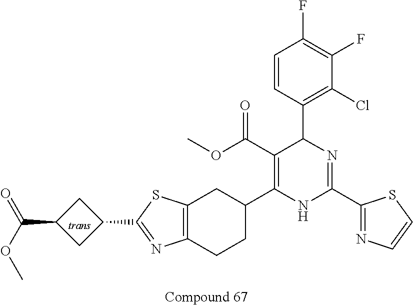 [SMILES: COC(=O)C1=C(C2CCc3nc([C@H]4C[C@H](C(=O)OC)C4)sc3C2)NC(c2nccs2)=NC1c1ccc(F)c(F)c1Cl ; "Compound 67"] |
| | | 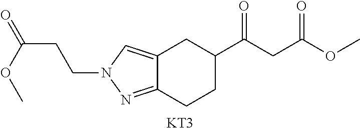 | AL6 | Ca1 | 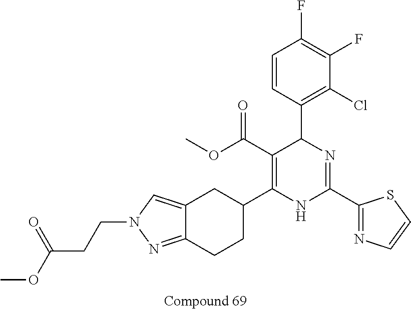 [SMILES: COC(=O)CCn1cc2c(n1)CCC(C1=C(C(=O)OC)C(c3ccc(F)c(F)c3Cl)N=C(c3nccs3)N1)C2 ; "Compound 69"] |
| 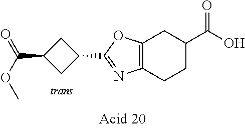 | A2 | 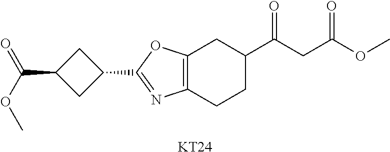 | AL6 | Ca1 | 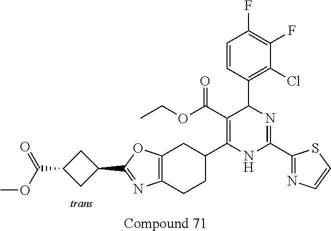 [SMILES: CCOC(=O)C1=C(C2CCc3nc([C@H]4C[C@H](C(=O)OC)C4)oc3C2)NC(c2nccs2)=NC1c1ccc(F)c(F)c1Cl ; "Compound 71"] |
| Acid 4 | A2 | 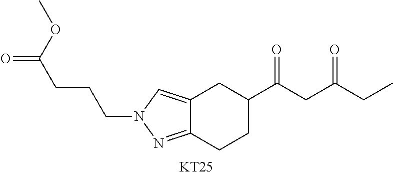 | AL6 | Ca1 | 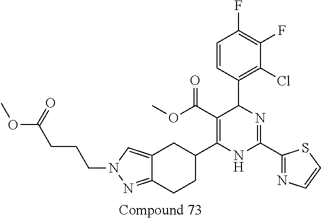 |
| | | 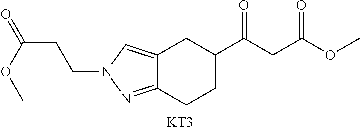 | AL7 | Ca1 | 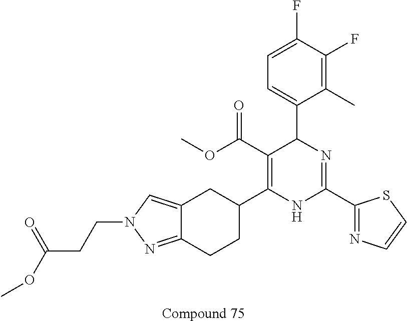 [SMILES: COC(=O)CCn1cc2c(n1)CCC(C1=C(C(=O)OC)C(c3ccc(F)c(F)c3C)N=C(c3nccs3)N1)C2 ; "Compound 75"] |
| 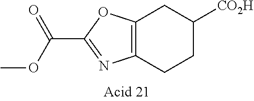 | A2 | 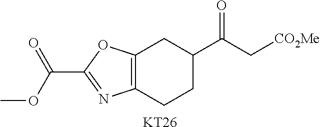 | AL6 | Ca1 | 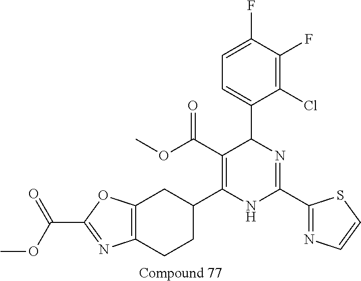 |
| | | 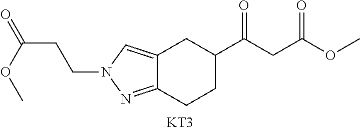 | AL3 | Ca1 | 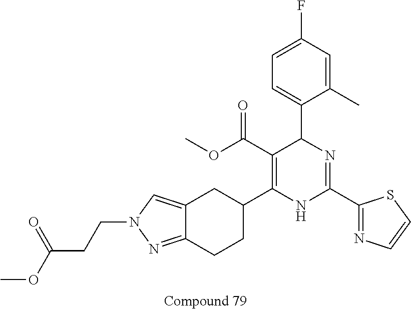 [SMILES: COC(=O)CCn1cc2c(n1)CCC(C1=C(C(=O)OC)C(c3ccc(F)cc3C)N=C(c3nccs3)N1)C2 ; "Compound 79"] |
| 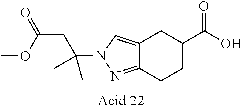 | A2 | 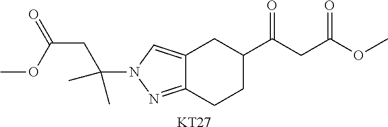 | AL6 | Ca1 | 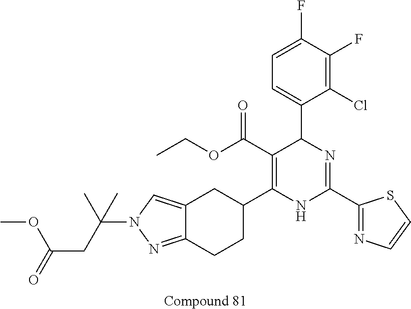 [SMILES: CCOC(=O)C1=C(C2CCc3nn(C(C)(C)CC(=O)OC)cc3C2)NC(c2nccs2)=NC1c1ccc(F)c(F)c1Cl ; "Compound 81"] |
| 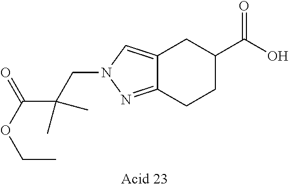 | A2 | 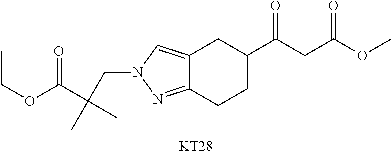 | AL6 | Ca1 | 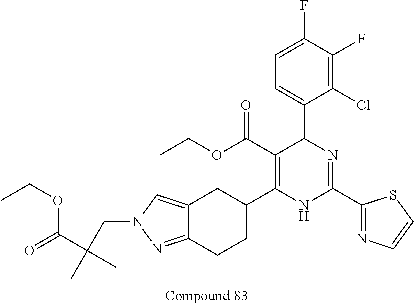 [SMILES: CCOC(=O)C1=C(C2CCc3nn(CC(C)(C)C(=O)OCC)cc3C2)NC(c2nccs2)=NC1c1ccc(F)c(F)c1Cl ; "Compound 83"] |
| 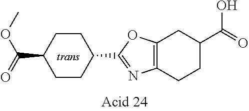 | A2 | 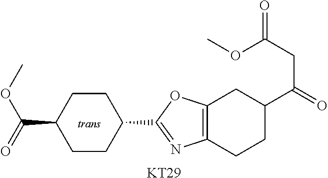 | AL6 | Ca1 | 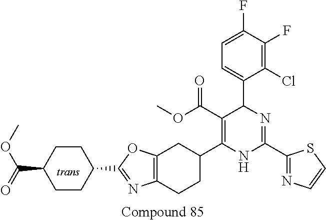 |
| 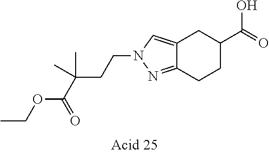 | A1 | 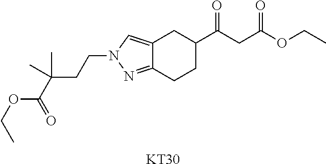 [SMILES: CCOC(=O)CC(=O)C1CCc2nn(CCC(C)(C)C(=O)OCC)cc2C1 ; "KT30"] | AL6 | Ca1 | 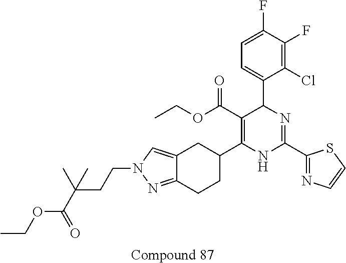 [SMILES: CCOC(=O)C1=C(C2CCc3nn(CCC(C)(C)C(=O)OCC)cc3C2)NC(c2nccs2)=NC1c1ccc(F)c(F)c1Cl ; "Compound 87"] |
| Acid 25 | A2 | 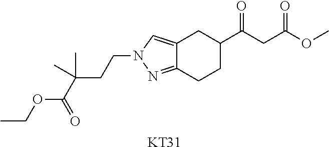 [SMILES: CCOC(=O)C(C)(C)CCn1cc2c(n1)CCC(C(=O)CC(=O)OC)C2 ; "KT31"] | AL6 | Ca1 | 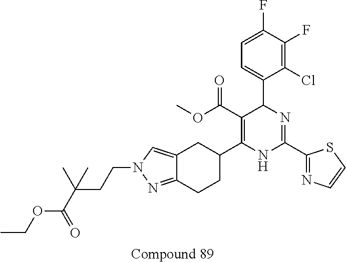 [SMILES: CCOC(=O)C(C)(C)CCn1cc2c(n1)CCC(C1=C(C(=O)OC)C(c3ccc(F)c(F)c3Cl)N=C(c3nccs3)N1)C2 ; "Compound 89"] |
| 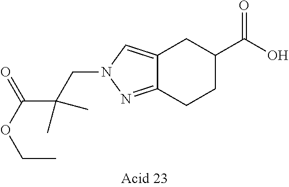 | A1 | 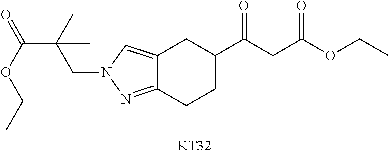 | AL7 | Ca1 | 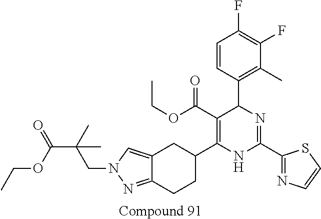 |
| | | 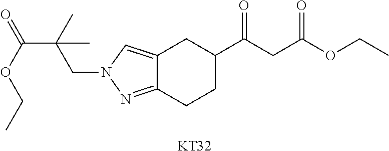 | AL6 | Ca2 | 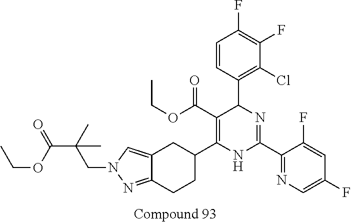 |
| 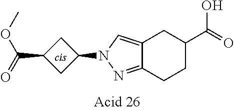 | A2 | 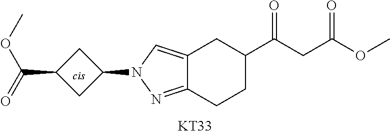 | AL6 | Ca1 | 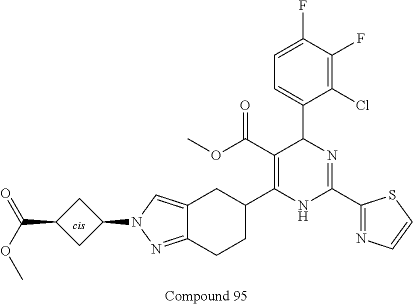 [SMILES: COC(=O)C1=C(C2CCc3nn([C@H]4C[C@@H](C(=O)OC)C4)cc3C2)NC(c2nccs2)=NC1c1ccc(F)c(F)c1Cl ; "Compound 95"] |

### Spectral Analyses of Ketoesters

### Intermediates KT5

### methyl 3-oxo-3-(2-(tetrahydro-2H-pyran-2-yl)-4,5,6,7-tetrahydro-2H-indazol-5-yl)propanoate and methyl 3-oxo-3-(1-(tetrahydro-2H-pyran-2-yl)-4,5,6,7-tetrahydro-2H-indazol-5-yl)propanoate

LC-MS (ESI): R_{T}=1.44 min, mass calcd. for C₁₆H₂₂N₂O₄ 306.2, m/z found 307.2 [M+H]⁺. ¹H NMR (300 MHz, CDCl₃) δ 7.34 (s, 1H), 5.28-5.19 (m, 1H), 4.09-3.98 (m, 1H), 3.74 (s, 3H), 3.67-3.63 (m, 1H), 3.59 (s, 2H), 2.91-2.64 (m, 5H), 2.24-2.00 (m, 4H), 1.76-1.58 (m, 4H).

### Intermediate KT6

### Ethyl 3-(2-(2-methoxy-2-oxoethyl)-4,5,6,7-tetrahydro-2H-indazol-5-yl)-3-oxopropanoate

LC-MS (ESI): R_{T}=1.932 min, mass calcd. for C₁₅H₂₀N₂O₅ 308.1, m/z found 309.0 [M+H]⁺. ¹H NMR (400 MHz, CDCl₃) δ 12.24 (s, 0.2H), 7.20 (s, 1H), 5.05 (s, 0.2H), 4.83 (d, J=2.0 Hz, 2H), 4.24-4.18 (m, 2H), 3.77 (s, 3H), 3.58 (s, 1.6H), 2.90-2.75 (m, 3H), 2.64-2.61 (m, 2H), 2.27-2.20 (m, 1H), 1.89-1.75 (m, 1H), 1.29 (t, J=7.2 Hz, 3H).

### Intermediate KT7

### Methyl 4-(5-(3-ethoxy-3-oxopropanoyl)-4,5,6,7-tetrahydro-2H-indazol-2-yl)butanoate

LC-MS (ESI): R_{T}=1.42 min, mass calcd. for C₁₇H₂₄N₂O₅S 336.2, m/z found 337.2 [M+H]⁺. ¹H NMR (300 MHz, DMSO-d₆) δ 7.42-7.40 (m, 1H), 4.15-4.11 (m, 4H), 3.82-3.70 (m, 2H), 3.64-3.55 (m, 3H), 3.38-3.34 (m, 1H), 2.86-2.62 (m, 3H), 2.31-2.25 (m, 2H), 2.16-2.11 (m, 1H), 2.02-1.94 (m, 2H), 1.66-1.59 (m, 1H), 1.25-1.16 (m, 4H).

### Intermediate KT8

### Methyl 3-(5-(3-ethoxy-3-oxopropanoyl)-4,5,6,7-tetrahydro-2H-indazol-2-yl) butanoate (stereopure)

Converted from Acid 5.

LC-MS (ESI): R_{T}=1.41 min, mass calcd. for C₁₇H₂₄N₂O₅ 336.2, m/z found 337.4 [M+H]⁺.

### Intermediate KT9

### Methyl 3-(5-(3-ethoxy-3-oxopropanoyl)-4,5,6,7-tetrahydro-2H-indazol-2-yl) butanoate (enantiomer of KT8)

Converted from Acid 6.

¹H NMR (300 MHz, CDCl₃) δ 7.16 (s, 1H), 4.72-4.66 (m, 1H), 4.24-4.17 (m, 2H), 3.68 (s, 3H), 3.57 (s, 2H), 3.03-2.93 (m, 1H), 2.89-2.61 (m, 5H), 2.25-2.19 (m, 1H), 1.85-1.71 (m, 2H), 1.52 (d, J=6.9 Hz, 3H), 1.31-1.25 (m, 3H).

### Intermediate KT10

### Methyl 3-(5-(3-ethoxy-3-oxopropanoyl)-4,5,6,7-tetrahydro-2H-indazol-2-yl)-2,2-dimethylpropanoate

LC-MS (ESI): R_{T}=2.445 min, mass calcd. for C₁₈H₂₆N₂O₅S 350.2, m/z found 351.0 [M+H]⁺.

### Intermediate KT11

### (trans)-methyl 3-(5-(3-methoxy-3-oxopropanoyl)-4,5,6,7-tetrahydro-2H-indazol-2-yl)cyclobutanecarboxylate

LC-MS (ESI): R_{T}=1.50 min, mass calcd. for C₁₈H₂₄N₂O₅ 348.2, m/z found 349.3 [M+H]⁺. ¹H NMR (400 MHz, CDCl₃) δ 12.23 (s, 0.2H), 7.14-7.13 (d, J=5.2 Hz, 1H), 5.05 (s, 0.2H), 4.95-4.91 (m, 1H), 4.23-4.18 (m, 2H), 3.74 (s, 3H), 3.57 (s, 1.6H), 3.22-3.13 (m, 1H), 2.95-2.66 (m, 9H), 2.26-2.22 (m, 1H), 1.89-1.75 (m, 1H), 1.32-1.26 (m, 3H).

### Intermediate KT12

### Ethyl 3-(2-(3-methoxy-3-oxopropyl)-7,7-dimethyl-4,5,6,7-tetrahydro-2H-indazol-5-yl)-3-oxopropanoate

LC-MS (ESI): R_{T}=1.47 min, mass calcd. for C₁₈H₂₆N₂O₅ 350.2, m/z found 351.4 [M+H]⁺. ¹H NMR (300 MHz, DMSO-d₆) δ 7.33 (d, J=2.4 Hz, 1H), 4.25-4.20 (m, 2H), 4.14-4.06 (m, 2H), 3.75-3.73 (m, 2H), 3.59-3.58 (m, 3H), 3.00-2.90 (m, 1H), 2.81-2.70 (m, 3H), 2.43-2.33 (m, 1H), 1.86-1.81 (m, 1H), 1.49-1.40 (m, 1H), 1.25-1.15 (m, 9H).

### Intermediate KT13

### Methyl 3-(2-(3-methoxy-3-oxopropyl)-3-methyl-4,5,6,7-tetrahydro-2H-indazol-5-yl)-3-oxopropanoate

LC-MS (ESI): R_{T}=1.987 min, mass calcd. for C₁₇H₂₄N₂O₅ 336.2, m/z found 337.1 [M+H]⁺. ¹H NMR (400 MHz, CDCl₃) δ 7.71 (s, 0.5H), 4.26-4.18 (m, 4H), 3.68 (s, 3H), 3.58 (s, 1.5H), 2.88 (t, J=7.2 Hz, 2H), 2.83-2.77 (m, 2H), 2.69-2.53 (m, 3H), 2.23-2.18 (m, 1H), 2.18 (s, 3H), 1.80-1.69 (m, 1H), 1.32-1.24 (m, 3H).

### Intermediate KT14

### Mixture of ethyl 3-oxo-3-(2-(tetrahydro-2H-pyran-2-yl)-4,5,6,7-tetrahydro-2H-indazol-5-yl)propanoate and ethyl 3-oxo-3-(1-(tetrahydro-2H-pyran-2-yl)-4,5,6,7-tetrahydro-2H-indazol-5-yl)propanoate

LC-MS (ESI): R_{T}=1.50 min, mass calcd. for C₁₇H₂₄N₂O₄ 320.2, m/z found 321.2 [M+H]⁺. ¹H NMR (300 MHz, CDCl₃) δ 12.24 (s, 0.2H), 7.34 (s, 1H), 5.28-5.19 (m, 1H), 5.05 (s, 0.2H), 4.21-4.19 (m, 2H), 4.13-3.99 (m, 1H), 3.89-3.63 (m, 1.7H), 3.57 (s, 1.6H), 3.36 (s, 0.3H), 2.92-2.64 (m, 4H), 2.45-2.10 (m, 2H), 1.81-1.57 (m, 6H), 1.30-1.27 (m, 3H).

### Intermediate KT15

### Ethyl 3-(2-(3-methoxy-3-oxopropyl)-4,5,6,7-tetrahydrobenzo[d]oxazol-5-yl)-3-oxopropanoate

LC-MS (ESI): R_{T}=1.42 min, mass calcd. for C₁₆H₂₁NO₆ 323.1, m/z found 324.2 [M+H]⁺. ¹H NMR (300 MHz, CDCl₃) δ 12.24 (s, 0.2H), 4.23-4.10 (m, 2H), 3.70 (s, 3H), 3.57-3.45 (m, 1.8H), 3.06-2.92 (m, 3H), 2.82-2.69 (m, 6H), 2.24-2.09 (m, 1H), 1.94-1.74 (m, 1H), 1.30-1.25 (m, 3H).

### Intermediates KT16

### Ethyl 3-(2-(3-methoxy-3-oxopropyl)-4,5,6,7-tetrahydrobenzo[d]oxazol-6-yl)-3-oxopropanoate

¹H NMR (300 MHz, DMSO-d₆) δ 4.11-4.04 (m, 2H), 3.76-3.74 (m, 2H), 3.59 (s, 2H), 3.58 (s, 1H), 3.05-2.97 (m, 1H), 2.94-2.89 (m, 2H), 2.74-2.68 (m, 4H), 2.40-2.38 (m, 2H), 2.14-2.08 (m, 1H), 1.71-1.59 (m, 1H), 1.19-1.14 (m, 3H).

### Intermediate KT17

### Ethyl 3-(2-(3-methoxy-3-oxopropyl)-4,5,6,7-tetrahydrobenzo[d]thiazol-5-yl)-3-oxopropanoate

LC-MS (ESI): R_{T}=1.53 min, mass calcd. for C₁₆H₂₁NO₅S 339.1, m/z found 340.2 [M+H]⁺. ¹H NMR (300 MHz, DMSO-d₆) δ 4.10 (q, J=7.2 Hz, 2H), 3.77 (d, J=3.3 Hz, 2H), 3.60 (s, 3H), 3.14 (t, J=7.2 Hz, 2H), 3.04-2.57 (m, 7H), 2.21-2.12 (m, 1H), 2.74-1.61 (m, 1H), 1.19 (t, J=7.2 Hz, 3H).

### Intermediate KT18

### Ethyl 3-(2-(3-methoxy-3-oxopropyl)-4,5,6,7-tetrahydrobenzo[d]thiazol-6-yl)-3-oxopropanoate

LC-MS (ESI): R_{T}=1.50 min, mass calcd. for C₁₆H₂₁NO₅S 339.1, m/z found 340.2 [M+H]⁺. ¹H NMR (300 MHz, DMSO-d₆) δ 4.13-4.06 (m, 2H), 3.77-3.75 (m, 2H), 3.60 (s, 3H), 3.15-3.10 (m, 2H), 2.96-2.89 (m, 2H), 2.82-2.67 (m, 5H), 2.20-2.12 (m, 1H), 1.77-1.68 (m, 1H), 1.20-1.15 (m, 3H).

### Intermediate KT19

### 3-[2-(2-Methoxycarbonyl-ethyl)-4,5,6,7-tetrahydro-2H-indazol-6-yl]-3-oxo-propionic acid ethyl ester

LC-MS (ESI): R_{T}=1.522 min, mass calcd. for C₁₆H₂₂N₂O₅ 322.2, m/z found 323.1 [M+H]⁺. ¹H NMR (300 MHz, CDCl₃) δ 12.21 (s, 0.2H), 7.13 (s, 1H), 5.03 (s, 0.2H), 4.32 (t, J=6.6 Hz, 2H), 4.19 (q, J=7.2 Hz, 2H), 3.68 (s, 3H), 3.56 (s, 1.6H), 2.95-2.81 (m, 4H), 2.77-2.64 (m, 2H), 2.56-2.45 (m, 1H), 2.16-2.11 (m, 1H), 1.76-1.62 (m, 1H), 1.31-1.22 (m, 3H).

### Intermediate KT20

### Ethyl 3-(1-cyano-2-(3-methoxy-3-oxopropyl)-4,5,6,7-tetrahydro-2H-isoindol-5-yl)-3-oxopropanoate

LC-MS (ESI): R_{T}=1.58 min, mass calcd. for C₁₈H₂₂N₂O₅ 346.2, m/z found 347.3 [M+H]⁺.

### Intermediate KT21

### Ethyl 3-(3-cyano-2-(3-methoxy-3-oxopropyl)-4,5,6,7-tetrahydro-2H-isoindol-5-yl)-3-oxopropanoate

LC-MS (ESI): R_{T}=2.271 min, mass calcd. for C₁₈H₂₂N₂O₅ 346.2, m/z found 347.1 [M+H]⁺.

### Intermediate KT22

### Methyl 3-(2-(3-methoxy-3-oxopropyl)-5,6,7,8-tetrahydroquinazolin-6-yl)-3-oxopropanoate

LC-MS (ESI): R_{T}=1.32 min, mass calcd. for C₁₆H₂₀N₂O₅ 320.1, m/z found 321.0 [M+H]⁺. ¹H NMR (400 MHz, CDCl₃) δ 12.18 (s, 0.2H), 8.37 (s, 0.8H), 8.35 (s, 0.2H), 5.08 (s, 0.2H), 3.76 (s, 2.4H), 3.75 (s, 0.6H), 3.68 (s, 3H), 3.62 (s, 1.6H), 3.23 (t, J=7.2 Hz, 2H), 3.01-2.84 (m, 7H), 2.32-2.26 (m, 1H), 1.92-1.84 (m, 1H).

### Intermediate KT23: (trans)-methyl 3-(6-(3-methoxy-3-oxopropanoyl)-4,5,6,7-tetrahydrobenzo[d]thiazol-2-yl)cyclobutanecarboxylate

Mass calcd. for C₁₇H₂₁NO₅S 351.1, m/z found 352.1 [M+H]⁺. ¹H NMR (400 MHz, CDCl₃) δ 12.16 (s, 0.2H), 5.07 (s, 0.2H), 3.95-3.87 (m, 1H), 3.75 (s, 3H), 3.72 (s, 3H), 3.60 (s, 1.6H), 3.28-3.21 (m, 1H), 3.02-2.96 (m, 3H), 2.92-2.86 (m, 1H), 2.82-2.69 (m, 3H), 2.63-2.56 (m, 2H), 2.29-2.24 (m, 1H), 1.92-1.82 (m, 1H).

### Intermediate KT24: (trans)-methyl 3-(6-(3-methoxy-3-oxopropanoyl)-4,5,6,7-tetrahydrobenzo[d]oxazol-2-yl)cyclobutanecarboxylate

LC-MS (ESI): R_{T}=1.47 min, mass calcd. for C₁₇H₂₁NO₆335.1, m/z found 335.9 [M+H]⁺. ¹H NMR (400 MHz, CDCl₃) δ 12.11 (s, 0.2H), 5.03 (s, 0.2H), 3.77-3.73 (m, 4H), 3.69 (s, 3H), 3.55 (s, 1.6H), 3.14-2.98 (m, 2H), 2.94-2.79 (m, 2H), 2.68-2.44 (m, 6H), 2.16-2.01 (m, 1H), 1.82-1.69 (m, 1H).

### Intermediate KT25: Methyl 4-(5-(3-methoxy-3-oxopropanoyl)-4,5,6,7-tetrahydro-2H-indazol-2-yl)butanoate

¹H NMR (400 MHz, DMSO-d₆) δ 7.39 (s, 1H), 4.22 (t, J=6.8 Hz, 0.3H), 3.98 (t, J=6.8 Hz, 1.7H), 3.81-3.67 (m, 2H), 3.64-3.51 (m, 6H), 2.84-2.54 (m, 3.3H), 2.45-2.43 (m, 0.7), 2.26 (t, J=7.2 Hz, 2H), 2.13-2.10 (m, 1H), 1.98-1.91 (m, 2H), 1.66-1.56 (m, 1H), 1.40-1.15 (m, 1H).

### Intermediate KT26: Methyl 6-(3-methoxy-3-oxopropanoyl)-4,5,6,7-tetrahydrobenzo[d]oxazole-2-carboxylate

LC-MS (ESI): R_{T}=0.90 min, mass calcd. for C₁₃H₁₅NO₆ 281.1, m/z found 281.9 [M+H]⁺. ¹H NMR (300 MHz, CDCl₃) δ 12.19 (s, 0.2H), 5.08 (s, 0.2H), 4.00 (s, 3H), 3.76 (s, 3H), 3.62 (s, 1.6H), 3.16-2.85 (m, 3H), 2.78-2.62 (m, 2H), 2.31-2.20 (m, 1H), 1.99-1.80 (m, 1H).

### Intermediate KT27: Methyl 3-(5-(3-methoxy-3-oxopropanoyl)-4,5,6,7-tetrahydro-2H-indazol-2-yl)-3-methylbutanoate

LC-MS (ESI): R_{T}=1.582 min, mass calcd. for C₁₇H₂₄N₂O₅ 336.2, m/z found 337.1 [M+H]⁺. ¹H NMR (400 MHz, CDCl₃) δ 7.27 (s, 1H), 3.75 (s, 3H), 3.59 (s, 5H), 2.89-2.81 (m, 4H), 2.71-2.62 (m, 2H), 2.25-2.18 (m, 1H), 1.88-1.71 (m, 2H), 1.68 (s, 6H).

### Intermediate KT28: Ethyl 3-(5-(3-methoxy-3-oxopropanoyl)-4,5,6,7-tetrahydro-2H-indazol-2-yl)-2,2-dimethylpropanoate

LC-MS (ESI): R_{T}=1.53 min, mass calcd. for C₁₈H₂₆N₂O₅ 350.2, m/z found 351.0 [M+H]⁺. ¹H NMR (400 MHz, CDCl₃) δ 12.14 (s, 0.2H), 7.09 (s, 0.8H), 7.07 (s, 0.2H), 5.06 (s, 0.2H), 4.23-4.14 (m, 4H), 3.75 (s, 2.4H), 3.74 (s, 0.6H), 3.59 (s, 1.6H), 2.87-2.56 (m, 5H), 2.23-2.10 (m, 1H), 1.88-1.72 (m, 1H), 1.26 (t, J=7.2 Hz, 3H), 1.19 (s, 6H).

### Intermediate KT29: (trans)-methyl 4-(6-(3-methoxy-3-oxopropanoyl)-4,5,6,7-tetrahydrobenzo[d]oxazol-2-yl)cyclohexanecarboxylate

LC-MS (ESI): R_{T}=1.30 min, mass calcd. for C₁₉H₂₅NO₆ 363.2, m/z found 363.9 [M+H]⁺.

### Intermediate KT30: Ethyl 4-(5-(3-ethoxy-3-oxopropanoyl)-4,5,6,7-tetrahydro-2H-indazol-2-yl)-2,2-dimethylbutanoate

¹H NMR (400 MHz, CDCl₃) δ 7.10 (s, 1H), 4.20 (q, J=7.2 Hz, 2H), 4.12 (q, J=7.2 Hz, 2H), 4.05-4.01 (m, 2H), 3.56 (s, 2H), 2.87-2.77 (m, 3H), 2.71-2.59 (m, 2H), 2.26-2.20 (m, 1H), 2.10-2.06 (m, 2H), 1.86-1.75 (m, 1H), 1.28-1.23 (m, 12H).

### Intermediate KT31: Ethyl 4-(5-(3-methoxy-3-oxopropanoyl)-4,5,6,7-tetrahydro-2H-indazol-2-yl)-2,2-dimethylbutanoate

¹H NMR (400 MHz, CDCl₃) 7.10 (s, 1H), 4.12 (q, J=7.2 Hz, 2H), 4.05-4.01 (m, 2H), 3.75 (s, 3H), 3.58 (s, 2H), 2.88-2.77 (m, 3H), 2.71-2.61 (m, 2H), 2.27-2.19 (m, 1H), 2.10-2.06 (m, 2H), 1.83-1.78 (m, 1H), 1.26-1.23 (m, 9H).

### Intermediate KT32: Ethyl 3-(5-(3-ethoxy-3-oxopropanoyl)-4,5,6,7-tetrahydro-2H-indazol-2-yl)-2,2-dimethylpropanoate

LC-MS (ESI): R_{T}=1.41 min, mass calcd. for C₁₉H₂₈N₂O₅ 364.2, m/z found 365.0 [M+H]⁺. ¹H NMR (400 MHz, CDCl₃) δ 12.23 (s, 0.2H), 7.09-7.07 (m, 1H), 5.04 (s, 0.2H), 4.23-4.14 (m, 6H), 3.57 (s, 1.6H), 2.87-2.76 (m, 3H), 2.69-2.56 (m, 2H), 2.24-2.10 (m, 1H), 1.89-1.72 (m, 1H), 1.32-1.25 (m, 6H), 1.19 (s, 6H).

### Intermediate KT33: (cis)-Methyl 3-(5-(3-methoxy-3-oxopropanoyl)-4,5,6,7-tetrahydro-2H-indazol-2-yl)cyclobutanecarboxylate

LC-MS (ESI): R_{T}=1.48 min, mass calcd. for C₁₇H₂₂N₂O₅ 334.2, m/z found 335.0 [M+H]⁺. ¹H NMR (400 MHz, CDCl₃) δ 12.2 (s, 0.1H), 7.27 (s, 1H), 5.06 (s, 0.1H), 4.68-4.59 (m, 1H), 3.75 (s, 3H), 3.72 (s, 3H), 3.60 (s, 1.8H), 2.97-2.63 (m, 10H), 2.24-2.20 (m, 1H), 1.84-1.77 (m, 1H).

### Spectral Analyses of Dihydropyrimidines

### Compound 2

### Ethyl 4-(3,4-difluoro-2-methylphenyl)-6-(2-(3-methoxy-3-oxopropyl)-4,5,6,7-tetrahydro-2H-indazol-5-yl)-2-(thiazol-2-yl)-1,4-dihydropyrimidine-5-carboxylate

LC-MS (ESI): R_{T}=2.920 min, mass calcd. for C₂₈H₂₉F₂N₅O₄S 569.2, m/z found 569.9 [M+H]⁺. ¹H NMR (400 MHz, DMSO-d₆) δ 8.17 (s, 0.8H), 7.82 (d, J=3.2 Hz, 0.2H), 7.75-7.74 (m, 0.8H), 7.48 (d, J=3.6 Hz, 0.2H), 7.41 (d, J=3.6 Hz, 0.8H), 7.24-7.12 (m, 1.2H), 7.03-6.33 (m, 2H), 5.98 (s, 0.4H), 5.96 (s, 0.4H), 5.89 (m, 0.2H), 4.41-4.32 (m, 3H), 4.07-4.00 (m, 2H), 3.71 (s, 3H), 3.10-2.88 (m, 4H), 2.85-2.66 (m, 2H), 2.58 (s, 2.4H), 2.45-2.42 (m, 0.6H), 2.28-1.99 (m, 2H), 1.11 (t, J=6.8 Hz, 3H).

A stereoisomeric mixture of ethyl 4-(3,4-difluoro-2-methylphenyl)-6-(2-(3-methoxy-3-oxopropyl)-4,5,6,7-tetrahydro-2H-indazol-5-yl)-2-(thiazol-2-yl)-1,4-dihydropyrimidine-5-carboxylate Compound 2 (420 mg, 0.82 mmol) was separated by Chiral Prep. HPLC (the first separation condition: Column: Chiralpak IA 5 μm 20*250 mm; Mobile Phase: Hex:EtOH=60:40 at 25 mL/min; Temp: 30° C.; Wavelength: 214 nm; the second separation condition: Column: Chiralpak IG 5 cm*250 mm; Mobile Phase: MeOH=100% at 60 mL/min; Temp: 35° C.; Wavelength: 254 nm; the third separation condition: Column: Chiralpak IG 5 cm*250 mm; Mobile Phase: Hex:EtOH:DEA=70:30:0.1 at 60 mL/min; Temp: 35° C.; Wavelength: 254 nm) to give the title compounds Compound 2a (55 mg, 13% yield, 100% stereopure), Compound 2b (60 mg, 14% yield, 100% stereopure), Compound 2c (65 mg, 15% yield, 100% stereopure) and Compound 2d (70 mg, 17% yield, 98.2% stereopure) as yellow solids

Compound 2b: LC-MS (ESI): R_{T}=3.936 min, mass calcd. for C₂₇H₂₆ClF₂N₅O₄S 569.2, m/z found 570.2 [M+H]⁺. Chiral HPLC (Column: Chiralpak IG 5 μm 4.6*250 mm; Mobile Phase: Hex:EtOH:DEA=80:20:0.2 at 1.0 mL/min; Temp: 30° C.; Wavelength: 254 nm, R_{T}=14.120 min). ¹H NMR (400 MHz, CDCl₃) δ 8.16 (s, 0.7H), 7.80 (d, J=3.2 Hz, 0.2H), 7.74 (d, J=2.8 Hz, 0.8H), 7.48 (d, J=3.2 Hz, 0.2H), 7.41 (d, J=3.2 Hz, 0.8H), 7.24 (s, 1H), 7.18-7.09 (m, 0.3H), 7.00-6.88 (m, 2H), 5.98 (s, 0.8 H), 5.89 (d, J=4.4 Hz, 0.2H), 4.40-4.33 (m, 3H), 4.09-3.94 (m, 2H), 3.71 (s, 3H), 3.10-3.01 (m, 1H), 2.91-2.88 (m, 3H), 2.85-2.70 (m, 2H), 2.58 (s, 1.2H), 2.57 (s, 1.2H), 2.45 (s, 0.6H), 2.18-2.10 (m, 1H), 2.04-1.88 (m, 1H), 1.10 (t, J=7.2 Hz, 3H).

Compound 2c: LC-MS (ESI): R_{T}=3.941 min, mass calcd. for C₂₇H₂₆ClF₂N₅O₄S 569.2, m/z found 570.2 [M+H]⁺. Chiral HPLC (Column: Chiralpak IA 5 μm 4.6*250 mm; Mobile Phase: Hex:EtOH=60:40 at 1.0 mL/min; Temp: 30° C.; Wavelength: 230 nm, R_{T}=6.077 min). ¹H NMR (400 MHz, CDCl₃) δ 8.17 (m, 0.7H), 7.80 (d, J=3.2 Hz, 0.2H), 7.74 (d, J=2.8 Hz, 0.8H), 7.48 (d, J=5.2 Hz, 0.2H), 7.41 (d, J=2.8 Hz, 0.8H), 7.21 (s, 1H), 7.17-7.13 (m, 0.3H), 7.04-6.98 (m, 1H), 6.95-6.88 (m, 1H), 5.96 (s, 0.8H), 5.88 (d, J=2.4 Hz, 0.2H), 4.44-4.33 (m, 2.8H), 4.11-3.96 (m, 2.2H), 3.71 (s, 3H), 2.99-2.80 (m, 5H), 2.69-2.63 (m, 1H), 2.58 (s, 1.2H), 2.57 (s, 1.2H), 2.45 (s, 0.6H), 2.31-2.24 (m, 1H), 2.17-2.02 (m, 1H), 1.11 (t, J=7.2 Hz, 3H).

### Compound 3

### Ethyl 4-(2-bromo-3,4-difluorophenyl)-6-(2-(3-methoxy-3-oxopropyl)-4,5,6,7-tetrahydro-2H-indazol-5-yl)-2-(thiazol-2-yl)-1,4-dihydropyrimidine-5-carboxylate

LC-MS (ESI): R_{T}=4.228 min, mass calcd. for C₂₇H₂₆BrF₂N₅O₄S 633.1, m/z found 633.8 [M+H]⁺. ¹H NMR (400 MHz, DMSO-d₆) δ 9.57 (d, J=3.2 Hz, 0.3H), 9.54 (d, J=3.6 Hz, 0.3H), 9.04 (s, 0.2H), 9.00 (s, 0.2H), 8.00-7.98 (m, 1.2H), 7.95 (d, J=3.2 Hz, 0.4H), 7.92 (d, J=2.8 Hz, 0.4H), 7.55-7.49 (m, 1H), 7.45 (s, 0.4H), 7.38 (d, J=8.4 Hz, 0.6H), 7.29-7.23 (m, 1H), 6.05 (d, J=6.8 Hz, 0.4H), 5.97-5.94 (m, 0.6H), 4.28-4.22 (m, 2H), 4.18-4.13 (m, 0.4H), 3.99-3.90 (m, 2.6H), 3.61 (s, 3H), 2.94-2.90 (m, 1H), 2.86-2.83 (m, 2H), 2.78-2.68 (m, 2H), 2.61-2.52 (m, 1H), 2.11-2.08 (m, 0.7H), 2.01-1.95 (m, 1H), 1.83-1.79 (m, 0.3H), 1.06-1.00 (m, 3H).

A stereoisomeric mixture of Compound 3 (440 mg, 0.69 mmol) was separated by chiral Prep. HPLC (the first separation condition: Column: Chiralpak IG 5 μm 20*250 mm; Mobile Phase: Hex:EtOH:DEA=70:30:0.3 at 15 mL/min; Temp: 30° C.; Wavelength: 214 nm; the second separation condition: Column: Chiralpak IG 5 μm 20*250 mm; Mobile Phase: Hex:EtOH:DEA=80:20:0.3 at 15 mL/min; Temp: 30° C.; Wavelength: 214 nm; the third separation condition: Column: Chiralpak IG 5 μm 20*250 mm; Mobile Phase: Hex:EtOH:DEA=80:20:0.3 at 15 mL/min; Temp: 30° C.; Wavelength: 214 nm) to give the title compounds Compound 3a (80 mg, 18% yield, 100% stereopure), Compound 3b (75 mg, 17% yield, 96.0% stereopure), Compound 3c (65 mg, 15% yield, 100% stereopure) and Compound 3d (60 mg, 14% yield, 100% stereopure). Compound 3b: LC-MS (ESI): R_{T}=4.101 min, mass calcd. for C₂₇H₂₆BrF₂N₅O₄S 634.4, m/z found 636.0 [M+H]⁺. Chiral HPLC (Column: Chiralpak IG 5 μm 4.6*250 mm; Mobile Phase: Hex:EtOH:DEA=70:30:0.2 at 1.0 mL/min; Temp: 30° C.; Wavelength: 230 nm, R_{T}=10.799 min). ¹H NMR (400 MHz, DMSO-d₆) δ 9.54 (d, J=3.2 Hz, 0.6H), 9.04 (s, 0.4H), 8.00-7.98 (m, 1.2H), 7.95 (d, J=3.2 Hz, 0.4H), 7.92 (d, J=2.8 Hz, 0.4H), 7.55-7.49 (m, 1H), 7.45 (s, 0.4H), 7.40 (s, 0.6H), 7.26-7.20 (m, 1H), 6.06 (s, 0.4H), 5.96 (d, J=3.2 Hz, 0.6H), 4.28-4.23 (m, 2H), 4.18-4.12 (m, 0.5H), 3.99-3.90 (m, 2.5H), 3.61 (s, 3H), 2.98-2.90 (m, 0.5H), 2.87-2.83 (m, 2.5H), 2.72-2.67 (m, 2H), 2.59-2.56 (m, 1H), 2.16-2.10 (m, 0.4H), 2.00-1.93 (m, 1H), 1.82-1.79 (m, 0.6H), 1.06-0.98 (m, 3H).

Compound 3d: LC-MS (ESI): R_{T}=4.120 min, mass calcd. for C₂₇H₂₆BrF₂N₅O₄S 634.4, m/z found 636.0 [M+H]⁺. Chiral HPLC (Column: Chiralpak IG 5 μm 4.6*250 mm; Mobile Phase: Hex:EtOH:DEA=70:30:0.2 at 1.0 mL/min; Temp: 30° C.; Wavelength: 230 nm, R_{T}=16.329 min). ¹H NMR (400 MHz, DMSO-d₆) δ 9.57 (d, J=3.2 Hz, 0.7H), 9.00 (s, 0.3H), 8.00-7.93 (m, 2H), 7.55-7.48 (m, 1H), 7.44 (s, 0.4H), 7.38 (s, 0.6H), 7.30-7.23 (m, 1H), 6.04 (s, 0.3H), 5.95 (d, J=3.2 Hz, 0.7H), 4.26-4.22 (m, 2H), 4.18-4.15 (m, 0.3H), 3.99-3.91 (m, 2.7H), 3.61 (s, 3H), 2.86-2.83 (m, 2H), 2.78-2.72 (m, 2H), 2.68-2.54 (m, 2H), 2.27-2.19 (m, 0.4H), 2.12-2.07 (m, 1H), 2.04-2.00 (m, 0.6H), 1.06-0.99 (m, 3H).

### Compound 4

### Ethyl 4-(2-chloro-4-fluorophenyl)-6-(2-(3-methoxy-3-oxopropyl)-4,5,6,7-tetrahydro-2H-indazol-5-yl)-2-(thiazol-2-yl)-1,4-dihydropyrimidine-5-carboxylate

LC-MS (ESI): R_{T}=2.598 min, mass calcd. for C₂₇H₂₇ClFN₅O₄S 571.2, m/z found 572.1 [M+H]⁺. ¹H NMR (400 MHz, CDCl₃) δ 8.19 (s, 0.5H), 7.82 (d, J=3.2 Hz, 0.5H), 7.76 (d, J=2.8 Hz, 0.5H), 7.47 (d, J=2.8 Hz, 0.5H), 7.42 (d, J=3.2 Hz, 0.5H), 7.40-7.33 (m, 1.5H), 7.23-7.19 (m, 0.8H), 7.16-7.12 (m, 1.2H), 6.99-6.91 (m, 1H), 6.24 (s, 0.25H), 6.22 (s, 0.25H), 6.11 (s, 0.5H), 4.38-4.34 (m, 2.5H), 4.13-4.01 (m, 2.5H), 3.71 (s, 3H), 3.12-3.03 (m, 1H), 2.94-2.84 (m, 4H), 2.70-2.59 (m, 1H), 2.31-1.94 (m, 2H), 1.13-1.08 (m, 3H).

A stereoisomeric mixture of ethyl 4-(2-chloro-4-fluorophenyl)-6-(2-(3-methoxy-3-oxopropyl)-4,5,6,7-tetrahydro-2H-indazol-5-yl)-2-(thiazol-2-yl)-1,4-dihydropyrimidine-5-carboxylate Compound 4 (597 mg, 1.043 mmol) was separated by chiral Prep. HPLC (the first separation condition: Column: Chiralpak IG 5 μm 20*250 mm; Mobile Phase: Hex:EtOH:DEA=60:40:0.2 at 15 mL/min; Temp: 30° C.; Wavelength: 230 nm; the second separation condition: Column: Chiralpak IA, 5 μm 4.6*250 mm; Mobile Phase: Hex:EtOH:DEA=70:30:0.3 at 25 mL/min; Temp: 30° C.; Wavelength: 230 nm; the third separation condition: Column: Chiralpak IG, 5 μm 4.6*250 mm; Mobile Phase: Hex:EtOH:DEA=65:35:0.3 at 15 mL/min; Temp: 30° C.; Wavelength: 214 nm) to afford the title compounds Compound 4a (123 mg, 21% yield, 100% stereopure), Compound 4b (112 mg, 19% yield, 95.7% stereopure), Compound 4c (109 mg, 18% yield, 100% stereopure) and Compound 4d (125 mg, 21% yield, 100% stereopure) as yellow solids.

Compound 4b: LC-MS (ESI): R_{T}=4.407 min, mass calcd. for C₂₇H₂₇ClFN₅O₄S 571.2, m/z found 572.2 [M+H]⁺. Chiral HPLC (Column: Chiralpak IA, 5 μm 4.6*250 mm; Mobile Phase: Hex:EtOH:DEA=70:30:0.2 at 1 mL/min; Temp: 30° C.; Wavelength: 230 nm; R_{T}=8.894 min). ¹H NMR (400 MHz, CDCl₃) δ 8.19 (s, 0.5H), 7.82 (d, J=3.2 Hz, 0.5H), 7.76 (d, J=3.2 Hz, 0.5H), 7.48 (d, J=2.8 Hz, 0.5H), 7.42 (d, J=2.8 Hz, 0.5H), 7.40-7.35 (m, 1.5H), 7.21 (s, 0.5H), 7.16-7.12 (m, 1.5H), 6.99-6.91 (m, 1H), 6.22 (s, 0.5H), 6.11 (d, J=2.4 Hz, 0.5H), 4.38-4.34 (m, 2.5H), 4.10-4.01 (m, 2.5H), 3.71 (s, 3H), 2.95-2.89 (m, 4H), 2.87-2.80 (m, 1H), 2.69-2.59 (m, 1H), 2.32-2.03 (m, 2H), 1.13-1.08 (m, 3H).

Compound 4c: LC-MS (ESI): R_{T}=4.415 min, mass calcd. for C₂₇H₂₇ClFN₅O₄S 571.2, m/z found 572.1 [M+H]⁺. Chiral HPLC (Column: Chiralpak IA, 5 μm 4.6*250 mm; Mobile Phase: Hex:EtOH:DEA=70:30:0.2 at 1 mL/min; Temp: 30° C.; Wavelength: 230 nm; R_{T}=7.471 min). ¹H NMR (400 MHz, CDCl₃) δ 8.20 (s, 0.5H), 7.83 (d, J=2.8 Hz, 0.5H), 7.77 (d, J=2.8 Hz, 0.5H), 7.48 (d, J=2.8 Hz, 0.5H), 7.43 (d, J=3.2 Hz, 0.5H), 7.41-7.36 (m, 1.5H), 7.22 (s, 0.5H), 7.16-7.13 (m, 1.5H), 7.00-6.92 (m, 1H), 6.23 (s, 0.5H), 6.11 (s, 0.5H), 4.42-4.345 (m, 2.5H), 4.18-4.00 (m, 2.5H), 3.72 (s, 3H), 2.97-2.80 (m, 5H), 2.70-2.60 (m, 1H), 2.35-2.06 (m, 2H), 1.14-1.09 (m, 3H).

Compound 4d: LC-MS (ESI): R_{T}=3.202 min, mass calcd. for C₂₇H₂₇ClFN₅O₄S 571.2, m/z found 572.1 [M+H]⁺. Chiral HPLC (Column: Chiralpak IG, 5 μm 4.6*250 mm; Mobile Phase: Hex:EtOH:DEA=75:25:0.2 at 1 mL/min; Temp: 30° C.; Wavelength: 230 nm; R_{T}=13.336 min). ¹H NMR (400 MHz, CDCl₃) δ 8.19 (s, 0.5H), 7.82 (d, J=2.8 Hz, 0.5H), 7.75 (d, J=3.2 Hz, 0.5H), 7.47 (d, J=3.2 Hz, 0.5H), 7.42 (d, J=2.8 Hz, 0.5H), 7.37-7.33 (m, 1.5H), 7.23 (s, 0.5H), 7.20 (s, 0.5H), 7.15-7.13 (m, 1H), 6.99-6.91 (m, 1H), 6.24 (s, 0.5H), 6.11 (d, J=2.8 Hz, 0.5H), 4.38-4.35 (m, 2.5H), 4.06-4.02 (m, 2.5H), 3.71 (s, 3H), 3.12-3.01 (m, 1H), 2.92-2.88 (m, 4H), 2.84-2.70 (m, 1H), 2.20-2.12 (m, 1H), 2.05-1.91 (m, 1H), 1.13-1.09 (m, 3H).

### Compound 5

### Ethyl 4-(4-fluoro-2-methylphenyl)-6-(2-(3-methoxy-3-oxopropyl)-4,5,6,7-tetrahydro-2H-indazol-5-yl)-2-(thiazol-2-yl)-1,4-dihydropyrimidine-5-carboxylate

LC-MS (ESI): R_{T}=4.243 min, mass calcd. for C₂₈H₃₀FN₅O₄S 551.6, m/z found 552.0 [M+H]⁺. ¹H NMR (400 MHz, DMSO-d₆) δ 9.50-9.47 (m, 0.8H), 8.94-8.89 (m, 0.2H), 7.98-7.89 (m, 2H), 7.45-7.37 (m, 1H), 7.35-7.28 (m, 0.8H), 7.24-7.19 (m, 0.2H), 7.04-6.99 (m, 2H), 5.85-5.84 (m, 0.2H), 5.72-5.70 (m, 0.8H), 4.26-4.22 (m, 2H), 3.97-3.89 (m, 3H), 3.61 (s, 3H), 2.94-2.90 (m, 0.5H), 2.85 (t, J=6.8 Hz, 2H), 2.80-2.77 (m, 0.5H), 2.74-2.65 (m, 2H), 2.56-2.52 (m, 4H), 2.11-1.97 (m, 1.5H), 1.82-1.76 (m, 0.5H), 1.04-0.99 (m, 3H).

A stereoisomeric mixture of ethyl 4-(4-fluoro-2-methylphenyl)-6-(2-(3-methoxy-3-oxopropyl)-4,5,6,7-tetrahydro-2H-indazol-5-yl)-2-(thiazol-2-yl)-1,4-dihydropyrimidine-5-carboxylate Compound 5 (434 mg, 99% purity, 0.780 mmol) was separated by chiral Prep. HPLC (the first separation condition: Column: Chiralpak IA 5 μm 30*250 mm; Mobile Phase: Hex:EtOH:DEA=70:30:0.3 at 25 mL/min; Temp: 30° C.; Wavelength: 214 nm; the second separation condition: Column: Chiralpak IG 5 μm 20*250 mm; Mobile Phase: Hex:EtOH:DEA=70:30:0.3 at 15 mL/min; Temp: 30° C.; Wavelength: 214 nm) to afford the title compounds Compound 5a (80 mg, 19% yield, 100% stereopure), Compound 5b (80 mg, 19% yield, 100% stereopure), Compound 5d (100 mg, 23% yield, 100% stereopure) and Compound 5c (113 mg, 26% yield, 97.6% stereopure) as yellow solids.

Compound 5a: LC-MS (ESI):R_{T}=4.073 min, mass calcd. for C₂₈H₃₀FN₅O₄S 551.6, m/z found 552.2 [M+H]⁺. Chiral HPLC (Column: Chiralpak IA 5 μm 4.6*250 mm; Mobile Phase: Hex:EtOH:DEA=70:30:0.2 at 1.0 mL/min; Temp: 30° C.; Wavelength: 230 nm, R_{T}=6.179 min). ¹H NMR (400 MHz, DMSO-d₆) δ9.47 (s, 0.8H), 8.94 (s, 0.2H), 7.98-7.90 (m, 2H), 7.45 (s, 0.2H), 7.40 (s, 0.8H), 7.32-7.28 (m, 0.8H), 7.22-7.19 (m, 0.2H), 7.06-6.97 (m, 2H), 5.85 (s, 0.2H), 5.71 (s, 0.8H), 4.28-4.23 (m, 2H), 4.16-4.12 (m, 0.2H), 3.97-3.86 (m, 2.8H), 3.61 (s, 3H), 2.94-2.83 (m, 3H), 2.75-2.66 (m, 2H), 2.56-2.50 (m, 4H), 2.10-2.03 (m, 0.2H), 2.01-1.94 (m, 1H), 1.81-1.79 (m, 0.8H), 1.05-0.99 (m, 3H).

Compound 5b: LC-MS (ESI):R_{T}=4.235 min, mass calcd. for C₂₈H₃₀FN₅O₄S 551.6, m/z found 552.0 [M+H]⁺. Chiral HPLC (Column: Chiralpak IG 5 μm 4.6*250 mm; Mobile Phase: Hex:EtOH:DEA=70:30:0.2 at 1.0 mL/min; Temp: 30° C.; Wavelength: 230 nm, R_{T}=10.966 min). ¹H NMR (400 MHz, DMSO-d₆) δ 9.49 (d, J=3.2 Hz, 0.8H), 8.95 (s, 0.2H), 7.98-7.89 (m, 2H), 7.45 (s, 0.2H), 7.40 (s, 0.8H), 7.32-7.28 (m, 0.8H), 7.22-7.18 (m, 0.2H), 7.06-6.99 (m, 2H), 5.85 (s, 0.2H), 5.71 (d, J=3.2 Hz, 0.8H), 4.28-4.23 (m, 2H), 4.18-4.11 (m, 0.2H), 3.97-3.85 (m, 2.8H), 3.61 (s, 3H), 2.94-2.83 (m, 3H), 2.75-2.66 (m, 2H), 2.58-2.50 (m, 4H), 2.14-2.07 (m, 0.2H), 2.01-1.93 (m, 1H), 1.81-1.78 (m, 0.8H), 1.04-0.99 (m, 3H).

Compound 5d: LC-MS (ESI):R_{T}=4.229 min, mass calcd. for C₂₈H₃₀FN₅O₄S 551.6, m/z found 552.0 [M+H]⁺. Chiral HPLC (Column: Chiralpak IG 5 μm 4.6*250 mm; Mobile Phase: Hex:EtOH:DEA=70:30:0.2 at 1.0 mL/min; Temp: 30° C.; Wavelength: 230 nm, R_{T}=14.019 min). ¹H NMR (400 MHz, DMSO-d₆) δ 9.51 (d, J=2.8 Hz, 0.8H), 8.91 (s, 0.1H), 7.99-7.91 (m, 2H), 7.44 (s, 0.2H), 7.37-7.31 (m, 1.6H), 7.24-7.20 (m, 0.2H), 7.09-6.96 (m, 2H), 5.84 (s, 0.2H), 5.70 (d, J=3.2 Hz, 0.8H), 4.26-4.22 (m, 2H), 3.97-3.87 (m, 3H), 3.61 (s, 3H), 2.94-2.90 (m, 0.2H), 2.86-2.83 (m, 2H), 2.81 (s, 0.2H), 2.77-2.73 (m, 2H), 2.67-2.62 (m, 0.6H), 2.60-2.56 (m, 4H), 2.12-1.97 (m, 2H), 1.05-1.01 (m, 3H).

### Compound 6

### Ethyl 4-(2-bromo-4-fluorophenyl)-6-(2-(3-methoxy-3-oxopropyl)-4,5,6,7-tetra-hydro-2H-indazol-5-yl)-2-(thiazol-2-yl)-1,4-dihydropyrimidine-5-carboxylate

LC-MS (ESI): R_{T}=4.025 min, mass calcd. for C₂₇H₂₇BrFN₅O₄S 615.1, m/z found 618.1 [M+H]⁺. ¹H NMR (400 MHz, DMSO-d₆) δ 9.42 (d, J=3.6 Hz, 0.4H), 9.40 (d, J=3.6 Hz, 0.3H), 8.98 (s, 0.15H), 8.93 (s, 0.15H), 8.00-7.91 (m, 2H), 7.57 (dd, J=8.8, 2.8 Hz, 1H), 7.44-7.31 (m, 2H), 7.29-7.25 (m, 1H), 6.03 (d, J=6.4 Hz, 0.3H), 5.94-5.92 (m, 0.7H), 4.29-4.22 (m, 2H), 4.18-4.10 (m, 0.4H), 3.99-3.86 (m, 2.6H), 3.61 (s, 3H), 2.94-2.90 (m, 0.5H), 2.87-2.83 (m, 2H), 2.79-2.52 (m, 3.5H), 2.27-1.78 (m, 2H), 1.07-0.99 (m, 3H).

A stereoisomeric mixture of ethyl 4-(2-bromo-4-fluorophenyl)-6-(2-(3-methoxy-3-oxopropyl)-4,5,6,7-tetrahydro-2H-indazol-5-yl)-2-(thiazol-2-yl)-1,4-dihydropyrimidine-5-carboxylate Compound 6 (440 mg, 0.715 mmol) was separated by chiral Prep. HPLC (the first separation condition: Column: Chiralpak IG 5 μm 20*250 mm; Mobile Phase: Hex:EtOH:DEA=60:40:0.3 at 15 mL/min; Temp: 30° C.; Wavelength: 214 nm; the second separation condition: Column: Chiralpak AD, 5 μm 20*250 mm; Mobile Phase: Hex:EtOH:DEA=85:10:0.3 at 15 mL/min; Temp: 30° C.; Wavelength: 214 nm; the third separation condition: Column: Chiralpak AD-H, 5 μm 20*250 mm; Mobile Phase: Hex:EtOH:DEA=85:15:0.3 at 15 mL/min; Temp: 30° C.; Wavelength: 214 nm) to afford Compound 6a (64 mg, 14% yield, 100% stereopure), Compound 6b (64 mg, 14% yield, 97.9% stereopure), Compound 6c (65 mg, 15% yield, 100% stereopure) and Compound 6d (68 mg, 15% yield, 100% stereopure) as yellow solids.

Compound 6b: LC-MS (ESI): R_{T}=3.429 min, mass calcd. for C₂₇H₂₇BrFN₅O₄S 615.1, m/z found 615.9 [M+H]⁺. Chiral HPLC (Column: Chiralpak AD-H 5 μm 4.6*250 mm; Mobile Phase: Hex:EtOH:DEA=85:15:0.2 at 1.0 mL/min; Wavelength: 230 nm; R_{T}=13.099 min). ¹H NMR (400 MHz, CD₃OD) δ 7.88-7.85 (m, 1H), 7.73-7.71 (m, 1H), 7.47-7.39 (m, 2.5H), 7.35 (s, 0.5H), 7.16-7.09 (m, 1H), 6.16 (s, 0.4H), 6.09 (s, 0.6H), 4.38-4.34 (m, 2.5H), 4.10-3.98 (m, 2.5H), 3.67 (s, 3H), 3.14-2.98 (m, 1H), 2.91-2.81 (m, 3H), 2.81-2.62 (m, 2H), 2.13-1.89 (m, 2H), 1.10 (t, J=6.8 Hz, 3H).

Compound 6c: LC-MS (ESI): R_{T}=3.463 min, mass calcd. for C₂₇H₂₇BrFN₅O₄S 615.1, m/z found 615.9 [M+H]⁺. Chiral HPLC (Column: Chiralpak IE 5 μm 4.6*250 mm; Mobile Phase: Hex:EtOH:DEA=70:30:0.2 at 1.0 mL/min; Wavelength: 230 nm; R_{T}=14.840 min). ¹H NMR (400 MHz, CD₃OD) δ 7.88 (s, 1H), 7.73-7.71 (m, 1H), 7.47-7.39 (m, 2.5H), 7.31 (s, 0.5H), 7.17-7.10 (m, 1H), 6.15 (s, 0.4H), 6.08 (s, 0.6H), 4.38-4.30 (m, 2.5H), 4.10-3.97 (m, 2.5H), 3.67 (s, 3H), 2.90-2.80 (m, 4H), 2.80-2.57 (m, 2H), 2.25-2.12 (m, 2H), 1.10 (t, J=7.2 Hz, 3H).

### Compound 7

LC-MS (ESI): R_{T}=4.435 min, mass calcd. for C₂₇H₂₇ClFN₅O₄S 571.2, m/z found 572.2 [M+1]⁺. ¹H NMR (400 MHz, DMSO-d₆) δ 9.55-9.52 (m, 0.7H), 9.02 (s, 0.15H), 8.97 (s, 0.15H), 8.00-7.92 (m, 2H), 7.45-7.34 (m, 3H), 7.32-7.23 (m, 1H), 6.12 (s, 0.15H), 6.11 (s, 0.15H), 6.02-6.00 (m, 0.7H), 4.28-4.23 (m, 2H), 4.19-4.11 (m, 0.3H), 4.00-3.91 (m, 2.7H), 3.62 (s, 3H), 2.96-2.83 (m, 3H), 2.80-2.66 (m, 3H), 2.18-1.92 (m, 1.5H), 1.84-1.73 (m, 0.5H), 1.06-0.99 (m, 3H).

A stereoisomeric mixture of ethyl 4-(2-chloro-3-fluorophenyl)-6-(2-(3-methoxy-3-oxopropyl)-4,5,6,7-tetrahydro-2H-indazol-5-yl)-2-(thiazol-2-yl)-1,4-dihydropyrimidine-5-carboxylate Compound 7 (510 mg) was separated by chiral Prep. HPLC (the first separation condition: Column: Chiralpak IA 5 μm 30*250 mm; Mobile Phase: Hex:EtOH:DEA=70:30:0.3 at 25 mL/min; Temp: 30° C.; Wavelength: 214 nm; the second separation condition: Column: Chiralpak IE 5 μm 20*250 mm; Mobile Phase: Hex:EtOH:DEA=70:30:0.3 at 12 mL/min; Temp: 30° C.; Wavelength: 214 nm; the third separation condition: Column: Chiralpak IG 5 μm 20*250 mm; Mobile Phase: CO₂: MeOH=70:30 at 50 g/min; Co-Solvent: MeOH; Col. Temp: 30° C.; Wavelength: 230 nm) to afford Compound 7a (62 mg, 95% purity, 100% stereopure), Compound 7b (76 mg, 96% purity, 99.8% stereopure), Compound 7c (58 mg, 90% purity, 100% stereopure, contained some ethyl ester) and Compound 7d (50 mg, 90% purity, 100% stereopure) as yellow solids.

Compound 7b: Chiral HPLC (Column: Chiralpak IG 5 μm 4.6*250 mm; Mobile Phase: CO₂: MeOH=70:30 at 3.0 g/min; Col. Temp: 40° C.; Wavelength: 230 nm, Back pressure: 100 bar, R_{T}=7.14 min).

Compound 7c: LC-MS (ESI): R_{T}=4.125 min, mass calcd. for C₂₇H₂₇ClFN₅O₄S 571.2, m/z found 571.9 [M+H]⁺. Chiral HPLC (Column: Chiralpak IE 5 μm 4.6*250 mm; Mobile Phase: Hex:EtOH:DEA=70:30:0.2 at 1.0 mL/min; Temp: 30° C.; Wavelength: 230 nm, R_{T}=15.248 min. The other peak (R_{T}=13.795 min) was ethyl ester).

### Compound 8

### Ethyl 4-(3-fluoro-2-methylphenyl)-6-(2-(3-methoxy-3-oxopropyl)-4,5,6,7-tetra hydro-2H-indazol-5-yl)-2-(thiazol-2-yl)-1,4-dihydropyrimidine-5-carboxylate

LC-MS (ESI): R_{T}=2.993 min, mass calcd. for C₂₈H₃₀FN₅O₄S 551.2, m/z found 552.2 [M+H]⁺. ¹H NMR (400 MHz, CDCl₃) δ 8.27 (s, 1H), 7.77 (s, 1H), 7.46 (s, 1H), 7.23 (d, J=8.8 Hz, 1H), 7.11 (s, 2H), 6.95-6.90 (m, 1H), 6.05 (s, 1H), 4.40-4.36 (m, 2.8H), 4.05-3.97 (m, 2.2H), 3.71 (s, 3H), 3.08-2.69 (m, 6H), 2.55 (s, 3H), 2.25-2.01 (m, 2H), 1.10 (t, J=7.2 Hz, 3H).

A stereoisomeric mixture of ethyl 4-(3-fluoro-2-methylphenyl)-6-(2-(3-methoxy-3-oxopropyl)-4,5,6,7-tetrahydro-2H-indazol-5-yl)-2-(thiazol-2-yl)-1,4-dihydropyrimidine-5-carboxylate Compound 8 (500 mg, 0.91 mmol) was separated by chiral prep. HPLC (the first separation condition: Column: Chiralpak ID 5 μm 20 mm*250 mm; Mobile Phase: Hex:EtOH:DEA=70:30:0.3 at 15 mL/min; Wavelength: 214 nm; the second separation condition: Column: Chiralpak IG 5 μm 4.6 mm*250 mm; Mobile Phase: Hex:IPA:DEA=60:40:0.3 at 15 mL/min; Wavelength: 214 nm; the third separation condition: Column: Chiralpak IG 5 μm 4.6 mm*250 mm; Mobile Phase: CO₂: IPA=70:30 at 50 g/min; Wavelength: 214 nm) to give the title compounds Compound 8a (85 mg, 37% yield, 100% stereopure), Compound 8b (90 mg, 39% yield, 100% stereopure), Compound 8c (100 mg, 26% yield, 100% stereopure) and Compound 8d (100 mg, 20% yield, 100% stereopure) as yellow solids.

Compound 8b: LC-MS (ESI): R_{T}=3.826 min, mass calcd. for C₂₈H₃₀FN₅O₄S 551.2, m/z found 552.2 [M+H]⁺. Chiral HPLC (Column: Chiralpak IG 5 μm 4.6*250 mm; Mobile Phase: CO₂:MeOH=70:30 at 3 g/min; Col. Temp: 40° C.; Back pressure:100 bar; Wavelength: 230 nm, R_{T}=6.29 min). ¹H NMR (400 MHz, DMSO-d₆) δ 9.51 (d, J=3.6 Hz, 0.8H), 9.00 (s, 0.2H), 7.99-7.96 (m, 1.6H), 7.94 (d, J=2.8 Hz, 0.2H), 7.90 (d, J=2.8 Hz, 0.2H), 7.46 (s, 0.2H), 7.40 (s, 0.8H), 7.25-7.16 (m, 1.8H), 7.08-7.02 (m, 1.2H), 5.90 (s, 0.2H), 5.77 (d, J=2.8 Hz, 0.8H), 4.28-4.23 (m, 2.2 H), 3.97-3.86 (m, 2.8H), 3.61 (s, 3H), 2.94-2.83 (m, 3H), 2.73-2.66 (m, 2H), 2.50-2.46 (m, 1H), 2.40 (s, 3H), 2.15-1.91 (m, 1.2H), 1.82-1.76 (m, 0.8H), 1.04-1.00 (m, 3H).

Compound 8c: LC-MS (ESI): R_{T}=3.364 min, mass calcd. for C₂₈H₃₀FN₅O₄S 551.2, m/z found 552.2 [M+H]⁺. Chiral HPLC (Column: Chiralpak IG 5 μm 4.6*250 mm; Mobile Phase: Hex:IPA:DEA=70:30:0.2 at 1.0 mL/min; Wavelength: 254 nm, R_{T}=11.869 min). ¹H NMR (400 MHz, DMSO-d₆) δ 9.53 (d, J=3.2 Hz, 0.8H), 8.94 (s, 0.2H), 7.99-7.97 (m, 1.6H), 7.94 (d, J=3.6 Hz, 0.2H), 7.91 (d, J=3.6 Hz, 0.2H), 7.45 (s, 0.2H), 7.37 (s, 0.8H), 7.25-7.19 (m, 1.8H), 7.10-7.02 (m, 1.2H), 5.89 (s, 0.2H), 5.76 (d, J=3.2 Hz, 0.8H), 4.28-4.22 (m, 2.2H), 4.00-3.88 (m, 2.8H), 3.61 (s, 3H), 2.84 (t, J=6.8 Hz, 2H), 2.81-2.55 (m, 4H), 2.41 (s, 3H), 2.26-1.97 (m, 2H), 1.04-1.00 (m, 3H).

### Compound 9

### Ethyl 4-(2-bromo-3-fluorophenyl)-6-(2-(3-methoxy-3-oxopropyl)-4,5,6,7-tetra hydro-2H-indazol-5-yl)-2-(thiazol-2-yl)-1,4-dihydropyrimidine-5-carboxylate

LC-MS (ESI): R_{T}=3.876 min, mass calcd. for C₂₇H₂₇BrFN₅O₄S 615.1, m/z found 616.1 [M+H]⁺. ¹H NMR (400 MHz, DMSO-d₆) δ 9.50 (d, J=3.2 Hz, 0.3H), 9.47 (d, J=3.6 Hz, 0.3H), 9.01 (s, 0.2H), 8.97 (s, 0.2H), 8.00-7.92 (m, 2H), 7.47-7.38 (m, 2H), 7.37-7.20 (m, 2H), 6.11-6.09 (m, 0.3H), 6.01-5.99 (m, 0.7H), 4.28-4.22 (m, 2H), 4.16 (br s, 0.5H), 3.98-3.90 (m, 2.5H), 3.61 (s, 3H), 2.94-2.65 (m, 6H), 2.12-1.83 (m, 2H), 1.05-0.97 (m, 3H).

A stereoisomeric mixture of ethyl 4-(2-bromo-3-fluorophenyl)-6-(2-(3-methoxy-3-oxopropyl)-4,5,6,7-tetrahydro-2H-indazol-5-yl)-2-(thiazol-2-yl)-1,4-dihydro pyrimidine-5-carboxylate Compound 9 (550 mg, 0.890 mmol) was separated by chiral prep. HPLC (the first separation condition: Column: Chiralpak IG 5 μm 20*250 mm; Mobile Phase: Hex:EtOH:DEA=60:40:0.3 at 15 mL/min; Wavelength: 214 nm; the second separation condition: Column: Chiralpak AD 5 μm 20*250 mm; Mobile Phase: Hex:EtOH:DEA=80:20:0.3 at 15 mL/min; Wavelength: 230 nm; the third separation condition: Column: Chiralpak IE 5 μm 20*250 mm; Mobile Phase: Hex:EtOH:DEA=70:30:0.3 at 14 mL/min; Wavelength: 230 nm) to give the title compounds Compound 9a (95 mg, 17% yield, 100% stereopure), Compound 9b (90 mg, 16% yield, 98.2% stereopure), Compound 9c (110 mg, 20% yield, 100% stereopure) and Compound 9d (90 mg, 16% yield, 100% stereopure).

Compound 9b: LC-MS (ESI): R_{T}=4.148 min, mass calcd. for C₂₇H₂₇BrFN₅O₄S 615.1, m/z found 615.9 [M+H]⁺. Chiral HPLC (Column: Chiralpak AD-H 5 μm 4.6*250 mm; Mobile Phase: Hex:EtOH:DEA=80:20:0.2 at 1.0 mL/min; Temp: 30° C.; Wavelength: 230 nm, R_{T}=10.670 min). ¹H NMR (400 MHz, DMSO-d₆) δ 9.48 (d, J=3.6 Hz, 0.6H), 9.02 (s, 0.4H), 7.99-7.92 (m, 2H), 7.47-7.40 (m, 2H), 7.33-7.20 (m, 2H), 6.11 (s, 0.4H), 6.00 (d, J=3.6 Hz, 0.6H), 4.28-4.15 (m, 2.4H), 3.98-3.90 (m, 2.6H), 3.61 (s, 3H), 2.98-2.69 (m, 6H), 2.14-1.79 (m, 2H), 1.05-0.97 (m, 3H).

Compound 9d: LC-MS (ESI): R_{T}=2.758 min, mass calcd. for C₂₇H₂₇BrFN₅O₄S 615.1, m/z found 616.0 [M+H]⁺. Chiral HPLC (Column: Chiralpak IE 5 μm 4.6*250 mm; Mobile Phase: Hex:EtOH:DEA=70:30:0.2 at 1.0 mL/min; Temp: 30° C.; Wavelength: 230 nm, R_{T}=15.451 min). ¹H NMR (400 MHz, DMSO-d₆) δ 9.51 (s, 0.7H), 8.97 (s, 0.3H), 8.00-7.92 (m, 2H), 7.47-7.41 (m, 1.4H), 7.38 (s, 0.6H), 7.32-7.23 (m, 2H), 6.10 (s, 0.3H), 5.99 (s, 0.7H), 4.28-4.18 (m, 2.4H), 3.99-3.92 (m, 2.6H), 3.61 (s, 3H), 2.85 (t, J=6.8 Hz, 2H), 2. 78-2.58 (m, 4H), 2.24-2.03 (m, 2H), 1.05-0.97 (m, 3H).

### Compound 10

### Ethyl 4-(2-chloro-3,4-difluorophenyl)-2-(3,5-difluoropyridin-2-yl)-6-(2-(3-methoxy-3-oxopropyl)-4,5,6,7-tetrahydro-2H-indazol-5-yl)-1,4-dihydropyrimidine-5-carboxylate

LC-MS (ESI): R_{T}=1.56 min, mass calcd. for C₂₉H₂₆ClF₄N₅O₄ 619.2, m/z found 620.2 [M+H]⁺. ¹H NMR (400 MHz, CD₃OD) δ 8.40-8.38 (m, 1H), 7.71-7.67 (m, 1H), 7.47-7.22 (m, 3H), 6.23 (d, J=6.4 Hz, 0.8H), 6.11 (s, 0.2H), 4.41-4.31 (m, 3H), 4.07-4.02 (m, 2H), 3.66 (s, 3H), 2.88-2.66 (m, 6H), 2.29-2.07 (m, 2H), 1.11 (t, J=7.2 Hz, 3H). A stereoisomeric mixture of ethyl 4-(2-chloro-3,4-difluorophenyl)-2-(3,5-difluoropyridin-2-yl)-6-(2-(3-methoxy-3-oxopropyl)-4,5,6,7-tetrahydro-2H-indazol-5-yl)-1,4-dihydropyrimidine-5-carboxylate Compound 10 (490 mg, 0.711 mmol, 90% purity) was separated by chiral Prep. HPLC (the first separation condition: Column: Chiralpak IA 5 μm 30*250 mm; Mobile Phase: Hex:IPA=80:20 at 25 mL/min; Temp: 30° C.; Wavelength: 230 nm; the second separation condition: Chiralpak ID 25*250 mm; Mobile Phase: MeOH=100% at 20 mL/min; Temp: 35° C.; Wavelength: 214 nm; the third separation condition: Chiralpak ID 25*250 mm; Mobile Phase: EtOH=100% at 10 mL/min; Temp: 35° C.; Wavelength: 214 nm) to afford the title compounds Compound 10a (91 mg, 19% yield, 100% stereopure), Compound 10b (82 mg, 17% yield, 100% stereopure), Compound 10c (102 mg, 20% yield, 100% stereopure) and Compound 10d (110 mg, 21% yield, 92% stereopure) as yellow solids.

Compound 10b: LC-MS (ESI): R_{T}=1.54 min, mass calcd. for C₂₉H₂₆ClF₄N₅O₄ 619.2, m/z found 620.3 [M+H]⁺. Chiral HPLC (Column: Chiralpak OD-H 5 μm 4.6*250 mm; Mobile Phase: Hex:EtOH=70:30 at 1.0 mL/min; Temp: 30° C.; Wavelength: 230 nm, R_{T}=8.703 min). ¹H NMR (400 MHz, CD₃OD) δ 8.38 (d, J=2.0 Hz, 1H), 7.71-7.67 (m, 1H), 7.40-7.24 (m, 3H), 6.24 (s, 0.7H), 6.12 (s, 0.3H), 4.38-4.32 (m, 3H), 4.07-4.02 (m, 2H), 3.67 (s, 3H), 3.01-2.96 (m, 1H), 2.88-2.74 (m, 5H), 2.15-2.07 (m, 2H), 1.11 (t, J=7.2 Hz, 3H).

Compound 10c: LC-MS (ESI): R_{T}=1.54 min, mass calcd. for C₂₉H₂₆ClF₄N₅O₄ 619.2, m/z found 620.3 [M+H]⁺. Chiral HPLC (Column: Chiralpak OD 3 μm 4.6*150 mm; Mobile Phase: Hex:EtOH=70:30 at 1.0 mL/min; Temp: 30° C.; Wavelength: 230 nm, R_{T}=4.569 min). ¹H NMR (400 MHz, CD₃OD) δ 8.40 (d, J=2.0 Hz, 1H), 7.72-7.67 (m, 1H), 7.41 (s, 0.8H), 7.32-7.22 (m, 2.2H), 6.23 (s, 0.7H), 6.11 (s, 0.3H), 4.37-4.34 (m, 3H), 4.07-4.02 (m, 2H), 3.67 (s, 3H), 2.91-2.71 (m, 6H), 2.24-2.11 (m, 2H), 1.12 (t, J=7.2 Hz, 3H).

### Compound 11

### Methyl 4-(2-chloro-3-fluorophenyl)-6-(2-(3-methoxy-3-oxopropyl)-4,5,6,7-tetra-hydro-2H-indazol-5-yl)-2-(thiazol-2-yl)-1,4-dihydropyrimidine-5-carboxylate

LC-MS (ESI): R_{T}=2.179 min, mass calcd. for C₂₆H₂₅ClFN₅O₄S 557.1, m/z found 558.2 [M+H]t. ¹H NMR (400 MHz, CDCl₃) δ 8.23 (s, 0.5H), 7.83-7.81 (m, 0.5H), 7.76-7.75 (m, 0.5H), 7.49-7.42 (m, 1.5H), 7.23-7.15 (m, 3H), 7.11-7.02 (m, 1H), 6.28 (d, J=7.2 Hz, 0.5H), 6.15-6.13 (m, 0.5H), 4.41-4.33 (m, 2.5H), 4.15-4.06 (m, 0.5H), 3.71 (s, 3H), 3.60 (s, 2.1H), 3.58 (s, 0.9H), 3.13-2.60 (m, 6H), 2.35-1.90 (m, 2H).

A stereoisomeric mixture of Compound 11 (580 mg, 1.04 mmol) was separated by chiral prep. HPLC (separation condition: Column: Chiralpak IG 5 μm 20*250 mm; Mobile Phase: Hex:EtOH:DEA=70:30:0.3 at 25 mL/min; Wavelength: 214 nm) to give Group 1 (280 mg) and Group 2 (200 mg) as yellow solids. Group 1 was separated by chiral prep. HPLC (separation condition: Column: Chiralpak IG 5 μm 20*250 mm; Mobile Phase: Hex:EtOH:DEA=70:30:0.3 at 15 mL/min; Wavelength: 214 nm) to give the title compounds Compound 11a (55.4 mg, 10% yield, 100% stereopure) and Compound 11b (65.7 mg, 11% yield, 98.9% stereopure). Group 2 was separated by chiral prep. HPLC (separation condition: Column: Chiralpak IA 5 μm 20*250 mm; Mobile Phase: Hex:EtOH:DEA=70:30:0.3 at 20 mL/min; Wavelength: 214 nm) to give the title compounds Compound 11e (70.5 mg, 12% yield, 100% stereopure) and Compound 11d (72.5 mg, 13% yield, 96.5% stereopure).

Compound 11b: LC-MS (ESI): R_{T}=3.984 min, mass calcd. for C₂₆H₂₅ClFN₅O₄S 557.1, m/z found 557.9 [M+H]⁺. Chiral HPLC (Column: Chiralpak IG 5 μm 4.6*250 mm; Mobile Phase: Hex:EtOH:DEA=70:30:0.2 at 1.0 mL/min; Temp: 30° C.; Wavelength: 230 nm, R_{T}=12.893 min). ¹H NMR (400 MHz, DMSO-d₆) δ 9.55 (d, J=3.2 Hz, 0.7H), 9.10 (s, 0.3H), 8.00-7.98 (m, 1.4H), 7.96-7.95 (m, 0.3H), 7.93-7.92 (m, 0.3H), 7.45-7.30 (m, 3H), 7.25-7.21 (m, 1H), 6.11 (s, 0.3H), 6.01-6.00 (d, J=3.6 Hz, 0.7H), 4.28-4.23 (m, 2H), 4.18-4.11 (m, 0.3H), 3.93-3.85 (m, 0.7H), 3.61 (s, 3H), 3.51 (s, 2.1H), 3.49 (s, 0.9H), 2.99-2.92 (m, 1H), 2.90-2.82 (m, 2H), 2.80-2.53 (m, 3H), 2.18-2.10 (m, 0.3H), 2.04-1.93 (m, 1H), 1.82-1.78 (m, 0.7H).

Compound 11c: LC-MS (ESI): R_{T}=4.183 min, mass calcd. for C₂₆H₂₅ClFN₅O₄S 557.1, m/z found 558.1 [M+H]⁺. Chiral HPLC (Column: Chiralpak IA 5 μm 4.6*250 mm; Mobile Phase: Hex:EtOH:DEA=70:30:0.2 at 1.0 mL/min; Temp: 30° C.; Wavelength: 230 nm, R_{T}=8.158 min). ¹H NMR (400 MHz, DMSO-d₆) δ 9.57 (d, J=3.6 Hz, 0.7H), 9.03 (s, 0.3H), 8.00-7.98 (m, 1.4H), 7.96-7.95 (m, 0.3H), 7.93-7.92 (m, 0.3H), 7.45-7.23 (m, 4H), 6.09 (s, 0.3H), 5.99 (d, J=3.6 Hz, 0.7H), 4.28-4.22 (m, 2H), 4.18-4.11 (m, 0.3H), 3.94-3.87 (m, 0.7H), 3.61 (s, 3H), 3.51 (s, 2.1H), 3.49 (s, 0.9H), 2.85 (t, J=6.8 Hz, 2H), 2.78-2.70 (m, 2H), 2.62-2.52 (m, 2H), 2.26-1.96 (m, 2H).

### Compound 12

### Methyl 4-(2-bromo-3-fluorophenyl)-6-(2-(3-methoxy-3-oxopropyl)-4,5,6,7-tetra-hydro-2H-indazol-5-yl)-2-(thiazol-2-yl)-1,4-dihydropyrimidine-5-carboxylate

LC-MS (ESI): R_{T}=3.866 min, mass calcd. for C₂₆H₂₅BrFN₅O₄S 601.1, m/z found 602.1 [M+H]⁺. ¹H NMR (400 MHz, CDCl₃) δ 8.22 (s, 0.4H), 7.82 (s, 0.5H), 7.75 (s, 0.5H), 7.52-7.42 (m, 1.6H), 7.26-7.15 (m, 3H), 7.08-7.00 (m, 1H), 6.27 (d, J=8.8 Hz, 0.5H), 6.12 (s, 0.5H), 4.39-4.35 (m, 2.5H), 4.11 (br s, 0.5H), 3.71 (s, 3H), 3.59 (d, J=4.8 Hz, 3H), 3.14-3.03 (m, 0.5H), 2.97-2.78 (m, 5H), 2.74-2.60 (m, 0.5H), 2.37-1.90 (m, 2H).

A stereoisomeric mixture of Compound 12 (620 mg, 1.03 mmol) was separated by chiral prep. HPLC (the first separation condition: Column: Chiralpak IA 5 μm 20*250 mm; Mobile Phase: EtOH: CO₂=70:30 at 45 g/min; Col. Temp: 40° C.; Wavelength: 230 nm, Back pressure: 100 bar; the second separation condition: Column: Chiralpak IG 5 μm 20*250 mm; Mobile Phase: EtOH: CO₂=40:60 at 50 g/min; Col. Temp: 40° C.; Wavelength: 214 nm, Back pressure: 100 bar; the third separation condition: Column: Chiralpak IG 5 μm 20*250 mm; Mobile Phase: Hex:EtOH:DEA=70:30:0.3 at 15 mL/min; Temp: 30° C.; Wavelength: 214 nm) to afford the title compounds Compound 12a (55 mg, 24% yield, 100% stereopure), Compound 12b (50 mg, 22% yield, 98.7% stereopure), Compound 12c (100 mg, 25% yield, 100% stereopure) and Compound 12d (80 mg, 13% yield, 100% stereopure).

Compound 12b: LC-MS (ESI): R_{T}=4.029 min, mass calcd. for C₂₆H₂₅BrFN₅O₄S 601.1, m/z found 601.8 [M+H]⁺. Chiral HPLC (Column: Chiralpak IG 5 μm 4.6*250 mm; Mobile Phase: Hex:EtOH:DEA=70:30:0.2 at 1.0 mL/min; Temp: 30° C.; Wavelength: 230 nm, R_{T}=13.190 min). ¹H NMR (400 MHz, CDCl₃) δ 8.22 (s, 0.5H), 7.82 (d, J=3.2 Hz, 0.5H), 7.75 (d, J=3.2 Hz, 0.5H), 7.50 (s, 0.5H), 7.47 (d, J=3.2 Hz, 0.5H), 7.42 (d, J=2.8 Hz, 0.5H), 7.30-7.28 (m, 0.3H), 7.24-7.15 (m, 2.7H), 7.08-7.00 (m, 1H), 6.28 (s, 0.5H), 6.12 (d, J=2.8 Hz, 0.5H), 4.37 (t, J=6.4 Hz, 2.5H), 4.13-4.08 (m, 0.5H), 3.71 (s, 3H), 3.60 (s, 1.4H), 3.58 (s, 1.6H), 3.13-3.03 (m, 1H), 2.92-2.71 (m, 5H), 2.21-2.09 (m, 1H), 2.07-1.90 (m, 1H).

Compound 12c: LC-MS (ESI): R_{T}=4.235 min, mass calcd. for C₂₆H₂₅BrFN₅O₄S 601.1, m/z found 602.0 [M+H]⁺. Chiral HPLC (Column: Chiralpak IG 5 μm 4.6*250 mm; Mobile Phase: CO₂:EtOH=40:60 at 3.0 g/min; Temp: 40° C.; Wavelength: 230 nm, Back pressure: 100 bar; R_{T}=5.25 min). ¹H NMR (400 MHz, CDCl₃) δ 8.22 (s, 0.5H), 7.82 (d, J=3.2 Hz, 0.5H), 7.75 (d, J=3.2 Hz, 0.5H), 7.50 (s, 0.5H), 7.47 (d, J=3.2 Hz, 0.5H), 7.42 (d, J=2.8 Hz, 0.5H), 7.30-7.28 (m, 0.4H), 7.25-7.16 (m, 2.6H), 7.08-6.99 (m, 1H), 6.26 (s, 0.5H), 6.11 (d, J=2.8 Hz, 0.5H), 4.43-4.34 (m, 2.5H), 4.19-4.08 (m, 0.5H), 3.71 (s, 3H), 3.60 (s, 1.5H), 3.58 (s, 1.5H), 2.96-2.79 (m, 5H), 2.70-2.60 (m, 1H), 2.37-2.02 (m, 2H).

### Compound 13

### Methyl 4-(2-bromo-3,4-difluorophenyl)-6-(2-(3-methoxy-3-oxopropyl)-4,5,6,7-tetrahydro-2H-indazol-5-yl)-2-(thiazol-2-yl)-1,4-dihydropyrimidine-5-carboxylate

LC-MS (ESI): R_{T}=4.098 min, mass calcd. for C₂₆H₂₄BrF₂N₅O₄S 619.1, m/z found 619.8 [M+H]⁺. ¹H NMR (400 MHz, DMSO-d₆) δ 9.59 (d, J=3.2 Hz, 0.4H), 9.57 (d, J=3.6 Hz, 0.2H), 9.10 (s, 0.2H), 9.05 (s, 0.2H), 8.01-7.92 (m, 2H), 7.55-7.48 (m, 1H), 7.45 (s, 0.3H), 7.39 (s, 0.2H), 7.38 (s, 0.5H), 7.30-7.20 (m, 1H), 6.05 (s, 0.2H), 6.03 (s, 0.2H), 5.96-5.93 (m, 0.6H), 4.28-4.22 (m, 2H), 4.18-4.12 (m, 0.4H), 3.93-3.86 (m, 0.6H), 3.61 (s, 3H), 3.51 (s, 2H), 3.49 (s, 1H), 2.98-2.96 (m, 0.5H), 2.93-2.90 (m, 2H), 2.86-2.71 (m, 2H), 2.65-2.56 (m, 1.5H), 2.12-2.05 (m, 0.7H), 2.02-1.93 (m, 1H), 1.82-1.78 (m, 0.3H).

A stereoisomeric mixture of Compound 13 (300 mg, 0.48 mmol) was separated by chiral Prep. HPLC (the first separation condition: Column: Chiralpak IG 5 μm 20*250 mm; Mobile Phase: Hex:EtOH:DEA=70:30:0.3 at 15 mL/min; Temp: 30° C.; Wavelength: 214 nm; the second separation condition: Column: Chiralpak IG 5 μm 20*250 mm; Mobile Phase: Hex:IPA:DEA=70:30:0.3 at 25 mL/min; Temp: 30° C.; Wavelength: 214 nm) to give the title compounds Compound 13a (50 mg, 16% yield, mixture of two stereoisomers), Compound 13c (80 mg, 26% yield, 100% stereopure) and Compound 13d (100 mg, 33% yield, 100% stereopure) as yellow solids.

Compound 13a (mixture of two stereoisomers): LC-MS (ESI): R_{T}=4.102 min, mass calcd. for C₂₆H₂₄BrF₂N₅O₄S 619.1, m/z found 622.0 [M+H]⁺. Chiral HPLC (Column: Chiralpak IE 5 μm 4.6*250 mm; Mobile Phase: Hex:EtOH:DEA=70:30:0.2 at 1.0 mL/min; Temp: 30° C.; Wavelength: 230 nm, R_{T}=13.222 min). ¹H NMR (400 MHz, DMSO-d₆) δ 9.60 (d, J=3.6 Hz, 0.7H), 9.13 (s, 0.3H), 8.01-7.99 (m, 1.2H), 7.95 (d, J=3.6 Hz, 0.4H), 7.93 (d, J=2.8 Hz, 0.4H), 7.53-7.48 (m, 1H), 7.45 (s, 0.3H), 7.40 (s, 0.7H), 7.26-7.18 (m, 1H), 6.05 (s, 0.3H), 5.95 (d, J=3.6 Hz, 0.7H), 4.27-4.23 (m, 2H), 4.17-4.13 (m, 0.3H), 3.92-3.87 (m, 0.7H), 3.61 (s, 3H), 3.51 (s, 2H), 3.49 (s, 1H), 3.00-2.91 (m, 1H), 2.87-2.83 (m, 2H), 2.79-2.69 (m, 2H), 2.66-2.56 (m, 1H), 2.15-2.11 (m, 0.3H), 2.00-1.95 (m, 1H), 1.83-1.78 (m, 0.7H).

Compound 13d: LC-MS (ESI): R_{T}=2.636 min, mass calcd. for C₂₆H₂₄BrF₂N₅O₄S 619.1, m/z found 622.0 [M+H]⁺. Chiral HPLC (Column: Chiralpak IE 5 μm 4.6*250 mm; Mobile Phase: Hex:EtOH:DEA=70:30:0.2 at 1.0 mL/min; Temp: 30° C.; Wavelength: 230 nm, R_{T}=15.485 min). ¹H NMR (400 MHz, DMSO-d₆) δ 9.61 (d, J=3.2 Hz, 0.7H), 9.07 (s, 0.3H), 8.01-7.99 (m, 1.2H), 7.95 (d, J=2.8 Hz, 0.4H), 7.93 (d, J=3.2 Hz, 0.4H), 7.55-7.49 (m, 1H), 7.45 (s, 0.3H), 7.38 (s, 0.7H), 7.30-7.26 (m, 0.7H), 7.24-7.21 (m, 0.3H), 6.03 (s, 0.3H), 5.94 (d, J=3.6 Hz, 0.7H), 4.28-4.22 (m, 2H), 4.18-4.12 (m, 0.3H), 3.93-3.87 (m, 0.7H), 3.61 (s, 3H), 3.51 (s, 2H), 3.50 (s, 1H), 2.86-2.83 (m, 2H), 2.77-2.71 (m, 3H), 2.68-2.54 (m, 1H), 2.26-2.21 (m, 0.4H), 2.12-2.06 (m, 1H), 2.02-1.97 (m, 0.6H).

### Compound 14

### Methyl 4-(2-chloro-4-fluorophenyl)-6-(2-(3-methoxy-3-oxopropyl)-4,5,6,7-tetrahydro-2H-indazol-5-yl)-2-(thiazol-2-yl)-1,4-dihydropyrimidine-5-carboxylate

LC-MS (ESI): R_{T}=3.834 min, mass calcd. for C₂₆H₂₅ClFN₅O₄S 558.0, m/z found 558.1 [M+H]⁺. ¹H NMR (400 MHz, DMSO-d₆) δ 9.50-9.47 (m, 0.7H), 9.05-9.00 (m, 0.3H), 8.00-7.92 (m, 2H), 7.45-7.35 (m, 3H), 7.26-7.21 (m, 1H), 6.05-6.04 (m, 0.3H), 5.96-5.93 (m, 0.7H), 4.28-4.19 (m, 2H), 4.15-4.09 (m, 0.3H), 3.95-3.84 (m, 0.7H), 3.61 (s, 3H), 3.51-3.50 (m, 3H), 2.97-2.82 (m, 3H), 2.78-2.58 (m, 3H), 2.23-1.92 (m, 1.7H), 1.83-1.76 (m, 0.3H).

A stereoisomeric mixture of Compound 14 (300 mg, 0.54 mmol) was separated by chiral Prep. HPLC (separation condition: Chiralpak IG 5 μm 20*250 mm; Mobile Phase: Hex:IPA:DEA=50:50:0.3 at 12 mL/min; Temp: 30° C.; Wavelength: 214 nm) to afford the title compounds Compound 14a (44 mg, 14% yield, 100% stereopure), Compound 14b (44 mg, 14% yield, 98% stereopure), Compound 14c (45 mg, 15% yield, 100% stereopure) and Compound 14d (45 mg, 15% yield, 100% stereopure) as yellow solids. Compound 14b: LC-MS (ESI): R_{T}=4.006 min, mass calcd. for C₂₆H₂₅ClFN₅O₄S 557.1, m/z found 557.9 [M+H]⁺. Chiral HPLC (Column: Chiralpak IG 5 μm 4.6*250 mm; Mobile Phase: Hex:IPA:DEA=50:50:0.2 at 1.0 mL/min; Temp: 30° C.; Wavelength: 230 nm, R_{T}=7.886 min). ¹H NMR (400 MHz, DMSO-d₆) δ 9.48 (br s, 0.7H), 9.06 (br s, 0.3H), 8.02-7.90 (m, 2H), 7.44-7.36 (m, 3H), 7.26-7.22 (m, 1H), 6.06 (s, 0.3H), 5.95 (br s, 0.7H), 4.29-4.07 (m, 2.4H), 3.93-3.83 (m, 0.6H), 3.61 (s, 3H), 3.51 (s, 3H), 2.99-2.84 (m, 4H), 2.75-2.62 (m, 2H), 2.13-1.89 (m, 1H), 1.84-1.75 (m, 1H).

Compound 14c: LC-MS (ESI): R_{T}=4.003 min, mass calcd. for C₂₆H₂₅ClFN₅O₄S 557.1, m/z found 557.9 [M+H]⁺. Chiral HPLC (Column: Chiralpak IG 5 μm 4.6*250 mm; Mobile Phase: Hex:IPA:DEA=50:50:0.2 at 1.0 mL/min; Temp: 30° C.; Wavelength: 230 nm, R_{T}=9.454 min). ¹H NMR (400 MHz, DMSO-d₆) δ 9.50 (d, J=3.6 Hz, 0.7H), 9.01 (br s, 0.3H), 8.00-7.98 (m, 1.5H), 7.96-7.92 (m, 0.5H), 7.44-7.38 (m, 3H), 7.26-7.20 (m, 1H), 6.03 (s, 0.3H), 5.93 (d, J=3.6 Hz, 0.7H), 4.28-4.11 (m, 2.4H), 3.93-3.86 (m, 0.6H), 3.61 (s, 3H), 3.51 (s, 2H), 3.50 (s, 1H), 2.87-2.83 (m, 2H), 2.78-2.55 (m, 4H), 2.14-1.95 (m, 2H).

### Compound 15

### Methyl 4-(2-bromo-4-fluorophenyl)-6-(2-(3-methoxy-3-oxopropyl)-4,5,6,7-tetrahydro-2H-indazol-5-yl)-2-(thiazol-2-yl)-1,4-dihydropyrimidine-5-carboxylate

LC-MS (ESI): R_{T}=3.261 min, mass calcd. for C₂₆H₂₅BrFN₅O₄S 601.1, m/z found 603.9 [M+H]⁺. Chiral HPLC (Column: Chiralpak IG 5 μm 4.6*250 mm; Mobile Phase: Hex:EtOH:DEA=60:40:0.2 at 1.0 mL/min; Temp: 30° C.; Wavelength: 230 nm, R_{T}=8.224 min, 9.835 min, 11.909 min and 15.349 min). ¹H NMR (400 MHz, CDCl₃) δ 8.21 (s, 0.5H), 7.82 (d, J=4.4 Hz, 0.5H), 7.75 (d, J=4.8 Hz, 0.5H), 7.49-7.47 (m, 1H), 7.42 (d, J=3.2 Hz, 0.5H), 7.38-7.31 (m, 2H), 7.24-7.20 (m, 0.7H), 7.16 (s, 0.3H), 7.04-6.95 (m, 1H), 6.20 (s, 0.2H), 6.18 (s, 0.3H), 6.06-6.04 (m, 0.5H), 4.41-4.34 (m, 2.5H), 4.13-4.04 (m, 0.5H), 3.71 (s, 3H), 3.60 (s, 2H), 3.59 (s, 1H), 3.13-2.59 (m, 6H), 2.37-1.91 (m, 2H).

A stereoisomeric mixture of Compound 15 (650 mg, 1.08 mmol) was separated by chiral Prep. HPLC (separation condition: Column: Chiralpak IG 5 μm 20*250 mm; Mobile Phase: Hex:EtOH:DEA=60:40:0.2 at 15 mL/min; Wavelength: 214 nm) to afford the title compounds Compound 15a (80 mg, 12% yield, 100% stereopure), Compound 15b (80 mg, 12% yield, 100% stereopure), Compound 15c (120 mg, 18% yield, 100% stereopure) and Compound 15d (120 mg, 18% yield, 99.5% stereopure) as yellow solids. Compound 15b: LC-MS (ESI): R_{T}=4.077 min, mass calcd. for C₂₆H₂₅BrFN₅O₄S 601.1, m/z found 604.0 [M+H]⁺. Chiral HPLC (Column: Chiralpak IG 5 μm 4.6*250 mm; Mobile Phase: Hex:EtOH:DEA=60:40:0.2 at 1.0 mL/min; Temp: 30° C.; Wavelength: 230 nm, R_{T}=9.960 min). ¹H NMR (400 MHz, CDCl₃) δ 8.21 (s, 0.5H), 7.82 (d, J=3.2 Hz, 0.5H), 7.75 (d, J=3.2 Hz, 0.5H), 7.47 (d, J=3.2 Hz, 1H), 7.42 (d, J=3.2 Hz, 0.5H), 7.34-7.31 (m, 2H), 7.24 (s, 0.5H), 7.20 (s, 0.5H), 7.04-6.95 (m, 1H), 6.20 (s, 0.5 H), 6.05 (d, J=2.4 Hz, 0.5H), 4.39-4.32 (m, 2.5H), 4.12-4.04 (m, 0.5H), 3.71 (s, 3H), 3.60 (s, 1.5H), 3.59 (s, 1.5H), 3.13-3.01 (m, 1H), 2.92-2.71 (m, 5H), 2.19-1.91 (m, 2H).

Compound 15d: LC-MS (ESI): R_{T}=4.314 min, mass calcd. for C₂₆H₂₅BrFN₅O₄S 601.1, m/z found 604.1 [M+H]⁺. Chiral HPLC (Column: Chiralpak IG 5 μm 4.6*250 mm; Mobile Phase: Hex:EtOH:DEA=60:40:0.2 at 1.0 mL/min; Temp: 30° C.; Wavelength: 230 nm, R_{T}=14.335 min). ¹H NMR (400 MHz, CDCl₃) δ 8.21 (s, 0.5H), 7.82 (d, J=3.2 Hz, 0.5H), 7.75 (d, J=3.2 Hz, 0.5H), 7.47 (d, J=2.8 Hz, 1H), 7.42 (d, J=3.2 Hz, 0.5H), 7.38-7.31 (m, 2H), 7.22 (s, 0.5H), 7.16 (s, 0.5H), 7.04-6.95 (m, 1H), 6.18 (s, 0.5H), 6.04 (d, J=2.4 Hz, 0.5H), 4.41-4.34 (m, 2.5H), 4.13-4.05 (m, 0.5H), 3.71 (s, 3H), 3.60 (s, 1.5H), 3.59 (s, 1.5H), 2.98-2.78 (m, 5H), 2.69-2.59 (m, 1H), 2.37-2.01 (m, 2H).

### Compound 16

### Methyl 4-(2-chloro-3,4-difluorophenyl)-6-(1-(tetrahydro-2H-pyran-2-yl)-4,5,6,7-tetrahydro-1H-indazol-5-yl)-2-(thiazol-2-yl)-1,4-dihydropyrimidine-5-carboxylate and methyl 4-(2-chloro-3,4-difluorophenyl)-6-(2-(tetrahydro-2H-pyran-2-yl)-4,5,6,7-tetrahydro-1H-indazol-5-yl)-2-(thiazol-2-yl)-1,4-dihydropyrimidine-5-carboxylate

LC-MS (ESI): R_{T}=1.86 and 1.88 min, mass calcd. for C₂₇H₂₆ClF₂N₅O₃S 573.1, m/z found 574.1 [M+H]⁺. ¹H NMR (300 MHz, CDCl₃) δ 8.27 (s, 0.4H), 7.96-7.77 (m, 1H), 7.56-7.35 (m, 2.6H), 7.18-7.04 (m, 2H), 6.22 (s, 0.6H), 6.09 (s, 0.4H), 5.31 (s, 1H), 4.37-4.30 (m, 0.6H), 4.17-4.02 (m, 2.4H), 3.66-3.57 (m, 3H), 3.06-2.68 (m, 4H), 2.44-2.16 (m, 2H), 1.73-1.60 (m, 6H).

### Compound 17

### Ethyl 4-(2-chloro-3,4-difluorophenyl)-6-(2-(2-methoxy-2-oxoethyl)-4,5,6,7-tetrahydro-2H-indazol-5-yl)-2-(thiazol-2-yl)-1,4-dihydropyrimidine-5-carboxylate

LC-MS (ESI): R_{T}=3.481 min, mass calcd. for C₂₆H₂₄ClF₂N₅O₄S 575.1, m/z found 576.2 [M+H]⁺. ¹H NMR (400 MHz, CDCl₃) δ 8.23 (s, 0.6H), 7.83 (s, 0.4H), 7.78 (d, J=3.2 Hz, 0.6H), 7.49 (s, 0.4H), 7.44 (d, J=3.2 Hz, 0.6H), 7.35 (s, 0.4H), 7.25-7.00 (m, 3H), 6.23 (d, J=6.4 Hz, 0.6H), 6.11 (s, 0.4H), 4.92-4.81 (m, 2H), 4.45-4.34 (m, 0.5H), 4.15-3.97 (m, 2.5H), 3.79 (s, 1.8H), 3.78 (s, 1.2H), 3.19-2.63 (m, 4H), 2.35-1.92 (m, 2H), 1.12 (t, J=7.6 Hz, 3H).

A stereoisomeric mixture of Compound 17 (200 mg, 0.34 mmol) was separated by chiral prep. HPLC (separation condition: Column: Chiralpak IG 5 μm 20*250 mm; Mobile Phase: Hex:EtOH:DEA=70:30:0.2 at 15 mL/min; Temp: 30° C.; Wavelength: 214 nm) to afford the title compounds Compound 17a (40.0 mg, 20% yield, 100% stereopure), Compound 17b (40.0 mg, 20% yield, 100% stereopure), Compound 17c (40.0 mg, 20% yield, 100% stereopure) and Compound 17d (40.0 mg, 20% yield, 100% stereopure) as yellow solids (contained ˜25% ethyl ester likely due to trans-esterification in reaction medium EtOH.

Compound 17b: LC-MS (ESI): R_{T}=2.591 min, mass calcd. for C₂₆H₂₄ClF₂N₅O₄S 575.1, m/z found 576.1 [M+H]⁺. Chiral HPLC (Column: Chiralpak IG 5 μm 4.6*250 mm; Mobile Phase: Hex:EtOH:DEA=70:30:0.2 at 1.0 mL/min; Temp: 30° C.; Wavelength: 230 nm, R_{T}=10.660 min). ¹H NMR (400 MHz, CDCl₃) δ 8.23 (s, 0.6H), 7.83 (d, J=3.2 Hz, 0.4H), 7.78 (d, J=2.8 Hz, 0.6H), 7.49 (d, J=2.8 Hz, 0.4H), 7.44 (d, J=2.4 Hz, 0.6H), 7.35 (s, 0.4H), 7.26 (s, 0.5H), 7.22 (s, 0.5H), 7.15-7.00 (m, 2H), 6.24 (s, 0.6H), 6.11 (s, 0.4H), 4.92-4.80 (m, 2H), 4.43-4.34 (m, 0.6H), 4.28-4.22 (m, 0.4H), 4.15-3.97 (m, 2.7H), 3.79 (s, 2.3H), 3.20-3.06 (m, 1H), 2.97-2.73 (m, 3H), 2.21-2.12 (m, 1H), 2.11-1.92 (m, 1H), 1.12 (t, J=7.2 Hz, 3H).

Compound 17c: LC-MS (ESI): R_{T}=2.591 min, mass calcd. for C₂₆H₂₄ClF₂N₅O₄S 575.1, m/z found 576.1 [M+H]⁺. Chiral HPLC (Column: Chiralpak IG 5 μm 4.6*250 mm; Mobile Phase: Hex:EtOH:DEA=70:30:0.2 at 1.0 mL/min; Temp: 30° C.; Wavelength: 230 nm, R_{T}=13.171 min). ¹H NMR (400 MHz, CDCl₃) δ 8.23 (s, 0.6H), 7.83 (d, J=3.2 Hz, 0.4H), 7.78 (d, J=3.2 Hz, 0.6H), 7.49 (d, J=2.8 Hz, 0.4H), 7.44 (d, J=2.8 Hz, 0.6H), 7.35 (s, 0.4H), 7.24 (s, 0.6H), 7.18-7.00 (m, 2.4H), 6.22 (s, 0.6H), 6.11 (d, J=2.4 Hz, 0.4H), 4.91-4.79 (m, 2H), 4.44-4.37 (m, 0.6H), 4.28-4.22 (m, 0.4H), 4.15-3.97 (m, 2.7H), 3.79 (s, 2.3H), 3.00-2.80 (m, 3H), 2.74-2.62 (m, 1H), 2.39-2.24 (m, 1H), 2.18-2.02 (m, 1H), 1.12 (t, J=6.8 Hz, 3H).

### Compound 18

### Ethyl 4-(2-chloro-3,4-difluorophenyl)-6-(2-(4-methoxy-4-oxobutyl)-4,5,6,7-tetrahydro-2H-indazol-5-yl)-2-(thiazol-2-yl)-1,4-dihydropyrimidine-5-carboxylate

LC-MS (ESI): R_{T}=3.954 min, mass calcd. for C₂₈H₂₈ClF₂N₅O₄S 603.2, m/z found 603.9 [M+H]⁺. ¹H NMR (400 MHz, DMSO-d₆) δ 9.59-9.56 (m, 0.6H), 9.02 (s, 0.2H), 8.96 (s, 0.2H), 8.00-7.98 (m, 1.3H), 7.95-7.92 (m, 0.7H), 7.52-7.45 (m, 1.3H), 7.38 (d, J=7.2 Hz, 0.7H), 7.30-7.22 (m, 1H), 6.07 (d, J=4.8 Hz, 0.3H), 5.97-5.95 (m, 0.7H), 4.20-4.13 (m, 0.4H), 4.06-3.88 (m, 4.6H), 3.59 (s, 3H), 2.98-2.54 (m, 4H), 2.31-2.28 (m, 2H), 2.15-1.93 (m, 3.7H), 1.83-1.79 (m, 0.3H), 1.06-0.99 (m, 3H).

A stereoisomeric mixture of Compound 18 (500 mg, 0.83 mmol) was separated by chiral Prep. HPLC (the first separation condition: Column: Chiralpak IG 5 μm 20*250 mm; Mobile Phase: CO₂:EtOH=70:30 at 50 g/min; Co-solvent: EtOH (0.2° C.); Col. Temp 41.1° C.; Wavelength: 214 nm; Back pressure: 100 bar; the second separation condition: Column: Chiralpak AD-H 5 μm 20*250 mm; Mobile Phase: Hex:EtOH:DEA=70:30:0.5 at 15 mL/min; Temp: 30° C.; Wavelength: 230 nm; the third separation condition: Column: Chiralpak IA 5 μm 20*250 mm; Mobile Phase: CO₂:IPA=70:30 at 50 g/min; Co-solvent: IPA (0.2° C.); Col. Temp 41.1° C.; Wavelength: 214 nm; Back pressure: 100 bar) to afford the title compounds Compound 18a (60 mg, 12% yield, 100% stereopure), Compound 18b (55 mg, 11% yield, 96.5% stereopure), Compound 18c (80 mg, 16% yield, 100% stereopure) and Compound 18d (75 mg, 15% yield, 100% stereopure).

Compound 18b: LC-MS (ESI): R_{T}=4.041 min, mass calcd. for C₂₈H₂₈ClF₂N₅O₄S 603.2, m/z found 604.1 [M+H]⁺. Chiral HPLC (Column: Chiralpak AD-H 5 μm 4.6*250 mm; Mobile Phase: Hex:EtOH:DEA=80:20:0.2 at 1.0 mL/min; Temp: 30° C.; Wavelength: 230 nm; R_{T}=9.737 min). ¹H NMR (400 MHz, DMSO-d₆) δ 9.58 (d, J=3.6 Hz, 0.6H), 9.03 (s, 0.4H), 8.00-7.92 (m, 2H), 7.52-7.45 (m, 1.4H), 7.39 (s, 0.6H), 7.27-7.22 (m, 1H), 6.07 (s, 0.3H), 5.97 (d, J=3.2 Hz, 0.7H), 4.17-4.15 (m, 0.3H), 4.06-3.91 (m, 4.7H), 3.59 (s, 3H), 2.99-2.57 (m, 4H), 2.32-2.28 (m, 2H), 2.14-2.09 (m, 0.3H), 2.02-1.94 (m, 3H), 1.83-1.79 (m, 0.7H), 1.06-0.99 (m, 3H).

Compound 18c: LC-MS (ESI): R_{T}=4.062 min, mass calcd. for C₂₈H₂₈ClF₂N₅O₄S 603.2, m/z found 604.1 [M+H]⁺. SFC (analytical condition: Column: Chiralpak IA 5 μm 4.6*250 mm; Mobile Phase: CO₂:MeOH=70:30 at 3.0 g/min; Col. Temp: 40° C.; Wavelength: 230 nm, R_{T}=4.38 min). ¹H NMR (400 MHz, DMSO-d₆) δ 9.60 (d, J=3.2 Hz, 0.7H), 8.97 (s, 0.3H), 8.01-7.92 (m, 2H), 7.52-7.45 (m, 1.3H), 7.37 (s, 0.7H), 7.29-7.24 (m, 1H), 6.06 (s, 0.3H), 5.96 (d, J=3.2 Hz, 0.7H), 4.19-4.17 (m, 0.3H), 4.06-3.90 (m, 4.7H), 3.59 (s, 3H), 2.79-2.51 (m, 4H), 2.31-2.28 (m, 2H), 2.12-1.97 (m, 4H), 1.06-0.99 (m, 3H).

### Compound 19

### Ethyl 4-(2-chloro-3,4-difluorophenyl)-6-(2-(4-methoxy-4-oxobutan-2-yl)-4,5,6,7-tetrahydro-2H-indazol-5-yl)-2-(thiazol-2-yl)-1,4-dihydropyrimidine-5-carboxylate

Converted from KT8

LC-MS (ESI): R_{T}=4.294 min, mass calcd. for C₂₈H₂₈ClF₂N₅O₄S 603.2, m/z found 604.2 [M+H]⁺. ¹H NMR (400 MHz, DMSO-d₆) δ 9.61-9.57 (m, 0.7H), 9.06-8.98 (m, 0.3H), 8.01-7.92 (m, 2H), 7.52-7.43 (m, 2H), 7.30-7.21 (m, 1H), 6.07-6.06 (m, 0.3H), 5.97-5.95 (m, 0.7H), 4.69-4.59 (m, 1H), 4.20 (br s, 0.4H), 3.98-3.88 (m, 2.6H), 3.58 (s, 3H), 2.94-2.54 (m, 6H), 2.24-1.78 (m, 2H), 1.41-1.38 (m, 3H), 1.06-0.98 (m, 3H).

A stereoisomeric mixture of ethyl 4-(2-chloro-3,4-difluorophenyl)-6-(2-(4-methoxy-4-oxobutan-2-yl)-4,5,6,7-tetrahydro-2H-indazol-5-yl)-2-(thiazol-2-yl)-1,4-dihydropyrimidine-5-carboxylate Compound 19 (850 mg, 1.41 mmol) was separated by chiral prep. HPLC (separation condition: column: Chiralpak IG 5 μm 20*250 mm; Mobile Phase: Hex:EtOH:DEA=60:40:0.3 at 15 mL/min, Temp: 30° C., Wavelength: 230 nm) to afford the title compounds Compound 19a (184 mg, 21% yield, 100% stereopure), Compound 19b (186 mg, 22% yield, 100% stereopure), Compound 19c (185 mg, 21% yield, 100% stereopure) and Compound 19d (186 mg, 22% yield, 99% stereopure) as yellow solids.

Compound 19b: LC-MS (ESI): R_{T}=4.287 min, mass calcd. for C₂₈H₂₈ClF₂N₅O₄S 603.2, m/z found 603.9 [M+H]⁺. Chiral HPLC (Column: Chiralpak IG 5 μm 4.6*250 mm; Mobile Phase: Hex:EtOH:DEA=60:40:0.2 at 1.0 mL/min; Temp: 30° C.; Wavelength: 230 nm, R_{T}=6.965 min). ¹H NMR (400 MHz, DMSO-d₆) δ 9.59 (d, J=3.2 Hz, 0.7H), 9.04 (s, 0.3H), 8.00-7.92 (m, 2H), 7.52-7.44 (m, 2H), 7.27-7.21 (m, 1H), 6.07 (s, 0.3H), 5.96 (d, J=3.6 Hz, 0.7H), 4.72-4.61 (m, 1H), 4.20-4.09 (m, 0.3H), 3.98-3.87 (m, 2.7H), 3.58 (s, 3H), 2.98-2.87 (m, 2H), 2.83-2.56 (m, 4H), 2.18-1.78 (m, 2H), 1.40 (d, J=7.2 Hz, 3H), 1.06-0.98 (m, 3H).

Compound 19c: LC-MS (ESI): R_{T}=4.602 min, mass calcd. for C₂₈H₂₈ClF₂N₅O₄S 603.2, m/z found 604.2 [M+H]⁺. Chiral HPLC (Column: Chiralpak IG 5 μm 4.6*250 mm; Mobile Phase: Hex:EtOH:DEA=60:40:0.2 at 1.0 mL/min; Temp: 30° C.; Wavelength: 230 nm, R_{T}=9.310 min). ¹H NMR (400 MHz, DMSO-d₆) δ 9.60 (d, J=3.6 Hz, 0.7H), 9.01 (s, 0.3H), 8.01-7.92 (m, 2H), 7.52-7.43 (m, 2H), 7.29-7.24 (m, 1H), 6.06 (s, 0.3H), 5.95 (d, J=3.6 Hz, 0.7H), 4.71-4.59 (m, 1H), 4.21-4.13 (m, 0.3H), 4.00-3.88 (m, 2.7H), 3.58 (s, 3H), 2.95-2.89 (m, 1H), 2.82-2.52 (m, 5H), 2.26-1.95 (m, 2H), 1.41-1.38 (m, 3H), 1.06-0.99 (m, 3H).

### Compound 20

### Ethyl 4-(2-chloro-3,4-difluorophenyl)-6-(2-(4-methoxy-4-oxobutan-2-yl)-4,5,6,7-tetrahydro-2H-indazol-5-yl)-2-(thiazol-2-yl)-1,4-dihydropyrimidine-5-carboxylate

Converted from KT9

LC-MS (ESI): R_{T}=3.909 min, mass calcd. for C₂₈H₂₈ClF₂N₅O₄S 603.2, m/z found 604.2 [M+H]⁺. ¹H NMR (400 MHz, DMSO-d₆) δ 9.61-9.57 (m, 0.6H), 9.07-8.98 (m, 0.3H), 8.01-7.92 (m, 2H), 7.52-7.42 (m, 2H), 7.29-7.21 (m, 1H), 6.07-6.06 (m, 0.3H), 5.97-5.95 (m, 0.7H), 4.70-4.61 (m, 1H), 4.20 (br s, 0.3H), 4.02-3.87 (m, 2.7H), 3.58 (s, 3H), 2.94-2.57 (m, 6H), 2.23-1.78 (m, 2H), 1.41-1.38 (m, 3H), 1.06-0.98 (m, 3H).

A stereoisomeric mixture of (S*)-ethyl 4-(2-chloro-3,4-difluorophenyl)-6-(2-(4-methoxy-4-oxobutan-2-yl)-4,5,6,7-tetrahydro-2H-indazol-5-yl)-2-(thiazol-2-yl)-1,4-dihydropyrimidine-5-carboxylate Compound 20 (850 mg, 1.41 mmol) was separated by chiral prep. HPLC (Column: Chiralpak IG 5 μm 20*250 mm; Mobile Phase: Hex:EtOH:DEA=70:30:0.3 at 15 mL/min; Wavelength: 214 nm; Column: Chiralpak AD 5 μm 20*250 mm; Hex:EtOH:DEA=80:20:0.3 at 15 mL/min; Wavelength: 214 nm; Column: Chiralpak IE 5 μm 20*250 mm; Hex:EtOH:DEA=70:30:0.3 at 14 mL/min; Wavelength: 214 nm) to afford the title compounds Compound 20a (146 mg, 17% yield, 100% stereopure), Compound 20b (146 mg, 17% yield, 96% stereopure), Compound 20c (146 mg, 17% yield, 100% stereopure) and Compound 20d (146 mg, 17% yield, 100% stereopure) as yellow solids.

Compound 20b: LC-MS (ESI): R_{T}=4.288 min, mass calcd. for C₂₈H₂₈ClF₂N₅O₄S 603.2, m/z found 603.9 [M+H]⁺. Chiral HPLC (Column: Chiralpak AD-H 5 μm 4.6*250 mm; Mobile Phase: Hex:EtOH:DEA=80:20:0.2 at 1.0 mL/min; Temp: 30° C.; Wavelength: 230 nm, R_{T}=8.669 min). ¹H NMR (400 MHz, DMSO-d₆) δ 9.59 (d, J=3.2 Hz, 0.7H), 9.07 (s, 0.3H), 8.00-7.92 (m, 2H), 7.52-7.44 (m, 2H), 7.26-7.21 (m, 1H), 6.07 (s, 0.3H), 5.97 (d, J=3.2 Hz, 0.7H), 4.69-4.60 (m, 1H), 4.19-4.11 (m, 0.3H), 3.98-3.87 (m, 2.7H), 3.58 (s, 3H), 2.98-2.86 (m, 2H), 2.83-2.52 (m, 4H), 2.16-1.77 (m, 2H), 1.40-1.39 (m, 3H), 1.06-0.98 (m, 3H).

Compound 20d: LC-MS (ESI): R_{T}=4.292 min, mass calcd. for C₂₈H₂₈ClF₂N₅O₄S 603.2, m/z found 603.9 [M+H]⁺. Chiral HPLC (Column: Chiralpak IE 5 μm 4.6*250 mm; Mobile Phase: Hex:EtOH:DEA=70:30:0.2 at 1.0 mL/min; Temp: 30° C.; Wavelength: 230 nm, R_{T}=11.608 min). ¹H NMR (400 MHz, DMSO-d₆) δ 9.61 (d, J=3.6 Hz, 0.7H), 8.99 (s, 0.3H), 8.01-7.92 (m, 2H), 7.52-7.42 (m, 2H), 7.29-7.24 (m, 1H), 6.06 (s, 0.3H), 5.95 (d, J=3.2 Hz, 0.7H), 4.70-4.60 (m, 1H), 4.22-4.14 (m, 0.3H), 4.00-3.88 (m, 2.7H), 3.58 (s, 3H), 2.94-2.88 (m, 1H), 2.83-2.54 (m, 5H), 2.26-1.95 (m, 2H), 1.39 (d, J=7.2 Hz, 3H), 1.06-0.99 (m, 3H).

### Compound 21

### Ethyl 4-(2-chloro-3,4-difluorophenyl)-6-(2-(3-methoxy-2,2-dimethyl-3-oxopropyl)-4,5,6,7-tetrahydro-2H-indazol-5-yl)-2-(thiazol-2-yl)-1,4-dihydropyrimidine-5-carboxylate

LC-MS (ESI): R_{T}=3.249 min, mass calcd. for C₂₉H₃₀ClF₂N₅O₄S 617.2, m/z found 618.0 [M+H]⁺. ¹H NMR (400 MHz, CDCl₃) δ 8.22 (s, 0.6H), 7.82 (s, 0.4H), 7.76 (d, J=2.8 Hz, 0.6H), 7.49 (d, J=3.2 Hz, 0.4H), 7.42 (d, J=3.2 Hz, 0.6H), 7.34 (s, 0.4H), 7.15-7.01 (m, 3H), 6.23 (d, J=4.8 Hz, 0.6H), 6.11 (d, J=2.4 Hz, 0.4H), 4.41-4.36 (m, 0.6H), 4.26 (s, 2H), 4.10-4.01 (m, 2.4H), 3.73 (s, 3H), 3.11-2.76 (m, 4H), 2.31-2.13 (m, 2H), 1.23 (s, 6H), 1.11 (t, J=6.8 Hz, 3H).

A stereoisomeric mixture of Compound 21 (370 mg, 0.60 mmol) was separated by chiral Prep. HPLC (the first separation condition: Column: Chiralpak IA 5 μm 20*250 mm; Mobile Phase: Hex:EtOH:DEA=80:20:0.3 at 25 mL/min; Temp: 30° C.; Wavelength: 214 nm; the second separation condition: Column: Chiralpak IF 5 μm 20*250 mm; Mobile Phase: Hex:EtOH:DEA=80:20:0.3 at 15 mL/min; Temp: 30° C.; Wavelength: 214 nm; the third separation condition: Chiralpak AD-H 5 μm 20*250 mm; Mobile Phase: Hex:EtOH:DEA=95:5:0.3 at 15 mL/min; Temp: 30° C.; Wavelength: 214 nm) to afford the title compounds Compound 21a (45 mg, 38% yield, 100% stereopure), Compound 21b (45 mg, 38% yield, 94.3% stereopure), Compound 21c (60 mg, 24% yield, 98.0% stereopure) and Compound 21d (60 mg, 31% yield, 100% stereopure).

Compound 21b: LC-MS (ESI): R_{T}=2.114 min, mass calcd. for C₂₉H₃₀ClF₂N₅O₄S 617.2, m/z found 618.1 [M+H]⁺. Chiral HPLC (Column: Chiralpak AD-H 5 μm 4.6*250 mm; Mobile Phase: Hex:EtOH:DEA=95:5:0.2 at 1.0 mL/min; Temp: 30° C.; Wavelength: 230 nm; R_{T}=15.008 min). ¹H NMR (400 MHz, DMSO-d₆) δ 9.58 (d, J=2.4 Hz, 0.6H), 9.07 (s, 0.4H), 7.99-7.93 (m, 1.7H), 7.73-7.67 (m, 0.3H), 7.51-7.45 (m, 1H) 7.35-7.22 (m, 2H), 6.07 (s, 0.3H), 5.96 (d, J=2.4 Hz, 0.7H), 4.15 (br s, 2.3H), 3.97-3.93 (m, 2.7H), 3.63 (s, 3H), 2.98-2.90 (m, 1.5H), 2.72-2.67 (m, 2H), 2.59-2.52 (m, 0.5H), 2.20-2.10 (m, 0.5H), 1.94-1.84 (m, 1H), 1.84-1.79 (m, 0.5H), 1.11 (s, 6H), 1.05-0.99 (m, 3H).

Compound 21c: LC-MS (ESI): R_{T}=2.103 min, mass calcd. for C₂₉H₃₀ClF₂N₅O₄S 617.2, m/z found 618.1 [M+H]⁺. Chiral HPLC (Column: Chiralpak IA 5 μm 4.6*250 mm; Mobile Phase: Hex:EtOH:DEA=80:20:0.2 at 1.0 mL/min; Temp: 30° C.; Wavelength: 230 nm; R_{T}=7.122 min). ¹H NMR (400 MHz, DMSO-d₆) δ 9.61 (d, J=3.6 Hz, 0.6H), 9.01 (s, 0.4H), 8.00 (q, J=3.2 Hz, 1.2H), 7.93 (q, J=2.8 Hz, 0.8H), 7.52-7.46 (m, 1H) 7.35 (s, 0.4H), 7.29-7.23 (m, 1.6H), 6.06 (s, 0.3H), 5.95 (d, J=3.6 Hz, 0.7H), 4.17-4.14 (m, 2.2H), 3.98-3.91 (m, 2.8H), 3.63 (s, 3H), 2.82-2.71 (m, 2H), 2.58-2.51 (m, 2H), 2.26-2.20 (m, 0.4H), 2.11-1.98 (m, 1.6H), 1.11 (s, 6H), 1.06-1.01 (m, 3H).

### Compound 22

### Ethyl 4-(2-chloro-3,4-difluorophenyl)-6-(2-((trans)-3-(methoxycarbonyl)cyclobutyl)-4,5,6,7-tetrahydro-2H-indazol-5-yl)-2-(thiazol-2-yl)-1,4-dihydropyrimidine-5-carboxylate

LC-MS (ESI): R_{T}=3.883 min, mass calcd. for C₂₉H₂₈ClF₂N₅O₄S 615.1, m/z found 616.2 [M+H]⁺. ¹H NMR (400 MHz, DMSO-d₆) δ 9.62-9.59 (m, 0.6H), 9.02 (s, 0.2H), 8.98 (s, 0.2H), 8.01-7.98 (m, 1.3H), 7.95-7.92 (m, 0.7H), 7.57-7.56 (d, J=3.6 Hz, 0.3H), 7.51-7.46 (m, 1.7H), 7.29-7.22 (m, 1H), 6.06 (d, J=6.4 Hz, 0.3H), 5.97-5.95 (m, 0.7H), 4.95-4.85 (m, 1H), 4.16 (s, 0.3H), 3.99-3.91 (m, 2.7H), 3.67 (s, 3H), 3.20-3.13 (m, 1H), 2.98-2.65 (m, 5H), 2.61-2.55 (m, 3H), 2.16-1.80 (m, 2H), 1.06-0.98 (m, 3H).

### Compound 23

### 4-(2-Chloro-3,4-difluoro-phenyl)-6-[2-(2-methoxycarbonyl-ethyl)-4,5,6,7-tetrahydro-2H-indazol-5-yl]-2-thiazol-2-yl-1,4-dihydro-pyrimidine-5-carboxylic acid ethyl ester

LC-MS (ESI): R_{T}=1.76 min and 1.78 min, mass calcd. for C₂₉H₃₀ClF₂N₅O₄S 617.2, m/z found 618.3 [M+H]⁺. ¹H NMR (400 MHz, DMSO-d₆) δ 9.58-9.57 (m, 0.7H), 9.16 (d, J=8.4 Hz, 0.3H), 8.00-7.93 (m, 2H), 7.75-7.66 (m, 0.3H), 7.55-7.45 (m, 1H), 7.37-7.21 (m, 1.7H), 6.06 (d, J=4.0 Hz, 0.3H), 5.97-5.95 (m, 0.7H), 4.29-4.18 (m, 2.8H), 4.03-3.89 (m, 2.2H), 3.61-3.60 (m, 3H), 2.98-2.88 (m, 0.4H), 2.84-2.73 (m, 2.6H), 2.64-2.58 (m, 0.4H), 2.43-2.39 (m, 0.6H), 2.11-1.91 (m, 1H), 1.70-1.61 (m, 0.6H), 1.50-1.44 (m, 0.4H), 1.33-1.18 (m, 6H), 1.06-0.92 (m, 4H).

A stereoisomeric mixture of 4-(2-chloro-3,4-difluoro-phenyl)-6-[2-(2-methoxycarbonyl-ethyl)-4,5,6,7-tetrahydro-2H-indazol-5-yl]-2-thiazol-2-yl-1,4-dihydro-pyrimidine-5-carboxylic acid ethyl ester Compound 23 (530 mg, 0.86 mmol) was separated by prep. HPLC (Column: NX-C18 5 μm 19 mm*150 mm; Flow rate: 15 ml/min, Mobile Phase A: Water (0.1% ammonium bicarbonate), Mobile Phase B: Acetonitrile, Gradient: 10-70% (% B); Wavelength: 214 nm) followed by chiral prep. HPLC (Column: Chiralpak IA 5 μm 20*250 mm; Mobile Phase: CO₂:EtOH=75:25 at 50 g/min; Co-solvent: EtOH; Col. Temp: 40° C.; Wavelength: 230 nm, Back pressure: 100 bar) to give the title compounds Compound 23a (170 mg, 32% yield, mixture of two stereoisomers), Compound 23c (100 mg, 19% yield, 100% stereopure) and Compound 23d (90 mg, 17% yield, 100% stereopure) as yellow solids.

Compound 23a: LC-MS (ESI): R_{T}=3.816 min, mass calcd. for C₂₉H₃₀ClF₂N₅O₄S 617.2, m/z found 618.1 [M+H]⁺. ¹H NMR (400 MHz, DMSO-d₆) δ 9.57 (d, J=3.2 Hz, 0.7H), 9.16 (s, 0.3H), 8.00-7.93 (m, 2H), 7.52-7.46 (m, 1H), 7.36 (s, 0.3H), 7.33 (s, 0.7H), 7.27-7.17 (m, 1H), 6.06 (s, 0.3H), 5.96 (d, J=3.6 Hz, 0.7H), 4.51-4.35 (m, 0.6H), 4.29-4.20 (m, 2.4H), 4.00-3.92 (m, 2H), 3.62 (s, 3H), 2.98-2.89 (m, 1H), 2.84-2.73 (m, 2.3H), 2.65-2.60 (m, 0.7H), 2.14-2.08 (m, 0.4H), 1.97-1.91 (m, 0.6H), 1.64-1.61 (m, 0.3H), 1.51-1.44 (m, 0.7H), 1.40-1.21 (m, 6H), 1.06-0.98 (m, 3H).

Compound 23c: LC-MS (ESI): R_{T}=2.249 min, mass calcd. for C₂₉H₃₀ClF₂N₅O₄S 617.2, m/z found 618.1 [M+H]⁺. Chiral HPLC (Column: Chiralpak IA 5 μm 4.6*250 mm; Mobile Phase: EtOH: CO₂=75:25 at 3.0 g/min; Wavelength: 230 nm, Back pressure; R_{T}=3.18 min). ¹H NMR (400 MHz, DMSO-d₆) δ 9.60 (s, 0.7H), 9.19 (s, 0.3H), 8.00-7.94 (m, 2H), 7.55-7.48 (m, 1H), 7.35-7.25 (m, 2H), 6.07 (s, 0.3H), 5.97 (s, 0.7H), 4.47 (s, 0.4H), 4.27-4.18 (m, 2.6H), 4.03-3.93 (m, 2H), 3.61 (s, 3H), 2.84-2.75 (m, 2.7H), 2.68-2.61 (m, 0.3H), 2.45-2.42 (m, 0.5H), 2.33-2.24 (m, 0.5H), 2.08-2.02 (m, 0.8H), 1.68-1.65 (m, 1.2H), 1.32-1.24 (m, 6H), 1.07-1.03 (m, 3H).

### Compound 24

### Ethyl 4-(2-chloro-3,4-difluorophenyl)-6-(2-(3-methoxy-3-oxopropyl)-3-methyl-4,5,6,7-tetrahydro-2H-indazol-5-yl)-2-(thiazol-2-yl)-1,4-dihydropyrimidine-5-carboxylate

LC-MS (ESI): R_{T}=4.208 min, mass calcd. for C₂₈H₂₈ClF₂N₅O₄S 603.2, m/z found 603.9 [M+H]⁺. ¹H NMR (400 MHz, CDCl₃) δ 8.25 (s, 0.3H), 8.21 (s, 0.3H), 7.82 (dd, J=2.8, 0.8 Hz, 0.4H), 7.76 (d, J=3.6 Hz, 0.6H), 7.49-7.48 (m, 0.4H), 7.43 (d, J=2.8 Hz, 0.6H), 7.37-7.34 (m, 0.4H), 7.19-7.00 (m, 2H), 6.24 (s, 0.3H), 6.22 (s, 0.3H), 6.12-6.10 (m, 0.4H), 4.44-4.33 (m, 0.6H), 4.30-4.25 (m, 2H), 4.11-3.97 (m, 2.4H), 3.70 (s, 3H), 3.04-2.46 (m, 6H), 2.23 (s, 1.5H), 2.21 (s, 1.3H), 2.18 (s, 0.5H), 2.16-1.90 (m, 1.7H), 1.14-1.10 (m, 3H).

### Compound 25

### Mixture of ethyl 4-(2-chloro-3,4-difluorophenyl)-6-(1-(tetrahydro-2H-pyran-2-yl)-4,5,6,7-tetrahydro-1H-indazol-5-yl)-2-(thiazol-2-yl)-1,4-dihydropyrimidine-5-carboxylate and ethyl 4-(2-chloro-3,4-difluorophenyl)-6-(2-(tetrahydro-2H-pyran-2-yl)-4,5,6,7-tetrahydro-1H-indazol-5-yl)-2-(thiazol-2-yl)-1,4-dihydropyrimidine-5-carboxylate

LC-MS (ESI): R_{T}=2.009 min, mass calcd. for C₂₈H₂₈ClF₂N₅O₃S 587.2, m/z found 588.0 [M+H]⁺. ¹H NMR (300 MHz, CDCl₃) δ 8.20 (s, 0.4H), 7.90-7.72 (m, 1H), 7.44-7.27 (m, 2H), 7.21 (s, 0.6H), 7.10-7.02 (m, 2H), 6.21-6.18 (m, 0.6H), 6.11-6.04 (m, 0.4H), 5.27 (s, 1H), 4.34 (br s, 0.4H), 4.12-3.93 (m, 3.6H), 3.74-3.58 (m, 1H), 3.11-2.65 (m, 4H), 2.11-1.96 (m, 4H), 1.57 (br s, 2H), 1.29-1.19 (m, 2H), 1.13-1.04 (m, 3H).

### Compound 26

### Ethyl 4-(2-chloro-3,4-difluorophenyl)-6-(2-(3-methoxy-3-oxopropyl)-4,5,6,7-tetrahydrobenzo[d]oxazol-5-yl)-2-(thiazol-2-yl)-1,4-dihydropyrimidine-5-carboxylate

The crude product was purified by silica gel column chromatography (petroleum ether:ethyl acetate=10:1 to 2:1) followed by C18 column (acetonitrile:water=5% to 70%) to give Compound 26a (370 mg, 16% yield, including two stereoisomers) and Compound 26b (350 mg, 15% yield, including two stereoisomers) as yellow solids.

Compound 26a: LC-MS (ESI): R_{T}=4.452 min, mass calcd. for C₂₇H₂₅ClF₂N₄O₅S 590.1, m/z found 591.0 [M+H]⁺. Chiral HPLC (Column: Chiralpak IE 5 μm 4.6*250 mm; Mobile Phase: Hex:EtOH:DEA=70:30:0.2 at 1.0 mL/min; Temp: 30° C.; Wavelength: 230 nm, R_{T}=12.192 min). ¹H NMR (400 MHz, CDCl₃) δ 8.21 (s, 0.4H), 7.82 (d, J=2.8 Hz, 0.6H), 7.77 (d, J=3.6 Hz, 0.4H), 7.49 (d, J=3.2 Hz, 0.6H), 7.44 (d, J=3.2 Hz, 0.4H), 7.37 (d, J=2.0 Hz, 0.6H), 7.13-7.00 (m, 2H), 6.22 (s, 0.4H), 6.09 (d, J=2.8 Hz, 0.6H), 4.56-4.49 (m, 0.4H), 4.24-4.17 (m, 0.6H), 4.10-3.98 (m, 2H), 3.73 (s, 3H), 3.17-3.11 (m, 0.6H), 3.10-3.06 (m, 2H), 3.00-2.95 (m, 0.4H), 2.84 (t, J=7.2 Hz, 2H), 2.80-2.67 (m, 3H), 2.23-2.13 (m, 1H), 2.10-2.00 (m, 0.4H), 1.98-1.91 (m, 0.6H), 1.13-1.09 (m, 3H).

Compound 26b: LC-MS (ESI): R_{T}=4.677 min, mass calcd. for C₂₇H₂₅ClF₂N₄O₅S 590.1, m/z found 591.0 [M+H]⁺. Chiral HPLC (Column: Chiralpak IE 5 μm 4.6*250 mm; Mobile Phase: Hex:EtOH:DEA=70:30:0.2 at 1.0 mL/min; Temp: 30° C.; Wavelength: 230 nm, R_{T}=12.733 min and 16.706 min). ¹H NMR (400 MHz, CDCl₃) δ ¹H NMR (400 MHz, CDCl₃) δ 8.18 (s, 0.4H), 7.83 (d, J=3.2 Hz, 0.6H), 7.77 (d, J=3.2 Hz, 0.4H), 7.50 (d, J=3.2 Hz, 0.6H), 7.44 (d, J=3.2 Hz, 0.4H), 7.35 (d, J=2.0 Hz, 0.6H), 7.16-7.11 (m, 1H), 7.09-7.01 (m, 1H), 6.23 (s, 0.4H), 6.13 (d, J=2.8 Hz, 0.6H), 4.52-4.45 (m, 0.4H), 4.22-4.15 (m, 0.6H), 4.09-3.97 (m, 2H), 3.72 (s, 3H), 3.07 (t, J=7.6 Hz, 2H), 3.03-2.95 (m, 0.6H), 2.87-2.75 (m, 4.4H), 2.71-2.64 (m, 0.4H), 2.55-2.50 (m, 0.6H), 2.37-2.23 (m, 1H), 2.17-2.05 (m, 1H), 1.12 (t, J=6.8 Hz, 3H).

A stereoisomeric mixture of Compound 26a (310 mg, 0.52 mmol) was separated by chiral prep. HPLC (separation condition: Column: Chiralpak IA 5 μm 20*250 mm; Mobile Phase: Hex:EtOH:DEA=70:30:0.3 at 25 mL/min; Wavelength: 214 nm) to afford the title compounds Compound 26c (113 mg, 36% yield, 100% stereopure) and Compound 26d (114 mg, 37% yield, 95.3% stereopure) as yellow solids.

Compound 26c: LC-MS (ESI): R_{T}=3.904 min, mass calcd. for C₂₇H₂₅ClF₂N₄O₅S 590.1, m/z found 591.0 [M+H]⁺. Chiral HPLC (Column: Chiralpak IA 5 μm 4.6*250 mm; Mobile Phase: Hex:EtOH:DEA=70:30:0.2 at 1.0 mL/min; Temp: 30° C.; Wavelength: 230 nm, R_{T}=7.713 min). ¹H NMR (400 MHz, CDCl₃) δ 8.20 (s, 0.4H), 7.82 (d, J=2.8 Hz, 0.6H), 7.77 (d, J=3.2 Hz, 0.4H), 7.49 (d, J=2.8 Hz, 0.6H), 7.44 (d, J=3.2 Hz, 0.4H), 7.37 (d, J=2.4 Hz, 0.6H), 7.13-7.00 (m, 2H), 6.22 (s, 0.4H), 6.09 (d, J=2.8 Hz, 0.6H), 4.56-4.49 (m, 0.4H), 4.24-4.17 (m, 0.6H), 4.10-3.96 (m, 2H), 3.73 (s, 3H), 3.18-3.10 (m, 0.6H), 3.09-3.06 (m, 2H), 3.00-2.95 (m, 0.4H), 2.84 (t, J=7.2 Hz, 2H), 2.80-2.67 (m, 3H), 2.23-2.13 (m, 1H), 2.10-2.00 (m, 0.4H), 1.98-1.91 (m, 0.6H), 1.13-1.09 (m, 3H).

The stereoisomeric mixture of Compound 26b (290 mg, 0.49 mmol) was separated by chiral prep. HPLC (the first separation condition: Column: Chiralpak IE 5 μm 20*250 mm; Mobile Phase: Hex:EtOH:DEA=70:30:0.3 at 12 mL/min; Wavelength: 214 nm; the second separation condition: Column: Chiralpak IC 5 μm 20*250 mm; Mobile Phase: Hex:EtOH:DEA=70:30:0.3 at 15 mL/min; Wavelength: 214 nm) to afford

Compound 26e (95 mg, 33% yield, 100% stereopure) and Compound 26f (120 mg, 41% yield, 100% stereopure).

Compound 26f: LC-MS (ESI): R_{T}=4.647 min, mass calcd. for C₂₇H₂₅ClF₂N₄O₅ S 590.1, m/z found 591.1 [M+H]⁺. Chiral HPLC (Column: Chiralpak IE 5 μm 4.6*250 mm; Mobile Phase: Hex:EtOH:DEA=70:30:0.2 at 1.0 mL/min; Temp: 30° C.; Wavelength: 230 nm, R_{T}=16.631 min). ¹H NMR (400 MHz, CDCl₃) δ 8.18 (s, 0.4H), 7.83 (d, J=3.2 Hz, 0.6H), 7.77 (d, J=3.2 Hz, 0.4H), 7.50 (d, J=2.8 Hz, 0.6H), 7.44 (d, J=3.2 Hz, 0.4H), 7.35 (d, J=2.4 Hz, 0.6H), 7.16-7.12 (m, 1H), 7.11-7.00 (m, 1H), 6.23 (s, 0.4H), 6.13 (d, J=2.8 Hz, 0.6H), 4.52-4.45 (m, 0.4H), 4.22-4.15 (m, 0.6H), 4.09-3.97 (m, 2H), 3.72 (s, 3H), 3.07 (t, J=7.2 Hz, 2H), 3.03-2.95 (m, 0.6H), 2.87-2.76 (m, 4.4H), 2.71-2.64 (m, 0.4H), 2.55-2.50 (m, 0.6H), 2.37-2.22 (m, 1H), 2.17-2.04 (m, 1H), 1.12 (t, J=7.2 Hz, 3H).

### Compound 27

### Ethyl 4-(2-chloro-3,4-difluorophenyl)-6-(2-(3-methoxy-3-oxopropyl)-4,5,6,7-tetrahydrobenzo[d]oxazol-6-yl)-2-(thiazol-2-yl)-1,4-dihydropyrimidine-5-carboxylate

LC-MS (ESI): R_{T}=4.282 min, mass calcd. for C₂₇H₂₅ClF₂N₄O₅S 591.0, m/z found 590.9 [M+H]⁺. ¹H NMR (400 MHz, CD₃OD) δ 7.90-7.88 (m, 1H), 7.75-7.72 (m, 1H), 7.30-7.23 (m, 2H), 6.19 (s, 0.1H), 6.17 (s, 0.2H), 6.12 (d, J=1.6 Hz, 0.7H), 4.52-4.45 (m, 0.3H), 4.26-4.18 (m, 0.7H), 4.07-4.00 (m, 2H), 3.69 (s, 2H), 3.68 (s, 1H), 3.23-3.16 (m, 0.3H), 3.08-3.01 (m, 2.4H), 2.93-2.91 (m, 0.3H), 2.84-2.79 (m, 2.5H), 2.69-2.60 (m, 2H), 2.56-2.54 (m, 0.5H), 2.24-2.18 (m, 0.8H), 2.12-2.04 (m, 1H), 1.94-1.90 (m, 0.2H), 1.13-1.08 (m, 3H).

A stereoisomeric mixture of ethyl 4-(2-chloro-3,4-difluorophenyl)-6-(2-(3-methoxy-3-oxopropyl)-4,5,6,7-tetrahydrobenzo[d]oxazol-6-yl)-2-(thiazol-2-yl)-1,4-dihydropyrimidine-5-carboxylate Compound 27 (1.0 g, 90% purity) was separated by chiral Prep. HPLC and Prep. HPLC (chiral Prep. HPLC separation condition: column: chiralpak IE 5 μm 20*250 mm; Mobile Phase: Hex:EtOH=75:25 at 15 mL/min; Temp: 30° C.; Wavelength: 230 nm; Prep. HPLC separation condition: column: Waters Xbrige C18 5 μm 19*150 mm, Mobile Phase A: Water (0.1% ammonium bicarbonate), Mobile Phase B: acetonitrile, UV: 214 nm, Flow rate: 15 mL/min, Gradient: 50-95% (% B)) to give Compound 27a (8.1 mg, 16% yield, 100% stereopure), Compound 27b (11.8 mg, 24% yield, 100% stereopure) and Part 1 (406 mg, 41% yield). The Part 1 was separated by SFC and Prep. HPLC (SFC separation condition: column: chiralpak IF 5 μm 20*250 mm; Mobile Phase: CO₂:EtOH=70:30 at 50 g/min; Co-solvent: MeOH (0.2° C.); Col. Temp 41.1° C.; Wavelength: 230 nm; Back pressure: 100 bar; Prep. HPLC separation condition: column: Waters Xbrige C18 5 μm 19*150 mm, Mobile Phase A: Water (0.1% ammonium hydroxide), Mobile Phase B: acetonitrile, UV: 214 nm, Flow rate: 15 mL/min, Gradient: 50-95% (% B)) to give Compound 27c (18.1 mg, 45% yield, 100% stereopure), Compound 27d (12.3 mg, 25%, 100% stereopure).

Compound 27b: LC-MS (ESI): R_{T}=4.280 min, mass calcd. for C₂₇H₂₅ClF₂N₄O₅S 591.0, m/z found 590.9 [M+H]⁺. Chiral HPLC (Column: Chiralpak IE 5 μm 4.6*250 mm; Mobile Phase: Hex:EtOH=75:25 at 1.0 mL/min; Temp: 30° C.; Wavelength: 230 nm, R_{T}=18.454 min). ¹H NMR (400 MHz, CD₃OD) δ 7.88-7.87 (m, 1H), 7.73-7.71 (m, 1H), 7.29-7.22 (m, 2H), 6.18 (s, 0.3H), 6.11 (s, 0.7H), 4.50-4.42 (m, 0.3H), 4.25-4.18 (m, 0.7H), 4.06-3.99 (m, 2H), 3.68 (s, 2H), 3.67 (s, 1H), 3.21-3.15 (m, 0.5H), 3.07-3.00 (m, 2.5H), 2.83-2.77 (m, 3H), 2.63-2.59 (m, 0.5H), 2.54-2.53 (m, 1.5H), 2.18-2.13 (m, 0.3H), 2.11-2.01 (m, 1H), 1.92-1.88 (m, 0.7H), 1.12-1.07 (m, 3H).

Compound 27d: LC-MS (ESI): R_{T}=4.283 min, mass calcd. for C₂₇H₂₅ClF₂N₄O₅S 591.0, m/z found 590.9 [M+H]⁺. Chiral HPLC (Column: Chiralpak IE 5 μm 4.6*250 mm; Mobile Phase: Hex:EtOH=75:25 at 1.0 mL/min; Temp: 30° C.; Wavelength: 230 nm, R_{T}=18.798 min). ¹H NMR (400 MHz, CD₃OD) δ 7.89-7.88 (m, 1H), 7.74-7.71 (m, 1H), 7.27-7.22 (m, 2H), 6.15 (s, 0.3H), 6.11 (s, 0.7H), 4.51-4.44 (m, 0.3H), 4.24-4.17 (m, 0.7H), 4.06-3.99 (m, 2H), 3.68 (s, 2H), 3.67 (s, 1H), 3.08-3.02 (m, 3H), 2.91-2.89 (m, 0.5H), 2.82-2.79 (m, 2H), 2.68-2.59 (m, 2.5H), 2.23-2.16 (m, 1H), 2.11-2.07 (m, 0.8H), 2.03-2.00 (m, 0.2H), 1.12-1.07 (m, 3H).

### Compound 28

### Ethyl 4-(2-chloro-3,4-difluorophenyl)-6-(2-(3-methoxy-3-oxopropyl)-4,5,6,7-tetrahydrobenzo[d]thiazol-5-yl)-2-(thiazol-2-yl)-1,4-dihydropyrimidine-5-carboxylate

The crude product was purified by silica gel column chromatography (petroleum ether:ethyl acetate=15:1 to 5:1) followed by C18 column (acetonitrile:water=5% to 75%) to give Compound 28a (750 mg, 19% yield, including two stereoisomers) and Compound 28b (600 mg, 15% yield, including two stereoisomers) as yellow solids.

Compound 28a: LC-MS (ESI): R_{T}=2.995 min, mass calcd. for C₂₇H₂₅ClF₂N₄O₄S₂ 606.1, m/z found 607.2 [M+H]⁺. Chiral HPLC (Column: Chiralpak IE 5 μm 4.6*250 mm; Mobile Phase: Hex:EtOH:DEA=70:30:0.2 at 1.0 mL/min; Temp: 30° C.; Wavelength: 230 nm, R_{T}=11.624 min and 12.046 min). ¹H NMR (400 MHz, CDCl₃) δ 8.23 (s, 0.4H), 7.82 (d, J=3.2 Hz, 0.5H), 7.75 (d, J=2.8 Hz, 0.5H), 7.48 (d, J=2.8 Hz, 0.5H), 7.44 (d, J=3.2 Hz, 0.5H), 7.37 (br s, 0.6H), 7.15-7.03 (m, 2H), 6.23 (s, 0.4H), 6.10 (d, J=2.4 Hz, 0.6H), 4.59-4.54 (m, 0.4H), 4.31-4.25 (m, 0.6H), 4.08-3.99 (m, 2H), 3.72 (s, 3H), 3.33-3.26 (m, 3H), 3.10-3.05 (m, 0.5H), 2.99-2.92 (m, 1.5H), 2.87-2.82 (m, 3H), 2.20-2.16 (m, 1H), 2.06-1.95 (m, 1H), 1.18 (td, J=7.2, 1.2 Hz, 3H).

Compound 28b: LC-MS (ESI): R_{T}=2.862 min, mass calcd. for C₂₇H₂₅ClF₂N₄O₄S₂ 606.1, m/z found 607.1 [M+H]⁺. Chiral HPLC (Column: Chiralpak IE 5 μm 4.6*250 mm; Mobile Phase: Hex:EtOH:DEA=70:30:0.2 at 1.0 mL/min; Temp: 30° C.; Wavelength: 230 nm, R_{T}=14.281 min and 17.634 min). ¹H NMR (400 MHz, CDCl₃) δ 8.22 (s, 0.4H), 7.82 (d, J=3.2 Hz, 0.6H), 7.75 (d, J=3.2 Hz, 0.4H), 7.49 (d, J=3.2 Hz, 0.6H), 7.44 (d, J=3.2 Hz, 0.4H), 7.36 (s, 0.6H), 7.18-7.13 (m, 1H), 7.11-7.01 (m, 1H), 6.24 (s, 0.4H), 6.14 (d, J=1.6 Hz, 0.6H), 4.56-4.50 (m, 0.4H), 4.27-4.22 (m, 0.6H), 4.09-3.97 (m, 2H), 3.71 (s, 3H), 3.27 (t, J=6.8 Hz, 2H), 3.21-3.10 (m, 1H), 2.97-2.93 (m, 2H), 2.85-2.81 (m, 3H), 2.31-2.24 (m, 1H), 2.19-2.16 (m, 0.6H), 2.09-2.03 (m, 0.4H), 1.12 (t, J=7.2 Hz, 3H).

The stereoisomeric mixture of Compound 28a (650 mg, 1.07 mmol) was separated by chiral prep. HPLC (separation condition: Column: Chiralpak IC 5 μm 20*250 mm; Mobile Phase: Hex:EtOH:DEA=70:30:0.3 at 12 mL/min; Wavelength: 214 nm) to afford Compound 28c (200 mg, 33% yield, 100% stereopure) and Compound 28d (220 mg, 37% yield, 100% stereopure) as yellow solids.

Compound 28c: LC-MS (ESI): R_{T}=3.296 min, mass calcd. for C₂₇H₂₅ClF₂N₄O₄S₂ 606.1, m/z found 607.1 [M+H]⁺. Chiral HPLC (Column: Chiralpak IC 5 μm 4.6*250 mm; Mobile Phase: Hex:EtOH:DEA=70:30:0.2 at 1.0 mL/min; Temp: 30° C.; Wavelength: 230 nm, R_{T}=6.649 min). ¹H NMR (400 MHz, CDCl₃) δ 8.24 (s, 0.5H), 7.82 (d, J=3.2 Hz, 0.5H), 7.75 (d, J=3.2 Hz, 0.5H), 7.49 (d, J=2.8 Hz, 0.5H), 7.44 (d, J=2.8 Hz, 0.5H), 7.38 (d, J=2.4 Hz, 0.5H), 7.13-7.01 (m, 2H), 6.23 (s, 0.5H), 6.10 (d, J=2.8 Hz, 0.5H), 4.60-4.55 (m, 0.5H), 4.31-4.25 (m, 0.5H), 4.08-4.01 (m, 2H), 3.72 (s, 3H), 3.33-3.26 (m, 3H), 3.10-3.05 (m, 0.5H), 2.99-2.92 (m, 1.5H), 2.87-2.82 (m, 3H), 2.21-2.15 (m, 1H), 2.06-1.95 (m, 1H), 1.11 (td, J=7.2, 1.2 Hz, 3H).

The stereoisomeric mixture of Compound 28b (500 mg, 0.823 mmol) was separated by chiral prep. HPLC (separation condition: Column: Chiralpak ID 5 μm 20*250 mm; Mobile Phase: Hex:EtOH:DEA=70:30:0.3 at 14 mL/min; Wavelength: 214 nm) to afford Compound 28e (170 mg, 34% yield, 100% stereopure) and Compound 28f (180 mg, 36% yield, 98.2% stereopure) as yellow solids.

Compound 28f: LC-MS (ESI): R_{T}=3.125 min, mass calcd. for C₂₇H₂₅ClF₂N₄O₄S₂ 606.1, m/z found 607.1 [M+H]⁺. Chiral HPLC (Column: Chiralpak IE 5 μm 4.6*250 mm; Mobile Phase: Hex:EtOH:DEA=70:30:0.2 at 1.0 mL/min; Temp: 30° C.; Wavelength: 230 nm, R_{T}=17.558 min). ¹H NMR (400 MHz, CDCl₃) δ 8.22 (s, 0.4H), 7.82 (d, J=3.2 Hz, 0.6H), 7.76 (d, J=3.2 Hz, 0.4H), 7.49 (d, J=2.8 Hz, 0.6H), 7.44 (d, J=3.2 Hz, 0.4H), 7.36 (d, J=2.4 Hz, 0.6H), 7.18-7.13 (m, 1H), 7.11-7.01 (m, 1H), 6.24 (s, 0.4H), 6.14 (d, J=2.4 Hz, 0.6H), 4.56-4.51 (m, 0.4H), 4.28-4.21 (m, 0.6H), 4.09-3.97 (m, 2H), 3.71 (s, 3H), 3.27 (td, J=7.2, 2.4 Hz, 2H), 3.22-3.10 (m, 1H), 2.97-2.90 (m, 2H), 2.85-2.81 (m, 3H), 2.31-2.24 (m, 1H), 2.19-2.11 (m, 0.6H), 2.09-2.03 (m, 0.4H), 1.12 (t, J=7.2 Hz, 3H).

### Compound 29

### Ethyl 4-(2-chloro-3,4-difluorophenyl)-6-(2-(3-methoxy-3-oxopropyl)-4,5,6,7-tetrahydrobenzo[d]thiazol-6-yl)-2-(thiazol-2-yl)-1,4-dihydropyrimidine-5-carboxylate

LC-MS (ESI): R_{T}=3.442 min, mass calcd. for C₂₇H₂₅ClF₂N₄O₄S₂ 606.1, m/z found 607.1 [M+H]⁺. ¹H NMR (400 MHz, DMSO-d₆) δ 9.67-9.64 (m, 0.8H), 9.25 (s, 0.1H), 9.20 (s, 0.1H), 8.00-7.93 (m, 2H), 7.53-7.43 (m, 1H), 7.29-7.24 (m, 1H), 6.08 (s, 0.1H), 6.06 (s, 0.1H), 5.97 (s, 0.8H), 4.31-4.25 (m, 0.2H), 4.10-3.92 (m, 2.8H), 3.62 (s, 3H), 3.21-3.19 (m, 0.2H), 3.17-3.14 (m, 2H), 3.08-2.83 (m, 2H), 2.80-2.74 (m, 3H), 2.72-2.61 (m, 0.8H), 2.26-1.84 (m, 2H), 1.07-0.99 (m, 3H).

A stereoisomeric mixture of ethyl 4-(2-chloro-3,4-difluorophenyl)-6-(2-(3-methoxy-3-oxopropyl)-4,5,6,7-tetrahydrobenzo[d]thiazol-6-yl)-2-(thiazol-2-yl)-1,4-dihydropyrimidine-5-carboxylate Compound 29 [2 batches, (325 mg, 99% purity) and (475 mg, 90% purity)] was separated by chiral Prep. HPLC (the first separation condition: Column: Chiralpak IE 5 μm 30*250 mm; Mobile Phase: Hex:EtOH:DEA=70:30:0.3 at 15 mL/min; Temp: 30° C.; Wavelength: 214 nm; the second separation condition: Column: Chiralpak IG 5 μm 20*250 mm; Mobile Phase: Hex:EtOH:DEA=70:30:0.3 at 15 mL/min; Temp: 30° C.; Wavelength: 214 nm) to afford Compound 29a (104 mg, 98% purity, 26% yield, 100% stereopure), Compound 29b (107 mg, 90% purity, 14% yield, 97.8% stereopure), Compound 29c (144 mg, 99% purity, 36% yield, 100% stereopure) and Compound 29d (150 mg, 96% purity, 36% yield, 97.6% stereopure) as yellow solids.

Compound 29b: LC-MS (ESI): R_{T}=4.425 min, mass calcd. for C₂₇H₂₅ClF₂N₄O₄S₂ 606.1, m/z found 606.8 [M+H]⁺. Chiral HPLC (Column: Chiralpak IE 5 μm 4.6*250 mm; Mobile Phase: Hex:EtOH:DEA=70:30:0.2 at 1.0 mL/min; Temp: 30° C.; Wavelength: 254 nm, R_{T}=13.738 min). ¹H NMR (400 MHz, DMSO-d₆) δ 9.63 (d, J=3.2 Hz, 0.8H), 9.24 (s, 0.2H), 8.00-7.92 (m, 2H), 7.49-7.42 (m, 1H), 7.27-7.21 (m, 1H), 6.07 (s, 0.2H), 5.96 (d, J=3.2 Hz, 0.8H), 4.30-4.23 (m, 0.2H), 4.08-4.04 (m, 0.8H), 3.99-3.91 (m, 2H), 3.61 (s, 3H), 3.18-3.14 (m, 3H), 3.05-2.89 (m, 1H), 2.81-2.76 (m, 3H), 2.70-2.61 (m, 1H), 2.27-2.19 (m, 0.2H), 2.07-1.94 (m, 1H), 1.88-1.84 (m, 0.8H), 1.06-0.98 (m, 3H).

Compound 29c: LC-MS (ESI): R_{T}=4.432 min, mass calcd. for C₂₇H₂₅ClF₂N₄O₄S₂ 606.1, m/z found 606.8 [M+H]⁺. Chiral HPLC (Column: Chiralpak IG 5 μm 4.6*250 mm; Mobile Phase: Hex:EtOH:DEA=70:30:0.2 at 1.0 mL/min; Temp: 30° C.; Wavelength: 230 nm, R_{T}=10.989 min). ¹H NMR (400 MHz, DMSO-d₆) δ 9.66 (d, J=3.6 Hz, 0.7H), 9.18 (s, 0.3H), 8.01-7.93 (m, 2H), 7.52-7.46 (m, 1H), 7.29-7.26 (m, 1H), 6.05 (s, 0.2H), 5.96 (d, J=3.6 Hz, 0.8H), 4.32-4.25 (m, 0.2H), 4.08-4.02 (m, 0.8H), 3.99-3.92 (m, 2H), 3.61 (s, 3H), 3.16 (t, J=7.2 Hz, 2H), 3.04-2.95 (m, 1H), 2.88-2.84 (m, 1H), 2.80-2.67 (m, 4H), 2.33-2.32 (m, 0.2H), 2.22-2.03 (m, 1.8H), 1.06-0.99 (m, 3H).

### Compound 30

### 4-(2-Chloro-3,4-difluoro-phenyl)-6-[2-(2-methoxycarbonyl-ethyl)-4,5,6,7-tetrahydro-2H-indazol-6-yl]-2-thiazol-2-yl-1,4-dihydro-pyrimidine-5-carboxylic acid ethyl ester

LC-MS (ESI): R_{T}=2.009 min, mass calcd. for C₂₇H₂₆ClF₂N₄O₄S 589.1, m/z found 590.1 [M+H]⁺. ¹H NMR (400 MHz, DMSO-d₆) δ 9.60 (dd, J=8.8, 3.6 Hz, 0.6H), 9.10 (s, 0.2H), 9.07 (s, 0.2H), 8.01-7.98 (m, 1.4H), 7.97-7.96 (m, 0.3H), 7.93-7.92 (m, 0.3H), 7.54-7.45 (m, 1H), 7.43 (s, 0.3H), 7.39 (s, 0.7H), 7.29-7.23 (m, 1H), 6.06 (d, J=3.6 Hz, 0.3H), 5.97 (d, J=3.6 Hz, 0.7H), 4.28-4.22 (m, 2.2H), 4.01-3.91 (m, 2.8H), 3.61 (s, 3H), 3.01-2.95 (m, 0.7H), 2.88-2.81 (m, 2.7H), 2.75-2.56 (m, 2.6H), 2.17-1.74 (m, 2H), 1.06-0.98 (m, 3H).

A stereoisomeric mixture of Compound 30 (1.00 g, 1.60 mmol, 95% purity) was separated by chiral prep. HPLC (the first purification condition: Column: Chiralpak IC 5 μm 20*250 mm; Mobile Phase: Hex:EtOH:DEA=70:30:0.3 at 25 mL/min; Temp: 30° C.; Wavelength: 214 nm; the second purification condition: Column: Chiralpak IE 5 μm 20*250 mm; Mobile Phase: Hex:EtOH=70:30 at 12 mL/min; Temp: 30° C.; Wavelength: 230 nm) to give the title compounds Compound 30a (200 mg, 21% yield, 100% stereopure), Compound 30b (180 mg, 19% yield, 100% stereopure), Compound 30c (150 mg, 16% yield, 100% stereopure) and Compound 30d (200 mg, 21% yield, 100% stereopure).

Compound 30a: LC-MS (ESI): R_{T}=4.250 min, mass calcd. for C₂₇H₂₆ClF₂N₅O₄S 589.1, m/z found 589.9 [M+H]⁺. Chiral HPLC (Column: Chiralpak IC 5 μm 4.6*250 mm; Mobile Phase: Hex:EtOH:DEA=70:30:0.2 at 1 mL/min; Wavelength: 230 nm, R_{T}=6.635 min). H NMR (400 MHz, CD₃OD) δ 7.89-7.87 (m, 1H), 7.73-7.71 (m, 1H), 7.38 (s, 0.4H), 7.32 (s, 0.6H), 7.27-7.24 (m, 2H), 6.19 (s, 0.4H), 6.12 (s, 0.6H), 4.39-4.34 (m, 2.4H), 4.16-4.09 (m, 0.6H), 4.06-3.99 (m, 2H), 3.68 (s, 1.8H), 3.67 (s, 1.2H), 3.18-2.96 (m, 1.4H), 2.87 (t, J=6.4 Hz, 2H), 2.82-2.56 (m, 2.6H), 2.08-1.94 (m, 1.4H), 1.88-1.83 (m, 0.6H), 1.12-1.07 (m, 3H).

Compound 30c: Chiral HPLC (Column: Chiralpak IE 5 μm 4.6*250 mm; Mobile Phase: Hex:EtOH=60:40 at 1 mL/min; Wavelength: 230 nm, R_{T}=9.025 min). ¹H NMR (400 MHz, DMSO-d₆) δ 9.62 (d, J=3.6 Hz, 0.7H), 9.07 (s, 0.3H), 8.01-7.93 (m, 2H), 7.50-7.39 (m, 2H), 7.29-7.26 (m, 1H), 6.06 (s, 0.3H), 5.97 (d, J=3.6 Hz, 0.7H), 4.28-4.22 (m, 2.4H), 4.02-3.94 (m, 2.6H), 3.61 (s, 3H), 2.88-2.57 (m, 6H), 2.17-1.95 (m, 2H), 1.06-0.99 (m, 3H).

### Compound 31

### Ethyl 4-(2-chloro-3,4-difluorophenyl)-6-(1-cyano-2-(3-methoxy-3-oxopropyl)-4,5,6,7-tetrahydro-2H-isoindol-5-yl)-2-(thiazol-2-yl)-1,4-dihydropyrimidine-5-carboxylate

LC-MS (ESI): R_{T}=4.210 min, mass calcd. for C₂₉H₂₆ClF₂N₅O₄S 613.1, m/z found 613.9 [M+H]⁺. ¹H NMR (400 MHz, CDCl₃) δ 8.19 (s, 0.5H), 7.83 (d, J=2.8 Hz, 0.5H), 7.76 (d, J=3.2 Hz, 0.5H), 7.49 (d, J=3.2 Hz, 0.5H), 7.44 (d, J=3.2 Hz, 0.5H), 7.35 (s, 0.5H), 7.18-7.03 (m, 2H), 6.71-6.63 (m, 1H), 6.22 (d, J=7.2 Hz, 0.5H), 6.11-6.10 (m, 0.5H), 4.36-4.27 (m, 2.5H), 4.07-3.97 (m, 2.5H), 3.72 (s, 3H), 3.06-2.58 (m, 6H), 2.23-1.87 (m, 2H), 1.11 (t, J=6.8 Hz, 3H).

### Compound 32

### ethyl 4-(2-chloro-3,4-difluorophenyl)-6-(3-cyano-2-(3-methoxy-3-oxopropyl)-4,5,6,7-tetrahydro-2H-isoindol-5-yl)-2-(thiazol-2-yl)-1,4-dihydropyrimidine-5-carboxylate

LC-MS (ESI): R_{T}=4.319 min, mass calcd. for C₂₉H₂₆ClF₂N₅O₄S 613.1, m/z found 613.9 [M+H]⁺. ¹H NMR (400 MHz, CDCl₃) δ 8.18 (d, J=10.8 Hz, 0.5H), 7.83 (t, J=3.2 Hz, 0.5H), 7.77 (dd, J=6.0, 3.2 Hz, 0.5H), 7.52-7.48 (m, 0.5H), 7.44 (dd, J=3.2, 1.6 Hz, 0.5H), 7.36 (dd, J=7.2, 2.8 Hz, 0.5H), 7.19-7.00 (m, 2H), 6.67 (d, J=2.0 Hz, 0.5H), 6.64 (d, J=2.0 Hz, 0.5H), 6.23 (d, J=4.0 Hz, 0.5H), 6.11 (dd, J=8.0, 2.4 Hz, 0.5H), 4.45-4.36 (m, 0.5H), 4.33-4.27 (m, 2H), 4.16-3.96 (m, 2.5H), 3.73 (s, 2H), 3.72 (s, 1H), 3.24-3.13 (m, 0.3H), 3.09-2.93 (m, 0.7H), 2.85-2.53 (m, 5H), 2.22-2.06 (m, 1H), 2.03-1.82 (m, 1H), 1.14-1.09 (m, 3H).

### Compound 33

### Methyl 4-(2-chloro-3,4-difluorophenyl)-6-(2-(3-methoxy-3-oxopropyl)-5,6,7,8-tetrahydroquinazolin-6-yl)-2-(thiazol-2-yl)-1,4-dihydropyrimidine-5-carboxylate

LC-MS (ESI): R_{T}=1.67 min, mass calcd. for C₂₇H₂₄ClF₂N₅O₄S 587.1, m/z found 587.9 [M+H]⁺. ¹H NMR (400 MHz, DMSO-d₆) δ 9.69 (s, 0.8H), 9.29 (d, J=9.2 Hz, 0.2H), 8.48-8.43 (m, 1H), 8.01-7.94 (m, 2H), 7.53-7.44 (m, 1H), 7.29-7.22 (m, 1H), 6.06 (d, J=12.0 Hz, 0.2H), 5.97 (s, 0.8H), 4.26-4.17 (m, 0.2H), 4.05-3.98 (m, 0.8H), 3.59 (s, 3H), 3.52-3.50 (m, 3H), 3.19-3.07 (m, 2.4H), 3.02-2.73 (m, 5.6H), 2.23-1.88 (m, 2H).

### Compound 67: (trans)-methyl 4-(2-chloro-3,4-difluorophenyl)-6-(2-(3-(methoxycarbonyl)cyclobutyl)-4,5,6,7-tetrahydrobenzo[d]thiazol-6-yl)-2-(thiazol-2-yl)-1,4-dihydropyrimidine-5-carboxylate

LC-MS (ESI): R_{T}=1.83 min, mass calcd. For C₂₈H₂₅ClF₂N₄O₄S₂ 618.1, m/z found 619.0 [M+H]⁺. ¹H NMR (400 MHz, CDCl₃) δ 8.25 (s, 0.4H), 7.84-7.75 (m, 1H), 7.53-7.49 (m, 0.6H), 7.45-7.42 (m, 1H), 7.14-7.00 (m, 2H), 6.23 (s, 0.2H), 6.20 (s, 0.2H), 6.11-6.09 (m, 0.6H), 4.54-4.46 (m, 0.4H), 4.27-4.20 (m, 0.6H), 3.99-3.90 (m, 1H), 3.74 (s, 3H), 3.62-3.59 (m, 3H), 3.38-3.12 (m, 2H), 3.05-2.83 (m, 3H), 2.80-2.73 (m, 2H), 2.67-2.63 (m, 2H), 2.21-2.17 (m, 1H), 1.99-1.93 (m, 1H).

Racemic compound 67 (700 mg, 99% purity) was separated by chiral Prep. HPLC (the first separation condition: Column: Chiralpak IE 5 μm 20*250 mm; Mobile Phase: Hex:EtOH:DEA=70:30:0.3 at 12 mL/min; Temp: 30° C.; Wavelength: 214 nm; the second separation condition: Column: Chiralpak IG 5 μm 20*250 mm; Mobile Phase: Hex:EtOH:DEA=60:40:0.3 at 30 mL/min; Temp: 30° C.; Wavelength: 214 nm); the third separation condition: Column: Chiralpak IC 5 μm 20*250 mm; Mobile Phase: Hex:IPA:DEA=70:30:0.3 at 15 mL/min; Temp: 30° C.; Wavelength: 230 nm) to afford 67m (110 mg), 67n (110 mg) and 67q (110 mg) and 67p (127 mg) as yellow solids.

Compound 67n: LC-MS (ESI): R_{T}=1.82 min, mass calcd. for C₂₈H₂₅ClF₂N₄O₄S₂ 618.1, m/z found 619.0 [M+H]⁺. Chiral analysis (Column: Chiralpak IE 5 μm 4.6*250 mm; Mobile Phase: Hex:EtOH:DEA=70:30:0.2 at 1.0 mL/min; Temp: 30° C.; Wavelength: 230 nm, R_{T}=15.499 min). ¹H NMR (400 MHz, CDCl₃) δ 8.25 (s, 0.4H), 7.82 (d, J=2.8 Hz, 0.6H), 7.75 (d, J=3.2 Hz, 0.4H), 7.49 (d, J=2.8 Hz, 0.6H), 7.44-7.41 (m, 1H), 7.09-7.03 (m, 2H), 6.22 (s, 0.4H), 6.09 (s, 0.6H), 4.51-4.44 (m, 0.4H), 4.25-4.18 (m, 0.6H), 3.99-3.91 (m, 1H), 3.74 (s, 3H), 3.61 (s, 1.8H), 3.58 (s, 1.2H), 3.38-3.22 (m, 2H), 3.04-2.86 (m, 3H), 2.79-2.72 (m, 2H), 2.68-2.61 (m, 2H), 2.22-2.16 (m, 1H), 2.09-2.07 (m, 0.4H), 1.98-1.94 (m, 0.6H).

Compound 67p: LC-MS (ESI): R_{T}=1.92 min, mass calcd. for C₂₈H₂₅ClF₂N₄O₄S₂ 618.1, m/z found 618.8 [M+H]⁺. Chiral analysis (Column: Chiralpak IC 5 μm 4.6*250 mm; Mobile Phase: Hex:IPA:DEA=70:30:0.2 at 1.0 mL/min; Temp: 30° C.; Wavelength: 254 nm, R_{T}=9.925 min). ¹H NMR (400 MHz, CDCl₃) δ 8.25 (s, 0.4H), 7.83 (d, J=2.8 Hz, 0.6H), 7.75 (d, J=2.8 Hz, 0.4H), 7.50 (d, J=3.2 Hz, 0.6H), 7.44 (d, J=3.2 Hz, 1H), 7.14-7.00 (m, 2H), 6.20 (s, 0.4H), 6.09 (d, J=2.0 Hz, 0.6H), 4.55-4.49 (m, 0.4H), 4.26-4.21 (m, 0.6H), 3.98-3.90 (m, 1H), 3.74 (s, 3H), 3.61 (s, 1.8H), 3.60 (s, 1.2H), 3.30-3.24 (m, 1H), 3.18-3.12 (m, 1H), 3.03-2.84 (m, 3H), 2.79-2.73 (m, 2H), 2.68-2.61 (m, 2H), 2.40-2.27 (m, 1H), 2.20-2.08 (m, 1H).

### Compound 69: Methyl 4-(2-chloro-3,4-difluorophenyl)-6-(2-(3-methoxy-3-oxopropyl)-4,5,6,7-tetrahydro-2H-indazol-5-yl)-2-(thiazol-2-yl)-1,4-dihydropyrimidine-5-carboxylate

LC-MS (ESI): R_{T}=1.63 min, mass calcd. for C₂₆H₂₄ClF₂N₅O₄S 575.1, m/z found 576.1 [M+H]⁺. ¹H NMR (400 MHz, DMSO-d₆) δ 9.64 (m, 0.7H), 9.15 (s, 0.15H), 9.10 (s, 0.15H), 8.02-7.93 (m, 2H), 7.52-7.44 (m, 1.3H), 7.40-7.38 (m, 0.7H), 7.30-7.23 (m, 1H), 6.07 (s, 0.15H), 6.05 (s, 0.15H), 5.97-5.95 (m, 0.7H), 4.29-4.23 (m, 2H), 4.19-4.12 (m, 0.3H), 3.94-3.88 (m, 0.7H), 3.62 (s, 3H), 3.52 (s, 2.1H), 3.50 (s, 0.9H), 3.02-2.82 (m, 3H), 2.80-2.69 (m, 2H), 2.66-2.54 (m, 1H), 2.16-2.06 (m, 0.7H), 2.00-1.93 (m, 1H), 1.83-1.79 (m, 0.3H).

Racemic compound 69 (800 mg, 90% purity, 1.25 mmol) was separated by chiral Prep. HPLC (the first separation condition: Column: Chiralpak IG 4.6 mm*250 mm Sum; Mobile Phase: Hex:EtOH:DEA=60:40:0.2 at 14 mL/min; Temp: 30° C.; Wavelength: 214 nm; the second separation condition: Column: Chiralpak IG 4.6 mm*250 mm Sum; Mobile Phase: CO₂:MeOH:DEA=70:30:0.3 at 50 g/min; Temp: 30° C.; Wavelength: 214 nm) to give the title compounds 69m (91 mg, 90% purity from HNMR, 11% yield, 100% stereopure), 69n (100 mg, 90% purity from HNMR, 13% yield, 98.7% stereopure), 69p (140 mg, 90% purity from HNMR, 18% yield, 99.5% stereopure) and 69q (140 mg, 90% purity from HNMR, 18% yield, 99.7% stereopure) as yellow solids.

Compound 69n: Chiral analysis (Column: Chiralpak IG 5 μm 4.6*250 mm; Mobile Phase: Hex:EtOH:DEA=80:20:0.2 at 1.0 mL/min; Temp: 30° C.; Wavelength: 230 nm, R_{T}=17.949 min). ¹H NMR (400 MHz, DMSO-d₆) δ 9.60 (d, J=4.0 Hz, 0.7H), 9.13 (s, 0.3H), 8.01-7.93 (m, 2H), 7.51-7.45 (m, 1.3H), 7.40 (s, 0.7H), 7.27-7.18 (m, 1H), 6.06 (s, 0.3H), 5.96 (d, J=3.6 Hz, 0.7H), 4.28-4.23 (m, 2H), 4.19-4.10 (m, 0.3H), 3.96-3.84 (m, 0.7), 3.61 (s, 3H), 3.51 (s, 2.1H), 3.50 (s, 0.9H), 3.00-2.80 (m, 3H), 2.76-2.63 (m, 2H), 2.62-2.53 (m, 1H), 2.20-2.07 (m, 0.3H), 2.04-1.91 (m, 1H), 1.85-1.76 (m, 0.7H).

Compound 69q: Chiral analysis (Column: Chiralpak IG 5 μm 4.6*250 mm; Mobile Phase: Hex:EtOH:DEA=60:40:0.2 at 1.0 mL/min; Temp: 30° C.; Wavelength: 280 nm, R_{T}=12.500 min). ¹H NMR (400 MHz, DMSO-d₆) δ 9.63 (d, J=3.6 Hz, 0.7H), 9.08 (s, 0.3H), 8.01-7.95 (m, 2H), 7.49-7.45 (m, 1.3H), 7.38 (s, 0.7H), 7.29-7.21 (m, 1H), 6.04 (s, 0.3H), 5.94 (d, J=3.6 Hz, 0.7H), 4.28-4.22 (m, 2H), 4.20-4.11 (m, 0.3H), 3.94-3.86 (m, 0.7), 3.61 (s, 3H), 3.51 (s, 2.1H), 3.50 (s, 0.9H), 2.85 (t, J=6.8 Hz, 2H), 2.77-2.71 (m, 2H), 2.69-2.58 (m, 1H), 2.55-2.53 (m, 1H), 2.26-2.18 (m, 0.3H), 2.14-2.04 (m, 1H), 2.00-1.96 (m, 0.7H).

Compound 71: Methyl 4-(2-chloro-3,4-difluorophenyl)-6-(2-((trans)-3-(methoxycarbonyl)cyclobutyl)-4,5,6,7-tetrahydrobenzo[d]oxazol-6-yl)-2-(thiazol-2-yl)-1,4-dihydropyrimidine-5-carboxylate, LC-MS (ESI): R_{T}=1.77 min, mass calcd. for C₂₈H₂₅ClF₂N₄O₅S 602.1, m/z found 602.8 [M+H]⁺. ¹H NMR (400 MHz, CDCl₃) δ 8.23 (s, 0.3H), 7.86-7.82 (m, 0.7H), 7.79-7.76 (m, 0.3H), 7.53-7.49 (m, 0.7H), 7.47-7.41 (s, 1H), 7.17-7.00 (m, 2H), 6.22-6.20 (m, 0.3H), 6.12-6.08 (m, 0.7H), 4.57-4.47 (m, 0.3H), 4.29-4.19 (m, 0.7H), 3.70 (s, 3H), 3.64-3.58 (m, 3H), 3.57-3.49 (m, 1H), 3.27-2.99 (m, 2H), 2.90-2.82 (m, 0.6H), 2.77-2.55 (m, 6.4H), 2.34-2.20 (m, 0.7H), 2.15-1.97 (m, 1H), 1.94-1.86 (m, 0.3H).

Racemic compound 71 (245 mg) was separated by chiral Prep. HPLC (the first separation condition: Column: Chiralpak IA 5 μm 20*250 mm; Mobile Phase: Hex:IPA:DEA=80:20:0.3 at 12 mL/min; Wavelength: 214 nm; the second separation condition: Column: Chiralpak IG 5 μm 20*250 mm; Mobile Phase: Hex:EtOH:DEA=80:20:0.3 at 35 mL/min; Wavelength: 254 nm; the third separation condition: Column: Superchiral S-OJ 5 μm 21*250 mm; Mobile Phase: Hex:EtOH:DEA=75:25:0.5 at 20 mL/min; Wavelength: 254 nm) to afford 71m (30 mg, 95% purity from NMR, 13% yield, 100% stereopure), 71n (23 mg, 95% purity from NMR, 10% yield, 100% stereopure), 71p (44 mg, 95% purity from NMR, 19% yield, 100% stereopure) and 71q (60 mg, 95% purity from NMR, 25% yield, 97.2% stereopure) as yellow solids.

Compound 71n: Chiral analysis (Column: Chiralcel OJ-H 3 μm 4.6*150 mm; Mobile Phase: Hex:EtOH:DEA=75:25:0.2 at 1.0 mL/min; Temp: 30° C.; Wavelength: 230 nm, R_{T}=6.511 min). ¹H NMR (400 MHz, CDCl₃) δ 8.23 (s, 0.3H), 7.83 (d, J=2.8 Hz, 0.7H), 7.78 (d, J=3.2 Hz, 0.3H), 7.51 (d, J=3.2 Hz, 0.7H), 7.45 (d, J=3.2 Hz, 0.3H), 7.43 (d, J=2.4 Hz, 0.7H), 7.13-7.00 (m, 2H), 6.22 (s, 0.3H), 6.10 (d, J=2.4 Hz, 0.7H), 4.54-4.46 (m, 0.3H), 4.28-4.19 (m, 0.7H), 3.71 (s, 3H), 3.62 (s, 2.1H), 3.59 (s, 0.9H), 3.57-3.50 (m, 1H), 3.27-3.10 (m, 2H), 2.90-2.84 (m, 1H), 2.75-2.57 (m, 6H), 2.17-2.07 (m, 1H), 2.05-1.96 (m, 0.4H), 1.94-1.86 (m, 0.6H).

Compound 71p: Chiral analysis (Column: Chiralcel OJ-H 3 μm 4.6*150 mm; Mobile Phase: Hex:EtOH:DEA=75:25:0.2 at 1.0 mL/min; Temp: 30° C.; Wavelength: 230 nm, R_{T}=9.679 min). ¹H NMR (400 MHz, CDCl₃) δ 8.24 (s, 0.3H), 7.84 (d, J=2.8 Hz, 0.7H), 7.78 (d, J=3.2 Hz, 0.3H), 7.51 (d, J=2.8 Hz, 0.7H), 7.48-7.42 (m, 1H), 7.15-7.00 (m, 2H), 6.20 (s, 0.3H), 6.10 (d, J=2.4 Hz, 0.7H), 4.59-4.48 (m, 0.3H), 4.29-4.19 (m, 0.7H), 3.70 (s, 3H), 3.62 (s, 2H), 3.60 (s, 1H), 3.57-3.48 (m, 1H), 3.21-3.10 (m, 1H), 3.09-2.99 (m, 1H), 2.78-2.56 (m, 7H), 2.32-2.19 (m, 1H), 2.14-1.99 (m, 1H).

### Compound 73: Methyl 4-(2-chloro-3,4-difluorophenyl)-6-(2-(4-methoxy-4-oxobutyl)-4,5,6,7-tetrahydro-2H-indazol-5-yl)-2-(thiazol-2-yl)-1,4-dihydropyrimidine-5-carboxylate

LC-MS (ESI): R_{T}=1.78 min, mass calcd. for C₂₇H₂₆ClF₂N₅O₄S 589.1, m/z found 589.8 [M+H]⁺. ¹H NMR (400 MHz, CDCl₃) δ 8.25 (s, 0.6H), 7.83 (d, J=2.8 Hz, 0.4H), 7.76 (d, J=3.2 Hz, 0.6H), 7.49 (d, J=2.8 Hz, 0.4H), 7.43-7.40 (m, 1H), 7.17-7.01 (m, 3H), 6.21 (d, J=6.8 Hz, 0.6H), 6.08 (t, J=3.2 Hz, 0.4H), 4.42-4.31 (m, 0.6H), 4.15-4.08 (m, 2.4H), 3.69-3.68 (m, 3H), 3.61-3.55 (m, 3H), 3.10-2.61 (m, 4H), 2.37-2.32 (m, 2H), 2.28-2.07 (m, 4H).

Racemic 73 (320 mg, 90% purity, 0.488 mmol) was separated by chiral Prep. HPLC (the first separation condition: Column: Chiralpak IG 5 um 20*250 mm; Mobile Phase: Hex:EtOH:DEA=70:30:0.3 at 15 mL/min; Temp: 30° C.; Wavelength: 230 nm; the second separation condition: Chiralpak IC 5 um 20*250 mm; Mobile Phase: Hex:IPA:DEA=70:30:0.3 at 13 mL/min; Temp: 30° C.; Wavelength: 214 nm) to afford the title compounds 73M (55 mg, 95% purity from HNMR, 18% yield, 100% stereopure), 73N (45 mg, 95% purity from HNMR, 15% yield, 99.2% stereopure), 73P (50 mg, 95% purity from HNMR, 16% yield, 99.8% stereopure), and 73Q (50 mg, 95% purity from HNMR, 16% yield, 99.9% stereopure) as yellow solids.

Compound 73n: LC-MS (ESI): R_{T}=1.65 min, mass calcd. for C₂₇H₂₆ClF₂N₅O₄S 589.1, m/z found 589.8 [M+H]⁺. Chiral analysis (Column: Chiralpak IG 5 μm 4.6*250 mm; Mobile Phase: Hex:EtOH:DEA=70:30:0.2 at 1.0 mL/min; Temp: 30° C.; Wavelength: 254 nm, R_{T}=11.878 min). ¹H NMR (400 MHz, CDCl₃) δ 8.25 (s, 0.6H), 7.82 (d, J=2.8 Hz, 0.4H), 7.76 (d, J=2.8 Hz, 0.6H), 7.48 (d, J=2.8 Hz, 0.4H), 7.43 (d, J=2.8 Hz, 0.6H), 7.39 (s, 0.4H), 7.17-7.01 (m, 3H), 6.22 (s, 0.6H), 6.09 (s, 0.4H), 4.41-4.32 (m, 0.6H), 4.15-4.06 (m, 2.4H), 3.69 (s, 3H), 3.62 (s, 1H), 3.59 (s, 2H), 3.13-3.01 (m, 1H), 2.92-2.71 (m, 3H), 2.36-2.33 (m, 2H), 2.21-2.10 (m, 3H), 2.07-1.91 (m, 1H).

Compound 73p: LC-MS (ESI): R_{T}=1.80 min, mass calcd. for C₂₇H₂₆ClF₂N₅O₄S 589.1, m/z found 589.9 [M+H]⁺. Chiral analysis (Column: Chiralpak IC 5 μm 4.6*250 mm; Mobile Phase: Hex:IPA:DEA=60:40:0.2 at 1.0 mL/min; Temp: 30° C.; Wavelength: 230 nm, R_{T}=9.131 min). ¹H NMR (400 MHz, CDCl₃) δ 8.25 (s, 0.6H), 7.83 (d, J=2.0 Hz, 0.4H), 7.76 (d, J=2.0 Hz, 0.6H), 7.48 (s, 0.4H), 7.45-7.39 (m, 1H), 7.17-7.00 (m, 3H), 6.20 (s, 0.6H), 6.08 (s, 0.4H), 4.43-4.33 (m, 0.6H), 4.17-4.05 (m, 2.4H), 3.69 (s, 3H), 3.61 (s, 0.9H), 3.59 (s, 2.1H), 3.01-2.82 (m, 3H), 2.71-2.61 (m, 1H), 2.36-2.33 (m, 2.2H), 2.26-2.05 (m, 3.8H).

### Compound 75: Methyl 4-(3,4-difluoro-2-methylphenyl)-6-(2-(3-methoxy-3-oxopropyl)-4,5,6,7-tetrahydro-2H-indazol-5-yl)-2-(thiazol-2-yl)-1,4-dihydropyrimidine-5-carboxylate

LC-MS (ESI): R_{T}=1.973 min, mass calcd. for C₂₇H₂₇F₂N₅O₄S 555.2, m/z found 556.1 [M+H]⁺. ¹H NMR (400 MHz, CDCl₃) δ 8.19 (s, 1H), 7.81-7.80 (m, 0.2H), 7.75-7.74 (m, 0.8H), 7.50-7.48 (m, 0.2H), 7.42-7.41 (m, 0.8H), 7.22-7.16 (m, 1H), 7.05-6.88 (m, 2H), 5.97 (s, 0.4H), 5.95 (s, 0.4H), 5.89-5.87 (m, 0.2H), 4.43-4.32 (m, 3H), 3.71 (s, 3H), 3.60 (s, 1.5H), 3.59 (s, 1.5H), 3.10-3.04 (m, 0.5H), 2.98-2.65 (m, 5.5H), 2.63-2.58 (m, 2.5H), 2.45-2.44 (m, 0.5H), 2.30-2.21 (m, 0.5H), 2.17-1.94 (m, 1.5H).

Racemic compound 75 (450 mg, 0.802 mmol, 99% purity) was separated by chiral Prep. HPLC (separation condition: Column: Chiralpak IG 5 μm 20*250 mm; Mobile Phase: Hex:EtOH:DEA=60:40:0.3 at 15 mL/min; Temp: 30° C.; Wavelength: 230 nm) to give the fraction A and the fraction B. The fraction B was separated by chiral Prep. HPLC (separation condition: separation condition: (Column: Chiralpak IA 5 μm 20*250 mm; Mobile Phase: Hex:EtOH=60:40 at 22 mL/min; Temp: 30° C.; Wavelength: 214 nm) to give the title compound 75p (80 mg, 97.8% purity, 17.6% yield, 100% stereopure) as yellow solids and the title compound 75q (75 mg, 100% purity, 16.8% yield, 100% stereopure) as yellow solids.

Intermediate 75p: LC-MS (ESI): R_{T}=1.878 min, mass calcd. for C₂₇H₂₇F₂N₅O₄S 555.2, m/z found 556.1 [M+H]⁺. Chiral analysis (Column: Chiralpak IA 5 μm 4.6*250 mm; Mobile Phase: CO₂:EtOH=75:25 at 3.0 g/min; Temp: 40° C.; Wavelength: 230 nm; R_{T}=4.68 min). ¹H NMR (400 MHz, CDCl₃) δ 8.20 (s, 1H), 7.80 (d, J=2.8 Hz, 0.2H), 7.74 (d, J=3.2 Hz, 0.8H), 7.49 (d, J=2.8 Hz, 0.2H), 7.41 (d, J=2.8 Hz, 0.8H), 7.22 (s, 0.8H), 7.14 (s, 0.2H), 7.06-6.94 (m, 1H), 6.92-6.88 (m, 1H), 5.95 (s, 0.8H), 5.88 (s, 0.2H), 4.42-4.33 (m, 2.8H), 4.01-3.94 (m, 0.2H), 3.71 (s, 3H), 3.59 (s, 3H), 2.99-2.79 (m, 5H), 2.69-2.62 (m, 1H), 2.58 (d, J=2.0 Hz, 2.5H), 2.45 (s, 0.5H), 2.33-2.22 (m, 1H), 2.15-2.01 (m, 1H).

### Compound 77: Methyl 6-(6-(2-chloro-3,4-difluorophenyl)-5-(methoxycarbonyl)-2-(thiazol-2-yl)-3,6-dihydropyrimidin-4-yl)-4,5,6,7-tetrahydrobenzo[d]oxazole-2-carboxylate

LC-MS (ESI): R_{T}=1.70 min, mass calcd. for C₂₄H₁₉ClF₂N₄O₅S 548.1, m/z found 548.8 [M+H]⁺. ¹H NMR (400 MHz, DMSO-d₆) δ 9.76-9.73 (m, 0.8H), 9.55 (s, 0.1H), 9.50 (s, 0.1H), 8.02-7.94 (m, 2H), 7.53-7.43 (m, 1H), 7.30-7.20 (m, 1H), 6.06 (s, 0.1H), 6.04 (s, 0.1H), 5.98-5.96 (m, 0.8H), 4.40-4.32 (m, 0.2H), 4.18-4.11 (m, 0.8H), 3.89 (s, 3H), 3.53-3.51 (m, 3H), 3.26-3.00 (m, 1H), 2.96-2.61 (m, 3H), 2.29-1.87 (m, 2H).

Racemic compound 77 (350 mg, 90% purity, 0.574 mmol) was separated by chiral prep. HPLC (Column: Chiralpak IA 5 μm 20*250 mm; Mobile Phase: Hex:IPA:DEA=80:20:0.3 at 25 mL/min; Temp: 30° C.; Wavelength: 214 nm) to give fraction A (150 mg, 100% stereopure) and fraction B (120 mg, 100% stereopure). Then fraction A was separated by chiral Prep. HPLC (Column: Chiralpak IE 5 μm 20*250 mm; Mobile Phase: Hex:EtOH=50:50 at 10 mL/min; Temp: 30° C.; Wavelength: 214 nm) to give 77a (65 mg, 90% purity from NMR, 19% yield, 100% stereopure, including 4% of ethyl ester exchanged product) as yellow solids and 77c (35 mg, 90% purity from NMR, 10% yield, 100% stereopure, including 8% of ethyl ester exchanged product) as yellow solids.

Compound 77c: LC-MS (ESI): R_{T}=1.47 min, mass calcd. for C₂₄H₁₉ClF₂N₄O₅S 548.1, m/z found 548.8 [M+H]⁺. Chiral analysis (Column: Chiralpak IE 5 μm 4.6*250 mm; Mobile Phase: Hex:EtOH=50:50 at 1.0 mL/min; Temp: 30° C.; Wavelength: 230 nm, R_{T}=13.639 min). ¹H NMR (400 MHz, DMSO-d₆) δ 9.75 (s, 0.8H), 9.55 (s, 0.2H), 8.02-7.94 (m, 2H), 7.51-7.43 (m, 1H), 7.30-7.20 (m, 1H), 6.06 (s, 0.2H), 5.97 (s, 0.8H), 4.41-4.33 (m, 0.2H), 4.22-4.13 (m, 0.8H), 3.89 (s, 3H), 3.53 (s, 2.4H), 3.51 (s, 0.6H), 3.26-3.19 (m, 0.8H), 3.04-2.99 (m, 0.2H), 2.95-2.89 (m, 1H), 2.74-2.61 (m, 2H), 2.31-2.24 (m, 0.2H), 2.07-1.89 (m, 1.8H).

### Compound 79: methyl 4-(4-fluoro-2-methylphenyl)-6-(2-(3-methoxy-3-oxopropyl)-4,5,6,7-tetrahydro-2H-indazol-5-yl)-2-(thiazol-2-yl)-1,4-dihydropyrimidine-5-carboxylate

LC-MS (ESI): R_{T}=1.67 min, mass calcd. for C₂₇H₂₈FN₅O₄S 537.2, m/z found 538.0 [M+H]⁺. ¹H NMR (400 MHz, DMSO-d₆) δ 9.55-9.52 (m, 0.8H), 9.03 (s, 0.1H), 8.98 (s, 0.1H), 7.99-7.90 (m, 2H), 7.45 (d, J=3.6 Hz, 0.2H), 7.39 (d, J=11.2 Hz, 0.8H), 7.35-7.23 (m, 0.8H), 7.17-7.07 (m, 0.2H), 7.02-6.96 (m, 2H), 5.84 (d, J=6.8 Hz, 0.2H), 5.76-5.69 (m, 0.8H), 4.28-4.13 (m, 2.2H), 3.92-3.85 (m, 0.8H), 3.62 (s, 3H), 3.50 (s, 3H), 2.95-2.57 (m, 6H), 2.51 (s, 3H), 2.16-1.91 (m, 1.7H), 1.81-1.78 (m, 0.3H). Racemic Compound 79 (400 mg, 0.744 mmol) was separated by chiral prep. HPLC (the first separation condition: Column: Chiralpak IA 5 μm 20*250 mm; Mobile Phase: Hex:EtOH:DEA=70:30:0.3 at 22 mL/min; Temp: 30° C.; Wavelength: 214 nm; the second separation condition: Column: Chiralpak IG 5 μm 20*250 mm; Mobile Phase: Hex:EtOH:DEA=60:40:0.3 at 15 mL/min; Temp: 30° C.; Wavelength: 214 nm) to give 79m (85 mg, 95% purity from NMR, 20% yield, 100% stereopure), 79n (35 mg, 95% purity from NMR, 8% yield, 100% stereopure), 79p (50 mg, 95% purity from NMR, 12% yield, 99.8% stereopure) and 79q (65 mg, 95% purity from NMR, 15% yield, 99.8% stereopure) as yellow solids.

Compound 79p: LC-MS (ESI): R_{T}=1.72 min, mass calcd. for C₂₇H₂₈FN₅O₄S 537.2, m/z found 538.0 [M+H]⁺. Chiral analysis (Column: Chiralpak IG 5 μm 4.6*250 mm; Mobile Phase: Hex:EtOH:DEA=60:40:0.2 at 1.0 mL/min; Temp: 30° C.; Wavelength: 230 nm, R_{T}=13.811 min). ¹H NMR (400 MHz, DMSO-d₆) δ 9.54 (d, J=3.2 Hz, 0.8H), 8.97 (s, 0.2H), 7.99-7.97 (m, 1.6H), 7.94 (d, J=3.2 Hz, 0.2H), 7.91 (d, J=3.2 Hz, 0.2H), 7.45 (s, 0.2H), 7.37 (s, 0.8H), 7.35-7.31 (m, 0.8H), 7.23-7.19 (m, 0.2H), 7.07-6.98 (m, 2H), 5.83 (s, 0.2H), 5.69 (d, J=3.2 Hz, 0.8H), 4.28-4.22 (m, 2H), 3.93-3.85 (m, 1H), 3.61 (s, 3H), 3.50 (s, 3H), 2.86-2.55 (m, 6H), 2.52 (s, 3H), 2.16-1.96 (m, 2H).

### Compound 81: Methyl 4-(2-chloro-3,4-difluorophenyl)-6-(2-(4-methoxy-2-methyl-4-oxobutan-2-yl)-4,5,6,7-tetrahydro-2H-indazol-5-yl)-2-(thiazol-2-yl)-1,4-dihydropyrimidine-5-carboxylate

LC-MS (ESI): R_{T}=4.558 min, mass calcd. for C₂₈H₂₈ClF₂N₅O₄S 603.2, m/z found 604.1 [M+H]⁺. ¹H NMR (400 MHz, CDCl₃) δ 8.26 (s, 0.6H), 7.83-7.82 (m, 0.4H), 7.75-7.74 (m, 0.6H), 7.50-7.48 (m, 0.4H), 7.43-7.39 (m, 1H), 7.34 (s, 0.3H), 7.33 (s, 0.3H), 7.30 (s, 0.4H), 7.17-7.00 (m, 2H), 6.21 (s, 0.3H), 6.20 (s, 0.3H), 6.09-6.08 (m, 0.4H), 4.43-4.32 (m, 0.6H), 4.13-4.05 (m, 0.4H), 3.63 (s, 3H), 3.61 (s, 1H), 3.59 (s, 1H), 3.58 (s, 1H), 3.15-2.60 (m, 6H), 2.35-1.87 (m, 2H), 1.71 (s, 3.6H), 1.70 (s, 2.4H).

Racemic compound 81 (900 mg, 1.48 mmol, 99.2% purity) was separated by chiral Prep. HPLC (the first separation condition: (Column: Chiralpak IA 5 μm 20*250 mm; Mobile Phase: Hex:EtOH:DEA=90:10:0.3 at 15 mL/min; Temp: 30° C.; Wavelength: 230 nm. the second separation condition: (Column: Chiralpak IG 5 μm 20*250 mm; Mobile Phase: Hex:EtOH:DEA=85:15:0.3 at 15 mL/min; Temp: 30° C.; Wavelength: 214 nm)) to give the title compound 81m (190 mg, 98% purity, 21% yield, 100% stereopure), 81n (170 mg, 98% purity, 19% yield, 99.8% stereopure), 81p (180 mg, 98.2% purity, 20% yield, 100% stereopure) and 81q (140 mg, 99% purity, 16% yield, 98.9% stereopure) as yellow solids.

Compound 81n: LC-MS (ESI): R_{T}=2.161 min, mass calcd. for C₂₈H₂₈ClF₂N₅O₄S 603.2, m/z found 604.1 [M+H]⁺. Chiral analysis (Column: Chiralpak IG 5 μm 4.6*250 mm; Mobile Phase: Hex:EtOH:DEA=85:15:0.2 at 1 mL/min; Temp: 30° C.; Wavelength: 280 nm; R_{T}=12.287 min). ¹H NMR (400 MHz, CDCl₃) δ 8.26 (s, 0.6H), 7.82 (d, J=3.2 Hz, 0.4H), 7.74 (d, J=3.2 Hz, 0.6H), 7.49 (d, J=3.2 Hz, 0.4H), 7.43-7.41 (m, 1H), 7.34 (s, 0.6H), 7.30 (s, 0.4H), 7.13-7.00 (m, 2H), 6.21 (s, 0.6H), 6.09 (d, J=2.8 Hz, 0.4H), 4.41-4.33 (m, 0.6H), 4.13-4.04 (m, 0.4H), 3.63 (s, 3H), 3.61 (s, 1H), 3.59 (s, 2H), 3.15-3.00 (m, 1H), 2.92 (s, 2H), 2.90-2.71 (m, 3H), 2.19-1.89 (m, 2H), 1.71 (s, 6H).

Compound 81p: LC-MS (ESI): R_{T}=2.131 min, mass calcd. for C₂₈H₂₈ClF₂N₅O₄S 603.2, m/z found 604.1 [M+H]⁺. Chiral analysis (Column: Chiralpak IA 5 μm 4.6*250 mm; Mobile Phase: Hex:EtOH:DEA=90:10:0.2 at 1 mL/min; Temp: 30° C.; Wavelength: 230 nm; R_{T}=11.142 min). ¹H NMR (400 MHz, CDCl₃) δ 8.26 (s, 0.6H), 7.83 (d, J=3.2 Hz, 0.4H), 7.75 (d, J=3.2 Hz, 0.6H), 7.49 (d, J=3.2 Hz, 0.4H), 7.43-7.41 (m, 1H), 7.32 (s, 1H), 7.15-7.01 (m, 2H), 6.20 (s, 0.6H), 6.08 (d, J=2.8 Hz, 0.4H), 4.42-4.35 (m, 0.6H), 4.13-4.04 (m, 0.4H), 3.63 (s, 3H), 3.61 (s, 1H), 3.59 (s, 2H), 2.99-2.83 (m, 5H), 2.73-2.58 (m, 1H), 2.36-2.03 (m, 2H), 1.70 (s, 6H).

### Compound 83: methyl 4-(2-chloro-3,4-difluorophenyl)-6-(2-(3-ethoxy-2,2-dimethyl-3-oxopropyl)-4,5,6,7-tetrahydro-2H-indazol-5-yl)-2-(thiazol-2-yl)-1,4-dihydropyrimidine-5-carboxylate

LC-MS (ESI): R_{T}=2.29 min, mass calcd. for C₂₉H₃₀ClF₂N₅O₄S 617.2, m/z found 617.8 [M+H]⁺. ¹H NMR (400 MHz, CDCl₃) δ 8.24 (s, 0.6H), 7.83 (d, J=2.8 Hz, 0.4H), 7.76 (d, J=3.6 Hz, 0.6H), 7.49 (d, J=3.2 Hz, 0.4H), 7.44-7.41 (m, 1H), 7.17-7.00 (m, 3H), 6.22 (s, 0.3H), 6.20 (s, 0.3H), 6.09-6.07 (m, 0.4H), 4.37-4.08 (m, 5H), 3.61-3.59 (m, 3H), 3.08-2.60 (m, 4H), 2.32-1.92 (m, 2H), 1.31-1.26 (m, 3H), 1.23 (s, 6H).

Compound 83 (570 mg, 95% purity, 0.876 mmol) was separated by chiral Prep. HPLC (the separation condition: Column: Chiralpak IG 5 μm 20*250 mm; Mobile Phase: Hex:EtOH:DEA=70:30:0.3 at 25 mL/min; Temp: 30° C.; Wavelength: 230 nm) to give Fractions I and Fractions II. Fractions II was separated by chiral Prep. HPLC (the separation condition: Column: Chiralpak IE 5 μm 20*250 mm; Mobile Phase: Hex:EtOH:DEA=70:30:0.3 at 15 mL/min; Temp: 35° C.; Wavelength: 230 nm) to give the title compound 83p (112 mg, 95% purity from NMR, 20% yield, 100% stereopure) and compound 83q (80 mg, 95% purity from NMR, 14% yield, 99.6% stereopure) as yellow solids.

Compound 83q: LC-MS (ESI): R_{T}=1.95 min, mass calcd. for C₂₉H₃₀ClF₂N₅O₄S 617.2, m/z found 617.8 [M+H]⁺. Chiral analysis (Column: Chiralpak IE 5 μm 4.6*250 mm; Mobile Phase: Hex:EtOH:DEA=70:30:0.2 at 1 mL/min; Temp: 30° C.; Wavelength: 230 nm, R_{T}=11.851 min). ¹H NMR (400 MHz, CDCl₃) δ 8.25 (s, 0.6H), 7.83 (d, J=2.8 Hz, 0.4H), 7.76 (d, J=3.2 Hz, 0.6H), 7.49 (d, J=2.8 Hz, 0.4H), 7.43 (d, J=3.2 Hz, 0.6H), 7.40 (s, 0.4H), 7.15-7.00 (m, 3H), 6.20 (s, 0.6H), 6.08 (d, J=3.2 Hz, 0.4H), 4.42-4.35 (m, 0.6H), 4.26-4.15 (m, 4H), 4.12-4.05 (m, 0.4H), 3.61 (s, 1H), 3.59 (s, 2H), 2.95-2.77 (m, 3H), 2.70-2.58 (m, 1H), 2.35-2.03 (m, 2H), 1.28 (t, J=7.2 Hz, 3H), 1.23 (s, 2H), 1.22 (s, 4H).

### Compound 85: (trans)-4-(6-(6-(2-chloro-3,4-difluorophenyl)-5-(methoxycarbonyl)-2-(thiazol-2-yl)-3,6-dihydropyrimidin-4-yl)-4,5,6,7-tetrahydrobenzo[d]oxazol-2-yl)cyclohexanecarboxylic acid

LC-MS (ESI): R_{T}=4.203 min, mass calcd. for C₂₉H₂₇ClF₂N₄O₅S 616.1, m/z found 616.9 [M+H]⁺. ¹H NMR (400 MHz, DMSO-d₆) δ 11.94 (br s, 1H), 9.67-9.65 (m, 0.8H), 9.35 (s, 0.1H), 9.29 (s, 0.1H), 8.01-7.92 (m, 2H), 7.49-7.45 (m, 1H), 7.27-7.22 (m, 1H), 6.05-6.03 (m, 0.2H), 5.96 (s, 0.8H), 4.33-4.28 (m, 0.2H), 4.11-4.04 (m, 0.8H), 3.52 (s, 3H), 3.11-3.04 (m, 0.3H), 2.96-2.89 (m, 0.7H), 2.80-2.68 (m, 2H), 2.63-2.56 (m, 2H), 2.27-2.21 (m, 1H), 2.07-1.96 (m, 6H), 1.52-1.42 (m, 4H).

Racemic Compound 85 (610 mg, 0.967 mmol) was separated by chiral Prep. HPLC (the first separation condition: Column: Chiralpak IG 5 um 20*250 mm; Mobile Phase: Hex:EtOH:DEA=60:40:0.3 at 14 mL/min; Temp: 30° C.; Wavelength: 214 nm; the second separation condition: Column: Chiralpak IA 5 um 20*250 mm; Mobile Phase: Hex:IPA:DEA=70:30:0.3 at 10 mL/min; Temp: 30° C.; Wavelength: 214 nm) to give the title Compound 85p (100 mg, 95% purity from HNMR, 100% stereopure) as yellow solids, Compound 85q (80 mg, 95% purity from HNMR, 99.7% stereopure) as yellow solids, Compound 85x (140 mg, 95% purity from HNMR, 100% stereopure) as yellow solids, and Compound 85y (150 mg, 95% purity from HNMR, 99.3% stereopure) as yellow solids.

Compound 85q: LC-MS (ESI): R_{T}=1.83 min, mass calcd. for C₃₀H₂₉ClF₂N₄O₅S 630.2, m/z found 630.8 [M+H]⁺. Chiral analysis (Column: Chiralpak IG 5 um 4.6*250 mm; Mobile Phase: Hex:EtOH:DEA=60:40:0.2 at 1 mL/min; Temp: 30° C.; Wavelength: 230 nm, RT=8.814 min). ¹H NMR (400 MHz, CDCl₃) δ 8.21 (s, 0.4H), 7.84 (d, J=2.8 Hz, 0.6H), 7.77 (d, J=2.8 Hz, 0.4H), 7.50 (d, J=2.8 Hz, 0.6H), 7.45-7.42 (m, 1H), 7.09-7.03 (m, 2H), 6.22 (s, 0.4H), 6.10 (d, J=2.4 Hz, 0.6H), 4.53-4.45 (m, 0.4H), 4.26-4.20 (m, 0.6H), 3.69 (s, 3H), 3.61 (s, 2.4H), 3.59 (s, 0.6H), 3.25-3.01 (m, 1H), 2.86-2.62 (m, 4H), 2.40-2.35 (m, 1H), 2.23-2.11 (m, 6H), 1.99-1.88 (m, 1H), 1.67-1.63 (m, 2H), 1.55-1.52 (m, 1H).

Compound 85y: LC-MS (ESI): R_{T}=1.83 min, mass calcd. for C₃₀H₂₉ClF₂N₄O₅S 630.2, m/z found 630.8 [M+H]⁺. Chiral analysis (Column: Chiralpak IG 5 um 4.6*250 mm; Mobile Phase: Hex:EtOH:DEA=60:40:0.2 at 1 mL/min; Temp: 30° C.; Wavelength: 230 nm, RT=10.545 min). ¹H NMR (400 MHz, CDCl₃) δ 8.22 (s, 0.4H), 7.84 (d, J=2.8 Hz, 0.6H), 7.76 (d, J=2.8 Hz, 0.4H), 7.51 (d, J=3.2 Hz, 0.6H), 7.45-7.44 (m, 1H), 7.12-7.03 (m, 2H), 6.20 (s, 0.4H), 6.10 (d, J=2.0 Hz, 0.6H), 4.56-4.50 (m, 0.4H), 4.28-4.21 (m, 0.6H), 3.69 (s, 3H), 3.61 (s, 2.4H), 3.60 (s, 0.6H), 3.07-3.00 (m, 1H), 2.74-2.62 (m, 4H), 2.39-2.11 (m, 8H), 1.66-1.60 (m, 2H), 1.55-1.52 (m, 1H).

### Compound 87: Ethyl 4-(2-chloro-3,4-difluorophenyl)-6-(2-(4-ethoxy-3,3-dimethyl-4-oxobutyl)-4,5,6,7-tetrahydro-2H-indazol-5-yl)-2-(thiazol-2-yl)-1,4-dihydropyrimidine-5-carboxylate

¹H NMR (400 MHz, CDCl₃) δ 8.23 (br s, 0.6H), 7.82 (d, J=2.8 Hz, 0.4H), 7.75 (d, J=3.2 Hz, 0.6H), 7.48-7.47 (m, 0.4H), 7.42 (d, J=3.2 Hz, 0.6H), 7.34 (br s, 0.4H), 7.16-7.03 (m, 3H), 6.22 (d, J=6.4 Hz, 0.6H), 6.10 (t, J=2.0 Hz, 0.4H), 4.43-4.32 (m, 0.6H), 4.17-4.02 (m, 6.4H), 3.06-2.66 (m, 4H), 2.22-1.96 (m, 4H), 1.29-1.23 (m, 9H), 1.11 (t, J=7.2 Hz, 3H).

Racemic Compound 87 (640 mg, 90% purity, 0.891 mmol) was separated by chiral Prep. HPLC (Column: Chiralpak IE 5 μm 20*250 mm; Mobile Phase: Hex:EtOH:DEA=60:40:0.3 at 12 mL/min; Temp: 30° C.; Wavelength: 214 nm) to afford compound 87q (140 mg, 90% purity from ¹H NMR, 22% yield, 99.6% stereopure) and other 3 diastereomers (400 mg, 63% yield) as yellow solids.

Compound 87q: LC-MS (ESI): R_{T}=2.127 min, mass calcd. For C₃₁H₃₄ClF₂N₅O₄S 645.2, m/z found 646.1 [M+H]⁺. Chiral analysis (Column: Chiralpak IE 5 μm 4.6*250 mm; Mobile Phase: Hex:EtOH:DEA=60:40:0.2 at 1 mL/min; Temp: 30° C.; Wavelength: 254 nm, R_{T}=9.365 min). ¹H NMR (400 MHz, CDCl₃) δ 8.23 (br s, 0.6H), 7.82 (d, J=3.2 Hz, 0.4H), 7.75 (d, J=2.8 Hz, 0.6H), 7.48 (d, J=3.2 Hz, 0.4H), 7.43 (d, J=3.2 Hz, 0.6H), 7.33 (br s, 0.4H), 7.16-7.03 (m, 3H), 6.22 (s, 0.6H), 6.10 (d, J=2.8 Hz, 0.4H), 4.43-4.36 (m, 0.6H), 4.17-4.12 (m, 2H), 4.09-3.99 (m, 4.4H), 2.96-2.83 (m, 3H), 2.70-2.59 (m, 1H), 2.32-2.22 (m, 1H), 2.17-2.03 (m, 3H), 1.29-1.25 (m, 9H), 1.11 (t, J=7.2 Hz, 3H).

### Compound 89: Methyl 4-(2-chloro-3,4-difluorophenyl)-6-(2-(4-ethoxy-3,3-dimethyl-4-oxobutyl)-4,5,6,7-tetrahydro-2H-indazol-5-yl)-2-(thiazol-2-yl)-1,4-dihydropyrimidine-5-carboxylate

¹H NMR (400 MHz, CDCl₃) 8.25 (br s, 0.6H), 7.83-7.81 (m, 0.4H), 7.75 (d, J=3.2 Hz, 0.6H), 7.49-7.47 (m, 0.4H), 7.42 (d, J=3.2 Hz, 0.6H), 7.40 (br s, 0.4H), 7.16-7.02 (m, 3H), 6.21 (d, J=7.2 Hz, 0.6H), 6.09-6.07 (m, 0.4H), 4.40-4.32 (m, 0.6H), 4.17-4.11 (m, 2H), 4.09-4.04 (m, 2.4H), 3.61 (s, 1.2H), 3.58 (d, J=2.8 Hz, 1.8H), 3.07-2.66 (m, 4H), 2.22-1.99 (m, 4H), 1.29-1.25 (m, 9H). Racemic Compound 89 (640 mg, 90% purity, 0.911 mmol) was separated by chiral Prep. HPLC (separation condition: Column: Chiralpak IA 5 μm 20*250 mm; Mobile Phase: Hex:EtOH:DEA=60:40:0.3 at 12 mL/min; Temp: 30° C.; Wavelength: 214 nm) to afford fraction 1 and fraction 2.

Fraction 2 (240 mg, 90% purity, 0.342 mmol) was further separated by chiral Prep. HPLC (separation condition: Column: Chiralpak IB 5 μm 20*250 mm; Mobile Phase: Hex:EtOH:DEA=90:10:0.3 at 15 mL/min; Temp: 30° C.; Wavelength: 230 nm) to afford the title compound 89p (100 mg, 90% purity from ¹H NMR, 42% yield, 100% stereopure) and the title compound 89q (100 mg, 90% purity from ¹H NMR, 42% yield, 99.8% stereopure) as yellow solids.

Compound 89p: LC-MS (ESI): R_{T}=2.079 min, mass calcd. for C₃₀H₃₂ClF₂N₅O₄S 631.2, m/z found 632.1 [M+H]⁺. Chiral analysis (Column: Chiralpak IB 5 μm 4.6*250 mm; Mobile Phase: Hex:EtOH:DEA=90:10:0.2 at 1 mL/min; Temp: 30° C.; Wavelength: 254 nm, R_{T}=8.491 min). ¹H NMR (400 MHz, CDCl₃) 8.25 (br s, 0.6H), 7.82 (d, J=2.8 Hz, 0.4H), 7.75 (d, J=3.2 Hz, 0.6H), 7.48 (d, J=2.8 Hz, 0.4H), 7.43 (d, J=3.2 Hz, 0.6H), 7.40 (br s, 0.4H), 7.16-7.02 (m, 3H), 6.20 (s, 0.6H), 6.08 (d, J=2.4 Hz, 0.4H), 4.41-4.34 (m, 0.6H), 4.17-4.11 (m, 2H), 4.09-4.03 (m, 2.4H), 3.61 (s, 1.2H), 3.59 (s, 1.8H), 2.96-2.79 (m, 3H), 2.70-2.60 (m, 1H), 2.33-2.22 (m, 1H), 2.14-2.06 (m, 3H), 1.29-1.25 (m, 9H).

### Compound 91: Ethyl 4-(3,4-difluoro-2-methylphenyl)-6-(2-(3-ethoxy-2,2-dimethyl-3-oxopropyl)-4,5,6,7-tetrahydro-2H-indazol-5-yl)-2-(thiazol-2-yl)-1,4-dihydropyrimidine-5-carboxylate

LC-MS (ESI): R_{T}=1.75 min, mass calcd. for C₃₁H₃₅F₂N₅O₄S 611.2, m/z found 612.0 [M+H]⁺. ¹H NMR (400 MHz, DMSO-d₆) δ 8.18 (s, 0.5H), 8.16 (s, 0.5H), 7.80 (d, J=2.8 Hz, 0.2H), 7.74 (d, J=3.2 Hz, 0.8H), 7.49 (d, J=6.0 Hz, 0.2H), 7.41 (d, J=2.8 Hz, 0.8H), 7.17-7.07 (m, 1H), 7.03-6.88 (m, 2H), 5.98 (s, 0.4H), 5.96 (s, 0.4H), 5.89 (s, 0.2H), 4.44-4.34 (m, 0.8H), 4.23-4.15 (m, 4.2H), 4.09-3.94 (m, 2H), 3.10-2.63 (m, 4H), 2.58 (s, 2.5H), 2.45 (s, 0.5H), 2.27-1.96 (m, 2H), 1.30-1.26 (m, 3H), 1.23-1.22 (m, 6H), 1.11 (t, J=6.8 Hz, 3H).

Racemic Compound 91 (470 mg, 0.73 mmol) was separated by chiral Prep. HPLC (Column: Chiralpak IG 5 um 20*250 mm; Mobile Phase: CO₂:IPA:DEA=70:30:0.3 at 50 g/min; Col. Temp: 40° C.; Wavelength: 214 nm, Back pressure: 100 bar) to give Compound 91c (110 mg, 90% purity from NMR, 22% yield, 99.5% stereopure), Compound 91d (100 mg, 90% purity from NMR, 20% yield, 100% stereopure) and the other two enantiomers as yellow solids.

Compound 91c: LC-MS (ESI): R_{T}=1.78 min, mass calcd. For C₃₁H₃₅F₂N₅O₄S 611.2, m/z found 611.9 [M+H]⁺. Chiral analysis (Column: Chiralpak IG 5 um 4.6*250 mm; Mobile Phase: CO₂:IPA:DEA=70:30:0.2 at 3 g/min; Col. Temp: 40° C.; Wavelength: 230 nm, Back pressure: 100 bar, R_{T}=3.98 min). ¹H NMR (400 MHz, CDCl₃) δ 8.17 (s, 1H), 7.80 (d, J=3.2 Hz, 0.2H), 7.74 (d, J=3.2 Hz, 0.8H), 7.49 (d, J=2.8 Hz, 0.2H), 7.40 (d, J=2.8 Hz, 0.8H), 7.14 (s, 1H), 7.07-6.88 (m, 2H), 5.96 (s, 0.8H), 5.89 (d, J=2.0 Hz, 0.2H), 4.44-4.37 (m, 0.8H), 4.23-4.14 (m, 4H), 4.09-3.98 (m, 2.2H), 2.98-2.87 (m, 3H), 2.70-2.63 (m, 1H), 2.58 (d, J=2.0 Hz, 2.5H), 2.45 (d, J=2.4 Hz, 0.5H), 2.28-2.22 (m, 1H), 2.15-2.06 (m, 1H), 1.28 (t, J=7.2 Hz, 3H), 1.22 (s, 6H), 1.11 (t, J=7.2 Hz, 3H).

### Compound 93: Ethyl 4-(2-chloro-3,4-difluorophenyl)-2-(3,5-difluoropyridin-2-yl)-6-(2-(3-ethoxy-2,2-dimethyl-3-oxopropyl)-4,5,6,7-tetrahydro-2H-indazol-5-yl)-1,4-dihydropyrimidine-5-carboxylate

LC-MS (ESI): R_{T}=1.88 min, mass calcd. for C₃₂H₃₂ClF₄N₅O₄ 661.2, m/z found 661.8 [M+H]⁺. ¹H NMR (400 MHz, CDCl₃) δ 8.70 (s, 0.4H), 8.69 (s, 0.4H), 8.27-8.21 (m, 1H), 7.78 (d, J=2.0 Hz, 0.2H), 7.32-7.25 (m, 1H), 7.17-7.00 (m, 3H), 6.35 (s, 0.4H), 6.33 (s, 0.4H), 6.09 (d, J=2.8 Hz, 0.2H), 4.46-4.37 (m, 0.8H), 4.25-4.15 (m, 4.2H), 4.10-3.97 (m, 2H), 3.08-2.59 (m, 4H), 2.27-1.94 (m, 2H), 1.31-1.26 (m, 3H), 1.23 (s, 1.2H), 1.22 (s, 4.8H), 1.12 (t, J=6.8 Hz, 3H). Racemic Compound 93 (900 mg, 1.36 mmol) was separated by chiral Prep. HPLC (the first separation condition: Column: Chiralpak ID 5 μm 20*250 mm; Mobile Phase: CO₂:IPA=75:25 at 50 g/min; Temp: 40° C.; Wavelength: 230 nm. the second separation condition: Column: Chiralpak OD 5 um 20*250 mm; Mobile Phase: Hexane:EtOH=95:5 at 60 mL/min; Temp: 35° C.; Wavelength: 254 nm) to afford Compound 93m (147 mg, 95% purity from NMR, 16% yield, 100% stereopure), 9311 (136 mg, 98% purity, 15% yield, 99.4% stereopure), 93p (150 mg, 17% yield, 100% stereopure) and 93q (178 mg, 20% yield, 99.5% stereopure) as yellow solids.

Compound 93p: Chiral analysis (Column: Chiralpak ID 5 μm 4.6*250 mm; Mobile Phase: CO₂:IPA=75:25 at 1 mL/min; Temp: 40° C.; Wavelength: 230 nm; R_{T}=6.2 min). ¹H NMR (400 MHz, CDCl₃) δ 8.68 (s, 0.8H), 8.27-8.22 (m, 1H), 7.78 (d, J=3.2 Hz, 0.2H), 7.32-7.22 (m, 1H), 7.17-7.00 (m, 3H), 6.33 (s, 0.8H), 6.09 (d, J=2.8 Hz, 0.2H), 4.46-4.38 (m, 0.8H), 4.24-4.14 (m, 4.2H), 4.10-3.97 (m, 2H), 2.97-2.82 (m, 3H), 2.67-2.58 (m, 1H), 2.34-2.20 (m, 1H), 2.12-2.02 (m, 1H), 1.30-1.25 (m, 3H), 1.23-1.19 (m, 6H), 1.12 (t, J=7.2 Hz, 3H).

### Compound 95: (cis)-Methyl 4-(2-chloro-3,4-difluorophenyl)-6-(2-(3-(methoxycarbonyl)cyclobutyl)-4,5,6,7-tetrahydro-2H-indazol-5-yl)-2-(thiazol-2-yl)-1,4-dihydropyrimidine-5-carboxylate

LC-MS (ESI): R_{T}=1.85 min, mass calcd. for C₂₈H₂₆ClF₂N₅O₄S 601.1, m/z found 601.8 [M+H]⁺. ¹H NMR (400 MHz, DMSO-d₆) δ 9.66-9.63 (m, 0.7H), 9.12-9.06 (m, 0.3H), 8.00-7.95 (m, 2H), 7.52-7.45 (m, 2H), 7.28-7.21 (m, 1H), 6.06-5.95 (m, 1H), 4.73-4.63 (m, 1H), 4.18-4.10 (m, 0.3H), 3.96-3.85 (m, 0.7H), 3.64 (s, 3H), 3.51 (s, 3H), 3.02-2.88 (m, 1.6H), 2.82-2.58 (m, 7.4H), 2.16-1.94 (m, 1.6H), 1.84-1.79 (m, 0.4H).

Racemic Compound 95 (900 mg, 90% purity, 1.35 mmol) was separated by chiral Prep. HPLC (separation condition: Column: Chiralpak IG 5 um 20*250 mm; Hex:EtOH:DEA=60:40:0.3 at 15 mL/min; Temp: 30° C.; Wavelength: 230 nm) to give fraction 1 (300 mg) and fraction 2 (350 mg). Fraction 2 was separated by chiral Prep. HPLC (separation condition: Column: IE 5 um 20*250 mm; Hex:EtOH:DEA=70:30:0.3, 14 mL/min, 214 nm) to give Compound 95p (120 mg, 95% purity from NMR, 14% yield, 100% stereopure) and 95q (120 mg, 95% purity from NMR, 14% yield, 98.5% stereopure) as yellow solids.

Compound 95q: Chiral analysis (Column: Chiralpak IE 5 um 4.6*250 mm; Hex:EtOH:DEA=70:30:0.2 at 1 mL/min; Temp: 30° C.; Wavelength: 230 nm, R_{T}=14.766 min). ¹H NMR (400 MHz, CDCl₃) δ 8.26 (s, 0.6H), 7.83 (d, J=2.8 Hz, 0.4H), 7.76 (d, J=3.2 Hz, 0.6H), 7.49 (d, J=2.8 Hz, 0.4H), 7.44-7.42 (m, 1H), 7.33 (s, 0.6H), 7.27 (s, 0.4H), 7.17-7.01 (m, 2H), 6.20 (s, 0.6H), 6.09-6.07 (d, J=2.4 Hz, 0.4H), 4.72-4.63 (m, 1H), 4.41-4.34 (m, 0.6H), 4.09-4.06 (m, 0.4H), 3.73 (s, 3H), 3.61 (s, 1.2H), 3.59 (s, 1.8H), 2.99-2.84 (m, 4H), 2.79-2.62 (m, 5H), 2.37-2.08 (m, 2H).

### Part VI: Hydrolysis of Esters

### Compound 34c: (Exemplified with Method SSS) 3-(5-(6-(2-chloro-3,4-difluorophenyl)-5-(ethoxycarbonyl)-2-(thiazol-2-yl)-3,6-dihydropyrimidin-4-yl)-4,5,6,7-tetrahydro-2H-indazol-2-yl)propanoic acid

To a solution of ethyl 4-(2-chloro-3,4-difluorophenyl)-6-(2-(3-methoxy-3-oxopropyl)-4,5,6,7-tetrahydro-2H-indazol-5-yl)-2-(thiazol-2-yl)-1,4-dihydropyrimidine-5-carboxylate Compound 1c (210 mg, 0.345 mmol) in tetrahydrofuran (1.8 mL), ethanol (0.6 mL) and water (0.6 mL) was added lithium hydroxide hydrate (30.5 mg, 0.69 mmol) at 0° C. After stirred at 0° C.; for 2 hours, the mixture was added with water (2 mL) and concentrated at room temperature under reduced pressure to remove volatiles. The residue was acidified with 1 M hydrochloride aqueous solution (1 mL) and purified by C18 column (acetonitrile:water=55% to 58%) to give the title compound (175 mg, 87% yield, 100% stereopure) as yellow solids. LC-MS (ESI): R_{T}=3.395 min, mass calcd. for C₂₆H₂₄ClF₂N₅O₄S 575.1, m/z found 576.2 [M+H]⁺. Chiral HPLC (Column: Chiralpak IA 5 μm 4.6*250 mm; Mobile Phase: Hex:EtOH:TFA=80:20:0.2 at 1.0 mL/min; Temp: 30° C.; Wavelength: 230 nm, R_{T}=8.542 min). ¹H NMR (400 MHz, DMSO-d₆) δ 12.24 (br s, 1H), 9.60 (s, 0.7H), 9.00 (s, 0.3H), 8.00-7.92 (m, 2H), 7.52-7.46 (m, 1H), 7.44 (s, 0.3H), 7.37 (s, 0.7H), 7.29-7.25 (m, 1H), 6.06 (s, 0.3H), 5.95 (s, 0.7H), 4.24-4.12 (m, 2.3H), 4.00-3.88 (m, 2.7H), 2.78-2.53 (m, 6H), 2.25-2.18 (m, 0.3H), 2.13-1.97 (m, 1.7H), 1.06-0.99 (m, 3H).

### Compound 34a: (Converted from Compound 1a)

LC-MS (ESI): R_{T}=3.164 min, mass calcd. for C₂₆H₂₄ClF₂N₅O₄S 575.1, m/z found 576.0 [M+H]⁺. Chiral HPLC (Column: Chiralpak IG 5 μm 4.6*250 mm; Mobile Phase: Hex:EtOH:TFA=80:20:0.2 at 1.0 mL/min; Temp: 30° C.; Wavelength: 254 nm, R_{T}=12.703 min). ¹H NMR (400 MHz, DMSO-d₆) δ 8.00-7.98 (m, 1.3H), 7.97-7.93 (m, 0.7H), 7.52-7.46 (m, 1H), 7.44 (s, 0.3H), 7.39 (s, 0.7H), 7.26-7.21 (m, 1H), 6.07 (s, 0.3H), 5.96 (s, 0.7H), 4.24-4.12 (m, 2.3H), 3.99-3.87 (m, 2.7H), 2.98-2.56 (m, 6H), 2.18-2.08 (m, 0.3H), 2.03-1.92 (m, 1H), 1.83-1.79 (m, 0.7H), 1.04 (t, J=7.2 Hz, 2.1H), 1.00 (t, J=7.2 Hz, 0.9H).

### Compound 34d: (Converted from Compound 1d)

LC-MS (ESI): R_{T}=3.160 min, mass calcd. for C₂₆H₂₄ClF₂N₅O₄S 575.1, m/z found 576.0 [M+H]⁺. Chiral HPLC (Column: Chiralpak IG 5 μm 4.6*250 mm; Mobile Phase: Hex:EtOH:TFA=80:20:0.2 at 1.0 mL/min; Temp: 30° C.; Wavelength: 254 nm, R_{T}=14.096 min). ¹H NMR (400 MHz, DMSO-d₆) δ 8.00-7.98 (m, 1.3H), 7.97-7.93 (m, 0.7H), 7.52-7.46 (m, 1H), 7.44 (s, 0.3H), 7.39 (s, 0.7H), 7.26-7.21 (m, 1H), 6.07 (s, 0.3H), 5.96 (s, 0.7H), 4.24-4.12 (m, 2.3H), 3.99-3.87 (m, 2.7H), 2.97-2.56 (m, 6H), 2.18-2.08 (m, 0.3H), 2.03-1.92 (m, 1H), 1.83-1.79 (m, 0.7H), 1.04 (t, J=7.2 Hz, 2.1H), 1.00 (t, J=7.2 Hz, 0.9H).

Similarly utilizing the above-mentioned analogous procedures of ester hydrolysis, the following acids could be prepared; these are indicated in Table 2 below, thereby related to the corresponding esters, which are listed with reference to their compound numbers (“Cpd.#”).

TABLE 2

| Ester | |
|---|---|
| Cpd. # | Acid |
| 1a 1c 1d | 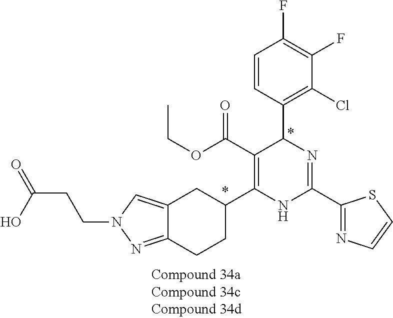 [SMILES: CCOC(=O)C1=C(C2CCc3nn(CCC(=O)O)cc3C2)NC(c2nccs2)=NC1c1ccc(F)c(F)c1Cl] |
| 2b 2c | 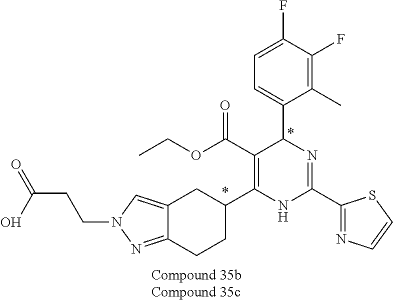 [SMILES: CCOC(=O)C1=C(C2CCc3nn(CCC(=O)O)cc3C2)NC(c2nccs2)=NC1c1ccc(F)c(F)c1C] |
| 3b 3d | 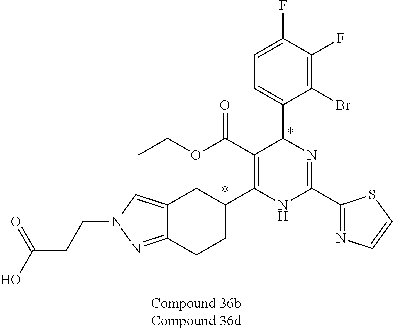 [SMILES: CCOC(=O)C1=C(C2CCc3nn(CCC(=O)O)cc3C2)NC(c2nccs2)=NC1c1ccc(F)c(F)c1Br] |
| 4b 4c 4d | 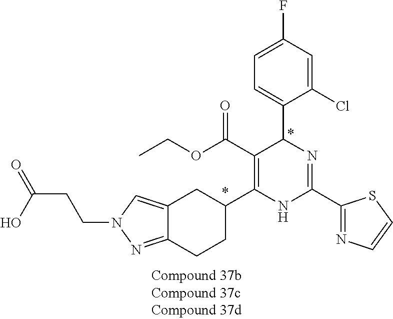 [SMILES: CCOC(=O)C1=C(C2CCc3nn(CCC(=O)O)cc3C2)NC(c2nccs2)=NC1c1ccc(F)cc1Cl] |
| 5a 5b 5d | 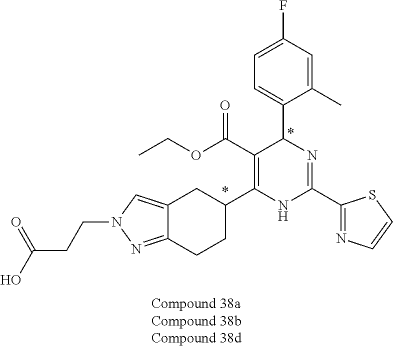 [SMILES: CCOC(=O)C1=C(C2CCc3nn(CCC(=O)O)cc3C2)NC(c2nccs2)=NC1c1ccc(F)cc1C] |
| 6b 6c | 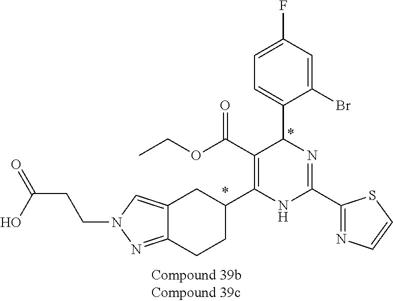 [SMILES: CCOC(=O)C1=C(C2CCc3nn(CCC(=O)O)cc3C2)NC(c2nccs2)=NC1c1ccc(F)cc1Br] |
| 7b 7c | 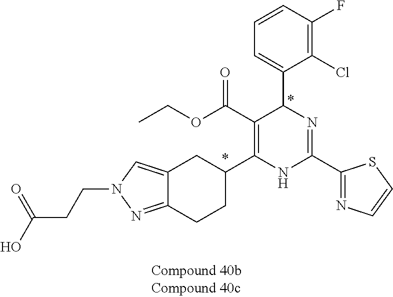 [SMILES: CCOC(=O)C1=C(C2CCc3nn(CCC(=O)O)cc3C2)NC(c2nccs2)=NC1c1cccc(F)c1Cl] |
| 8b 8c | 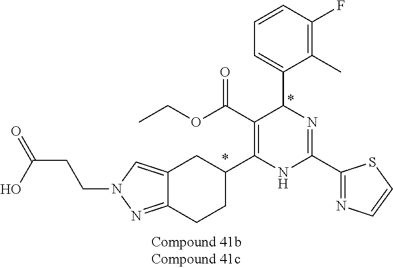 [SMILES: CCOC(=O)C1=C(C2CCc3nn(CCC(=O)O)cc3C2)NC(c2nccs2)=NC1c1cccc(F)c1C] |
| 9b 9d | 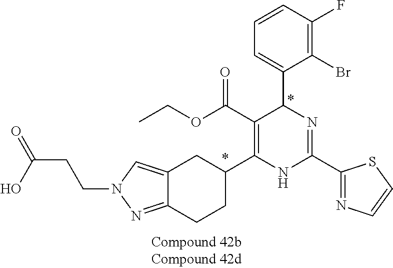 [SMILES: CCOC(=O)C1=C(C2CCc3nn(CCC(=O)O)cc3C2)NC(c2nccs2)=NC1c1cccc(F)c1Br] |
| 10b 10c | 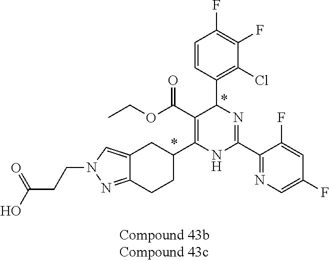 [SMILES: CCOC(=O)C1=C(C2CCc3nn(CCC(=O)O)cc3C2)NC(c2ncc(F)cc2F)=NC1c1ccc(F)c(F)c1Cl] |
| 11b 11c | 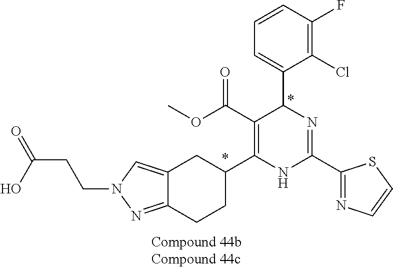 [SMILES: COC(=O)C1=C(C2CCc3nn(CCC(=O)O)cc3C2)NC(c2nccs2)=NC1c1cccc(F)c1Cl] |
| 12b 12c | 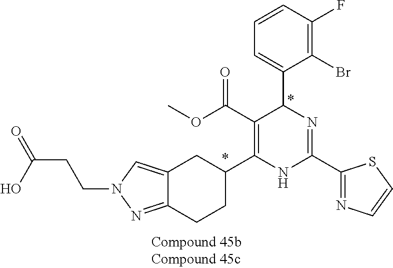 [SMILES: COC(=O)C1=C(C2CCc3nn(CCC(=O)O)cc3C2)NC(c2nccs2)=NC1c1cccc(F)c1Br] |
| 13a 13d | 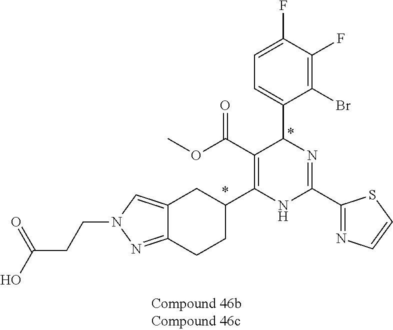 [SMILES: COC(=O)C1=C(C2CCc3nn(CCC(=O)O)cc3C2)NC(c2nccs2)=NC1c1ccc(F)c(F)c1Br] |
| 14b 14c | 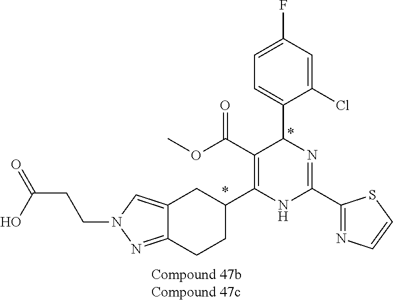 [SMILES: COC(=O)C1=C(C2CCc3nn(CCC(=O)O)cc3C2)NC(c2nccs2)=NC1c1ccc(F)cc1Cl] |
| 15b 15d | 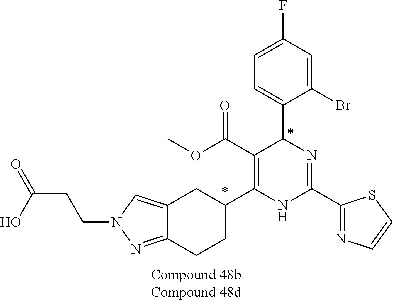 [SMILES: COC(=O)C1=C(C2CCc3nn(CCC(=O)O)cc3C2)NC(c2nccs2)=NC1c1ccc(F)cc1Br] |
| 17b 17c | 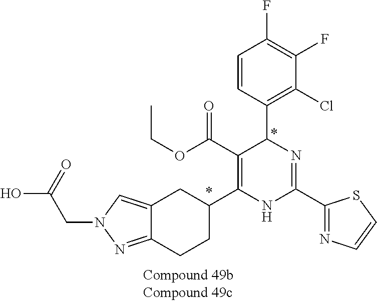 [SMILES: CCOC(=O)C1=C(C2CCc3nn(CC(=O)O)cc3C2)NC(c2nccs2)=NC1c1ccc(F)c(F)c1Cl] |
| 18b 18c | 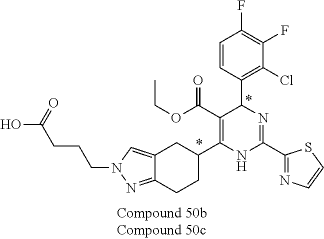 [SMILES: CCOC(=O)C1=C(C2CCc3nn(CCCC(=O)O)cc3C2)NC(c2nccs2)=NC1c1ccc(F)c(F)c1Cl] |
| 19b 19c 20b 20d | 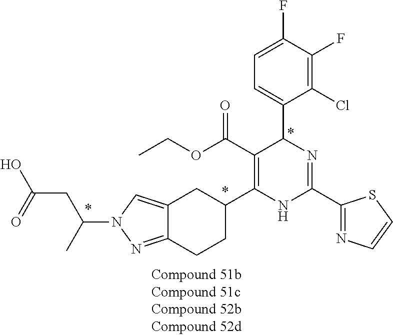 [SMILES: CCOC(=O)C1=C(C2CCc3nn(C(C)CC(=O)O)cc3C2)NC(c2nccs2)=NC1c1ccc(F)c(F)c1Cl] |
| 21b 21c | 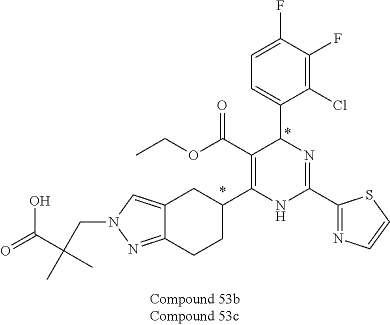 [SMILES: CCOC(=O)C1=C(C2CCc3nn(CC(C)(C)C(=O)O)cc3C2)NC(c2nccs2)=NC1c1ccc(F)c(F)c1Cl] |
| 22 | 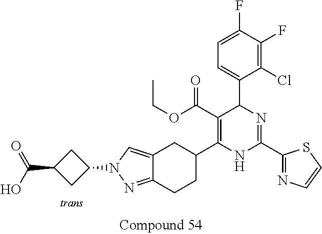 [SMILES: CCOC(=O)C1=C(C2CCc3nn([C@H]4C[C@H](C(=O)O)C4)cc3C2)NC(c2nccs2)=NC1c1ccc(F)c(F)c1Cl] |
| 23a 23c | 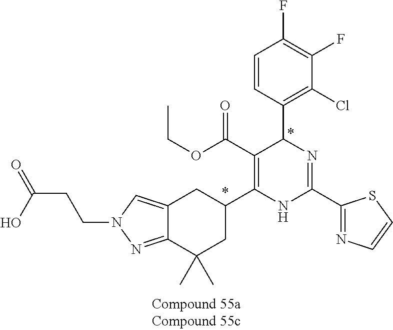 [SMILES: CCOC(=O)C1=C(C2Cc3cn(CCC(=O)O)nc3C(C)(C)C2)NC(c2nccs2)=NC1c1ccc(F)c(F)c1Cl] |
| 24 | 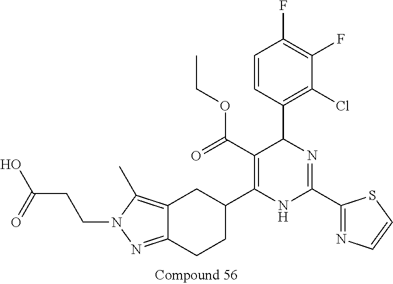 [SMILES: CCOC(=O)C1=C(C2CCc3nn(CCC(=O)O)c(C)c3C2)NC(c2nccs2)=NC1c1ccc(F)c(F)c1Cl] |
| 26c 26f | 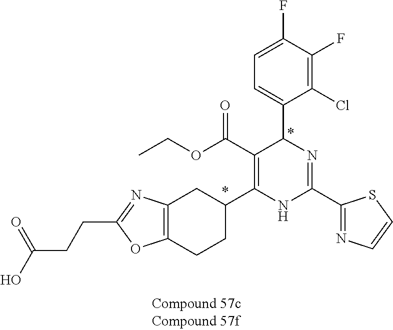 [SMILES: CCOC(=O)C1=C(C2CCc3oc(CCC(=O)O)nc3C2)NC(c2nccs2)=NC1c1ccc(F)c(F)c1Cl] |
| 27b 27d | 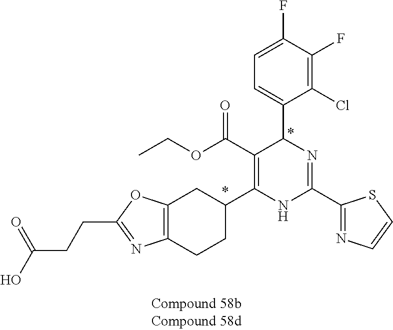 [SMILES: CCOC(=O)C1=C(C2CCc3nc(CCC(=O)O)oc3C2)NC(c2nccs2)=NC1c1ccc(F)c(F)c1Cl] |
| 28c 28f | 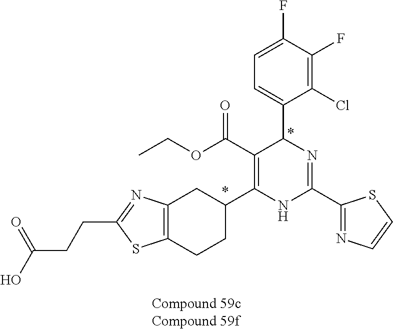 [SMILES: CCOC(=O)C1=C(C2CCc3sc(CCC(=O)O)nc3C2)NC(c2nccs2)=NC1c1ccc(F)c(F)c1Cl] |
| 29b 29c | 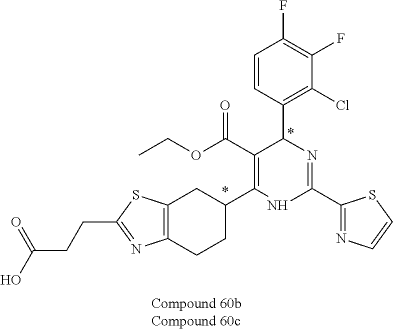 [SMILES: CCOC(=O)C1=C(C2CCc3nc(CCC(=O)O)sc3C2)NC(c2nccs2)=NC1c1ccc(F)c(F)c1Cl] |
| 30a 30c | 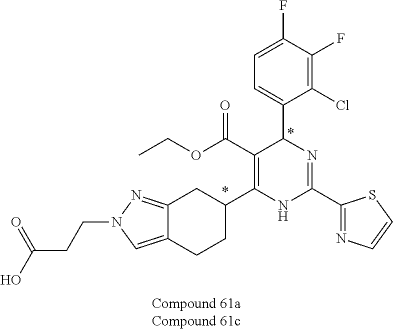 [SMILES: CCOC(=O)C1=C(C2CCc3cn(CCC(=O)O)nc3C2)NC(c2nccs2)=NC1c1ccc(F)c(F)c1Cl] |
| 31 | 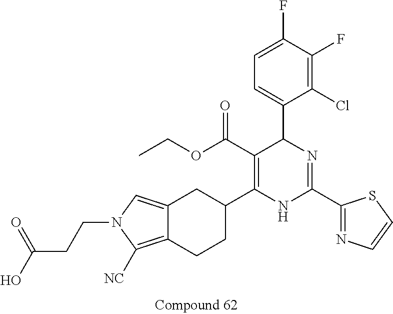 [SMILES: CCOC(=O)C1=C(C2CCc3c(cn(CCC(=O)O)c3C#N)C2)NC(c2nccs2)=NC1c1ccc(F)c(F)c1Cl] |
| 32 | 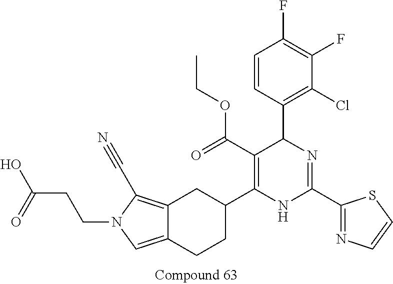 [SMILES: CCOC(=O)C1=C(C2CCc3cn(CCC(=O)O)c(C#N)c3C2)NC(c2nccs2)=NC1c1ccc(F)c(F)c1Cl] |
| 33 | 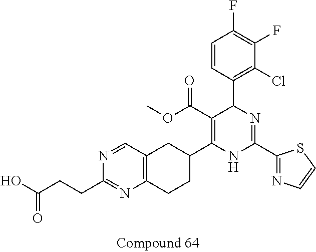 [SMILES: COC(=O)C1=C(C2CCc3nc(CCC(=O)O)ncc3C2)NC(c2nccs2)=NC1c1ccc(F)c(F)c1Cl] |
| 67n 67p | 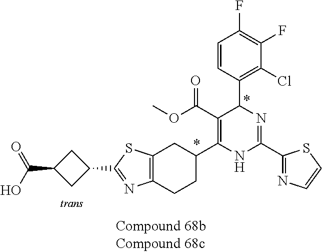 [SMILES: COC(=O)C1=C(C2CCc3nc([C@H]4C[C@H](C(=O)O)C4)sc3C2)NC(c2nccs2)=NC1c1ccc(F)c(F)c1Cl] |
| 69n 69q | 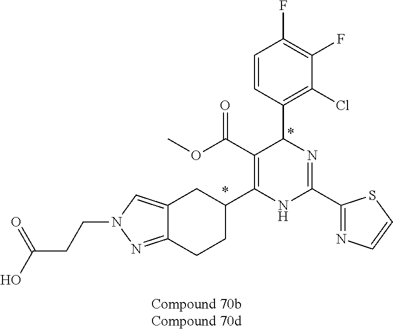 [SMILES: COC(=O)C1=C(C2CCc3nn(CCC(=O)O)cc3C2)NC(c2nccs2)=NC1c1ccc(F)c(F)c1Cl] |
| 71n 71p | 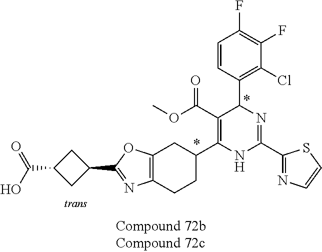 [SMILES: COC(=O)C1=C(C2CCc3nc([C@H]4C[C@H](C(=O)O)C4)oc3C2)NC(c2nccs2)=NC1c1ccc(F)c(F)c1Cl] |
| 73n 73p | 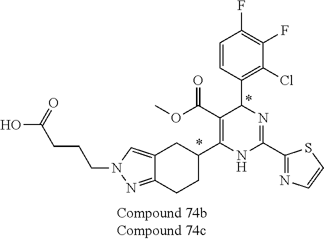 [SMILES: COC(=O)C1=C(C2CCc3nn(CCCC(=O)O)cc3C2)NC(c2nccs2)=NC1c1ccc(F)c(F)c1Cl] |
| 75p | 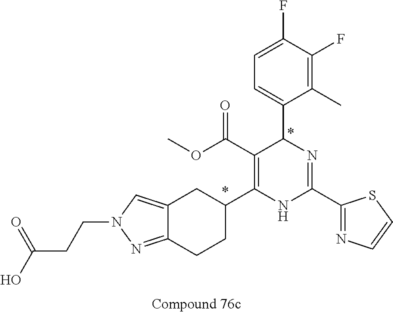 [SMILES: COC(=O)C1=C(C2CCc3nn(CCC(=O)O)cc3C2)NC(c2nccs2)=NC1c1ccc(F)c(F)c1C] |
| 77c | 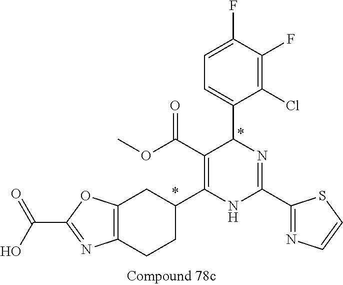 [SMILES: COC(=O)C1=C(C2CCc3nc(C(=O)O)oc3C2)NC(c2nccs2)=NC1c1ccc(F)c(F)c1Cl] |
| 79p | 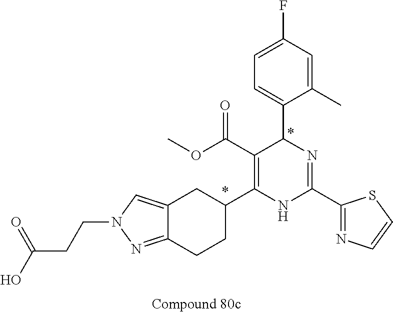 [SMILES: COC(=O)C1=C(C2CCc3nn(CCC(=O)O)cc3C2)NC(c2nccs2)=NC1c1ccc(F)cc1C] |
| 81n 81p | 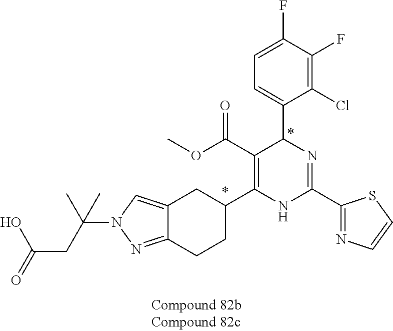 [SMILES: COC(=O)C1=C(C2CCc3nn(C(C)(C)CC(=O)O)cc3C2)NC(c2nccs2)=NC1c1ccc(F)c(F)c1Cl] |
| 83q | 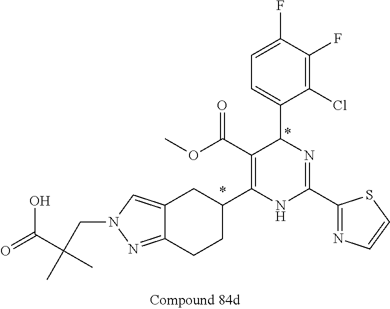 [SMILES: COC(=O)C1=C(C2CCc3nn(CC(C)(C)C(=O)O)cc3C2)NC(c2nccs2)=NC1c1ccc(F)c(F)c1Cl] |
| 85q 85y | 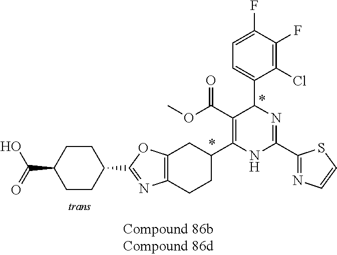 [SMILES: COC(=O)C1=C(C2CCc3nc([C@H]4CC[C@H](C(=O)O)CC4)oc3C2)NC(c2nccs2)=NC1c1ccc(F)c(F)c1Cl] |
| 87q | 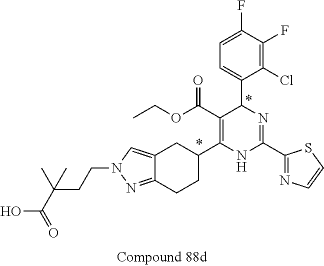 [SMILES: CCOC(=O)C1=C(C2CCc3nn(CCC(C)(C)C(=O)O)cc3C2)NC(c2nccs2)=NC1c1ccc(F)c(F)c1Cl] |
| 89p | 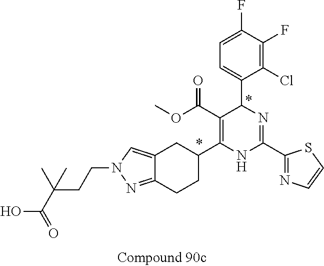 [SMILES: COC(=O)C1=C(C2CCc3nn(CCC(C)(C)C(=O)O)cc3C2)NC(c2nccs2)=NC1c1ccc(F)c(F)c1Cl] |
| 91c | 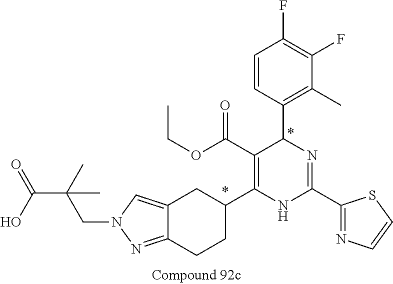 [SMILES: CCOC(=O)C1=C(C2CCc3nn(CC(C)(C)C(=O)O)cc3C2)NC(c2nccs2)=NC1c1ccc(F)c(F)c1C] |
| 93p | 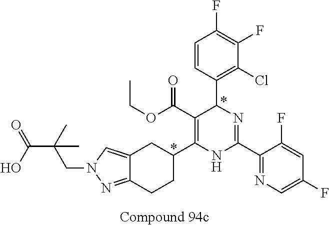 [SMILES: CCOC(=O)C1=C(C2CCc3nn(CC(C)(C)C(=O)O)cc3C2)NC(c2ncc(F)cc2F)=NC1c1ccc(F)c(F)c1Cl] |
| 95q | 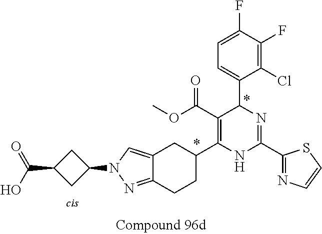 [SMILES: COC(=O)C1=C(C2CCc3nn([C@H]4C[C@@H](C(=O)O)C4)cc3C2)NC(c2nccs2)=NC1c1ccc(F)c(F)c1Cl] |

### Compound 35b

### 3-(5-(6-(3,4-Difluoro-2-methylphenyl)-5-(ethoxycarbonyl)-2-(thiazol-2-yl)-3,6-dihydropyrimidin-4-yl)-4,5,6,7-tetrahydro-2H-indazol-2-yl)propanoic acid

Converted from Compound 2b

LC-MS (ESI): R_{T}=3.441 min, mass calcd. for C₂₇H₂₇F₂N₅O₄S 555.1, m/z found 556.2 [M+H]⁺. Chiral HPLC (Column: Chiralpak IA 5 μm 4.6*250 mm; Mobile Phase: Hex:EtOH:TFA=90:10:0.2 at 1.0 mL/min; Temp: 30° C.; Wavelength: 230 nm, R_{T}=17.801 min). ¹H NMR (400 MHz, CD₃OD) δ 7.86 (d, J=3.2 Hz, 1H), 7.70 (d, J=2.8 Hz, 1H), 7.40 (br s, 1H), 7.12-7.00 (m, 2H), 5.91 (s, 1H), 4.36-4.33 (m, 2.6H), 4.03 (q, J=7.2 Hz, 2H), 3.12-2.63 (m, 6.4H), 2.52 (s, 3H), 2.20-1.87 (m, 2H), 1.10 (t, J=7.2 Hz, 3H).

Compound 35c:

### 3-(5-(6-(3,4-Difluoro-2-methylphenyl)-5-(ethoxycarbonyl)-2-(thiazol-2-yl)-3,6-dihydropyrimidin-4-yl)-4,5,6,7-tetrahydro-2H-indazol-2-yl)propanoic acid

Converted from Compound 2c

LC-MS (ESI): R_{T}=3.416 min, mass calcd. for C₂₇H₂₇F₂N₅O₄S 555.2, m/z found 556.1 [M+H]⁺. Chiral HPLC (Column: Chiralpak IA 5 μm 4.6*250 mm; Mobile Phase: Hex:EtOH:TFA=80:20:0.2 at 1.0 mL/min; Temp: 30° C.; Wavelength: 254 nm, R_{T}=8.068 min). ¹H NMR (400 MHz, CD₃OD) δ 7.87 (d, J=3.6 Hz, 1H), 7.71 (d, J=3.2 Hz, 1H), 7.36 (br s, 1H), 7.20-7.04 (m, 2H), 5.90 (s, 1H), 4.36-4.32 (m, 2.6H), 4.03 (q, J=7.2 Hz, 2H), 2.98-2.57 (m, 6.4H), 2.52 (s, 3H), 2.26-2.11 (m, 2H), 1.10 (t, J=7.2 Hz, 3H)

### Compound 36b

### 3-(5-(6-(2-Bromo-3,4-difluorophenyl)-5-(ethoxycarbonyl)-2-(thiazol-2-yl)-3,6-dihydropyrimidin-4-yl)-4,5,6,7-tetrahydro-2H-indazol-2-yl)propanoic acid

Converted from Compound 3b

LC-MS (ESI): R_{T}=3.232 min, mass calcd. for C₂₆H₂₄BrF₂N₅O₄S 620.5, m/z found mass 619.8 [M+H]⁺. Chiral HPLC (Column: Chiralpak IE 5 μm 4.6*250 mm; Mobile Phase: Hex:EtOH:TFA=80:20:0.2 at 1.0 mL/min; Temp: 30° C.; Wavelength: 230 nm, R_{T}=14.788 min). ¹H NMR (400 MHz, DMSO-d₆) δ 9.54 (d, J=3.2 Hz, 0.6H), 9.03 (s, 0.4H), 8.00-7.92 (m, 2H), 7.56-7.48 (m, 1H), 7.44 (s, 0.4H), 7.39 (s, 0.6H), 7.25-7.21 (m, 1H), 6.06 (s, 0.4H), 5.96 (d, J=3.2 Hz, 0.6H), 4.23-4.19 (m, 2H), 4.15-4.13 (m, 0.4H), 3.97-3.90 (m, 2.6H), 2.98-2.84 (m, 2H), 2.77-2.67 (m, 4H), 2.15-2.11 (m, 0.4H), 2.00-1.94 (m, 1H), 1.82-1.79 (m, 0.6H), 1.06-0.98 (m, 3H).

### Compound 36d

### 3-(5-(6-(2-Bromo-3,4-difluorophenyl)-5-(ethoxycarbonyl)-2-(thiazol-2-yl)-3,6-dihydropyrimidin-4-yl)-4,5,6,7-tetrahydro-2H-indazol-2-yl)propanoic acid

Converted from Compound 3d

LC-MS (ESI): R_{T}=3.274 min, mass calcd. for C₂₆H₂₄BrF₂N₅O₄S 620.5, m/z found mass 622.0 [M+H]⁺. Chiral HPLC (Column: Chiralpak IE 5 μm 4.6*250 mm; Mobile Phase: Hex:EtOH:TFA=80:20:0.2 at 1.0 mL/min; Temp: 30° C.; Wavelength: 230 nm, R_{T}=15.122 min). ¹H NMR (400 MHz, DMSO-d₆) δ 9.56 (d, J=1.6 Hz, 0.6H), 8.99 (s, 0.4H), 7.99-7.92 (m, 2H), 7.55-7.49 (m, 1H), 7.44 (s, 0.4H), 7.37 (s, 0.6H), 7.30-7.24 (m, 1H), 6.05 (s, 0.4H), 5.95 (d, J=2.8 Hz, 0.6H), 4.22-4.18 (m, 2H), 3.97-3.93 (m, 3H), 2.77-2.67 (m, 4H), 2.60-2.54 (m, 2H), 2.26-2.20 (m, 0.4H), 2.12-2.00 (m, 1.6H), 1.06-1.02 (m, 3H).

### Compound 37b

### 3-(5-(6-(2-Chloro-4-fluorophenyl)-5-(ethoxycarbonyl)-2-(thiazol-2-yl)-3,6-dihydropyrimidin-4-yl)-4,5,6,7-tetrahydro-2H-indazol-2-yl)propanoic acid

Converted from Compound 4b

LC-MS (ESI): R_{T}=4.125 min, mass calcd. for C₂₆H₂₅ClFN₅O₄S 557.1, m/z found 558.2 [M+H]⁺. Chiral HPLC (Column: Chiralpak IE 5 μm 4.6*250 mm; Mobile Phase: Hex:EtOH:TFA=80:20:0.2 at 1.0 mL/min; Wavelength: 254 nm; R_{T}=15.023 min). ¹H NMR (400 MHz, CD₃OD) δ 7.93-7.87 (m, 1H), 7.74-7.70 (m, 1H), 7.46-7.42 (m, 1H), 7.38 (s, 1H), 7.25-7.22 (m, 1H), 7.09-7.05 (m, 1H), 6.14 (s, 1H), 4.34 (t, J=6.8 Hz, 2H), 4.22-4.13 (m, 0.5H), 4.07-4.00 (m, 2.5H), 3.06-2.62 (m, 6H), 2.19-1.90 (m, 2H), 1.12-1.08 (m, 3H).

Compound 37c:

### 3-(5-(6-(2-Chloro-4-fluorophenyl)-5-(ethoxycarbonyl)-2-(thiazol-2-yl)-3,6-dihydropyrimidin-4-yl)-4,5,6,7-tetrahydro-2H-indazol-2-yl)propanoic acid

Converted from Compound 4c

LC-MS (ESI): R_{T}=2.567 min, mass calcd. for C₂₆H₂₅ClFN₅O₄S 557.1, m/z found 558.2 [M+H]⁺. Chiral HPLC (Column: Chiralpak IE 5 μm 4.6*250 mm; Mobile Phase: Hex:EtOH:TFA=80:20:0.2 at 1.0 mL/min; Wavelength: 254 nm; R_{T}=15.560 min). ¹H NMR (400 MHz, CD₃OD) δ 7.88 (s, 1H), 7.72 (s, 1H), 7.48-7.44 (m, 1H), 7.36 (br s, 1H), 7.25-7.22 (m, 1H), 7.10-7.06 (m, 1H), 6.13 (s, 1H), 4.35-4.32 (m, 2.5H), 4.05-4.00 (m, 2.5H), 2.89-2.81 (m, 4H), 2.74-2.61 (m, 2H), 2.25-2.13 (m, 2H), 1.12-1.09 (m, 3H).

### Compound 37d

### 3-(5-(6-(2-Chloro-4-fluorophenyl)-5-(ethoxycarbonyl)-2-(thiazol-2-yl)-3,6-dihydropyrimidin-4-yl)-4,5,6,7-tetrahydro-2H-indazol-2-yl)propanoic acid

Converted from Compound 4d

LC-MS (ESI): R_{T}=2.837 min, mass calcd. for C₂₆H₂₅ClFN₅O₄S 557.1, m/z found 558.2 [M+H]⁺. Chiral HPLC (Column: Chiralpak IE 5 μm 4.6*250 mm; Mobile Phase: Hex:EtOH:TFA=80:20:0.2 at 1.0 mL/min; Wavelength: 254 nm; R_{T}=14.115 min). ¹H NMR (400 MHz, CD₃OD) δ 7.88 (s, 1H), 7.72 (s, 1H), 7.48-7.45 (m, 1H), 7.36 (br s, 1H), 7.25-7.23 (m, 1H), 7.10-7.06 (m, 1H), 6.13 (s, 1H), 4.35-4.32 (m, 2.5H), 4.05-4.00 (m, 2.5H), 2.89-2.81 (m, 4H), 2.76-2.63 (m, 2H), 2.26-2.13 (m, 2H), 1.12-1.09 (m, 3H).

### Compound 38a

### 3-(5-(5-(Ethoxycarbonyl)-6-(4-fluoro-2-methylphenyl)-2-(thiazol-2-yl)-3,6-dihydropyrimidin-4-yl)-4,5,6,7-tetrahydro-2H-indazol-2-yl)propanoic acid

Converted from Compound 5a

LC-MS (ESI):R_{T}=3.076 min, mass calcd. for C₂₇H₂₈FN₅O₄S 537.6, m/z found 538.0 [M+H]⁺. ¹H NMR (400 MHz, DMSO-d₆) δ 9.48 (s, 0.8H), 8.94 (s, 0.2H), 7.98-7.89 (m, 2H), 7.45 (s, 0.2H), 7.39 (s, 0.8H), 7.32-7.28 (m, 0.8H), 7.22-7.19 (m, 0.2H), 7.07-6.97 (m, 2H), 5.85 (s, 0.2H), 5.71 (s, 0.8H), 4.24-4.19 (m, 2H), 3.98-3.86 (m, 3H), 2.95-2.88 (m, 1H), 2.75-2.66 (m, 4H), 2.56-2.53 (m, 4H), 2.12-2.08 (m, 0.2H), 2.05-1.92 (m, 1H), 1.81-1.79 (m, 0.8H), 1.05-0.99 (m, 3H).

### Compound 38b

### 3-(5-(5-(Ethoxycarbonyl)-6-(4-fluoro-2-methylphenyl)-2-(thiazol-2-yl)-3,6-dihydropyrimidin-4-yl)-4,5,6,7-tetrahydro-2H-indazol-2-yl)propanoic acid

Converted from Compound 5b

LC-MS (ESI): R_{T}=4.023 min, mass calcd. for C₂₇H₂₈FN₅O₄S 537.6, m/z found 538.2 [M+H]⁺. ¹H NMR (400 MHz, DMSO-d₆) δ 9.48 (s, 0.8H), 8.94 (s, 0.2H), 7.98-7.89 (m, 2H), 7.45 (s, 0.2H), 7.40 (s, 0.8H), 7.32-7.28 (m, 0.8H), 7.23-7.19 (m, 0.2H), 7.07-6.99 (m, 2H), 5.85 (s, 0.2H), 5.72 (s, 0.8H), 4.25-4.19 (m, 2H), 3.98-3.86 (m, 3H), 2.95-2.86 (m, 1H), 2.76-2.66 (m, 4H), 2.56-2.50 (m, 4H), 2.16-2.10 (m, 0.2H), 2.04-1.94 (m, 1H), 1.81-1.79 (m, 0.8H), 1.05-0.99 (m, 3H).

### Compound 38d

### 3-(5-(5-(Ethoxycarbonyl)-6-(4-fluoro-2-methylphenyl)-2-(thiazol-2-yl)-3,6-dihydropyrimidin-4-yl)-4,5,6,7-tetrahydro-2H-indazol-2-yl)propanoic acid

Converted from Compound 5d

LC-MS (ESI): R_{T}=4.019 min, mass calcd. for C₂₇H₂₈FN₅O₄S 537.6, m/z found 538.2 [M+H]⁺. Chiral HPLC (Column: Chiralpak IA 5 μm 4.6*250 mm; Mobile Phase: Hex:EtOH:TFA=80:20:0.2 at 1.0 mL/min; Temp: 30° C.; Wavelength: 230 nm, R_{T}=8.000 min). ¹H NMR (400 MHz, DMSO-d₆) δ 9.50 (d, J=2.8 Hz, 0.8H), 8.89 (s, 0.2H), 7.99-7.89 (m, 2H), 7.44 (s, 0.2H), 7.36-7.32 (m, 1.6H), 7.25-7.22 (m, 0.2H), 7.07-6.97 (m, 2H), 5.84 (s, 0.2H), 5.71 (s, 0.8H), 4.25-4.19 (m, 2H), 3.99-3.91 (m, 3H), 2.82-2.73 (m, 4H), 2.69-2.61 (m, 1H), 2.58-2.53 (m, 4H), 2.14-1.99 (m, 2H), 1.05-1.00 (m, 3H).

### Compound 39b

### 3-(5-(6-(2-Bromo-4-fluorophenyl)-5-(ethoxycarbonyl)-2-(thiazol-2-yl)-3,6-dihydropyrimidin-4-yl)-4,5,6,7-tetrahydro-2H-indazol-2-yl)propanoic acid

Converted from Compound 6b

LC-MS (ESI): R_{T}=2.912 min, mass calcd. for C₂₆H₂₅BrFN₅O₄S 601.1, m/z found 604.0 [M+H]⁺. Chiral HPLC (Column: Chiralpak IE 5 μm 4.6*250 mm; Mobile Phase: Hex:EtOH:TFA=80:20:0.2 at 1.0 mL/min; Temp: 30° C.; Wavelength: 230 nm; R_{T}=15.155 min). ¹H NMR (400 MHz, CD₃OD) δ 7.87 (d, J=2.8 Hz, 1H), 7.71 (d, J=3.2 Hz, 1H), 7.48-7.35 (m, 3H), 7.15-7.10 (m, 1H), 6.12 (br s, 1H), 4.40-4.27 (m, 2.5H), 4.09-3.94 (m, 2H), 3.01-2.62 (m, 6H), 2.18-1.88 (m, 2H), 1.10 (t, J=7.2 Hz, 3H).

### Compound 39c

### 3-(5-(6-(2-Bromo-4-fluorophenyl)-5-(ethoxycarbonyl)-2-(thiazol-2-yl)-3,6-dihydropyrimidin-4-yl)-4,5,6,7-tetrahydro-2H-indazol-2-yl)propanoic acid

Converted from Compound 6c

LC-MS (ESI): R_{T}=2.909 min, mass calcd. for C₂₆H₂₅BrFN₅O₄S 601.1, m/z found 602.1 [M+H]⁺. Chiral HPLC (Column: Chiralpak IE 5 μm 4.6*250 mm; Mobile Phase: Hex:EtOH:TFA=80:20:0.2 at 1.0 mL/min; Temp: 30° C.; Wavelength: 230 nm; R_{T}=15.766 min). ¹H NMR (400 MHz, CD₃OD) δ 7.88 (d, J=2.8 Hz, 1H), 7.72 (d, J=2.8 Hz, 1H), 7.47-7.29 (m, 3H), 7.15-7.11 (m, 1H), 6.10 (br s, 1H), 4.43-4.27 (m, 2.5H), 4.12-3.92 (m, 2.5H), 2.89-2.52 (m, 6H), 2.29-2.10 (m, 2H), 1.10 (t, J=6.8 Hz, 3H).

### Compound 40b

### 3-(5-(6-(2-Chloro-3-fluorophenyl)-5-(ethoxycarbonyl)-2-(thiazol-2-yl)-3,6-dihydropyrimidin-4-yl)-4,5,6,7-tetrahydro-2H-indazol-2-yl)propanoic acid

Converted from Compound 7b

LC-MS (ESI): R_{T}=3.283 min, mass calcd. for C₂₆H₂₅ClFN₅O₄S 557.1, m/z found 558.1 [M+H]⁺. Chiral HPLC (Column: Chiralpak IE 5 μm 4.6*250 mm; Mobile Phase: Hex:EtOH:TFA=80:20:0.2 at 1.0 mL/min; Temp: 30° C.; Wavelength: 254 nm, R_{T}=min). ¹H NMR (400 MHz, DMSO-d₆) δ 12.32 (s, 1H), 9.52 (d, J=3.6 Hz, 0.7H), 9.01 (s, 0.3H), 8.00-7.92 (m, 2H), 7.45-7.30 (m, 3H), 7.26-7.23 (m, 1H), 6.13 (s, 0.3H), 6.02 (d, J=3.6 Hz, 0.7H), 4.25-4.20 (m, 2H), 4.17-4.12 (m, 0.3H), 3.98-3.88 (m, 2.7H), 2.97-2.87 (m, 1H), 2.84-2.83 (m, 0.2H), 2.79-2.66 (m, 4H), 2.59-2.54 (m, 0.8H), 2.18-2.08 (m, 0.4H), 2.01-1.94 (m, 1H), 1.83-1.80 (m, 0.6H), 1.05-0.97 (m, 3H).

### Compound 40c

### 3-(5-(6-(2-Chloro-3-fluorophenyl)-5-(ethoxycarbonyl)-2-(thiazol-2-yl)-3,6-dihydropyrimidin-4-yl)-4,5,6,7-tetrahydro-2H-indazol-2-yl)propanoic acid

Converted from Compound 7c

LC-MS (ESI): R_{T}=3.265 min, mass calcd. for C₂₆H₂₅ClFN₅O₄S 557.1, m/z found 558.1 [M+H]⁺. Chiral HPLC (Column: Chiralpak IA 5 μm 4.6*250 mm; Mobile Phase: Hex:EtOH:TFA=80:20:0.2 at 1.0 mL/min; Temp: 30° C.; Wavelength: 254 nm, R_{T}=8.108 min). ¹H NMR (400 MHz, DMSO-d₆) δ 12.31 (s, 1H), 9.54 (d, J=3.2 Hz, 0.7H), 8.96 (s, 0.3H), 8.00-7.92 (m, 2H), 7.45-7.26 (m, 4H), 6.11 (s, 0.3H), 6.01 (d, J=3.2 Hz, 0.7H), 4.25-4.19 (m, 2H), 4.16-4.13 (m, 0.3H), 3.99-3.89 (m, 2.7H), 2.82-2.74 (m, 4H), 2.68-2.58 (m, 2H), 2.26-2.20 (m, 0.3H), 2.12-2.00 (m, 1.7H), 1.05-0.98 (m, 3H).

### Compound 41b

### 3-(5-(5-(Ethoxycarbonyl)-6-(3-fluoro-2-methylphenyl)-2-(thiazol-2-yl)-3,6-dihydropyrimidin-4-yl)-4,5,6,7-tetrahydro-2H-indazol-2-yl)propanoic acid

Converted from Compound 8b

LC-MS (ESI): R_{T}=3.286 min, mass calcd. for C₂₇H₂₈FN₅O₄S 537.1, m/z found 538.2 [M+H]⁺). ¹H NMR (400 MHz, DMSO-d₆) δ 12.16 (s, 1H), 9.50 (d, J=3.6 Hz, 0.8H), 8.97 (s, 0.2H), 7.98-7.90 (m, 2H), 7.45 (s, 0.2H), 7.39 (s, 0.8H), 7.25-7.16 (m, 1.8H), 7.08-7.02 (m, 1.2H), 5.91 (s, 0.2H), 5.77 (d, J=2.8 Hz, 0.8H), 4.25-4.19 (m, 2.2H), 3.98-3.86 (m, 2.8H), 2.97-2.84 (m, 1.3H), 2.77-2.66 (m, 4H), 2.60-2.54 (m, 0.7H), 2.45 (s, 0.6H), 2.40 (s, 2.4H), 2.15-1.94 (m, 1.2H), 1.83-1.79 (m, 0.8H), 1.04-0.98 (m, 3H).

### Compound 41c

### 3-(5-(5-(Ethoxycarbonyl)-6-(3-fluoro-2-methylphenyl)-2-(thiazol-2-yl)-3,6-dihydropyrimidin-4-yl)-4,5,6,7-tetrahydro-2H-indazol-2-yl)propanoic acid

Converted from Compound 8c

LC-MS (ESI): R_{T}=2.674 min, mass calcd. for C₂₇H₂₈FN₅O₄S 537.1, m/z found 538.2 [M+H]⁺. Chiral HPLC (Column: Chiralpak IA 5 μm 4.6*250 mm; Mobile Phase: Hex:EtOH:TFA=70:30:0.2 at 1 mL/min; Wavelength: 230 nm, R_{T}=5.391 min)). ¹H NMR (400 MHz, DMSO-d₆) δ 9.53 (s, 0.8H), 8.93 (s, 0.2H), 7.99-7.96 (m, 1.6H), 7.94 (d, J=3.6 Hz, 0.2H), 7.90 (d, J=3.2 Hz, 0.2H), 7.44 (s, 0.2H), 7.36 (s, 0.8H), 7.25-7.19 (m, 1.8H), 7.10-7.02 (m, 1.2H), 5.89 (s, 0.2H), 5.76 (s, 0.8H), 4.24-4.18 (m, 2.2H), 4.01-3.88 (m, 2.8H), 2.81-2.72 (m, 4H), 2.63-2.55 (m, 0.8H), 2.45 (s, 0.8H), 2.41 (s, 3H), 2.29-2.22 (m, 0.4H), 2.12-1.97 (m, 2H), 1.04-1.00 (m, 3H).

### Compound 42b

### 3-(5-(6-(2-bromo-3-fluorophenyl)-5-(ethoxycarbonyl)-2-(thiazol-2-yl)-3,6-dihydropyrimidin-4-yl)-4,5,6,7-tetrahydro-2H-indazol-2-yl)propanoic acid

Converted from Compound 9b

LC-MS (ESI): R_{T}=4.137 min, mass calcd. for C₂₆H₂₅BrFN₅O₄S 601.1, m/z found 602.0 [M+H]⁺. Chiral HPLC (Column: Chiralpak IE 5 μm 4.6*250 mm; Mobile Phase: Hex:EtOH:TFA=80:20:0.2 at 1.0 mL/min; Temp: 30° C.; Wavelength: 230 nm, R_{T}=15.856 min). ¹H NMR (400 MHz, DMSO-d₆) δ 9.00 (br s, 1H), 8.00-7.91 (m, 2H), 7.47-7.39 (m, 2H), 7.32-7.21 (m, 2H), 6.11 (s, 0.4H), 6.00 (s, 0.6H), 4.22-4.13 (m, 2.4H), 3.98-3.88 (m, 2.6H), 2.98-2. 66 (m, 6H), 2.33-1.79 (m, 2H), 1.05-0.97 (m, 3H).

### Compound 42d

### 3-(5-(6-(2-bromo-3-fluorophenyl)-5-(ethoxycarbonyl)-2-(thiazol-2-yl)-3,6-dihydropyrimidin-4-yl)-4,5,6,7-tetrahydro-2H-indazol-2-yl)propanoic acid

Converted from Compound 9d

LC-MS (ESI): R_{T}=4.127 min, mass calcd. for C₂₆H₂₅BrFN₅O₄S 601.1, m/z found 602.1 [M+H]⁺. Chiral HPLC (Column: Chiralpak IA 5 μm 4.6*250 mm; Mobile Phase: Hex:EtOH:TFA=80:20:0.2 at 1.0 mL/min; Temp: 30° C.; Wavelength: 230 nm, R_{T}=8.827 min). ¹H NMR (400 MHz, DMSO-d₆) δ 8.97 (br s, 1H), 7.99-7.91 (m, 2H), 7.47-7.42 (m, 1.3H), 7.37 (s, 0.7H), 7.32-7.23 (m, 2H), 6.10 (s, 0.3H), 6.00 (s, 0.7H), 4.23-4.14 (m, 2.4H), 4.00-3.89 (m, 2.6H), 2.79-2. 58 (m, 6H), 2.33-2.03 (m, 2H), 1.05-0.98 (m, 3H).

### Compound 43b

### 3-(5-(6-(2-Chloro-3,4-difluorophenyl)-2-(3,5-difluoropyridin-2-yl)-5-(ethoxycarbonyl)-3,6-dihydropyrimidin-4-yl)-4,5,6,7-tetrahydro-2H-indazol-2-yl)propanoic acid

Converted from Compound 10b.

LC-MS (ESI): R_{T}=3.056 min, mass calcd. for C₂₈H₂₄ClF₄N₅O₄ 605.2, m/z found 605.9 [M+H]⁺. ¹H NMR (400 MHz, DMSO-d₆) δ 11.84 (s, 1H), 8.77 (s, 1H), 8.36-8.32 (m, 1H), 7.67-7.60 (m, 1H), 7.46 (s, 1H), 7.42-7.39 (m, 1H), 6.24 (s, 1H), 4.22 (t, J=6.8 Hz, 2H), 4.10-3.98 (m, 3H), 2.95-2.88 (m, 1H), 2.80-2.70 (m, 4H), 2.63-2.54 (m, 1H), 2.19-2.08 (m, 1H), 2.00-1.91 (m, 1H), 1.03 (t, J=7.2 Hz, 3H).

### Compound 43c

### 3-(5-(6-(2-Chloro-3,4-difluorophenyl)-2-(3,5-difluoropyridin-2-yl)-5-(ethoxycarbonyl)-3,6-dihydropyrimidin-4-yl)-4,5,6,7-tetrahydro-2H-indazol-2-yl)propanoic acid

Converted from Compound 10c.

LC-MS (ESI): R_{T}=3.090 min, mass calcd. for C₂₈H₂₄ClF₄N₅O₄ 605.2, m/z found 605.9 [M+H]⁺. ¹H NMR (400 MHz, DMSO-d₆ (one drop of D₂O)) S 8.69 (d, J=2.4 Hz, 1H), 8.24-8.18 (m, 1H), 7.60-7.54 (m, 1H), 7.45 (s, 1H), 7.42-7.38 (m, 1H), 6.22 (s, 1H), 4.20 (t, J=6.4 Hz, 2H), 4.12-4.04 (m, 1H), 3.99 (q, J=6.8 Hz, 2H), 2.79-2.67 (m, 5H), 2.63-2.54 (m, 1H), 2.15-2.06 (m, 1H), 2.04-1.97 (m, 1H), 1.01 (t, J=6.8 Hz, 3H).

### Compound 44b

### 3-(5-(6-(2-Chloro-3-fluorophenyl)-5-(methoxycarbonyl)-2-(thiazol-2-yl)-3,6-dihydropyrimidin-4-yl)-4,5,6,7-tetrahydro-2H-indazol-2-yl)propanoic acid

Converted from Compound 11b

LC-MS (ESI): R_{T}=3.357 min, mass calcd. for C₂₅H₂₃ClFN₅O₄S 544.0, m/z found 543.9 [M+H]⁺. Chiral HPLC (Column: Chiralpak IE 5 μm 4.6*250 mm; Mobile Phase: Hex:EtOH:TFA=80:20:0.2 at 1.0 mL/min; Temp: 30° C.; Wavelength: 230 nm, R_{T}=17.037 min). ¹H NMR (400 MHz, DMSO-d₆) δ 8.00-7.97 (m, 1.4H), 7.96-7.95 (m, 0.3H), 7.93-7.92 (m, 0.3H), 7.45-7.30 (m, 3H), 7.25-7.21 (m, 1H), 6.11 (s, 0.3H), 6.00 (s, 0.7H), 4.24-4.17 (m, 2H), 4.13-4.12 (m, 0.3H), 3.93-3.86 (m, 0.7H), 3.51 (s, 2.1H), 3.49 (s, 0.9H), 2.99-2.91 (m, 1.6H), 2.84-2.76 (m, 2.4H), 2.74-2.54 (m, 2H), 2.16-2.11 (m, 0.3H), 2.03-1.93 (m, 1H), 1.82-1.79 (m, 0.7H).

### Compound 44c

### 3-(5-(6-(2-Chloro-3-fluorophenyl)-5-(methoxycarbonyl)-2-(thiazol-2-yl)-3,6-dihydropyrimidin-4-yl)-4,5,6,7-tetrahydro-2H-indazol-2-yl)propanoic acid

Converted from Compound 11c.

LC-MS (ESI): R_{T}=3.883 min, mass calcd. for C₂₅H₂₃ClFN₅O₄S 544.0, m/z found 544.1 [M+H]⁺. Chiral HPLC (Column: Chiralpak IE 5 μm 4.6*250 mm; Mobile Phase: Hex:EtOH:TFA=80:20:0.2 at 1.0 mL/min; Temp: 30° C.; Wavelength: 230 nm, R_{T}=min). ¹H NMR (400 MHz, DMSO-d₆) δ 8.00-7.98 (m, 1.4H), 7.96-7.95 (m, 0.3H), 7.93-7.92 (m, 0.3H), 7.45-7.32 (m, 3H), 7.28-7.23 (m, 1H), 6.09 (s, 0.3H), 5.99 (s, 0.7H), 4.24-4.18 (m, 2.3H), 3.93-3.87 (m, 0.7H), 3.51 (s, 2.1H), 3.50 (s, 0.9H), 2.78-2.70 (m, 4H), 2.67-2.53 (m, 2H), 2.28-2.22 (m, 0.3H), 2.14-2.05 (m, 1H), 2.01-1.97 (m, 0.7H).

### Compound 45b

### 3-(5-(6-(2-Bromo-3-fluorophenyl)-5-(methoxycarbonyl)-2-(thiazol-2-yl)-3,6-dihydropyrimidin-4-yl)-4,5,6,7-tetrahydro-2H-indazol-2-yl)propanoic acid

Converted from Compound 12b

LC-MS (ESI): R_{T}=3.139 min, mass calcd. for C₂₅H₂₃BrFN₅O₄S 587.1, m/z found 588.0 [M+H]⁺. Chiral HPLC (Column: Chiralpak IE 5 μm 4.6*250 mm; Mobile Phase: Hex:EtOH:TFA=80:20:0.2 at 1.0 mL/min; Temp: 30° C.; Wavelength: 230 nm, R_{T}=12.134 min). ¹H NMR (400 MHz, CD₃OD) δ 7.87 (d, J=2.8 Hz, 1H), 7.71 (s, 1H), 7.45-7.32 (m, 2H), 7.24 (d, J=7.2 Hz, 1H), 7.11 (s, 1H), 6.22-6.15 (m, 1H), 4.34 (t, J=6.8 Hz, 2.5H), 4.08 (s, 0.5H), 3.57 (s, 3H), 3.05-2.86 (m, 1H), 2.79 (t, J=6.8 Hz, 4H), 2.73-2.56 (m, 1H), 2.17-2.06 (m, 1.5H), 1.98-1.87 (m, 0.5H).

### Compound 45c

### 3-(5-(6-(2-Bromo-3-fluorophenyl)-5-(methoxycarbonyl)-2-(thiazol-2-yl)-3,6-dihydropyrimidin-4-yl)-4,5,6,7-tetrahydro-2H-indazol-2-yl)propanoic acid

Converted from Compound 12c

LC-MS (ESI): R_{T}=3.919 min, mass calcd. for C₂₅H₂₃BrFN₅O₄S 587.1, m/z found 590.0 [M+H]⁺. Chiral HPLC (Column: Chiralpak IA 5 μm 4.6*250 mm; Mobile Phase: Hex:EtOH:TFA=80:20:0.2 at 1.0 mL/min; Temp: 30° C.; Wavelength: 230 nm, R_{T}=9.151 min). ¹H NMR (400 MHz, CD₃OD) δ 7.88 (s, 1H), 7.71 (s, 1H), 7.44-7.34 (m, 2H), 7.28-7.26 (m, 1H), 7.12 (s, 1H), 6.22-6.13 (m, 1H), 4.34 (s, 2.5H), 4.07 (br s, 0.5H), 3.57 (s, 3H), 2.90-2.72 (m, 5.4H), 2.60 (br s, 0.6H), 2.21 (br s, 2H).

### Compound 46a (Mixture of Two Stereoisomers)

### 3-(5-(6-(2-Bromo-3,4-difluorophenyl)-5-(methoxycarbonyl)-2-(thiazol-2-yl)-3,6-dihydropyrimidin-4-yl)-4,5,6,7-tetrahydro-2H-indazol-2-yl)propanoic acid

Converted from Compound 13a

LC-MS (ESI): R_{T}=3.117 min, mass calcd. for C₂₅H₂₂BrF₂N₅O₄S 605.1, m/z found mass 605.8 [M+H]⁺. ¹H NMR (400 MHz, DMSO-d₆) δ 9.56 (br s, 0.5H), 9.13 (br s, 0.5H), 8.01-7.98 (m, 1.2H), 7.96 (d, J=3.2 Hz, 0.4H), 7.93 (d, J=3.2 Hz, 0.4H), 7.55-7.48 (m, 1H), 7.44 (s, 0.4H), 7.39 (s, 0.6H), 7.27-7.23 (m, 0.6H), 7.21-7.18 (m, 0.4H), 6.05 (s, 0.4H), 5.95 (s, 0.6H), 4.24-4.16 (m, 2H), 4.15-4.11 (m, 0.4H), 3.93-3.87 (m, 0.6H), 3.51 (s, 2H), 3.49 (s, 1H), 2.99-2.80 (m, 1H), 2.75-2.68 (m, 4H), 2.62-2.51 (m, 1H), 2.18-2.11 (m, 0.3H), 2.00-1.93 (m, 1H), 1.83-1.71 (m, 0.7H).

### Compound 46d

### 3-(5-(6-(2-Bromo-3,4-difluorophenyl)-5-(methoxycarbonyl)-2-(thiazol-2-yl)-3,6-dihydropyrimidin-4-yl)-4,5,6,7-tetrahydro-2H-indazol-2-yl)propanoic acid

Converted from Compound 13d

LC-MS (ESI): R_{T}=3.572 min, mass calcd. for C₂₅H₂₂BrF₂N₅O₄S 605.1, m/z found mass 605.8 [M+H]⁺. Chiral HPLC (Column: Chiralpak IC 5 μm 4.6*250 mm; Mobile Phase: Hex:EtOH:TFA=80:20:0.2 at 1.0 mL/min; Temp: 30° C.; Wavelength: 254 nm, R_{T}=8.748 min). ¹H NMR (400 MHz, DMSO-d₆) δ 9.59 (s, 0.5H), 9.06 (s, 0.5H), 8.01-7.98 (m, 1.2H), 7.95 (d, J=3.6 Hz, 0.4H), 7.93 (d, J=3.6 Hz, 0.4H), 7.55-7.49 (m, 1H), 7.44 (s, 0.3H), 7.37 (s, 0.7H), 7.30-7.26 (m, 0.7H), 7.24-7.21 (m, 0.3H), 6.03 (s, 0.3H), 5.94 (s, 0.7H), 4.24-4.16 (m, 2H), 4.14-4.12 (m, 0.3H), 3.94-3.87 (m, 0.7H), 3.51 (s, 2H), 3.50 (s, 1H), 2.77-2.67 (m, 4H), 2.67-2.62 (m, 1H), 2.58-2.52 (m, 1H), 2.27-2.19 (m, 0.3H), 2.12-2.06 (m, 1H), 2.02-1.97 (m, 0.7H).

### Compound 47b

### 3-(5-(6-(2-Chloro-4-fluorophenyl)-5-(methoxycarbonyl)-2-(thiazol-2-yl)-3,6-dihydropyrimidin-4-yl)-4,5,6,7-tetrahydro-2H-indazol-2-yl)propanoic acid

Converted from Compound 14b.

LC-MS (ESI): R_{T}=3.885 min, mass calcd. for C₂₅H₂₃ClFN₅O₄S 544.0, m/z found 544.1 [M+H]⁺. Chiral HPLC (Column: Chiralpak IE 5 μm 4.6*250 mm; Mobile Phase: Hex:EtOH:TFA=80:20:0.2 at 1.0 mL/min; Temp: 30° C.; Wavelength: 230 nm, R_{T}=min). ¹H NMR (400 MHz, DMSO-d₆) δ 9.00 (br s, 1H), 7.99-7.94 (m, 1.5H), 7.92-7.91 (m, 0.5H), 7.44-7.35 (m, 3H), 7.25-7.19 (m, 1H), 6.05 (s, 0.3H), 5.95 (s, 0.7H), 4.22-4.09 (m, 2.3H), 3.93-3.84 (m, 0.7H), 3.51 (s, 2H), 3.49 (s, 1H), 2.96-2.61 (m, 4H), 2.44-2.31 (m, 2H), 2.10-1.89 (m, 1.4H), 1.83-1.72 (m, 0.6H).

### Compound 47c

### 3-(5-(6-(2-Chloro-4-fluorophenyl)-5-(methoxycarbonyl)-2-(thiazol-2-yl)-3,6-dihydropyrimidin-4-yl)-4,5,6,7-tetrahydro-2H-indazol-2-yl)propanoic acid

Converted from Compound 14c.

LC-MS (ESI): R_{T}=3.192 min, mass calcd. for C₂₅H₂₃ClFN₅O₄S 544.0, m/z found 544.1 [M+H]⁺. Chiral HPLC (Column: Chiralpak IE 5 μm 4.6*250 mm; Mobile Phase: Hex:EtOH:TFA=80:20:0.2 at 1.0 mL/min; Temp: 30° C.; Wavelength: 230 nm, R_{T}=16.668 min). ¹H NMR (400 MHz, DMSO-d₆) δ 9.53 (br s, 0.7H), 9.00 (br s, 0.3H), 8.06-7.96 (m, 2H), 7.51-7.41 (m, 3H), 7.31-7.25 (m, 1H), 6.09 (s, 0.3H), 5.98 (s, 0.7H), 4.33-4.16 (m, 2.3H), 3.99-3.89 (m, 0.7H), 3.50 (s, 3H), 2.85-2.51 (m, 6H), 2.21-1.97 (m, 2H).

### Compound 48b

### 3-(5-(6-(2-Bromo-4-fluorophenyl)-5-(methoxycarbonyl)-2-(thiazol-2-yl)-3,6-dihydropyrimidin-4-yl)-4,5,6,7-tetrahydro-2H-indazol-2-yl)propanoic acid

Converted from Compound 15b.

LC-MS (ESI): R_{T}=4.077 min, mass calcd. for C₂₆H₂₅BrFN₅O₄S 601.1, m/z found 604.0 [M+H]⁺. Chiral HPLC (Column: Chiralpak IG 5 μm 4.6*250 mm; Mobile Phase: Hex:EtOH:DEA=60:40:0.2 at 1.0 mL/min; Temp: 30° C.; Wavelength: 230 nm, R_{T}=9.960 min). ¹H NMR (400 MHz, CDCl₃) δ 8.21 (s, 0.5H), 7.82 (d, J=3.2 Hz, 0.5H), 7.75 (d, J=3.2 Hz, 0.5H), 7.47 (d, J=3.2 Hz, 1H), 7.42 (d, J=3.2 Hz, 0.5H), 7.34-7.31 (m, 2H), 7.24 (s, 0.5H), 7.20 (s, 0.5H), 7.04-6.95 (m, 1H), 6.20 (s, 0.5H), 6.05 (d, J=2.4 Hz, 0.5H), 4.39-4.32 (m, 2.5H), 4.12-4.04 (m, 0.5H), 3.71 (s, 3H), 3.60 (s, 1.5H), 3.59 (s, 1.5H), 3.13-3.01 (m, 1H), 2.92-2.71 (m, 5H), 2.19-1.91 (m, 2H).

### Compound 48d

### 3-(5-(6-(2-Bromo-4-fluorophenyl)-5-(methoxycarbonyl)-2-(thiazol-2-yl)-3,6-dihydropyrimidin-4-yl)-4,5,6,7-tetrahydro-2H-indazol-2-yl)propanoic acid

Converted from Compound 15d

LC-MS (ESI): R_{T}=4.314 min, mass calcd. for C₂₆H₂₅BrFN₅O₄S 601.1, m/z found 604.1 [M+H]⁺. Chiral HPLC (Column: Chiralpak IG 5 μm 4.6*250 mm; Mobile Phase: Hex:EtOH:DEA=60:40:0.2 at 1.0 mL/min; Temp: 30° C.; Wavelength: 230 nm, R_{T}=14.335 min). ¹H NMR (400 MHz, CDCl₃) δ 8.21 (s, 0.5H), 7.82 (d, J=3.2 Hz, 0.5H), 7.75 (d, J=3.2 Hz, 0.5H), 7.47 (d, J=2.8 Hz, 1H), 7.42 (d, J=3.2 Hz, 0.5H), 7.38-7.31 (m, 2H), 7.22 (s, 0.5H), 7.16 (s, 0.5H), 7.04-6.95 (m, 1H), 6.18 (s, 0.5H), 6.04 (d, J=2.4 Hz, 0.5H), 4.41-4.34 (m, 2.5H), 4.13-4.05 (m, 0.5H), 3.71 (s, 3H), 3.60 (s, 1.5H), 3.59 (s, 1.5H), 2.98-2.78 (m, 5H), 2.69-2.59 (m, 1H), 2.37-2.01 (m, 2H).

### Compound 49b

### 6-(2-Carboxymethyl-4,5,6,7-tetrahydro-2H-indazol-5-yl)-4-(2-chloro-3,4-difluoro-phenyl)-2-thiazol-2-yl-1,4-dihydro-pyrimidine-5-carboxylic acid

Converted from 17b

LC-MS (ESI): R_{T}=4.124 min, mass calcd. for C₂₅H₂₂ClF₂N₅O₄S 561.1, m/z found 562.1 [M+H]⁺. Chiral HPLC (Column: Chiralpak IC 5 μm 4.6*250 mm; Mobile Phase: Hex:EtOH:TFA=90:10:0.2 at 0.5 mL/min, Temp: 30° C.; Wavelength: 230 nm, R_{T}=25.969 min). ¹H NMR (400 MHz, CD₃OD) δ 7.88 (s, 1H), 7.72 (s, 1H), 7.38 (s, 1H), 7.27-7.25 (m, 2H), 6.15 (s, 1H), 4.78 (s, 2H), 4.22 (br s, 1H), 4.03 (q, J=8.4 Hz, 2H), 3.09-2.66 (m, 4H), 2.19-1.85 (m, 2H), 1.11 (t, J=5.6 Hz, 3H).

### Compound 49c

### 6-(2-Carboxymethyl-4,5,6,7-tetrahydro-2H-indazol-5-yl)-4-(2-chloro-3,4-difluoro-phenyl)-2-thiazol-2-yl-1,4-dihydro-pyrimidine-5-carboxylic acid

Converted from 17c.

LC-MS (ESI): R_{T}=2.762 min, mass calcd. for C₂₅H₂₂ClF₂N₅O₄S 561.1, m/z found 562.1 [M+H]⁺. Chiral HPLC (Column: Chiralpak IA 5 μm 4.6*250 mm; Mobile Phase: Hex:EtOH:TFA=80:20:0.2 at 1 mL/min, Temp: 30° C.; Wavelength: 230 nm, R_{T}=8.830 min). ¹H NMR (400 MHz, CD₃OD) S 7.88 (d, J=3.2 Hz, 1H), 7.73 (s, 1H), 7.36 (s, 1H), 7.30-7.24 (m, 2H), 6.13 (s, 1H), 4.78 (s, 2H), 4.20 (br s, 1H), 4.03 (q, J=7.2 Hz, 2H), 2.88-2.65 (m, 4H), 2.25-2.12 (m, 2H), 1.10 (t, J=7.2 Hz, 3H).

### Compound 50b

### 4-(5-(6-(2-Chloro-3,4-difluorophenyl)-5-(ethoxycarbonyl)-2-(thiazol-2-yl)-3,6-dihydropyrimidin-4-yl)-4,5,6,7-tetrahydro-2H-indazol-2-yl)butanoic acid

Converted from Compound 18b.

LC-MS (ESI): R_{T}=3.513 min, mass calcd. for C₂₇H₂₆ClF₂N₅O₄S 589.1, m/z found 590.1 [M+H]⁺. Chiral HPLC (Column: Chiralpak IA 5 μm 4.6*250 mm; Mobile Phase: Hex:EtOH:TFA=80:20:0.2 at 1.0 mL/min; Temp: 30° C.; Wavelength: 230 nm; R_{T}=8.918 min). ¹H NMR (400 MHz, DMSO-d₆) δ 9.03 (br s, 1H), 8.00-7.92 (m, 2H), 7.52-7.39 (m, 2H), 7.27-7.21 (m, 1H), 6.07 (s, 0.3H), 5.97 (s, 0.7H), 4.20-4.13 (m, 0.4H), 4.05-3.91 (m, 4.6H), 2.99-2.59 (m, 4H), 2.22-2.18 (m, 2H), 2.13-1.92 (m, 3.3H), 1.83-1.80 (m, 0.7H), 1.06-0.99 (m, 3H).

### Compound 50c

### 4-(5-(6-(2-Chloro-3,4-difluorophenyl)-5-(ethoxycarbonyl)-2-(thiazol-2-yl)-3,6-dihydropyrimidin-4-yl)-4,5,6,7-tetrahydro-2H-indazol-2-yl)butanoic acid

Converted from Compound 18c.

LC-MS (ESI): R_{T}=3.519 min, mass calcd. for C₂₇H₂₆ClF₂N₅O₄S 589.1, m/z found 590.1 [M+H]⁺. Chiral HPLC (Column: Chiralpak IA 5 μm 4.6*250 mm; Mobile Phase: Hex:EtOH:TFA=80:20:0.2 at 1.0 mL/min; Temp: 30° C.; Wavelength: 230 nm; R_{T}=8.790 min). ¹H NMR (400 MHz, DMSO-d₆) δ 8.98 (br s, 1H), 8.01-7.92 (m, 2H), 7.52-7.37 (m, 2H), 7.30-7.24 (m, 1H), 6.06 (s, 0.3H), 5.96 (s, 0.7H), 4.22-4.16 (m, 0.4H), 4.05-3.90 (m, 4.6H), 2.79-2.55 (m, 4H), 2.27-1.91 (m, 6H), 1.06-0.99 (m, 3H).

### Compound 51b

### 3-(5-(6-(2-chloro-3,4-difluorophenyl)-5-(ethoxycarbonyl)-2-(thiazol-2-yl)-3,6-dihydropyrimidin-4-yl)-4,5,6,7-tetrahydro-2H-indazol-2-yl)butanoic acid

Converted from Compound 19b.

LC-MS (ESI): R_{T}=4.337 min, mass calcd. for C₂₇H₂₆ClF₂N₅O₄S 589.1, m/z found 590.1 [M+H]⁺. Chiral HPLC (Column: Chiralpak IF 5 μm 4.6*250 mm; Mobile Phase: Hex:EtOH:TFA=90:10:0.2 at 1.0 mL/min; Temp: 30° C.; Wavelength: 230 nm, R_{T}=17.215 min). ¹H NMR (400 MHz, DMSO-d₆) δ 9.64 (s, 0.7H), 9.02 (s, 0.3H), 8.00-7.92 (m, 2H), 7.52-7.43 (m, 2H), 7.27-7.21 (m, 1H), 6.07 (s, 0.3H), 5.97 (s, 0.7H), 4.66-4.57 (m, 1H), 4.20-4.11 (m, 0.4H), 3.99-3.87 (m, 2.6H), 2.97-2.54 (m, 6H), 2.18-1.78 (m, 2H), 1.41-1.38 (m, 3H), 1.06-0.99 (m, 3H).

Compound 51c:

### 3-(5-(6-(2-chloro-3,4-difluorophenyl)-5-(ethoxycarbonyl)-2-(thiazol-2-yl)-3,6-dihydropyrimidin-4-yl)-4,5,6,7-tetrahydro-2H-indazol-2-yl)butanoic acid

Converted from Compound 19c.

LC-MS (ESI): R_{T}=4.333 min, mass calcd. for C₂₇H₂₆ClF₂N₅O₄S 589.1, m/z found 590.1 [M+H]⁺. Chiral HPLC (Column: Chiralpak IA 5 μm 4.6*250 mm; Mobile Phase: Hex:EtOH:TFA=80:20:0.2 at 1.0 mL/min; Temp: 30° C.; Wavelength: 230 nm, R_{T}=7.095 min). ¹H NMR (400 MHz, DMSO-d₆) δ 9.06 (br s, 1H), 8.01-7.92 (m, 2H), 7.52-7.41 (m, 2H), 7.30-7.23 (m, 1H), 6.06 (s, 0.3H), 5.95 (s, 0.7H), 4.66-4.56 (m, 1H), 4.23-4.15 (m, 0.3H), 4.00-3.88 (m, 2.7H), 2.83-2.50 (m, 6H), 2.27-1.97 (m, 2H), 1.40-1.37 (m, 3H), 1.06-0.99 (m, 3H).

### Compound 52b

### 3-(5-(6-(2-chloro-3,4-difluorophenyl)-5-(ethoxycarbonyl)-2-(thiazol-2-yl)-3,6-dihydropyrimidin-4-yl)-4,5,6,7-tetrahydro-2H-indazol-2-yl)butanoic acid

Converted from Compound 20b.

LC-MS (ESI): R_{T}=3.704 min, mass calcd. for C₂₇H₂₆ClF₂N₅O₄S 589.1, m/z found 589.9 [M+H]⁺. Chiral HPLC (Column: Chiralpak IE 5 μm 4.6*250 mm; Mobile Phase: Hex:EtOH:TFA=80:20:0.2 at 1.0 mL/min; Temp: 30° C.; Wavelength: 230 nm, R_{T}=12.828 min). ¹H NMR (400 MHz, DMSO-d₆) δ 9.15 (br s, 1H), 8.00-7.92 (m, 2H), 7.52-7.43 (m, 2H), 7.27-7.19 (m, 1H), 6.07 (s, 0.3H), 5.97 (s, 0.7H), 4.67-4.55 (m, 1H), 4.20-4.12 (m, 0.4H), 3.99-3.89 (m, 2.6H), 2.97-2.55 (m, 6H), 2.18-1.78 (m, 2H), 1.40-1.38 (m, 3H), 1.06-0.98 (m, 3H).

### Compound 52d

### 3-(5-(6-(2-chloro-3,4-difluorophenyl)-5-(ethoxycarbonyl)-2-(thiazol-2-yl)-3,6-dihydropyrimidin-4-yl)-4,5,6,7-tetrahydro-2H-indazol-2-yl)butanoic acid

Converted from Compound 20d.

LC-MS (ESI): R_{T}=3.728 min, mass calcd. for C₂₇H₂₆ClF₂N₅O₄S 589.1, m/z found 589.9 [M+H]⁺. Chiral HPLC (Column: Chiralpak IE 5 μm 4.6*250 mm; Mobile Phase: Hex:EtOH:TFA=80:20:0.2 at 1.0 mL/min; Temp: 30° C.; Wavelength: 230 nm, R_{T}=11.606 min). ¹H NMR (400 MHz, DMSO-d₆) δ 8.00-7.93 (m, 2H), 7.52-7.40 (m, 2H), 7.29-7.26 (m, 1H), 6.06 (s, 0.3H), 5.96 (s, 0.7H), 4.65-4.54 (m, 1H), 4.23-4.14 (m, 0.4H), 3.98-3.95 (m, 2.6H), 2.89-2.54 (m, 6H), 2.27-1.96 (m, 2H), 1.38 (d, J=6.4 Hz, 3H), 1.06-1.02 (m, 3H).

### Compound 53b

### 3-(5-(6-(2-Chloro-3,4-difluorophenyl)-5-(ethoxycarbonyl)-2-(thiazol-2-yl)-3,6-dihydropyrimidin-4-yl)-4,5,6,7-tetrahydro-2H-indazol-2-yl)-2,2-dimethylpropanoic acid

Converted from 21b.

LC-MS (ESI): R_{T}=3.808 min, mass calcd. for C₂₈H₂₈ClF₂N₅O₄S 603.2, m/z found 603.9 [M+H]⁺. ¹H NMR (400 MHz, DMSO-d₆) δ 9.07 (br s, 1H), 8.00-7.92 (m, 2H), 7.51-7.45 (m, 1H), 7.37 (s, 0.4H), 7.32 (s, 0.6H), 7.26-7.21 (m, 1H), 6.07 (s, 0.4H), 5.96 (s, 0.6H), 4.15-4.13 (m, 2.4H), 3.98-3.87 (m, 2.6H), 2.99-2.81 (m, 1.4H), 2.73-2.67 (m, 2H), 2.62-2.55 (m, 0.6H), 2.17-2.10 (m, 0.3H), 2.03-1.92 (m, 1H), 1.83-1.78 (m, 0.7H), 1.06-0.98 (m, 9H).

### Compound 53c

### 3-(5-(6-(2-Chloro-3,4-difluorophenyl)-5-(ethoxycarbonyl)-2-(thiazol-2-yl)-3,6-dihydropyrimidin-4-yl)-4,5,6,7-tetrahydro-2H-indazol-2-yl)-2,2-dimethylpropanoic acid

Converted from Compound 21c.

LC-MS (ESI): R_{T}=3.830 min, mass calcd. for C₂₈H₂₈ClF₂N₅O₄S 603.2, m/z found 603.9 [M+H]⁺. Chiral HPLC (Column: Chiralpak IA 5 μm 4.6*250 mm; Mobile Phase: Hex:IPA:TFA=80:20:0.2 at 1.0 mL/min; Temp: 30° C.; Wavelength: 254 nm; R_{T}=6.848 min). ¹H NMR (400 MHz, DMSO-d₆) δ 9.02 (br s, 1H), 7.99 (q, J=3.2 Hz, 1.3H), 7.93 (q, J=2.8 Hz, 0.7H), 7.52-7.45 (m, 1H), 7.37 (s, 0.3H), 7.29-7.23 (m, 1.7H), 6.06 (s, 0.3H), 5.95 (s, 0.7H), 4.22-4.13 (m, 2.3H), 4.00-3.88 (m, 2.7H), 2.83-2.71 (m, 2.5H), 2.68-2.53 (m, 1H), 2.18-2.14 (m, 0.5H), 2.09-1.96 (m, 2H), 1.08-0.99 (m, 9H).

### Compound 54

### (trans)-3-(5-(6-(2-Chloro-3,4-difluorophenyl)-5-(ethoxycarbonyl)-2-(thiazol-2-yl)-3,6-dihydropyrimidin-4-yl)-4,5,6,7-tetrahydro-2H-indazol-2-yl)cyclobutanecarboxylic acid

Converted from Compound 22.

LC-MS (ESI): R_{T}=3.390 min, mass calcd. for C₂₈H₂₆ClF₂N₅O₄S 601.1, m/z found 602.1 [M+H]⁺. ¹H NMR (400 MHz, CD₃OD) δ 7.88 (s, 1H), 7.73 (s, 1H), 7.50-7.40 (m, 1H), 7.31-7.25 (m, 2H), 6.17-6.11 (m, 1H), 4.99 (s, 1H), 4.36 (s, 0.5H), 4.06-4.01 (m, 2.5H), 3.17-3.12 (m, 1H), 2.90-2.58 (m, 8H), 2.23-1.92 (m, 2H), 1.12-1.09 (m, 3H).

### Compounds 55a: (Mixture of Two Stereoisomers)

### 3-(5-(6-(2-chloro-3,4-difluorophenyl)-5-(ethoxycarbonyl)-2-(thiazol-2-yl)-3,6-dihydropyrimidin-4-yl)-7,7-dimethyl-4,5,6,7-tetrahydro-2H-indazol-2-yl)propanoic acid

Converted from Compound 23a.

LC-MS (ESI): R_{T}=2.778 min, mass calcd. for C₂₈H₂₈ClF₂N₅O₄S 603.1, m/z found 604.1 [M+H]⁺. ¹H NMR (400 MHz, DMSO-d₆) δ 9.58 (s, 0.7H), 9.15 (s, 0.3H), 8.00-7.93 (m, 2H), 7.53-7.46 (m, 1H), 7.35 (s, 0.3H), 7.32 (s, 0.7H), 7.27-7.19 (m, 1H), 6.06 (s, 0.3H), 5.96 (s, 0.7H), 4.52-4.47 (m, 0.3H), 4.26-4.19 (m, 2.7H), 4.00-3.92 (m, 2H), 2.97-2.89 (m, 1H), 2.77-2.60 (m, 3H), 2.15-2.08 (m, 0.4H), 1.97-1.90 (m, 0.6H), 1.64-1.61 (m, 0.3H), 1.50-1.43 (m, 0.7H), 1.33-1.21 (m, 6H), 1.06-0.98 (m, 3H).

### Compound 55c

### 3-(5-(6-(2-chloro-3,4-difluorophenyl)-5-(ethoxycarbonyl)-2-(thiazol-2-yl)-3,6-dihydropyrimidin-4-yl)-7,7-dimethyl-4,5,6,7-tetrahydro-2H-indazol-2-yl)propanoic acid

Converted from Compound 23c.

LC-MS (ESI): R_{T}=3.560 min, mass calcd. for C₂₈H₂₈ClF₂N₅O₄S 603.1, m/z found 604.1 [M+H]⁺. Chiral HPLC (Column: Chiralpak IA 5 μm 4.6*250 mm; Mobile Phase: Hex:EtOH:TFA=80:20:0.2 at 1.0 mL/min; Temp: 30° C.; Wavelength: 254 nm, R_{T}=6.037 min). ¹H NMR (400 MHz, DMSO-d₆) δ 12.29 (s, 1H), 9.59 (s, 0.6H), 9.17 (s, 0.4H), 8.00-7.93 (m, 2H), 7.55-7.47 (m, 1H), 7.35-7.21 (m, 2H), 6.07 (s, 0.4H), 5.97 (s, 0.6H), 4.51-4.43 (m, 0.4H), 4.25-4.19 (m, 2.6H), 4.03-3.90 (m, 2H), 2.83-2.65 (m, 3H), 2.46-2.41 (m, 1H), 2.32-2.26 (m, 0.2H), 2.09-2.02 (m, 0.8H), 1.71-1.64 (m, 1H), 1.34-1.24 (m, 6H), 1.06-0.99 (m, 3H).

### Compound 56

### 3-(5-(6-(2-Chloro-3,4-difluorophenyl)-5-(ethoxycarbonyl)-2-(thiazol-2-yl)-3,6-dihydropyrimidin-4-yl)-3-methyl-4,5,6,7-tetrahydro-2H-indazol-2-yl)propanoic acid

Converted from Compound 24.

LC-MS (ESI): R_{T}=3.196 min, mass calcd. for C₂₇H₂₆ClF₂N₅O₄S 589.1, m/z found 589.9 [M+H]⁺. ¹H NMR (400 MHz, CD₃OD) δ 7.89-7.86 (m, 1H), 7.72 (s, 1H), 7.31-7.24 (m, 2H), 6.17-6.10 (m, 1H), 4.39-4.23 (m, 2.5H), 4.06-4.00 (m, 2.5H), 2.92-2.59 (m, 6H), 2.27-2.16 (m, 4H), 2.10-2.02 (m, 1H), 1.11 (t, J=7.2 Hz, 3H).

### Compound 57c

### 3-(5-(6-(2-Chloro-3,4-difluorophenyl)-5-(ethoxycarbonyl)-2-(thiazol-2-yl)-3,6-dihydropyrimidin-4-yl)-4,5,6,7-tetrahydrobenzo[d]oxazol-2-yl)propanoic acid

Converted from Compound 26c

LC-MS (ESI): R_{T}=3.770 min, mass calcd. for C₂₆H₂₃ClF₂N₄O₅S 576.1, m/z found 577.0 [M+H]⁺. Chiral HPLC (Column: Chiralpak IA 5 μm 4.6*250 mm; Mobile Phase: Hex:EtOH:TFA=80:20:0.2 at 1.0 mL/min; Temp: 30° C.; Wavelength: 254 nm, R_{T}=8.107 min). ¹H NMR (400 MHz, CD₃OD) δ 7.89 (d, J=2.8 Hz, 1H), 7.74 (d, J=2.8 Hz, 1H), 7.31-7.22 (m, 2H), 6.18 (s, 0.3H), 6.12 (s, 0.7H), 4.50-4.38 (m, 0.3H), 4.23-4.12 (m, 0.7H), 4.03 (q, J=6.8 Hz, 2H), 3.10-3.01 (m, 2.6H), 2.91-2.84 (m, 0.6H), 2.82-2.74 (m, 2.8H), 2.73-2.62 (m, 2H), 2.28-2.07 (m, 1.3H), 2.03-1.91 (m, 0.7H), 1.11 (t, J=7.2 Hz, 3H).

### Compound 57f

### 3-(5-(6-(2-Chloro-3,4-difluorophenyl)-5-(ethoxycarbonyl)-2-(thiazol-2-yl)-3,6-dihydropyrimidin-4-yl)-4,5,6,7-tetrahydrobenzo[d]oxazol-2-yl)propanoic acid

Converted from 26f.

LC-MS (ESI): R_{T}=3.716 min, mass calcd. for C₂₆H₂₃ClF₂N₄O₅S 576.1, m/z found 577.0 [M+H]⁺. Chiral HPLC (Column: Chiralpak IA 5 μm 4.6*250 mm; Mobile Phase: Hex:EtOH:TFA=80:20:0.2 at 1.0 mL/min; Temp: 30° C.; Wavelength: 230 nm, R_{T}=8.530 min). ¹H NMR (400 MHz, CD₃OD) δ 7.89 (d, J=3.2 Hz, 1H), 7.74 (s, 1H), 7.31-7.21 (m, 2H), 6.17 (s, 0.3H), 6.12 (s, 0.7H), 4.49-4.38 (m, 0.3H), 4.22-4.11 (m, 0.7H), 4.03 (q, J=6.8 Hz, 2H), 3.03 (t, J=7.2 Hz, 2H), 2.94-2.88 (m, 0.7H), 2.84-2.72 (m, 4.7H), 2.50-2.45 (m, 0.6H), 2.34-2.22 (m, 1.3H), 2.19-2.10 (m, 0.7H), 1.11 (t, J=7.2 Hz, 3H).

### Compound 58b

### 3-(6-(6-(2-Chloro-3,4-difluorophenyl)-5-(ethoxycarbonyl)-2-(thiazol-2-yl)-3,6-dihydropyrimidin-4-yl)-4,5,6,7-tetrahydrobenzo[d]oxazol-2-yl)propanoic acid

Converted from Compound 27b.

LC-MS (ESI): R_{T}=3.744 min, mass calcd. for C₂₆H₂₃ClF₂N₄O₅S 577.0, m/z found 576.9 [M+H]⁺. Chiral HPLC (Column: Chiralpak IC 5 μm 4.6*250 mm; Mobile Phase: Hex:EtOH:TFA=80:20:0.2 at 0.5 mL/min; Temp: 30° C.; Wavelength: 230 nm, R_{T}=8.023 min). ¹H NMR (400 MHz, CD₃OD) δ 7.89 (s, 1H), 7.73 (s, 1H), 7.26-7.25 (m, 2H), 6.12 (s, 1H), 4.52-4.42 (m, 0.3H), 4.27-4.17 (m, 0.7H), 4.05-4.03 (m, 2H), 3.22-3.14 (m, 0.5H), 3.04-3.03 (m, 2.5H), 2.83-2.76 (m, 3H), 2.64-2.52 (m, 2H), 2.16-2.04 (m, 1.3H), 1.96-1.88 (m, 0.7H), 1.12-1.10 (m, 3H).

### Compound 58d

### 3-(6-(6-(2-Chloro-3,4-difluorophenyl)-5-(ethoxycarbonyl)-2-(thiazol-2-yl)-3,6-dihydropyrimidin-4-yl)-4,5,6,7-tetrahydrobenzo[d]oxazol-2-yl)propanoic acid

Converted from Compound 27 d.

LC-MS (ESI): R_{T}=3.761 min, mass calcd. for C₂₆H₂₃ClF₂N₄O₅S 577.0, m/z found 576.9 [M+H]⁺. Chiral HPLC (Column: Chiralpak IB 5 μm 4.6*250 mm; Mobile Phase: Hex:EtOH:TFA=90:10:0.2 at 1.0 mL/min; Temp: 30° C.; Wavelength: 230 nm, R_{T}=10.312 min). ¹H NMR (400 MHz, CD₃OD) δ 7.89 (s, 1H), 7.74 (s, 1H), 7.28-7.26 (m, 2H), 6.13 (s, 1H), 4.54-4.44 (m, 0.3H), 4.28-4.17 (m, 0.7H), 4.05-4.03 (m, 2H), 3.04-3.02 (m, 3H), 2.96-2.89 (m, 0.5H), 2.70-2.76 (m, 2H), 2.67-2.58 (m, 2.5H), 2.27-2.18 (m, 1.2H), 2.12-2.08 (m, 0.8H), 1.11-1.10 (m, 3H).

### Compound 59c

### 3-(5-(6-(2-Chloro-3,4-difluorophenyl)-5-(ethoxycarbonyl)-2-(thiazol-2-yl)-3,6-dihydropyrimidin-4-yl)-4,5,6,7-tetrahydrobenzo[d]thiazol-2-yl)propanoic acid

Converted from Compound 28c.

LC-MS (ESI): R_{T}=3.728 min, mass calcd. for C₂₆H₂₃ClF₂N₄O₄S₂ 592.1, m/z found 593.1 [M+H]⁺. Chiral HPLC (Column: Chiralpak IA 5 μm 4.6*250 mm; Mobile Phase: Hex:EtOH:TFA=80:20:0.2 at 1.0 mL/min; Temp: 30° C.; Wavelength: 230 nm, R_{T}=7.881 min). ¹H NMR (400 MHz, CDCl₃ added one drop of D₂O) S 7.82 (d, J=3.2 Hz, 0.6H), 7.76 (d, J=2.8 Hz, 0.4H), 7.49 (d, J=3.2 Hz, 0.6H), 7.44 (d, J=3.2 Hz, 0.4H), 7.17-7.03 (m, 2H), 6.24 (s, 0.4H), 6.11 (s, 0.6H), 4.61-4.51 (m, 0.4H), 4.32-4.22 (m, 0.6H), 4.08-3.97 (m, 2H), 3.36-3.18 (m, 3H), 3.12-3.04 (m, 0.6H), 3.01-2.77 (m, 4.4H), 2.23-2.11 (m, 1H), 2. 07-1.93 (m, 1H), 1.10 (t, J=7.2 Hz, 3H).

### Compound 59f

### 3-(5-(6-(2-Chloro-3,4-difluorophenyl)-5-(ethoxycarbonyl)-2-(thiazol-2-yl)-3,6-dihydropyrimidin-4-yl)-4,5,6,7-tetrahydrobenzo[d]thiazol-2-yl)propanoic acid

Converted from Compound 28f.

LC-MS (ESI): R_{T}=3.672 min, mass calcd. for C₂₆H₂₃ClF₂N₄O₄S₂ 592.1, m/z found 593.1 [M+H]⁺. Chiral HPLC (Column: Chiralpak IB 5 μm 4.6*250 mm; Mobile Phase: Hex:EtOH:TFA=90:10:0.2 at 1.0 mL/min; Temp: 30° C.; Wavelength: 230 nm, R_{T}=14.065 min). ¹H NMR (400 MHz, CD₃OD) 7.89 (d, J=2.8 Hz, 1H), 7.74 (s, 1H), 7.31-7.25 (m, 2H), 6.18 (s, 0.3H), 6.13 (s, 0.7H), 4.48 (br s, 0.3H), 4.24-4.19 (m, 0.7H), 4.00-4.00 (m, 2H), 3.23 (t, J=7.2 Hz, 2H), 3.12-2.79 (m, 4H), 2.73 (t, J=7.2 Hz, 2H), 2.26-2.14 (m, 2H), 1.11 (t, J=6.8 Hz, 3H).

### Compound 60b

### 3-(6-(6-(2-Chloro-3,4-difluorophenyl)-5-(ethoxycarbonyl)-2-(thiazol-2-yl)-3,6-dihydropyrimidin-4-yl)-4,5,6,7-tetrahydrobenzo[d]thiazol-2-yl)propanoic acid

Converted from Compound 29b.

LC-MS (ESI): R_{T}=3.649 min, mass calcd. for C₂₆H₂₃ClF₂N₄O₄S₂ 592.1, m/z found 593.0 [M+H]⁺. Chiral HPLC (Column: Chiralpak IE 5 μm 4.6*250 mm; Mobile Phase: Hex:EtOH:TFA=80:20:0.2 at 1.0 mL/min; Temp: 30° C.; Wavelength: 254 nm, R_{T}=17.982 min). ¹H NMR (400 MHz, DMSO-d₆) δ 9.64 (br s, 0.8H), 9.24 (br s, 0.2H), 8.01-7.93 (m, 2H), 7.50-7.43 (m, 1H), 7.28-7.24 (m, 1H), 6.07 (s, 0.2H), 5.97 (s, 0.8H), 4.31-4.24 (m, 0.2H), 4.09-4.03 (m, 0.8H), 4.00-3.92 (m, 2H), 3.15-3.01 (m, 3H), 2.94-2.79 (m, 2H), 2.70-2.66 (m, 3H), 2.27-2.21 (m, 0.2H), 2.11-1.96 (m, 1H), 1.88-1.85 (m, 0.8H), 1.07-0.99 (m, 3H).

### Compound 60c

### 3-(6-(6-(2-Chloro-3,4-difluorophenyl)-5-(ethoxycarbonyl)-2-(thiazol-2-yl)-3,6-dihydropyrimidin-4-yl)-4,5,6,7-tetrahydrobenzo[d]thiazol-2-yl)propanoic acid

Converted from E Compound 29c.

LC-MS (ESI): R_{T}=3.560 min, mass calcd. for C₂₆H₂₃ClF₂N₄O₄S₂ 592.1, m/z found 593.1 [M+H]⁺. Chiral HPLC (Column: Chiralpak OJ-H 5 μm 4.6*250 mm; Mobile Phase: Hex:EtOH:TFA=80:20:0.2 at 1.0 mL/min; Temp: 30° C.; Wavelength: 254 nm, R_{T}=10.160 min). ¹H NMR (400 MHz, DMSO-d₆) δ 8.01-7.93 (m, 2H), 7.53-7.46 (m, 1H), 7.29-7.26 (m, 1H), 6.05 (s, 0.2H), 5.96 (s, 0.8H), 4.30-4.28 (m, 0.2H), 4.08-4.03 (m, 0.8H), 3.99-3.92 (m, 2H), 3.13-3.09 (m, 2H), 3.04-2.97 (m, 1H), 2.88-2.85 (m, 1H), 2.79-2.64 (m, 4H), 2.34-2.33 (m, 0.2H), 2.22-2.02 (m, 1.8H), 1.07-0.99 (m, 3H).

### Compound 61a

### 3-(6-(6-(2-Chloro-3,4-difluorophenyl)-5-(ethoxycarbonyl)-2-(thiazol-2-yl)-3,6-dihydropyrimidin-4-yl)-4,5,6,7-tetrahydro-2H-indazol-2-yl)propanoic acid

Converted from Compound 30a

LC-MS (ESI): R_{T}=3.669 min, mass calcd. for C₂₆H₂₄ClF₂N₅O₄S 575.1, m/z found 575.9 [M+H]⁺. Chiral HPLC (Column: Chiralpak IC 5 μm 4.6*250 mm; Mobile Phase: Hex:EtOH:TFA=80:20:0.2 at 1.0 mL/min; Temp: 30° C.; Wavelength: 230 nm, R_{T}=6.908 min). ¹H NMR (400 MHz, DMSO-d₆) δ 9.07 (br s, 1H), 8.00-7.92 (m, 2H), 7.53-7.47 (m, 1H), 7.42 (s, 0.3H), 7.38 (s, 0.7H), 7.28-7.25 (m, 1H), 6.07 (s, 0.3H), 5.97 (s, 0.7H), 4.23-4.19 (m, 2.3H), 3.99-3.91 (m, 2.7H), 3.07-2.72 (m, 2H), 2.74-2.60 (m, 4H), 2.08-2.01 (m, 0.3H), 1.91-1.86 (m, 1H), 1.77-1.74 (m, 0.7H), 1.06-0.98 (m, 3H).

### Compound 61c

### 3-(6-(6-(2-Chloro-3,4-difluorophenyl)-5-(ethoxycarbonyl)-2-(thiazol-2-yl)-3,6-dihydropyrimidin-4-yl)-4,5,6,7-tetrahydro-2H-indazol-2-yl)propanoic acid

Converted from Compound 30c.

LC-MS (ESI): R_{T}=3.650 min, mass calcd. for C₂₆H₂₄ClF₂N₅O₄S 575.1, m/z found 575.9 [M+H]⁺. Chiral HPLC (Column: Chiralpak IC 5 μm 4.6*250 mm; Mobile Phase: Hex:EtOH:TFA=80:20:0.2 at 1.0 mL/min; Temp: 30° C.; Wavelength: 230 nm, R_{T}=8.230 min). ¹H NMR (400 MHz, DMSO-d₆) δ 8.01-7.92 (m, 2H), 7.52-7.43 (m, 1.3H), 7.38 (s, 0.7H), 7.29-7.26 (m, 1H), 6.06 (s, 0.3H), 5.97 (s, 0.7H), 4.23-4.18 (m, 2.3H), 4.00-3.93 (m, 2.7H), 2.89-2.54 (m, 6H), 2.20-2.11 (m, 0.3H), 2.01-1.92 (m, 1.7H), 1.06-0.99 (m, 3H).

### Compound 62

### 3-(5-(6-(2-Chloro-3,4-difluorophenyl)-5-(ethoxycarbonyl)-2-(thiazol-2-yl)-3,6-dihydropyrimidin-4-yl)-1-cyano-4,5,6,7-tetrahydro-2H-isoindol-2-yl)propanoic acid

Converted from Compound 31.

LC-MS (ESI): R_{T}=4.620 min, mass calcd. for C₂₈H₂₄ClF₂N₅O₄S 599.1, m/z found 599.9 [M+H]⁺. ¹H NMR (400 MHz, DMSO-d₆) δ 8.00-7.93 (m, 2H), 7.53-7.45 (m, 1H), 7.29-7.22 (m, 1H), 7.01-6.94 (m, 1H), 6.06 (d, J=6.8 Hz, 0.3H), 5.95 (d, J=5.6 Hz, 0.7H), 4.21-4.15 (m, 2.4H), 3.98-3.87 (m, 2.6H), 2.90-2.58 (m, 6H), 2.17-1.76 (m, 2H), 1.06-0.98 (m, 3H).

### Compound 63

### 3-(6-(6-(2-Chloro-3,4-difluorophenyl)-5-(ethoxycarbonyl)-2-(thiazol-2-yl)-3,6-dihydropyrimidin-4-yl)-1-cyano-4,5,6,7-tetrahydro-2H-isoindol-2-yl)propanoic acid

Converted from Compound 32.

LC-MS (ESI): R_{T}=3.521 min, mass calcd. for C₂₈H₂₄ClF₂N₅O₄S 599.1, m/z found 599.9 [M+H]⁺. ¹H NMR (400 MHz, CD₃OD) δ 7.78 (t, J=2.8 Hz, 1H), 7.63 (t, J=2.4 Hz, 1H), 7.20-7.12 (m, 2H), 6.73 (s, 1H), 6.05 (s, 1H), 4.21-4.18 (m, 2.5H), 4.15-3.98 (m, 0.5H), 3.93 (q, J=7.2 Hz, 2H), 3.04-2.76 (m, 2H), 2.70-2.67 (m, 2H), 2.64-2.43 (m, 2H), 2.06-1.75 (m, 2H), 1.01 (t, J=7.2 Hz, 3H).

### Compound 64

### 3-(6-(6-(2-Chloro-3,4-difluorophenyl)-5-(methoxycarbonyl)-2-(thiazol-2-yl)-3,6-dihydropyrimidin-4-yl)-5,6,7,8-tetrahydroquinazolin-2-yl)propanoic acid

Converted from Compound 33.

LC-MS (ESI): R_{T}=3.683 min, mass calcd. for C₂₆H₂₂ClF₂N₅O₄S 573.1, m/z found 573.9 [M+H]⁺. ¹H NMR (400 MHz, DMSO-d₆+ one drop of D₂O) S 8.47 (s, 0.5H), 8.43 (s, 0.5H), 8.00-7.92 (m, 2H), 7.52-7.45 (m, 1H), 7.29-7.25 (m, 1H), 6.06 (d, J=12.0 Hz, 0.2H), 5.97 (s, 0.8H), 4.22 (br s, 0.2H), 4.02 (br s, 0.8H), 3.52 (s, 3H), 3.19-3.13 (m, 0.4H), 3.04 (t, J=7.2 Hz, 2H), 2.99-2.75 (m, 3.6H), 2.71 (t, J=7.2 Hz, 2H), 2.24-1.89 (m, 2H).

### Compound 68b: (trans)-3-(-6-(-6-(2-chloro-3,4-difluorophenyl)-5-(methoxycarbonyl)-2-(thiazol-2-yl)-3,6-dihydropyrimidin-4-yl)-4,5,6,7-tetrahydrobenzo[d]thiazol-2-yl)cyclobutanecarboxylic acid, from compound 67n

LC-MS (ESI): R_{T}=3.545 min, mass calcd. for C₂₇H₂₃ClF₂N₄O₄S₂ 604.1, m/z found 605.1 [M+H]⁺. Chiral analysis (Column: Chiralpak IE 5 μm 4.6*250 mm; Mobile Phase: Hex:EtOH:TFA=70:30:0.2 at 1.0 mL/min; Temp: 30° C.; Wavelength: 230 nm, R_{T}=11.294 min). ¹H NMR (400 MHz, DMSO-d₆) δ 9.69 (br s, 1H), 7.99-7.43 (m, 2H), 7.49-7.43 (m, 1H), 7.26-7.23 (m, 1H), 6.05 (s, 0.2H), 5.97 (s, 0.8H), 4.32-4.28 (m, 0.2H), 4.09-4.03 (m, 0.8H), 3.84-3.76 (m, 1H), 3.52 (s, 3H), 3.22-3.07 (m, 2H), 2.95-2.82 (m, 2H), 2.73-2.67 (m, 1H), 2.63-2.56 (m, 2H), 2.46-2.41 (m, 2H), 2.27-2.23 (m, 0.2H), 2.08-2.05 (m, 1H), 1.89-1.86 (m, 0.8H).

### Compound 68c: (trans)-3-(6-(-6-(2-chloro-3,4-difluorophenyl)-5-(methoxycarbonyl)-2-(thiazol-2-yl)-3,6-dihydropyrimidin-4-yl)-4,5,6,7-tetrahydrobenzo[d]thiazol-2-yl)cyclobutanecarboxylic acid, from compound 67p

LC-MS (ESI): R_{T}=3.614 min, mass calcd. for C₂₇H₂₃ClF₂N₄O₄S₂ 604.1, m/z found 604.8 [M+H]⁺. Chiral analysis (Column: Chiralpak IA 5 μm 4.6*250 mm; Mobile Phase: Hex:IPA:TFA=70:30:0.2 at 1.0 mL/min; Temp: 30° C.; Wavelength: 230 nm, R_{T}=5.423 min). ¹H NMR (DMSO-d₆, 400 MHz) δ 8.00-7.90 (m, 2H), 7.51-7.44 (m, 1H), 7.29-7.23 (m, 1H), 6.04 (s, 0.2H), 5.96 (s, 0.8H), 4.32-4.28 (m, 0.2H), 4.09-4.03 (m, 0.8H), 3.84-3.77 (m, 1H), 3.53 (s, 3H), 3.16-3.10 (m, 1H), 3.06-2.99 (m, 1H), 2.92-2.88 (m, 1H), 2.81-2.67 (m, 2H), 2.63-2.57 (m, 2H), 2.45-2.39 (m, 2H), 2.24-2.13 (m, 1H), 2.06-2.01 (m, 1H).

### Compound 70b: 3-(-5-(6-(2-chloro-3,4-difluorophenyl)-5-(methoxycarbonyl)-2-(thiazol-2-yl)-3,6-dihydropyrimidin-4-yl)-4,5,6,7-tetrahydro-2H-indazol-2-yl)propanoic acid

purified by Prep. HPLC (Column: waters-2 Xbridge C18 (5 μm 19*150 mm), Mobile phase A: water (0.1% ammonium bicarbonate), Mobile phase B: acetonitrile, UV: 214 nm, Flowrate: 15 mL/min, Gradient: 5-70% (% B)) to give the title compound (44.5 mg, 50% yield, 99.5% purity) as yellow solids. LC-MS (ESI): RT=3.501 min, mass calcd. for C₂₅H₂₂ClF₂N₅O₄S 561.1, m/z found 561.9 [M+H]⁺. ¹HNMR (400 MHz, DMSO-d₆) δ 8.01-7.93 (m, 2H), 7.52-7.44 (m, 1.3H), 7.39 (s, 0.7H), 7.26-7.18 (m, 1H), 6.06 (s, 0.3H), 5.96 (s, 0.7H), 4.23-4.18 (m, 2H), 4.16-4.11 (m, 0.3H), 3.93-3.85 (m, 0.7H), 3.51 (s, 2H), 3.50 (s, 1H), 2.98-2.87 (m, 1H), 2.83-2.70 (m, 4H), 2.69-2.64 (m, 1H), 2.17-2.09 (m, 0.3H), 2.00-1.93 (m, 1H), 1.82-1.79 (m, 0.7H).

### Compound 70d: 3-(-5-(6-(2-chloro-3,4-difluorophenyl)-5-(methoxycarbonyl)-2-(thiazol-2-yl)-3,6-dihydropyrimidin-4-yl)-4,5,6,7-tetrahydro-2H-indazol-2-yl)propanoic acid

purified by Prep. HPLC (Column: waters-2 Xbridge C18 (5 μm 19*150 mm), Mobile phase A: water (0.1% ammonium bicarbonate), Mobile phase B: acetonitrile, UV: 214 nm, Flowrate: 15 mL/min, Gradient: 5-85% (% B)) to give the title compound (86 mg, 69% yield, 99.4% purity) as yellow solids. LC-MS (ESI): RT=3.129 min, mass calcd. for C₂₅H₂₂ClF₂N₅O₄S 561.1, m/z found 562.2 [M+H]⁺. ¹HNMR (400 MHz, DMSO-d₆) δ 8.01-7.93 (m, 2H), 7.52-7.44 (m, 1.3H), 7.37 (s, 0.7H), 7.29-7.21 (m, 1H), 6.04 (s, 0.3H), 5.94 (s, 0.7H), 4.23-4.18 (m, 2H), 4.16-4.12 (m, 0.3H), 3.94-3.87 (m, 0.7H), 3.51 (s, 2H), 3.50 (s, 1H), 2.84-2.61 (m, 5H), 2.60-2.51 (m, 1H), 2.23-1.91 (m, 2H).

### Compound 72b: (trans)-3-(6-(6-(2-Chloro-3,4-difluorophenyl)-5-(methoxycarbonyl)-2-(thiazol-2-yl)-3,6-dihydropyrimidin-4-yl)-4,5,6,7-tetrahydrobenzo[d]oxazol-2-yl)cyclobutanecarboxylic acid

LC-MS (ESI): R_{T}=4.342 min, mass calcd. for C₂₇H₂₃ClF₂N₄O₅S 588.1, m/z found 588.9 [M+H]⁺. Chiral analysis (Column: Chiralpak IC 5 μm 4.6*250 mm; Mobile Phase: Hex:EtOH:TFA=80:20:0.2 at 1.0 mL/min; Temp: 30° C.; Wavelength: 254 nm, R_{T}=8.207 min). ¹H NMR (400 MHz, CD₃OD) δ 7.89 (d, J=3.2 Hz, 1H), 7.77-7.71 (m, 1H), 7.30-7.22 (m, 2H), 6.17 (s, 0.3H), 6.10 (s, 0.7H), 4.53-4.44 (m, 0.3H), 4.28-4.18 (m, 0.7H), 3.63-3.52 (m, 4H), 3.25-3.12 (m, 1.8H), 3.07-3.01 (m, 0.5H), 2.85-2.80 (m, 0.7H), 2.63-2.55 (m, 6H), 2.22-2.04 (m, 1.3H), 1.96-1.88 (m, 0.7H).

### Compound 72c: (trans)-3-(6-(6-(2-Chloro-3,4-difluorophenyl)-5-(methoxycarbonyl)-2-(thiazol-2-yl)-3,6-dihydropyrimidin-4-yl)-4,5,6,7-tetrahydrobenzo[d]oxazol-2-yl)cyclobutanecarboxylic acid

LC-MS (ESI): R_{T}=4.364 min, mass calcd. for C₂₇H₂₃ClF₂N₄O₅S 588.1, m/z found 588.9 [M+H]⁺. Chiral analysis (Column: Chiralpak IA 5 μm 4.6*250 mm; Mobile Phase: Hex:IPA:TFA=80:20:0.2 at 1.0 mL/min; Temp: 30° C.; Wavelength: 254 nm, R_{T}=8.266 min). ¹H NMR (400 MHz, CD₃OD) δ 7.90 (d, J=2.8 Hz, 1H), 7.78-7.71 (m, 1H), 7.30-7.21 (m, 2H), 6.15 (s, 0.3H), 6.11 (s, 0.7H), 4.54-4.45 (m, 0.3H), 4.27-4.17 (m, 0.7H), 3.62-3.52 (m, 4H), 3.22-3.13 (m, 1H), 3.09-3.01 (m, 0.7H), 2.96-2.90 (m, 0.5H), 2.71-2.55 (m, 6.8H), 2.28-2.16 (m, 1.3H), 2.14-2.05 (m, 0.7H).

### Compound 74b: 4-(5-(6-(2-chloro-3,4-difluorophenyl)-5-(methoxycarbonyl)-2-(thiazol-2-yl)-3,6-dihydropyrimidin-4-yl)-4,5,6,7-tetrahydro-2H-indazol-2-yl)butanoic acid

LC-MS (ESI): R_{T}=3.703 min, mass calcd. for C₂₆H₂₄ClF₂N₅O₄S 575.1, m/z found 575.9 [M+H]⁺. Chiral analysis (Column: Chiralpak ID 5 μm 4.6*250 mm; Mobile Phase: Hex:IPA:TFA=70:30:0.2 at 1.0 mL/min; Temp: 30° C.; Wavelength: 230 nm, R_{T}=11.467 min). ¹H NMR (400 MHz, CD₃OD) δ 7.90 (s, 1H), 7.74 (s, 1H), 7.44 (s, 0.5H), 7.36 (s, 0.5H), 7.27 (d, J=6.4 Hz, 2H), 6.19 (s, 0.5H), 6.13 (s, 0.5H), 4.37 (br s, 0.5H), 4.16-4.04 (m, 2.5H), 3.60 (s, 3H), 3.10-2.65 (m, 4H), 2.31-2.27 (m, 2H), 2.15-2.11 (m, 3.5H), 1.95 (br s, 0.5H).

### Compound 74c: 4-(5-(6-(2-chloro-3,4-difluorophenyl)-5-(methoxycarbonyl)-2-(thiazol-2-yl)-3,6-dihydropyrimidin-4-yl)-4,5,6,7-tetrahydro-2H-indazol-2-yl)butanoic acid

LC-MS (ESI): R_{T}=3.733 min, mass calcd. for C₂₆H₂₄ClF₂N₅O₄S 575.1, m/z found 575.9 [M+H]⁺. Chiral analysis (Column: Chiralpak AD-H 5 μm 4.6*250 mm; Mobile Phase: Hex:EtOH:TFA=70:30:0.2 at 1.0 mL/min; Temp: 30° C.; Wavelength: 230 nm, R_{T}=7.733 min). ¹H NMR (400 MHz, CD₃OD) δ 7.90 (d, J=2.4 Hz, 1H), 7.74 (s, 1H), 7.43-7.23 (m, 3H), 6.18 (s, 0.5H), 6.12 (s, 0.5H), 4.38 (br s, 0.5H), 4.15-4.05 (m, 2.5H), 3.60 (s, 3H), 2.97-2.62 (m, 4H), 2.31-2.23 (m, 3.5H), 2.16-2.11 (m, 2.5H).

### Compound 76c: 3-(5-(6-(3,4-difluoro-2-methylphenyl)-5-(methoxycarbonyl)-2-(thiazol-2-yl)-3,6-dihydropyrimidin-4-yl)-4,5,6,7-tetrahydro-2H-indazol-2-yl)propanoic acid

LC-MS (ESI): R_{T}=3.124 min, mass calcd. for C₂₆H₂₅F₂N₅O₄S 541.2, m/z found 542.2 [M+H]⁺. Chiral analysis (Column: Chiralpak IA 5 μm 4.6*250 mm; Mobile Phase: Hex:EtOH:TFA=80:20:0.2 at 1 mL/min; Temp: 30° C.; Wavelength: 254 nm; R_{T}=9.160 min). ¹H NMR (400 MHz, CD₃OD) δ 7.87 (d, J=3.2 Hz, 1H), 7.71 (d, J=2.8 Hz, 1H), 7.37 (s, 1H), 7.23-7.01 (m, 2H), 5.89 (s, 1H), 4.35-4.32 (m, 2.5H), 4.15-3.90 (m, 0.5H), 3.58 (s, 3H), 2.95-2.68 (m, 6H), 2.51 (s, 3H), 2.28-2.03 (m, 2H).

### Compound 78c: lithium 6-(6-(2-chloro-3,4-difluorophenyl)-5-(methoxycarbonyl)-2-(thiazol-2-yl)-3,6-dihydropyrimidin-4-yl)-4,5,6,7-tetrahydrobenzo[d]oxazole-2-carboxylate

To the solution of compound 77c (30.0 mg) in tetrahydrofuran (0.7 mL), methanol (0.2 mL) and water (0.1 mL) was added lithium hydroxide monohydrate (4 mg, 0.095 mmol) under nitrogen atmosphere at room temperature. After stirred at room temperature for 1 hour, the mixture was concentrated at room temperature under reduced pressure to remove the volatile, then purified by C18 column (acetonitrile:water=5% to 40%) to give the title compound (25 mg, 98.6% purity, 93% yield) as yellow solids. LC-MS (ESI): R_{T}=3.453 min, mass calcd. for C₂₃H₁₆ClF₂N₄O₅S.Li 540.1, m/z found 534.9 ([(M-Li⁺)+2H]⁺). ¹H NMR (400 MHz, CD₃OD) δ 7.91-7.89 (m, 1H), 7.75-7.73 (m, 1H), 7.32-7.26 (m, 2H), 6.18 (s, 0.3H), 6.11 (s, 0.7H), 4.55-4.47 (m, 0.3H), 4.30-4.23 (m, 0.7H), 3.60 (s, 2.1H), 3.59 (s, 0.9H), 3.29-3.26 (m, 0.7H), 3.12-3.10 (m, 0.6H), 2.93-2.88 (m, 0.7H), 2.71 (s, 0.6H), 2.62 (s, 1.4H), 2.27-2.06 (m, 1.3H), 1.98-1.90 (m, 0.7H).

### Compound 80c: 3-(5-(6-(4-fluoro-2-methylphenyl)-5-(methoxycarbonyl)-2-(thiazol-2-yl)-3,6-dihydropyrimidin-4-yl)-4,5,6,7-tetrahydro-2H-indazol-2-yl)propanoic acid

LC-MS (ESI): R_{T}=3.529 min, mass calcd. for C₂₆H₂₆FN₅O₄S 523.2, m/z found 524.2 [M+H]⁺. Chiral analysis (Column: Chiralpak IA 5 μm 4.6*250 mm; Mobile Phase: Hex:EtOH:TFA=80:20:0.2 at 1.0 mL/min; Temp: 30° C.; Wavelength: 254 nm, R_{T}=9.05 min). ¹H NMR (400 MHz, CD₃OD) δ 7.87 (d, J=2.8 Hz, 1H), 7.71 (d, J=3.2 Hz, 1H), 7.36 (br s, 2H), 6.92-6.85 (m, 2H), 5.89 (s, 1H), 4.35-4.32 (m, 2.5H), 4.01 (br s, 0.5H), 3.57 (s, 3H), 2.92-2.70 (m, 6H), 2.57 (s, 3H), 2.25-2.12 (m, 2H).

### Compound 82b: 3-((5-(6-(2-chloro-3,4-difluorophenyl)-5-(methoxycarbonyl)-2-(thiazol-2-yl)-3,6-dihydropyrimidin-4-yl)-4,5,6,7-tetrahydro-2H-indazol-2-yl)-3-methylbutanoic acid

LC-MS (ESI): R_{T}=3.535 min, mass calcd. for C₂₇H₂₆ClF₂N₅O₄S 589.1, m/z found 590.2 [M+H]⁺. Chiral analysis (Column: Chiralpak IC 5 μm 4.6*250 mm; Mobile Phase: Hex:IPA:TFA=80:20:0.2 at 1 mL/min; Temp: 30° C.; Wavelength: 280 nm; R_{T}=15.988 min). ¹H NMR (400 MHz, CD₃OD) δ 7.87 (s, 1H), 7.72 (s, 1H), 7.55 (s, 0.5H), 7.48 (s, 0.5H), 7.29-7.19 (m, 2H), 6.17 (s, 0.5H), 6.10 (s, 0.5H), 4.35 (br s, 0.5H), 4.05 (br s, 0.5H), 3.58 (s, 3H), 2.94-2.66 (m, 6H), 2.21-2.04 (m, 1.5H), 1.96-1.84 (m, 0.5H), 1.70 (s, 6H).

### Compound 82c: 3-(5-(-6-(2-chloro-3,4-difluorophenyl)-5-(methoxycarbonyl)-2-(thiazol-2-yl)-3,6-dihydropyrimidin-4-yl)-4,5,6,7-tetrahydro-2H-indazol-2-yl)-3-methylbutanoic acid

LC-MS (ESI): R_{T}=3.589 min, mass calcd. for C₂₇H₂₆ClF₂N₅O₄S 589.1, m/z found 590.2 [M+H]⁺. Chiral analysis (Column: Chiralcel OX-H 5 μm 4.6*250 mm; Mobile Phase: Hex:EtOH:TFA=80:20:0.2 at 1 mL/min; Temp: 30° C.; Wavelength: 254 nm; R_{T}=10.773 min). ¹H NMR (400 MHz, CD₃OD) δ 7.88 (s, 1H), 7.72 (s, 1H), 7.55 (s, 0.5H), 7.45 (s, 0.5H), 7.31-7.20 (m, 2H), 6.15 (s, 0.5H), 6.09 (s, 0.5H), 4.34 (br s, 0.5H), 4.11-3.98 (m, 0.5H), 3.58 (s, 3H), 2.98-2.57 (m, 6H), 2.28-2.06 (m, 2H), 1.69 (s, 6H).

### Compound 84d: 3-(5-(6-(2-chloro-3,4-difluorophenyl)-5-(methoxycarbonyl)-2-(thiazol-2-yl)-3,6-dihydropyrimidin-4-yl)-4,5,6,7-tetrahydro-2H-indazol-2-yl)-2,2-dimethylpropanoic acid

LC-MS (ESI): R_{T}=3.700 min, mass calcd. for C₂₇H₂₆ClF₂N₅O₄S 589.1, m/z found 590.2 [M+H]⁺. Chiral analysis (Column: Chiralpak IA 5 um 4.6*250 mm; Mobile Phase: Hex:IPA:TFA=80:20:0.2 at 1 mL/min; Temp: 30° C.; Wavelength: 254 nm, R_{T}=7.563 min). ¹H NMR (400 MHz, CD₃OD) δ 7.89 (s, 1H), 7.74-7.71 (m, 1H), 7.39 (s, 0.5H), 7.28-7.25 (m, 2.5H), 6.15 (s, 0.5H), 6.09 (s, 0.5H), 4.36-4.23 (m, 2.5H), 4.09-4.01 (m, 0.5H), 3.58 (s, 3H), 2.91-2.58 (m, 4H), 2.25-2.08 (m, 2H), 1.18 (s, 6H).

### Compound 86b: (trans)-4-(6-(6-(2-chloro-3,4-difluorophenyl)-5-(methoxycarbonyl)-2-(thiazol-2-yl)-3,6-dihydropyrimidin-4-yl)-4,5,6,7-tetrahydrobenzo[d]oxazol-2-yl)cyclohexanecarboxylic acid

LC-MS (ESI): R_{T}=3.668 min, mass calcd. for C₂₉H₂₇ClF₂N₄O₅S 616.1, m/z found 617.2 [M+H]⁺. ¹H NMR (400 MHz, CD₃OD) δ 7.92 (d, J=2.4 Hz, 1H), 7.76 (br s, 1H), 7.31-7.24 (m, 2H), 6.20-6.13 (m, 1H), 4.51-4.48 (m, 0.3H), 4.28-4.19 (m, 0.7H), 3.61 (s, 3H), 3.25-3.15 (m, 1H), 3.06-3.03 (m, 0.4H), 2.85-2.80 (m, 1.6H), 2.68-2.57 (m, 2H), 2.40-2.32 (m, 1H), 2.21-2.13 (m, 5.4H), 1.95-1.92 (m, 0.6H), 1.66-1.52 (m, 4H).

### Compound 86d: (trans)-4-(6-(6-(2-chloro-3,4-difluorophenyl)-5-(methoxycarbonyl)-2-(thiazol-2-yl)-3,6-dihydropyrimidin-4-yl)-4,5,6,7-tetrahydrobenzo[d]oxazol-2-yl)cyclohexanecarboxylic acid

LC-MS (ESI): R_{T}=3.909 min, mass calcd. for C₂₉H₂₇ClF₂N₄O₅S 616.1, m/z found 617.2 [M+H]⁺. ¹H NMR (400 MHz, CD₃OD) δ 7.92 (d, J=2.8 Hz, 1H), 7.77 (d, J=2.8 Hz, 1H), 7.29-7.28 (m, 2H), 6.18 (s, 0.3H), 6.13 (s, 0.7H), 4.53-4.46 (m, 0.3H), 4.28-4.19 (m, 0.7H), 3.61 (s, 3H), 3.10-3.03 (m, 0.6H), 2.97-2.92 (m, 0.4H), 2.88-2.59 (m, 4H), 2.41-2.32 (m, 1H), 2.26-2.08 (m, 6H), 1.69-1.50 (m, 4H).

### Compound 88d: 4-(5-(6-(2-Chloro-3,4-difluorophenyl)-5-(ethoxycarbonyl)-2-(thiazol-2-yl)-3,6-dihydropyrimidin-4-yl)-4,5,6,7-tetrahydro-2H-indazol-2-yl)-2,2-dimethylbutanoic acid

LC-MS (ESI): R_{T}=3.220 min, mass calcd. for C₂₉H₃₀ClF₂N₅O₄S 617.2, m/z found 617.9 [M+H]⁺. Chiral analysis (Column: Chiralpak IC 5 μm 4.6*250 mm; Mobile Phase: Hex:EtOH:TFA=90:10:0.2 at 1 mL/min; Temp: 30° C.; Wavelength: 230 nm, R_{T}=11.515 min). ¹H NMR (400 MHz, CD₃OD) δ 8.10-8.09 (m, 1H), 8.05-8.02 (m, 1H), 7.80 (s, 1H), 7.40-7.32 (m, 2H), 6.27 (s, 1H), 4.35-4.31 (m, 2H), 4.27-4.20 (m, 1H), 4.08 (q, J=7.2 Hz, 2H), 3.01-2.82 (m, 4H), 2.37-2.21 (m, 2H), 2.15-2.11 (m, 2H), 1.27 (s, 6H), 1.13 (t, J=7.2 Hz, 3H).

### Compound 90c: 4-(5-(6-(2-Chloro-3,4-difluorophenyl)-5-(methoxycarbonyl)-2-(thiazol-2-yl)-3,6-dihydropyrimidin-4-yl)-4,5,6,7-tetrahydro-2H-indazol-2-yl)-2,2-dimethylbutanoic acid

LC-MS (ESI): R_{T}=2.620 min, mass calcd. for C₂₈H₂₈ClF₂N₅O₄S 603.2, m/z found 603.9 [M+H]⁺. Chiral analysis (Column: Chiralpak IC 5 μm 4.6*250 mm; Mobile Phase: Hex:EtOH:TFA=80:20:0.2 at 1 mL/min; Temp: 30° C.; Wavelength: 230 nm, R_{T}=6.533 min). ¹H NMR (400 MHz, CD₃OD) 8.06-8.02 (m, 1H), 7.97-7.92 (m, 1H), 7.72 (s, 1H), 7.36-7.29 (m, 2H), 6.22 (s, 1H), 4.32-4.19 (m, 3H), 3.62 (s, 3H), 2.99-2.79 (m, 4H), 2.34-2.20 (m, 2H), 2.14-2.10 (m, 2H), 1.27 (s, 6H).

### Compound 92c: 3-(5-(6-(3,4-difluoro-2-methylphenyl)-5-(ethoxycarbonyl)-2-(thiazol-2-yl)-3,6-dihydropyrimidin-4-yl)-4,5,6,7-tetrahydro-2H-indazol-2-yl)-2,2-dimethylpropanoic acid

LC-MS (ESI): R_{T}=3.995 min, mass calcd. for C₂₉H₃₁F₂N₅O₄S 583.2, m/z found 584.0 [M+H]⁺. Chiral analysis (Column: Chiralpak IA 5 um 4.6*250 mm; Mobile Phase: Hex:IPA:TFA=80:20:0.2 at 1 mL/min; Temp: 30° C.; Wavelength: 254 nm, R_{T}=6.344 min). ¹H NMR (400 MHz, CD₃OD) δ 7.87 (d, J=2.8 Hz, 1H), 7.70 (s, 1H), 7.39-7.01 (m, 3H), 5.91-5.87 (m, 1H), 4.37 (br s, 0.6H), 4.24 (s, 2H), 4.06-4.01 (m, 2.4H), 2.86-2.74 (m, 3.6H), 2.68-2.49 (m, 3.4H), 2.26-2.07 (m, 2H), 1.18 (s, 6H), 1.10 (t, J=7.2 Hz, 3H).

### Compound 94c: 3-(5-(6-(2-Chloro-3,4-difluorophenyl)-2-(3,5-difluoropyridin-2-yl)-5-(ethoxycarbonyl)-3,6-dihydropyrimidin-4-yl)-4,5,6,7-tetrahydro-2H-indazol-2-yl)-2,2-dimethylpropanoic acid hydrochloride

LC-MS (ESI): R_{T}=2.595 min, mass calcd. for C₃₀H₂₉Cl₂F₄N₅O₄ 669.2, m/z found 633.9 [M-Cl]⁺. Chiral analysis (Column: Chiralpak IG 5 μm 4.6*250 mm; Mobile Phase: Hex:IPA:TFA=60:40:0.2 at 1.0 mL/min; Temp: 30° C.; Wavelength: 230 nm, R_{T}=4.928 min). ¹H NMR (400 MHz, DMSO-d₆) δ 11.84 (br s, 1H), 8.76 (s, 1H), 8.35-8.31 (m, 1H), 7.66-7.60 (m, 1H), 7.45-7.40 (m, 2H), 6.25 (s, 1H), 4.15-3.99 (m, 5H), 2.88-2.56 (m, 4H), 2.26-2.22 (m, 1H), 2.02-2.00 (m, 1H), 1.07-1.02 (m, 9H).

### Compound 96d: (cis)-3-(5-(6-(2-Chloro-3,4-difluorophenyl)-5-(methoxycarbonyl)-2-(thiazol-2-yl)-3,6-dihydropyrimidin-4-yl)-4,5,6,7-tetrahydro-2H-indazol-2-yl)cyclobutanecarboxylic acid

LC-MS (ESI): R_{T}=3.543 min, mass calcd. for C₂₇H₂₄ClF₂N₅O₄S 587.1, m/z found 588.2 [M+H]⁺. Chiral analysis (Column: Chiralpak IC 5 um 4.6*250 mm; Mobile Phase: Hex:EtOH:TFA=80:20:0.2 at 1 mL/min; Temp: 30° C.; Wavelength: 254 nm, R_{T}=9.237 min). ¹H NMR (400 MHz, CD₃OD) δ 7.89 (s, 1H), 7.73 (s, 1H), 7.56-7.41 (m, 1H), 7.31-7.20 (m, 2H), 6.16-6.10 (m, 1H), 4.75-4.63 (m, 1H), 4.34 (br s, 0.5H), 4.06 (br s, 0.5H), 3.59 (s, 3H), 2.99-2.60 (m, 9H), 2.27-2.08 (m, 2H).

### Part VII: Conversion

### Compound 65

### Methyl 4-(2-chloro-3,4-difluorophenyl)-6-(4,5,6,7-tetrahydro-1H-indazol-5-yl)-2-(thiazol-2-yl)-1,4-dihydropyrimidine-5-carboxylate

To a mixture of Compound 16 (700 mg, 1.22 mmol) in acetone (5 mL) was added concentrated hydrochloric acid (3 mL) at 0° C. After stirred at room temperature for 1 hour, the mixture was concentrated under reduced pressure to give a remainder. It was dissolved in ethyl acetate (30 mL) and washed with saturated sodium bicarbonate aqueous solution (30 mL). The aqueous layer was extracted with ethyl acetate (60 mL) twice. The combined organic layers were washed with brine (20 mL), dried over Na₂SO₄₍ₛ₎ and filtered. The filtrate was concentrated to leave a residue which was purified by C18 column (acetonitrile:water=40% to 70%) to give the title compound (160 mg, 27% yield) as yellow solids. LC-MS (ESI): R_{T}=1.66 min, mass calcd. for C₂₂H₁₈ClF₂N₅O₂S 489.1, m/z found 490.3 [M+H]⁺.

A stereoisomeric mixture of Compound 65 (160 mg, 0.33 mmol) was separated by chiral prep. HPLC (Column: Chiralpak OD-H 5 μm 20*250 mm; Mobile Phase: Hex:EtOH:DEA=90:10:0.3 at 15 mL/min; Temp: 30° C.; Wavelength: 214 nm) to give the title compounds Compound 65a (36 mg, 22% yield, 100% stereopure) and Compound 65b (29 mg, 18% yield, 100% stereopure) as yellow solids.

Compound 65a: LC-MS (ESI): R_{T}=3.361 min, mass calcd. for C₂₂H₁₈ClF₂N₅O₂S 489.1, m/z found 489.9 [M+H]⁺. Chiral HPLC (Column: Chiralpak OD-H 5 μm 4.6*250 mm; Mobile Phase: Hex:EtOH:DEA=80:20:0.2 at 1 mL/min; Wavelength: 230 nm, R_{T}=7.334 min). ¹H NMR (400 MHz, DMSO-d₆) δ 12.29 (br s, 1H), 9.61-9.57 (m, 0.7H), 9.12-9.06 (m, 0.3H), 8.00-7.93 (m, 2H), 7.52-7.45 (m, 1H), 7.36-7.23 (m, 2H), 6.05 (d, J=6.4 Hz, 0.3H), 5.98-5.95 (m, 0.7H), 4.17 (br s, 0.3H), 3.95-3.89 (m, 0.7H), 3.52 (s, 3H), 2.99-2.59 (m, 4H), 2.25-1.99 (m, 1.7H), 1.83-1.81 (m, 0.3H).

By ultilizing the analogous procedure of the above-mentioned deprotection conditions, the following compounds were prepared.

TABLE 3

| Precursor | Compound |
|---|---|
| Compound 16 | 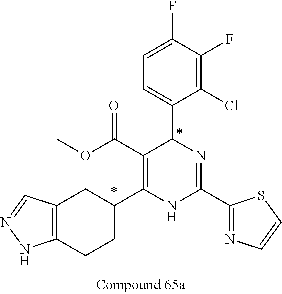 [SMILES: COC(=O)C1=C(C2CCc3[nH]ncc3C2)NC(c2nccs2)=NC1c1ccc(F)c(F)c1Cl] |
| Compound 25 | 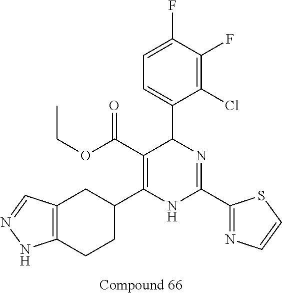 [SMILES: CCOC(=O)C1=C(C2CCc3[nH]ncc3C2)NC(c2nccs2)=NC1c1ccc(F)c(F)c1Cl] |

### Compound 66

### Ethyl 4-(2-chloro-3,4-difluorophenyl)-6-(4,5,6,7-tetrahydro-1H-indazol-5-yl)-2-(thiazol-2-yl)-1,4-dihydropyrimidine-5-carboxylate

Converted from Compound 25.

LC-MS (ESI): R_{T}=3.241 min, mass calcd. for C₂₃H₂₀ClF₂N₅O₂S 503.1, m/z found 503.9 [M+H]⁺. ¹H NMR (400 MHz, DMSO-d₆) δ 12.2 (br s, 1H), 9.61-9.56 (m, 0.7H), 9.06-9.01 (m, 0.3H), 8.00-7.98 (m, 1.4H), 7.96-7.92 (m, 0.6H), 7.54-7.44 (m, 1H), 7.38-7.20 (m, 2H), 6.07 (d, J=4.4 Hz, 0.3H) 5.97-5.95 (m, 0.7H), 4.22-4.12 (m, 0.3H), 3.99-3.88 (m, 2.7H), 2.98-2.58 (m, 4H), 2.19-1.92 (m, 1.6H), 1.84-1.78 (m, 0.4H), 1.07-0.98 (m, 3H).

### Example 2: Anti-Viral Assay in HepG2.2.15 Cells

### 1. Materials and Equipments

### 1.1. Cell Line

HepG2.2.15 (the HepG2.2.15 cell line can be produced by transfection of the HepG2 cell line as described in Sells, Chen, and Acs 1987 (Proc. Natl. Acad. Sci. USA 84: 1005-1009), and the HepG2 cell line is available from ATCC® under number HB-8065™)

### Reagents

DMEM/F12 (INVITROGEN-11330032)
FBS (GIBCO-10099-141)
Dimethyl sulfoxide (DMSO) (SIGMA-D2650)
Penicillin-streptomycin solution (HYCLONE-SV30010)
NEAA (INVITROGEN-1114050)
L-Glutamine (INVITROGEN-25030081)
Geneticin Selective Antibiotic (G418, 500 mg/ml) (INVITROGEN-10131027)
Trypsinase digestion solution (INVITROGEN-25300062)
CCK8 (BIOLOTE-35004)
QIAamp 96 DNA Blood Kit (12) (QIAGEN-51162)
FastStart Universal Probe Mast Mix (ROCHE-04914058001)

### 1.2. Consumables

96-well cell culture plate (COSTAR-3599)
Micro Amp Optical 96-well reaction plate (APPLIED BIOSYSTEMS-4306737)
Micro Amp Optical 384-well reaction plate (APPLIED BIOSYSTEMS)

### 1.3. Equipment

Plate reader (MOLECULAR DEVICES, SPECTRAMAX M2e)
Centrifuge (BECKMAN, ALLEGRA-X15R)
Real Time PCR system (APPLIED BIOSYSTEMS, QUANTSTUDIO 6)
Real Time PCR system (APPLIED BIOSYSTEMS, 7900HT)

### 2. Methods

### 2.1. HBV Inhibitory Activity and Cytotoxicity Determination

Seed the cell HepG2.2.15 cells into 96-well plate in 2% FBS culture medium at the density of 40,000 cells/well and 5,000 cells/well for HBV inhibitory activity and cytotoxicity determination, respectively. After seeding, incubate the cell plates at 37° C., 5% CO2 overnight. The next day, medium containing compounds is added to the cell to treat the cells for 6 days with medium refreshed once in the middle of the treatment. 8 dose points with 3 folds dilution of each compound were adopted and the highest concentration of the compounds is 10 uM and 100 uM for HBV inhibitory activity and cytotoxicity determination, respectively.

After 6 days of compounds treatment, add 20 μl CCK-8 reagents to each well of cytotoxicity assay plates, incubate the plate at 37° C., 5% CO₂ for 2.5 h and measure the absorbance at 450 nm wavelength, at the same time read the absorbance at 630 nm wavelength as reference.

The HBV DAN change in the cell culture medium induced by the compounds is measured by q-PCR method. Briefly, the HBV DNA in the culture medium is extracted using QIAamp 96 DNA Blood Kit according to the manual and then quantified by q-PCR using the primers and probe in the table below.

TABLE 4

| Primers or | | SEQ ID |
|---|---|---|
| Probe | Sequence | NO: |
| HBV-Fw | GTGTCTGCGGCGTTTTATCA | 1 |
| HBV-Rev | GACAAACGGGCAACATACCTT | 2 |
| HBV-Probe | CCTCTKCATCCTGCTGCTATGCCTCATC | 3 |
| With Fam | | |
| reporter and | | |
| BHQ1 quencher | | |

### 2.2. DATA Analysis

EC₅₀ and CC₅₀ values are calculated by the GRAPHPAD PRISM software. If the CV % of DMSO controls is below 15% and the reference compounds shows expected activity or cytotoxicity, the data of this batch of experiment is considered qualified.

### 2.3. Results

See Table 5

TABLE 5

| Compound# | CC₅₀(uM) | EC₅₀(uM) |
|---|---|---|
| 34a | 27.5 | 0.3847 |
| 34c | 14.9 | <0.0046 |
| 34d | 20.6 | 0.1464 |
| 35b | 20.7 | 0.0958 |
| 35c | 14.4 | <0.0046 |
| 36b | 11.7 | 0.0965 |
| 36d | 9.7 | 0.0879 |
| 37b | 19.6 | 0.1746 |
| 37c | 21.1 | 0.0222 |
| 37d | 18.9 | 0.8804 |
| 38a | 28.2 | 0.0145 |
| 38b | 22.9 | 0.6673 |
| 38c | 19.6 | 0.0146 |
| 39b | 13.1 | 0.1646 |
| 39c | 11.9 | 0.0145 |
| 40b | 21.5 | 0.4796 |
| 40c | 12.5 | 0.0331 |
| 41b | 6.6 | 0.2919 |
| 41c | 14.4 | 0.0304 |
| 42b | 10.2 | 0.1311 |
| 42d | 12.7 | 0.0242 |
| 43b | 31.3 | 0.1251 |
| 43c | 29.9 | 0.0059 |
| 44b | 21 | 0.3832 |
| 44c | 36.7 | 0.0329 |
| 45b | 18.2 | 0.213 |
| 45c | 32.2 | 0.0473 |
| 46a | 17.3 | 0.2187 |
| 46d | 10.7 | 0.0136 |
| 47b | 37.7 | 0.4702 |
| 47c | >100 | 0.1466 |
| 48b | 80 | 0.6 |
| 48d | 34.3 | 0.03 |
| 49b | 21.2 | 0.6467 |
| 49c | 25.4 | 0.1097 |
| 50b | 12.6 | 0.1164 |
| 50c | 11.9 | <0.0046 |
| 51b | 7.7 | 0.0117 |
| 51c | 8.6 | 0.0097 |
| 52b | 5.8 | 0.0027 |
| 52d | 10.5 | 0.0124 |
| 53a | 7.2 | 0.1319 |
| 53b | 11.9 | 0.1037 |
| 53c | 10.9 | <0.0046 |
| 54 | 16 | 0.0198 |
| 55a | 8.5 | 0.081 |
| 55c | 7.1 | 0.0095 |
| 57c | 7.7 | 0.0125 |
| 57f | 12.6 | 0.0908 |
| 58b | 9.8 | 0.0062 |
| 58d | 10.8 | 0.0092 |
| 59c | 4.1 | 0.1319 |
| 59f | 5.8 | 0.2095 |
| 60b | 3.4 | 0.0241 |
| 60c | 5.5 | 0.0135 |
| 61a | 4.8 | 0.0612 |
| 61c | 29 | 0.1249 |
| 62 | 5 | 0.0069 |
| 63 | 5.1 | 0.4363 |
| 64 | 26.4 | 0.07 |
| 65a | 11.8 | 0.0563 |
| 65b | 6.3 | 2.674 |
| 66 | 6.4 | 0.0647 |
| 68b | 8.3 | 0.009 |
| 68c | 3.6 | 0.00645 |
| 70b | 22.7 | 0.083 |
| 70d | 24.3 | 0.00803 |
| 72b | 7 | 0.0215 |
| 72c | 8.7 | 0.0902 |
| 74B | 19.6 | 0.0661 |
| 74c | 18.2 | 0.0091 |
| 76c | 16.1 | 0.013 |
| 78c | 52 | 0.0571 |
| 80c | 33.2 | 0.0724 |
| 82b | 15.9 | 0.0861 |
| 82c | 15.1 | 0.0065 |
| 84d | 16.4 | 0.0188 |
| 86b | 8.7 | 0.0640 |
| 86d | 4.3 | 0.0614 |
| 88d | 9.8 | 0.0208 |
| 90c | 13.3 | 0.0249 |
| 92c | 9.3 | 0.0079 |
| 94c | 16.5 | 0.0071 |
| 96d | 19.3 | 0.0247 |

1. A compound of formula (I) 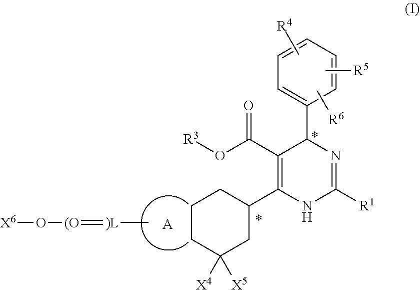
including the deuterated isomers, stereoisomers and the tautomeric forms thereof,
wherein A is a 5 or 6-membered aromatic ring, which comprises heteroatoms independently chosen from among S, O and N, wherein the number of said heteroatoms independently chosen from among S, O and N is one or two, wherein said 5 or 6-membered aromatic ring is optionally substituted with one or more from C1-C4 alkyl and cyano,
wherein L is C1-C6 alkyl,
wherein X⁶ is H or C1-C6 alkyl,
wherein R⁴, R⁵ and R⁶ each independently are chosen from among halogen, H and C1-C3 alkyl,
wherein R³ is C1-C4 alkyl,
wherein R¹ is selected from thiazolyl and pyridyl, each optionally substituted with one or more halogen; and
wherein X⁴ and X⁵ each independently are chosen from among H and C1-C4 alkyl,
or a pharmaceutically acceptable salt or a solvate thereof.

2. The compound of claim 1, wherein L is a straight-chain hydrocarbon, or a branched-chain hydrocarbon, or a cyclic-chain hydrocarbon, or X⁶—O—(O═C)L′, wherein L′ is C1-C5 alkyl, including C3-C5 cycloalkyl.

3. The compound of claim 1, wherein L is a straight-chain hydrocarbon, or a branched-chain hydrocarbon, or a cyclic-chain hydrocarbon, or X⁶—O—(O═C)L′, wherein L′ is C1-C5 alkyl, including C3-C6 cycloalkyl.

4. The compound of claim 1, wherein ring A is pyrazolyl, pyrrolyl, pyrimidyl, oxazolyl or thiazolyl.

5. The compound of claim 1, wherein R¹ is thiazolyl.

6. The compound of claim 1, which is selected from the compounds satisfying the following formulae: 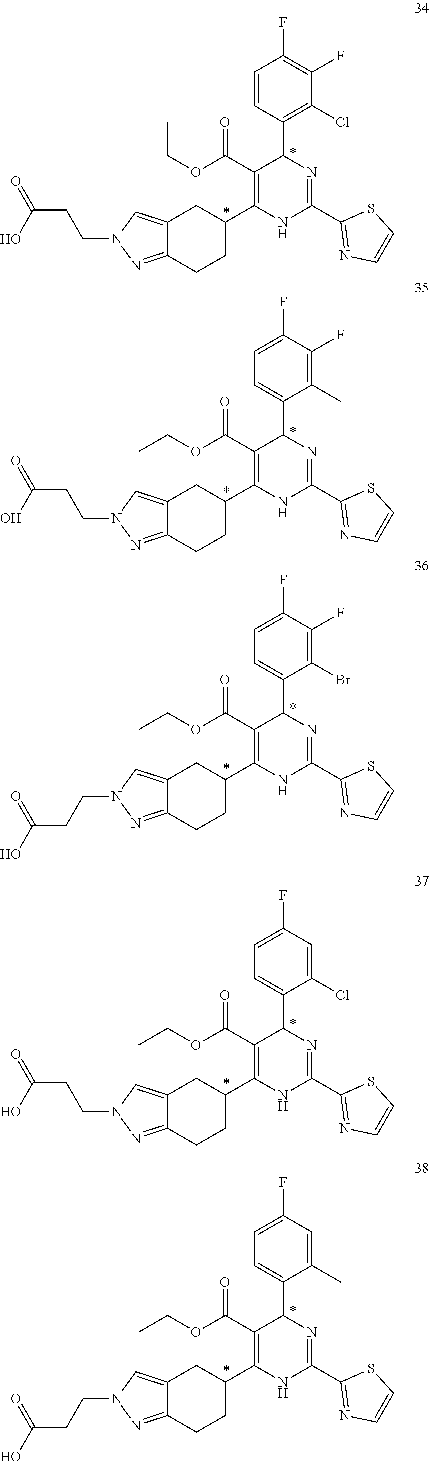 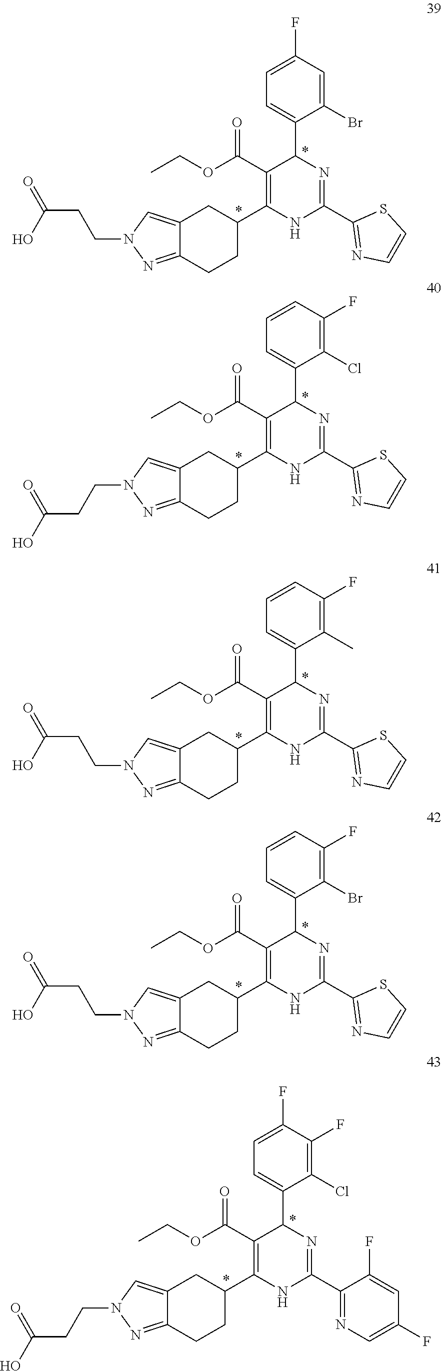 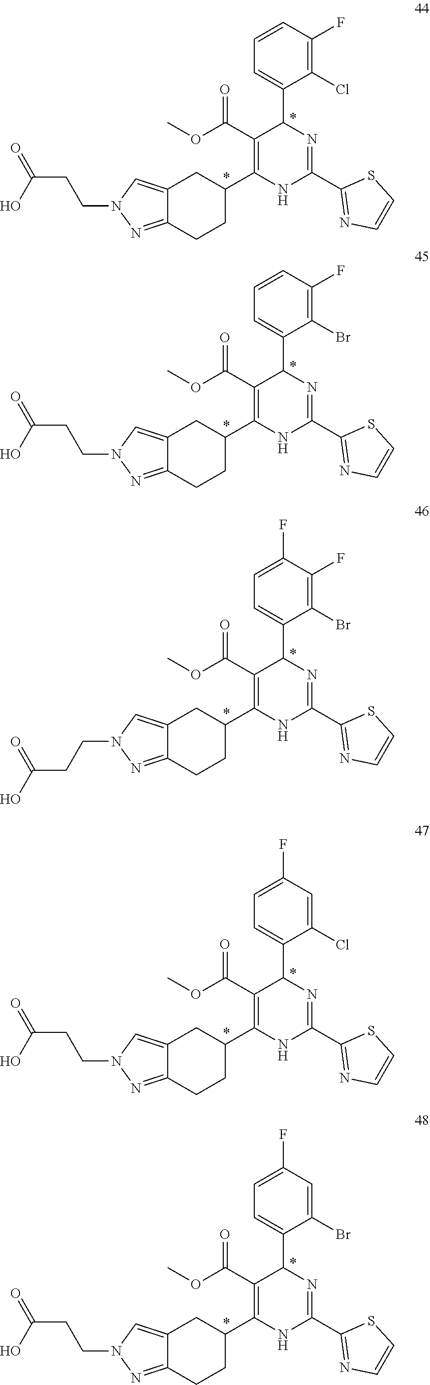 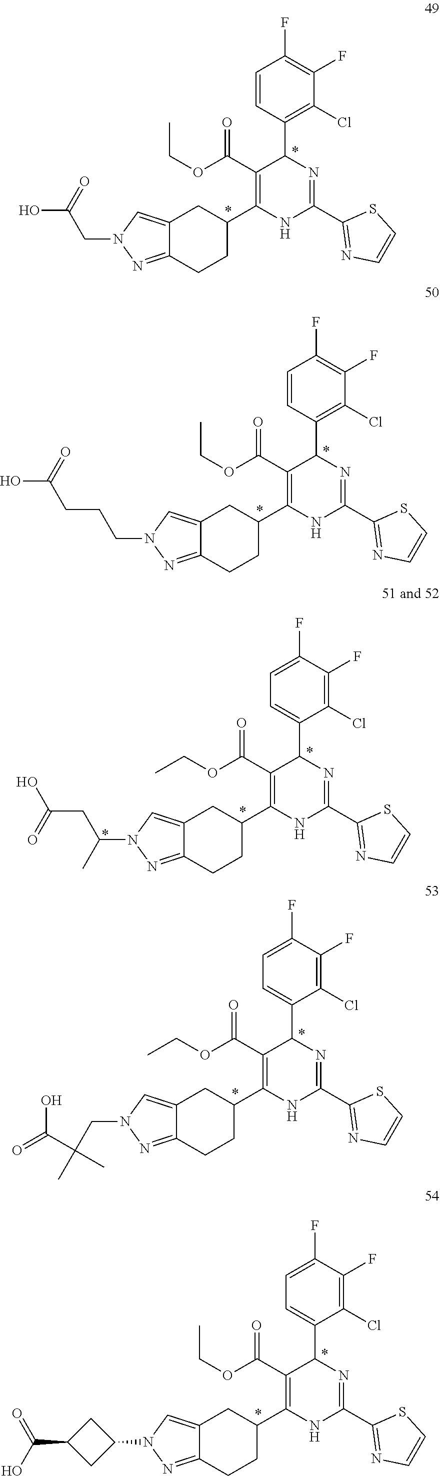 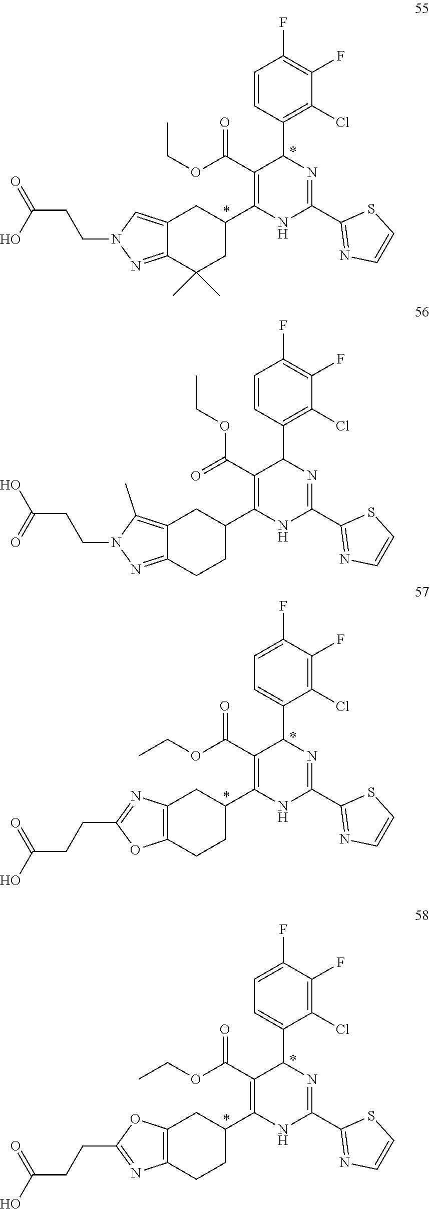 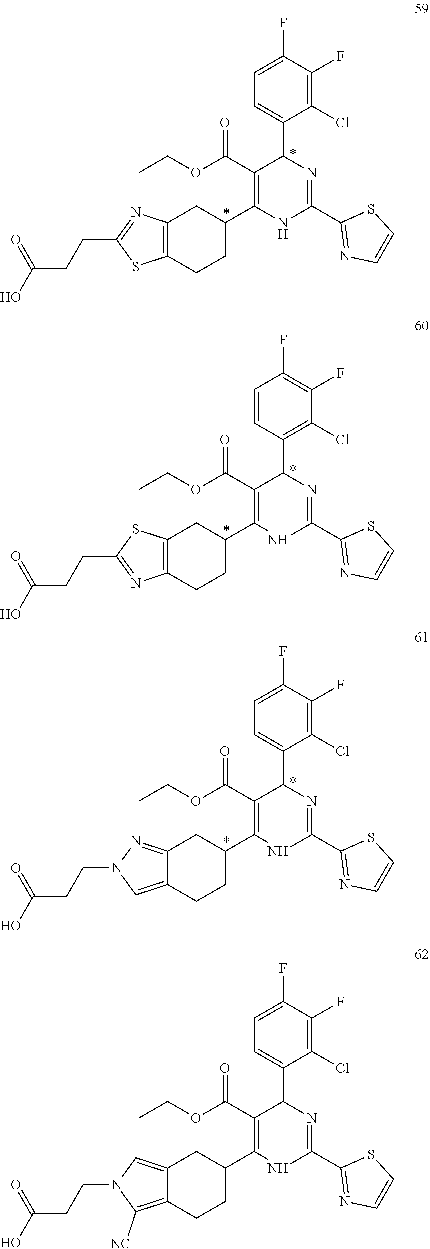 [SMILES: CCOC(=O)C1=C(C2CCc3sc(CCC(=O)O)nc3C2)NC(c2nccs2)=NC1c1ccc(F)c(F)c1Cl ; CCOC(=O)C1=C(C2CCc3nc(CCC(=O)O)sc3C2)NC(c2nccs2)=NC1c1ccc(F)c(F)c1Cl ; CCOC(=O)C1=C(C2CCc3cn(CCC(=O)O)nc3C2)NC(c2nccs2)=NC1c1ccc(F)c(F)c1Cl ; CCOC(=O)C1=C(C2CCc3c(cn(CCC(=O)O)c3C#N)C2)NC(c2nccs2)=NC1c1ccc(F)c(F)c1Cl] 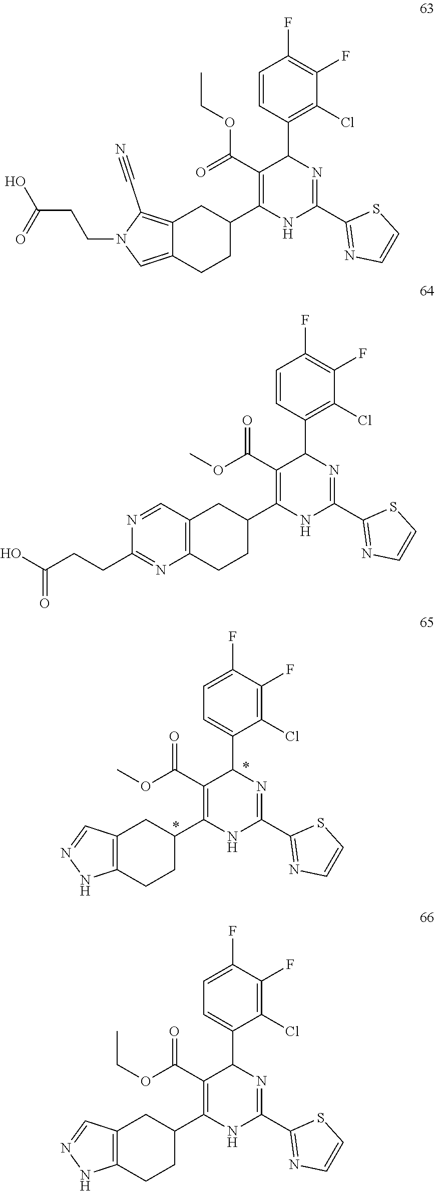 [SMILES: CCOC(=O)C1=C(C2CCc3cn(CCC(=O)O)c(C#N)c3C2)NC(c2nccs2)=NC1c1ccc(F)c(F)c1Cl ; COC(=O)C1=C(C2CCc3nc(CCC(=O)O)ncc3C2)NC(c2nccs2)=NC1c1ccc(F)c(F)c1Cl ; COC(=O)C1=C(C2CCc3[nH]ncc3C2)NC(c2nccs2)=NC1c1ccc(F)c(F)c1Cl ; CCOC(=O)C1=C(C2CCc3[nH]ncc3C2)NC(c2nccs2)=NC1c1ccc(F)c(F)c1Cl]
or a pharmaceutically acceptable salt or a solvate thereof.

7. The compound of claim 1, which is selected from the compounds satisfying the following formulae: 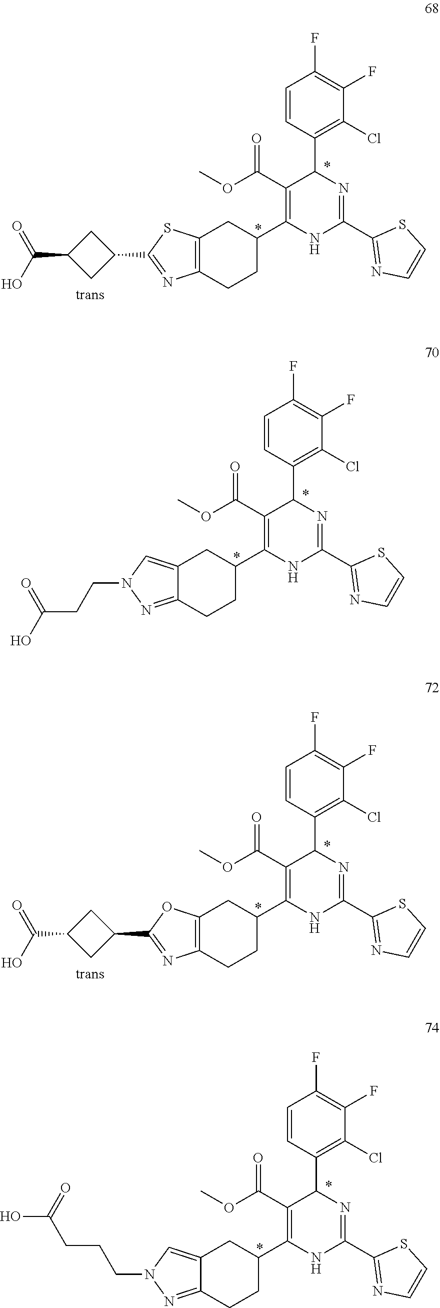 [SMILES: COC(=O)C1=C(C2CCc3nc([C@H]4C[C@H](C(=O)O)C4)sc3C2)NC(c2nccs2)=NC1c1ccc(F)c(F)c1Cl ; COC(=O)C1=C(C2CCc3nn(CCC(=O)O)cc3C2)NC(c2nccs2)=NC1c1ccc(F)c(F)c1Cl ; COC(=O)C1=C(C2CCc3nc([C@H]4C[C@H](C(=O)O)C4)oc3C2)NC(c2nccs2)=NC1c1ccc(F)c(F)c1Cl ; COC(=O)C1=C(C2CCc3nn(CCCC(=O)O)cc3C2)NC(c2nccs2)=NC1c1ccc(F)c(F)c1Cl] 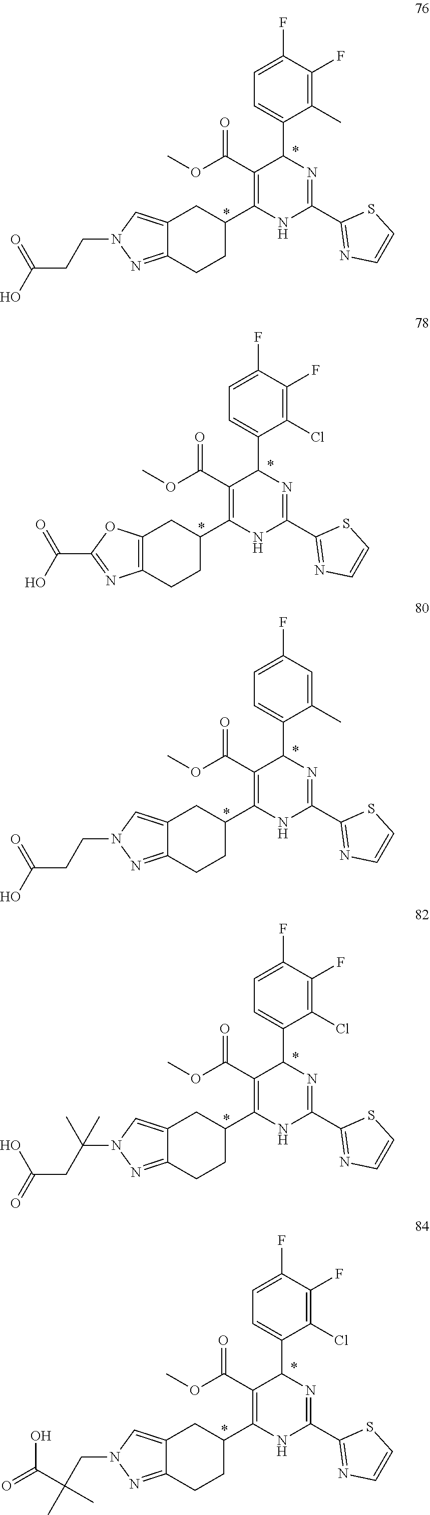 [SMILES: COC(=O)C1=C(C2CCc3nn(CCC(=O)O)cc3C2)NC(c2nccs2)=NC1c1ccc(F)c(F)c1C ; COC(=O)C1=C(C2CCc3nc(C(=O)O)oc3C2)NC(c2nccs2)=NC1c1ccc(F)c(F)c1Cl ; COC(=O)C1=C(C2CCc3nn(CCC(=O)O)cc3C2)NC(c2nccs2)=NC1c1ccc(F)cc1C ; COC(=O)C1=C(C2CCc3nn(C(C)(C)CC(=O)O)cc3C2)NC(c2nccs2)=NC1c1ccc(F)c(F)c1Cl ; COC(=O)C1=C(C2CCc3nn(CC(C)(C)C(=O)O)cc3C2)NC(c2nccs2)=NC1c1ccc(F)c(F)c1Cl] 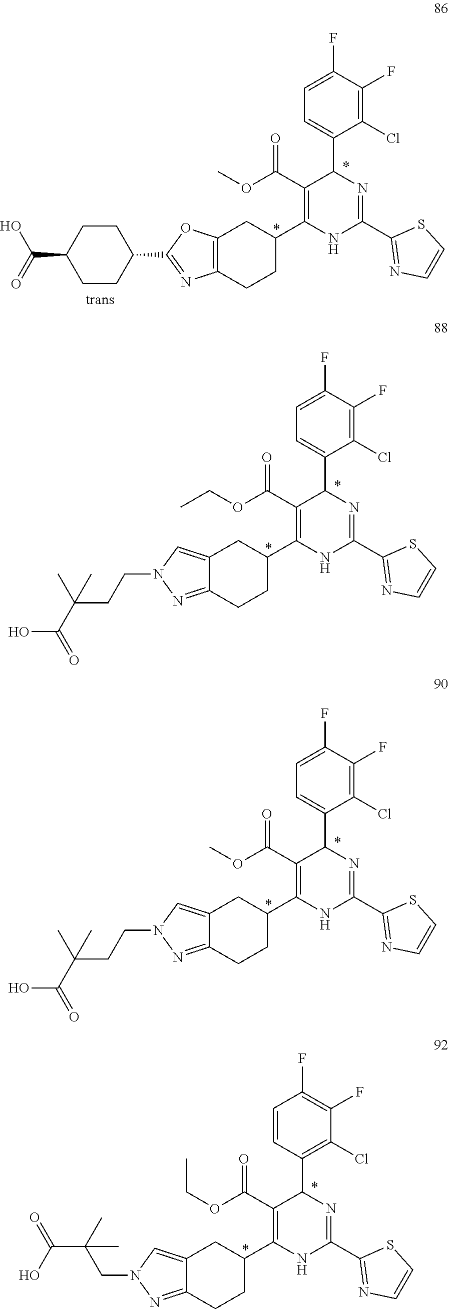 [SMILES: COC(=O)C1=C(C2CCc3nc([C@H]4CC[C@H](C(=O)O)CC4)oc3C2)NC(c2nccs2)=NC1c1ccc(F)c(F)c1Cl ; CCOC(=O)C1=C(C2CCc3nn(CCC(C)(C)C(=O)O)cc3C2)NC(c2nccs2)=NC1c1ccc(F)c(F)c1Cl ; COC(=O)C1=C(C2CCc3nn(CCC(C)(C)C(=O)O)cc3C2)NC(c2nccs2)=NC1c1ccc(F)c(F)c1Cl ; CCOC(=O)C1=C(C2CCc3nn(CC(C)(C)C(=O)O)cc3C2)NC(c2nccs2)=NC1c1ccc(F)c(F)c1Cl] 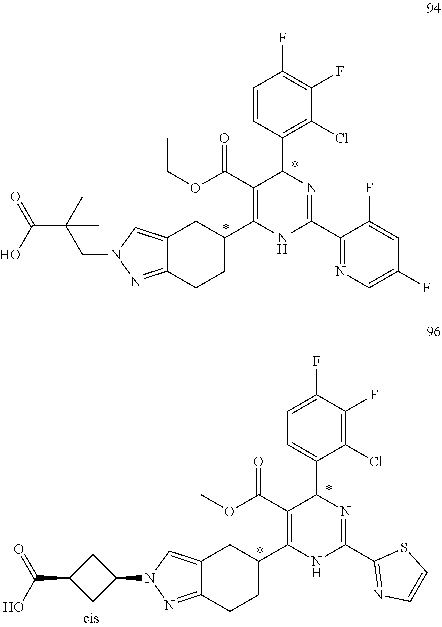 [SMILES: CCOC(=O)C1=C(C2CCc3nn(CC(C)(C)C(=O)O)cc3C2)NC(c2ncc(F)cc2F)=NC1c1ccc(F)c(F)c1Cl ; COC(=O)C1=C(C2CCc3nn([C@H]4C[C@@H](C(=O)O)C4)cc3C2)NC(c2nccs2)=NC1c1ccc(F)c(F)c1Cl]
or a pharmaceutically acceptable salt or a solvate thereof.

8. The compound of claim 1, which is of formula (I-a), (I-b), (I-c), (I-d), (I-e), (I-f), (I-g) or (I-h): 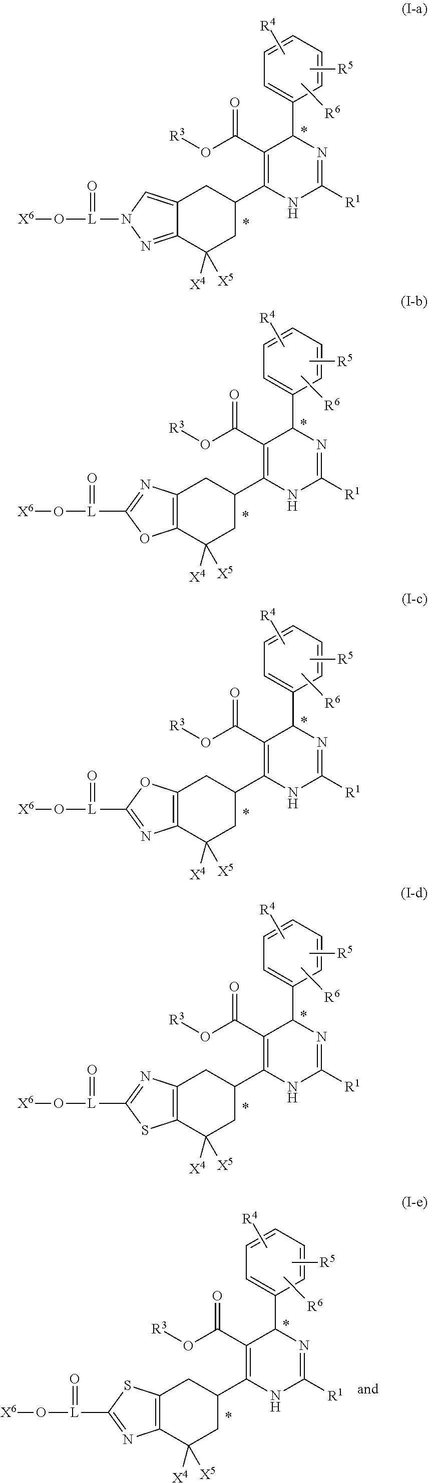 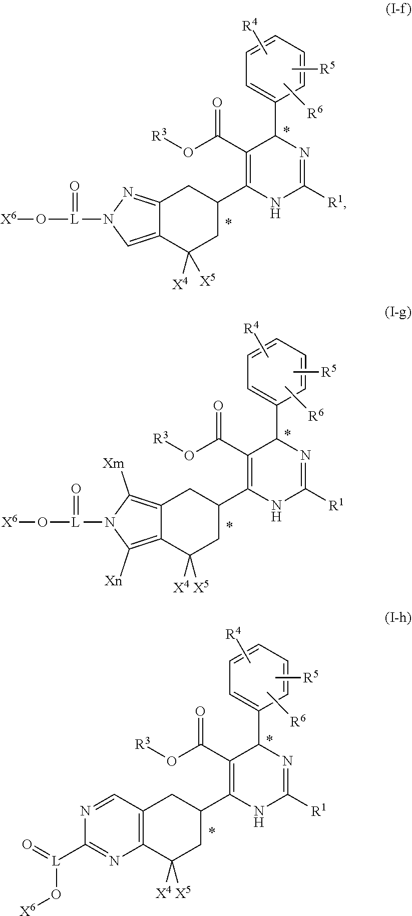
or a pharmaceutically acceptable salt or a solvate thereof.

9. The compound of claim 1,
wherein A is a 5-membered aromatic ring, which comprises N as heteroatoms, wherein the number of said N heteroatoms is two, wherein said 5-membered aromatic ring is optionally substituted with one or more substituents selected from C1-C4 alkyl and cyano,
wherein L is C3 alkyl,
wherein X⁶ is H,
wherein R⁴, R⁵ and R⁶ each independently are chosen from among CH₃, F, Cl and Br, more particularly from F and Cl,
and wherein R³ is C1-C3 alkyl,
and
wherein X⁴ and X⁵ each independently are chosen from among H and C1 alkyl.

10. The compound of claim 1, which is an HBV inhibitor.

11. The compound of claim 1, which is an HBV inhibitor with an EC50 equal to or lower than 1 μM on HepG2.2.15 cell line.

12. A pharmaceutical composition, which comprises the compound of claim 1 and which further comprises at least one pharmaceutically acceptable carrier.

13. (canceled)

14. (canceled)

15. (canceled)

16. (canceled)

17. A process for producing the compound of formula I of claim 1, wherein said process comprises reacting the compound of formula III with the compound of formula IV and the compound of formula V to produce the compound of formula I: 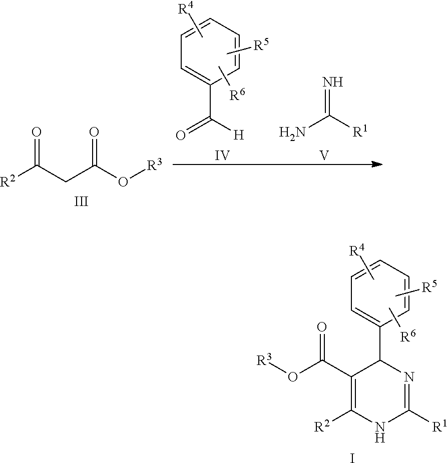
wherein R²— is the group: 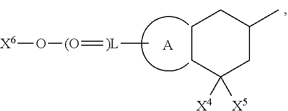
and wherein R¹, R³, R⁴, R⁵, R⁶, A, L, X⁴, X⁵, and X⁶ are as defined in any one of the claims 1 to 11.

18. A method of preventing or treating a hepatitis virus infection or a hepatitis virus induced disease in mammal in need thereof, comprising administering an effective amount of the compound of claim 1.

19. The method of claim 18, further comprising administering a second compound,
wherein said second compound is an HBV inhibitor which is chosen from among:
cytokines having HBV replication inhibition activity,
antibodies having immune checkpoint modulation activity,
substituted pyrimidines having HBV capsid assembly inhibition activity or having TLR agonist activity,
antiretroviral nucleoside analogues, and
the combinations thereof.

20. The method of claim 19, wherein said second compound is an HBV inhibitor which is chosen from among:
interferon, interferon-alpha, pegylated interferon, pegylated interferon-alpha,
anti-PD1 antibodies,
substituted pyrimidines having HBV capsid assembly inhibition activity or having TLR7 and/or TLR8 and/or TLR9 agonist activity,
lamivudine, adefovir dipivoxil, tenofovir disoproxil fumarate), and
the combinations thereof.

21. The method of claim 18, wherein the compound of claim 1 and the second compound are administered simultaneously, separately or sequentially.
